(19) United States
(12) Patent Application Publication
(10) Pub. No.: US 2022/0340875 A1
(43) Pub. Date: Oct. 27, 2022

(54) METHODS OF CULTURING AND/OR EXPANDING STEM CELLS AND/OR LINEAGE COMMITTED PROGENITOR CELLS USING AMIDO COMPOUNDS

(71) Applicant: IDEAYA BIOSCIENCES, INC., South San Francisco, CA (US)

(72) Inventors: Muzaffar ALAM, South San Francisco, CA (US); Fred ASWAD, South San Francisco, CA (US); Hilary Plake BECK, South San Francisco, CA (US); Michael Patrick DILLON, South San Francisco, CA (US); Marcos GONZALEZ-LOPEZ, South San Francisco, CA (US); Yujiro HATA, South San Francisco, CA (US); Alice Chen RICO, South San Francisco, CA (US); James Clifford SUTTON, Jr., South San Francisco, CA (US)

(21) Appl. No.: 17/260,936

(22) PCT Filed: Jul. 18, 2019
(86) PCT No.: PCT/US2019/042487

§ 371 Date: Jan. 15, 2021

Related U.S. Application Data
(60) Provisional application No. 62/700,568, filed on Jul. 19, 2018.
(60) Provisional application No. 62/739,491, filed on Oct. 1, 2018.

(51) Int. Cl. C12N 5/0789; A61K 35/28

(52) U.S. Cl. CPC C12N 5/0647; A61K 35/28; C12N 2501/999; C12N 2501/15; C12N 2501/71; C12N 2501/125; C12N 2501/42; C12N 2501/2306; C12N 2501/26; C12N 2501/145; C12N 2501/73

(57) ABSTRACT

Provided are methods for expanding stem cells and/or lineage committed progenitor cells, such as hematopoietic stems cells and/or lineage committed progenitor cells, at least in part, by using compounds that antagonize AhR. The compounds are represented by formulae:

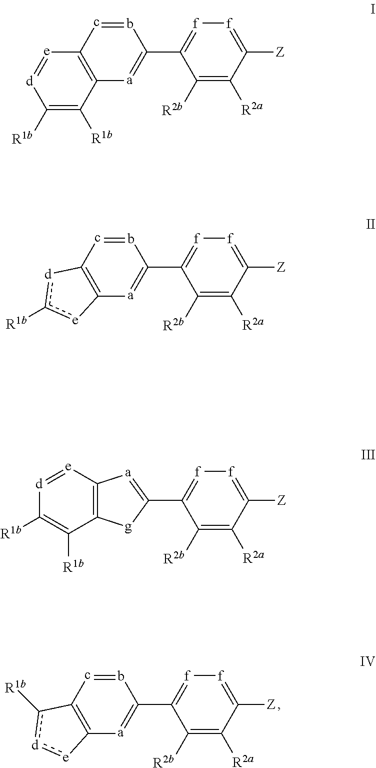

wherein the letters and symbols a, b, c, d, e, f, g, Z, R^{1b}, R^{2a} and R^{2b} have the meanings provided in the specification. Also provided are compositions comprising stem cells and/or lineage committed progenitor cells expanded by methods disclosed herein and methods for the treatment of diseases treatable by same.

### CROSS-REFERENCES TO RELATED APPLICATIONS

This application is the U.S. National Stage entry under § 371 of International Application No. PCT/US2019/042487, which claims the benefit of priority under 35 U.S.C § 119(e) to U.S. Provisional Application Ser. No. 62/700,568, filed Jul. 19, 2018, and Provisional Application Ser. No. 62/739,491, filed Oct. 1, 2018, the disclosures of which are incorporated herein by reference in their entireties.

### FIELD

The present disclosure relates to methods for expanding stem cells and/or lineage committed progenitor cells, such as hematopoietic stems cells and/or lineage committed progenitor cells, at least in part, by using compounds that antagonize AhR. Further provided are compositions comprising stem cells and/or lineage committed progenitor cells expanded by methods disclosed herein and methods for the treatment of diseases treatable by same.

### BACKGROUND OF THE INVENTION

Embryonic stem cells (ESCs) are pluripotent stem cells (PSC) capable of regenerating cells of all tissues from an individual. Prior to generation of lineage-committed and terminally differentiated cells, ESCs undergo a series of division and differentiation events, gradually losing their pluripotent and proliferative capabilities. Other stem cells derived from ESCs such as hematopoietic stem cells (HSCs) and mesenchymal stem cells (MSCs), have themselves multipotent and replicative capabilities, albeit in a more lineage committed fashion compared with ESCs. The self-renewal and multipotent properties of stem cells make these cells attractive candidates for the treatment of a wide range of diseases in the context of both cell and gene therapy strategies.

For example, HSCs can regenerate all cells of the blood compartment and are therefore of significant therapeutic value for the treatment of blood-based deficiencies resulting from congenital disorders and myeloablative treatments used to treat leukemias, lymphomas and other life-threatening conditions. HSCs can also be genetically modified to correct congenital defects that cause a range of hematological deficiencies such as anemia, sickle cell disease and hemophilia. Treatment with HSCs involves replacement of a patient's HSCs via autologous or allogeneic bone marrow transplantation (BMT) of HSCs or genetically modified HSCs. However, the success of a BMT is limited by the multipotency and number of HSCs available for transplantation. Therefore, there is a significant need for methods and reagents that can enhance the quality and quantity of HSCs.

The aryl hydrocarbon receptor (AhR) is a helix-loop-helix ligand-activated transcription factor that mediates biological responses to aromatic hydrocarbons. AhR is localized in the cytoplasm, where upon binding to a hydrocarbon-based ligand agonist, it migrates to the nucleus and forms a heterodimer with aryl hydrocarbon receptor nuclear translocator (ARNT). Formation of the AhR/ARNT complexes subsequently enables binding to and transcription of the xenobiotic response element (XRE) and associated genes. AhR can also activate non-XRE dependent protein-protein interaction pathways. Through its XRE-dependent and independent activity, AhR modulates numerous key pathways responsible for proliferation and differentiation of stem cells (SC).

SCs, including ESCs, MSCs, and HSCs amongst other cells analyzed, express high levels of AhR. Pharmacological inhibition of AhR significantly enhances maintenance of pluripotency during stem cell division and proliferation (see Boitano A. E. et al., Aryl hydrocarbon receptor antagonists promote the expansion of human hematopoietic stem cells. Science. 329, 1345-1348, 2010). For example, inhibition of AhR enhances the proliferative potential of stem cells such as MSC and HSCs (Boitano A. E. infra). Taken together, these results demonstrate that pharmacological inhibition of AhR can be used to greatly expand stem cells while maintaining their pluripotency. Additional factors such as inhibitors of TGF-beta, histone demethylase, histone deacetylation, p38 signaling, beta-catenin signaling and members of the ikaros family of transcription factors can also enhance HSC expansion (see de Almeida D. C., et al., Role of aryl hydrocarbon receptor in mesenchymal stromal cell activation: A minireview. World J. Stem Cells. 9, 152-158, 2017). It is postulated that a combination of these factors with an AhR inhibitor would yield an additive or synergistic effect on HSC expansion. Other factors such as Notch agonists work in concert with AhR inhibitors such as SR-1 to enhance HSC expansion (see PCT Publication No. WO/2013/086436).

While AhR antagonists that can enhance HSC expansion are described in the art, there remains a need for AhR antagonists that exhibit a more potent and less cytotoxic profile for use in expansion of HSCs and stem cells broadly. The present disclosure fulfills this and related needs.

### BRIEF SUMMARY OF THE INVENTION

Provided are methods for expanding of stem cells and/or lineage committed progenitor cells, at least in part, by using amido compounds that antagonize AhR. The compounds are represented by formulae I, II, III, and IV:

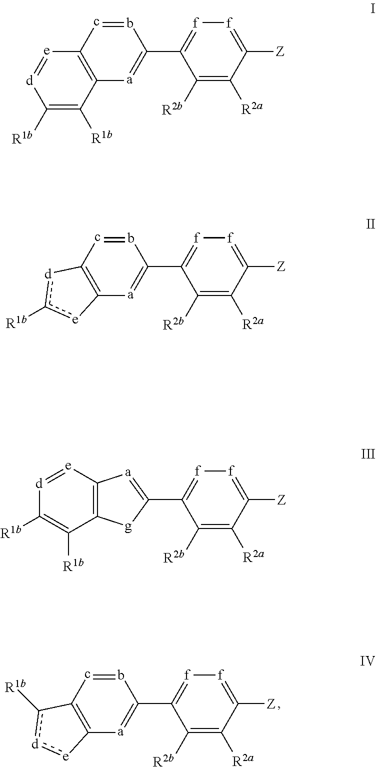

wherein the letters and symbols a, b, c, d, e, f, g, Z, R^{1b}, R^{2a} and R^{2b} have the meanings provided below. Also provided are compositions comprising stem cells and/or lineage committed progenitor cells expanded by methods disclosed herein and methods for the treatment of diseases treatable by same.

Also provided are methods for producing genetically modified stem cells comprising culturing naturally occurring stem cells in vitro or ex vivo with amido compounds that antagonize AhR. The compounds are represented by formulae I, II, III, and IV above.

In one aspect, provided herein is a method for producing an expanded population of stem cells and/or lineage committed progenitor cells in vitro or ex vivo comprising culturing a population of the stem cells and/or lineage committed progenitor cells in a medium comprising a compound of formula I, II, III or IV:

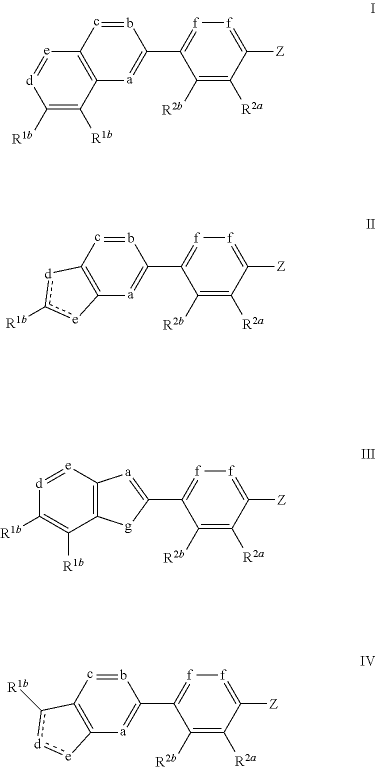

or a pharmaceutically acceptable salt, solvate or hydrate thereof, wherein
one of the dashed bonds is a single bond and the other of the dashed bonds is a double bond;
each of ring vertices a, b and c is independently selected from the group consisting of C(R^{1a}) and N;
each of ring vertices d and e is independently selected from the group consisting of C(R^{1b}), N, NH, N(C₁₋₄ alkyl) and N(C₁₋₄ haloalkyl) provided at least of d and e is other than C(R^{1b});
each ring vertex f is selected from the group consisting of C(R^{2c}), C(R^{2d}) and N;
ring vertex g is selected from the group consisting of O, S and N(R^{1a});
Z is selected from the group consisting of:

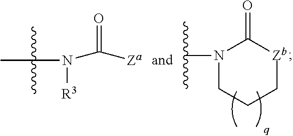

Z^{a} is selected from the group consisting of:
   (i) a 5- or 6-membered heteroaryl group having at least one nitrogen atom as a ring member, which is substituted with from 1 to 4 R⁴;
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
   (iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, phenyl and 5- or 6-membered heteroaryl;
and wherein the aliphatic and cyclic portions of R^{d}, R^{e} and R^{f} are optionally further substituted with from one to three halogen, hydroxy, methyl, amino, methylamino, dimethylamino and carboxylic acid groups; and wherein the compound of formula I, II, III, or IV antagonizes the activity of aryl hydrocarbon receptor;
and

the stem cells and/or lineage committed progenitor cells are cultured under conditions allowing expansion of the stem cells and/or progenitor cells.

In some embodiments, according to any of the methods described above, the method further comprises differentiating the expanded stem cells to lineage committed progenitor cells thereof under conditions that cause differentiation of the expanded stem cells to lineage committed progenitor cells thereof.

In some embodiments, according to any of the methods described above, the method is carried out ex vivo.

In some embodiments, according to any of the methods described above, the stem cells and lineage committed progenitor cells are human cells.

In some embodiments, according to any of the methods described above, the stem cells and/or lineage committed progenitor cells are hematopoietic stem cells and/or lineage committed hematopoietic progenitor cells.

In some embodiments, according to any of the methods described above, the stem cells and/or lineage committed progenitor cells are genetically modified hematopoietic stem cells and/or lineage committed genetically modified hematopoietic progenitor cells thereof.

In some embodiments, according to any of the methods described above, the genetically modified hematopoietic stem cells and/or lineage committed genetically modified hematopoietic progenitor cells thereof comprise an exogenous nucleic acid.

In some embodiments, according to any of the methods described above, the method further comprises culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits TGFβ signaling.

In some embodiments, according to any of the methods described above, the method further comprises culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone demethylase inhibitor.

In some embodiments, according to any of the methods described above, the method further comprises culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone deacetylase inhibitor.

In some embodiments, according to any of the methods described above, the method further comprises culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits p38 signaling.

In some embodiments, according to any of the methods described above, the method further comprises culturing the cells in the presence of a Notch agonist. In some embodiments, the Notch Agonist is Delta-^{ext-IgG}.

In some embodiments, according to any of the methods described above, the hematopoietic stem cells and genetically modified hematopoietic stem cells are enriched in Endothelial Protein C Receptor (EPCR+) and/or CD34+, CD38+, CD90+, CD45RA+, CD133 and/or CD49f+. In some embodiments, the hematopoietic stem cells and genetically modified hematopoietic stem cells consist essentially of CD34+ cells.

In some embodiments, according to any of the methods described above, the method further comprises culturing the population of:
(i) hematopoietic and/or lineage committed hematopoietic progenitor cells; or
(ii) genetically modified hematopoietic stem cells and/or lineage committed genetically modified hematopoietic progenitor cells;
in the presence of a sufficient amount of one or more of IL6, Flt-3-L, TPO, and SCF.

In some embodiments, according to any of the methods described above, the amount of compound of formula I, II, III, or IV in the cell culture is from about 100 pm to about 10 μm.

In some embodiments, according to any of the methods described above, the stem cells and/or lineage committed progenitor cells are cultured in the presence of a compound of formula I, II, III, or IV from about 2 to about 35 days.

In some embodiments, according to any of the methods described above, the starting cell population is cultured in the presence of a compound of formula I, II, III, or IV during a time sufficient for about 2- to 50,000-fold expansion of hematopoietic cells and/or lineage committed hematopoietic progenitor cells or genetically modified hematopoietic stem cells and/or lineage committed genetically modified hematopoietic progenitor cells thereof, preferably the hematopoietic cells are CD34+ cells, as compared to a population of hematopoietic stem cells and/or lineage committed hematopoietic progenitor cells or genetically modified hematopoietic stem cells and/or lineage committed genetically modified hematopoietic progenitor cells thereof cultured under the same conditions in the absence of a compound of formula I, II, III, or IV.

In some embodiments, according to any of the methods described above, the hematopoietic stem cells and/or lineage committed hematopoietic progenitor cells or genetically modified hematopoietic stem cells and/or lineage committed genetically modified hematopoietic progenitor cells are contacted with said one or more agents simultaneously.

In some embodiments, according to any of the methods described above, the hematopoietic stem cells and/or lineage committed hematopoietic progenitor cells or genetically modified hematopoietic stem cells and/or lineage committed genetically modified hematopoietic progenitor cells are contacted with said one or more agents at different times.

In some embodiments, according to any of the methods described above, the compound is a compound of formula I having a structure of formula Ia, Ib, Ic, Id, Ie, If, or Ig:

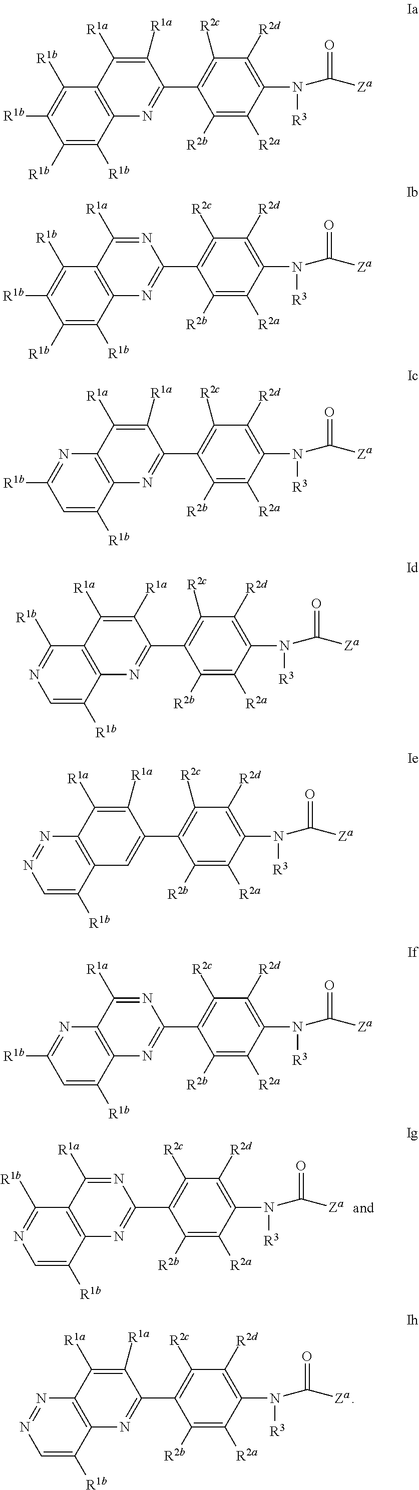

In some embodiments, Z^{a} is pyrazole or pyridine, each of which is optionally substituted with from 1 to 3 R⁴. In some embodiments, the compound is a compound of formula Ia, Ib, If or Ig having a structure of formula Ia1, Ib1, If1, or Ig1:

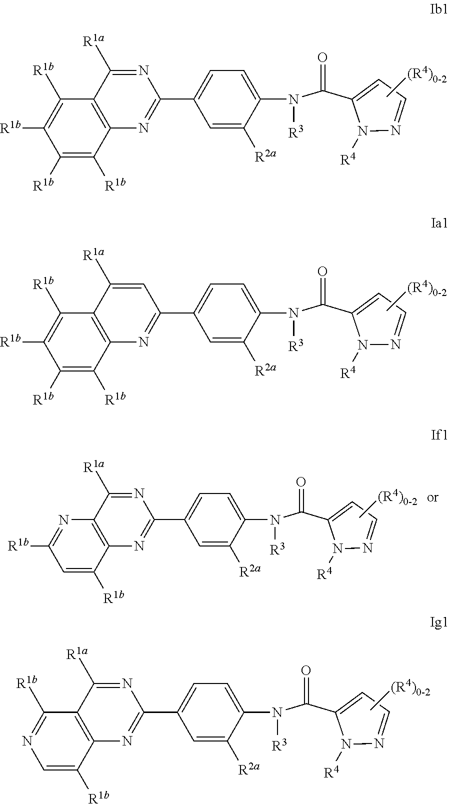

wherein:
each R^{1a} and R^{1b} is independently selected from the group consisting of H, halogen,
—R^{c}, —OC(O)N^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
   R^{2a} is selected from the group consisting of H, F and CH₃;
   each R⁴ is independently selected from the group consisting of hydrogen,
halogen, —CN, —R^{f}, —CO₂R^{d}, —CONR^{d}R^{e}, —NR^{e}C(O)₂R^{f}, —NR^{d}C(O)NR^{d}R^{e},
—NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

In some embodiments, according to any of the methods described above, the compound is selected from Table 1.

In some embodiments, according to any of the methods described above, the compound is selected from Table 1 and has +++ or ++++ activity.

In another aspect, provided herein is an ex vivo or in vitro composition comprising a cell population of expanded hematopoietic stem cells and/or lineage committed hematopoietic progenitor cells and a compound of formula I, II, III, IV, Ia, Ib, Ic, Id, Ie, If Ig, Ih, Ia1, Ib1, If1, Ig1 or a compound disclosed in Table 1.

In another aspect, provided herein is a composition comprising a cell population of expanded hematopoietic stem cells and/or lineage committed hematopoietic progenitor cells thereof obtained or obtainable by culturing ex vivo a starting population of cells comprising hematopoietic stem cells and/or lineage committed hematopoietic progenitor cells according to any of the methods described above.

In some embodiments, according to any of the compositions described above, the composition is substantially free of a compound of formula I, II, III, IV, Ia, Ib, Ic, Id, Ie, If Ig, Ih, Ia1, Ib1, If1, Ig1 or a compound disclosed in Table 1 and/or any other component of the cell culture medium.

In some embodiments, according to any of the compositions described above, the composition further comprises a pharmaceutically acceptable medium.

In some embodiments, according to any of the compositions described above, the composition is suspended in a pharmaceutically acceptable medium suitable for transplantation into a patient in need thereof.

In another aspect, provided herein is a method of treating a disease treatable by hematopoietic stem cell and/or lineage committed hematopoietic progenitor cell therapy comprising administering to a patient in need thereof a composition according to any of the compositions described above. In some embodiments, the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome. In some embodiments, the disease is selected from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E).

### DETAILED DESCRIPTION OF THE INVENTION

Before the present invention is further described, it is to be understood that the invention is not limited to the particular embodiments set forth herein, and it is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements or use of a “negative” limitation.

The publications discussed herein are provided solely for their disclosure prior to the filing date of the present application. Further, the dates of publication provided may be different from the actual publication dates, which may need to be independently confirmed.

### Definitions

Unless otherwise indicated, the following terms are intended to have the meaning set forth below. Other terms are defined elsewhere throughout the specification.

“Ex vivo” as used herein, refers to a process in which cells are removed from a living organism and are propagated outside the organism. Methods of ex vivo culturing stem cells from different tissues are well known in the art of cell culturing. See for example, the text book “Culture of Animal Cells-A Manual of Basic Technique” by Freshney, Wiley-Liss, N.Y. (2016), Seventh Edition, the teachings of which are hereby incorporated by reference.

“In vitro” refers to a process by which cells known to propagate only in vitro, such as various cell lines, are cultured.

“Stem cell” as used herein, refers to a mammalian cell that can undergo self-renewal and can differentiate into multiple cell types. The term as used herein, encompasses naturally occurring and non-naturally occurring (for example induced pluripotent stems cells and genetically modified stem cells) pluripotent and multipotent stems cells, unless otherwise stated. Thus, the term stem cells covers pluripotent stem cells i.e., induced pluripotent stem cells (iPSCs) and embryonic stem cells (ESCs); multipotent stem cells i.e., hematopoietic stem cells (HSCs), mesenchymal stem cells (MSCs), neuronal stem cells (NSCs), and epidermal stem cell; and genetically modified variants of each of the aforementioned stem cell type. As used herein “pluripotent,” “totipotent” or “omnipotent” stem cells refer to cells that can self-renew and differentiate to produce all of the cell types that make up the body. Multipotent stem cells also self-renew but unlike pluripotent stem cells, they form some but not all of the cell types in the body. In some embodiments, the stem cells are human cells.

“Multipotent” when used in reference to a “multipotent cell” refers to a cell that has the developmental potential to differentiate into multiple different cell types which may or may not be in the same lineage. For example, HSC can differentiate into multiple different types of blood cells (red, white, platelets, etc.).

“Induced pluripotent stem cell” or “iPSC” means PSC that is derived from a cell that is not a PSC (i.e., from a cell this is differentiated relative to a PSC). iPSCs can be derived from multiple different cell types, including terminally differentiated cells. iPSCs have an ES cell-like morphology and express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference.

“Hematopoietic stem cells” refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. In addition, HSCs also refer to long-term repopulating HSCs (LT-HSCs) and short-term repopulating HSCs (ST-HSCs). LT-HSCs and ST-HSCs are differentiated, based on functional potential and on cell surface marker expression. In addition, ST-HSCs are less quiescent and more proliferative than LT-HSCs under homeostatic conditions. However, LT-HSC have greater self-renewal potential (i.e., they survive throughout adulthood and can be serially transplanted through successive recipients) whereas ST-HSCs have limited self-renewal (i.e., they survive for only a limited period of time, and do not possess serial transplantation potential). Any of these HSCs can be used in the methods described herein.

“Mesenchymal stem cell” are cells that can renew and differentiate to give rise to cells of a mesenchymal cell lineage (e.g., bone, cartilage, muscle, and fat cells).

“Naturally-occurring” as used herein and as applied to a nucleic acid, polypeptide, cell, or an organism, refers to a nucleic acid, polypeptide, cell, or organism that is found in nature. For example, a polypeptide or polynucleotide sequence that is commonly present in an organism (including viruses) that can be isolated from a source in nature and which has not been intentionally modified by a human in the laboratory is naturally occurring.

“Non-naturally occurring” as used herein in reference to a stem cell refers to a stem cell that has at least one genetic or physical alteration not normally found in a naturally occurring stem cell. Genetic alterations include, for example, modifications introducing expressible nucleic acids encoding polypeptides or signalling polypeptides (for example, fluorescent polypeptides), other nucleic acid additions, nucleic acid deletions and/or other functional disruption of genetic material. Such modifications include, for example, coding regions and functional fragments thereof, for heterologous, homologous or both heterologous and homologous polypeptides. Additional modifications include, for example, non-coding regulatory regions in which the modifications alter expression of a gene.

“Genetically modified stem cell(s)” refers to a naturally occurring stem cell that has exogenous nucleic acid introduced inside the cell and/or one or more endogenous genes, either completely or partially, deleted or replaced, and/or one or more endogenous gene mutated. All of the aforementioned modifications occur artificially i.e., are man-made.

“Exogenous” refers to any material introduced from outside an organism, cell, or system.

“Endogenous” refers to any material that occurs naturally in an organism, cell, or system.

“Nucleic acid” or “polynucleotide” refers to any nucleic acid molecule, including, without limitation, DNA, RNA, and hybrids thereof. The nucleic acid bases that form nucleic acid molecules can be the bases A, T, G, C, and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Lineage committed progenitor cells” or “progenitor cells” means multipotent cells that can differentiate into one or more cells types in a particular lineage. For example, in the case of HSC stem cells, the lineage committed progenitor cells are lymphoid and myeloid progenitor cells. “Lymphoid and/or myeloid progenitor cells” may also be referred to herein as progen(ies) of HSC. Other lineage committed progenitor cells include, but are not limited to, hepatic progenitor cells, pancreatic progenitor cells, bronchiolar progenitor cells, alveolar progenitor cells, and endothelial progenitor cells. The phrase “stem cells and/or lineage committed progenitor cells” as used herein in reference to naturally occurring lineage committed progenitor cells includes lineage committed progenitor cells obtained directly from their source and/or derived from cultured stem cells, unless stated otherwise. For example, “hematopoietic stem cells and/or lineage committed hematopoietic progenitor cells” in reference to lineage committed hematopoietic progenitor cells include lineage committed hematopoietic progenitor cells that are obtained directly from their source (e.g., bone marrow, umblical cord etc.) and/or derived from cultured hematopoietic stem cells. In a subembodiment, the progentor cells are derived from cultured stem cells.

“Expansion” or “expanded” in the context of cells refers to increase in the number of a characteristic cell type or cell types relative to the number of corresponding cell type or cell types in the original population using any of the methods disclosed herein. Additionally, the phrase is used herein to describe a process of cell expansion that is substantially free of cell differentiation. “Substantially free” as used herein means less than about 95%, 90%, 85%, 80%, 75%, 70%, 65%, 60%, 55%, 50%, 45%, 40%, 35%, 30%, 25%, 20%, 15%, or 10% of expanded population of cells have undergone differentiation. “About” as used herein means + or −10% deviation from each of the above listed initial value.

“Differentiation” or “differentiate” or “differentiating” as used herein refers to the developmental process of lineage commitment. A “lineage” refers to a pathway of cellular development, in which precursor or progenitor cells undergo progressive physiological changes to become a specified cell type having a characteristic function (e.g., nerve cell, muscle cell, T cell, B cell, erythrocytes, or endothelial cell). Differentiation occurs in stages, whereby cells gradually become more specific until they reach full maturity, which is also referred to as “terminal differentiation.” A terminally differentiated cell is a cell that has committed to a specific lineage and has reached the end stage of differentiation (i.e., a cell that has fully matured).

“Maintaining functional potential of stem cell” or “maintain functional potential of stem cell” refers to functional properties of stem cells which include the ability to self-renew and to differentiate into their respective progeny cells. For example, a HSC will have the ability to 1) maintain its multipotency i.e., be able to differentiate into its progenitor cells (such as common erythroid progenitor, common lymphoid progenitor) and ultimately all types of blood cells including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), and 2) self-renew (which refers to the ability of hematopoietic stem cells to give rise to daughter cells that have equivalent potential as the mother cell, and further that this ability can repeatedly occur throughout the lifetime of an individual without exhaustion). In case of mesenchymal stem cells, the MSCs will maintain their ability to self-renew and to differentiate to give rise to their progeny cells e.g., connective tissue, bone, cartilage. As used herein, mesenchymal stem cells include, without limitation, CD34⁻ stem cells.

“Self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

“Population of stem cells” as used herein means a collection of two or more stem cells. Preferably, the population consists of at least twenty cells, or at least one hundred cells, or at least one thousand cells, or more.

“Agent that reduces the activity of an ikaros family transcription factor” refers to any agent (e.g., a small molecule, proteins (including antibodies and peptides), miRNA, and interfering nucleic acids) that regulates the activity of any member of ikaros pathway which results in reduction in the activity of an ikaros family transcription factor in the cell, such as ikaros, aiolos, and helios. Exemplary agents capable of reducing the level of an ikaros transcription factor include compounds that activate ubiquitin ligases, such as cereblon-containing ubiquitin E3 ligases that mediate the polyubiquitination of an ikaros family transcription factor. Polyubiquitination of ikaros family transcription factors leads to degradation of the transcription factor.

“Agent that inhibits histone demethylation” refers to any agent (e.g., a small molecule, proteins (including antibodies and peptides), miRNA, and interfering nucleic acids) that inhibits the activity of a histone demethylase by directly binding to the histone demethylase or by modulating the activity of any member of histone demethylase pathway that results in histone demethylation. Histone demethylases include lysine-specific demethylases, such as LSD1 and LSD2, as well as other FAD-dependent histone demethylases. Histone demethylases also include dioxygenases that utilize divalent iron (Fe+²) to catalyze oxidative demethylation of histone residues, such as Alk B, and Jumonji C (JmjC) domain-containing histone demethylases, such as JHDM1, JHDM2, and members of the JMJD2 superfamily of histone demethylases. Other enzymes that convert methylated histone residues into reactive intermediates that subsequently undergo oxidative demethylation include monoamine oxidases. Exemplary assays that can be used to elucidate the biological activity of a histone demethylation inhibitor include, without limitation, cell-based growth inhibition assays and dissociation-enhanced lanthanide fluorescence assays as described in U.S. Pat. No. 8,735,622, time-resolved fluorescence resonance energy transfer assays as described in WO 2014/151945, as well as mass spectrometry-based assays and coupled-enzyme formaldehyde dehydrogenase assays as described in WO 2010/043866.

“Agent that inhibits TGFβ signaling” refers to any agent (e.g., a small molecule, proteins (including antibodies and peptides), miRNA and interfering nucleic acids) that reduces the activity of TGFβ receptor by directly binding to a TGFβ receptor or by modulating the activity of any member of TGFβ receptor pathway such as a SMAD, Activin, Nodal, bone morphogenetic protein (BMP), growth and differentiation factor (GDF), or Müllerian inhibitory factor (MIF) or substances that promote the ubiquitination and degradation of above proteins. Exemplary assays that can be used to determine the inhibitory activity of a TGFβ signaling pathway inhibitor include, without limitation, electrophoretic mobility shift assays, antibody super shift assays, as well as TGFβ-inducible gene reporter assays, as described in WO 2006/012954.

“Agent that inhibits p38 signaling” refers to any agent (e.g., a small molecule, protein (including antibodies and peptides), miRNA, interfering nucleic acids) that reduces the activity of the p38 mitogen activated protein kinases (MAPKs, e.g., p38^{a}, p38, p38y, or p388) by directly binding to a p38 kinase or by modulating the activity of any member of p38 kinase pathway. Exemplary assays that can be used to determine the inhibitory activity of an agent that inhibits the p38 signaling pathway include, without limitation, fluorescence anisotropy competitive binding assays, as well as time-resolved fluorescence resonance energy transfer assays, as described in WO 2006/012954.

“Agent that inhibits histone deacetylation” refers to any agent (e.g., a small molecule, proteins (including antibodies and peptides), miRNA, and interfering nucleic acids) that reduces the activity of histone deacetylase by directly binding to a histone deacetylase or by modulating the activity of any member of histone deacetylase pathway that results in histone deacetylation. As used herein, the term “histone deacetylase” refer to any one of a family of enzymes that catalyze the removal of acetyl groups from the £-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

“Agent that inhibits a protein that promotes β-catenin degradation” refers to any agent (e.g., a small molecule, proteins (including antibodies and peptides), miRNA, interfering nucleic acids) that reduces the rate or extent of β-catenin degradation, e.g., by attenuating the catalysis of phosphorylation of serine and/or threonine residues that would otherwise render β-catenin a substrate for ubiquitination and proteasome-mediated degradation (for example, at residues Ser33, Ser37 and/or at Thr41). By extending the half-life of functional β-catenin, these agents promote a concomitant increase in the rate or extent of transcription of a gene that is transcribed due to the activity of the β-catenin transcription co-activator. Exemplary agents that inhibit β-catenin phosphorylation include agonists of the canonical β-catenin/Wnt signaling pathway, a signal transduction cascade that causes inhibition of glycogen synthase kinase 3 (GSK3) by providing a substrate that competes with β-catenin for phosphorylation.

“A Wnt signaling agonist” refers to an agonist of the canonical Wnt signaling pathway (e.g., a small molecule, protein (including antibodies and peptides), miRNA, interfering nucleic acids) and further includes Wnt proteins or other compounds that bind directly to the Frizzled and LRP5/6 co-receptor proteins to promote an increase in the concentration of β-catenin in the nucleus of a mammalian cell. Alternatively, a β-catenin/Wnt pathway agonist may function by inhibiting one or more secreted Frizzled-related proteins (SFRPs) or Wnt inhibitory protein (WIF), which bind and sequester Wnt proteins from the endogenous Wnt co-receptors. Exemplary methods that can be used to determine the activity of a β-catenin/Wnt pathway agonist include, without limitation, monitoring the expression of a reporter gene under the control of a TCF/LEF family transcription factor, as well as TOPflash luciferase reporter assays, as described in US 2014/0044763.

“Notch agonist” refers to an agent (e.g., a small molecule, proteins (including antibodies and peptides), miRNA, and interfering nucleic acids) that promotes activation of Notch pathway function. The term “Notch pathway function” as used herein refers to a function mediated by the Notch signal transduction pathway including, but not limited to, nuclear translocation of the intracellular domain of Notch, nuclear translocation of RBP-JK or its Drosophila homolog Suppressor of Hairless; activation of bHLH genes of the Enhancer of Split complex, e.g., Mastermind; activation of the HES-1 gene or the KBF2 (also referred to as CBF1) gene; inhibition of Drosophila neuroblast segregation; and binding of Notch to a Delta protein, a Jagged/Serrate protein, Fringe, Deltex or RBP-JK Suppressor of Hairless, or homologs or analogs thereof. The Notch signal transduction cascade and the phenotypes effected by Notch signaling are described, e.g., in Kopan et al. Cell 137:216 (2009) and Jarriault et al. Molecular Cell Biology 18:7423 (1998), the disclosures of each of which are incorporated herein by reference. Examples of Notch agonists are described, e.g., in US 2014/0369973 and in U.S. Pat. No. 7,399,633, the disclosures of each of which are incorporated herein by reference.

“Proteins”, “peptide”, or polypeptide” refers to a polymer of amino acids, including modified amino acids (e.g., phosphorylated, glycated, glycosylated, etc.) and amino acid analogs. Protein is often used in reference to relatively large polypeptides, whereas peptide is often used in reference to small polypeptides, but usage of these terms in the art overlaps.

“Interfering nucleic acids” refers to any double stranded or single stranded nucleic acids (e.g., DNA or RNA) sequence capable, either directly or indirectly (i.e., upon conversion), of inhibiting gene expression by mediating RNA interference and/or preventing gene transcription or translation. Interfering RNA includes but is not limited to small interfering RNA (“siRNA”) and small hairpin RNA (“shRNA”). RNA interference refers to the selective degradation of a sequence-compatible messenger RNA transcript. In other embodiments, an interfering nucleic acid is a DNA sequence (for example, an antisense oligonucleotide DNA sequence) capable, either directly or indirectly, of inhibiting gene expression or protein translation by binding to genomic DNA sequences or transcribed mRNA.

“shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

“RNAi” (RNA interference) refers to a post-transcriptional silencing mechanism initiated by small double-stranded RNA molecules that suppress expression of genes with sequence homology.

“miRNA” refers to small, non-coding, single stranded RNA, typically between 18-23 nucleotide in length. miRNA is capable of regulating gene expression by modulating the stability and translation of mRNA.

“Small molecule” refers to a synthetic organic molecule having a molecular weight less than 1,500 Da, more preferably less than 1,000 Da, most preferably less than 500 Da.

“Mammal” refers to a vertebrate such as a primate, rodent, domestic animal (e.g., pets or livestock) or game animal, preferably a human.

“Administration”, “administer” and the like, as they apply to, for example, a subject, cell, tissue, organ, or biological fluid, refer to contact of, for example, expanded stem cells produced by the methods disclosed herein or a pharmaceutical composition comprising same to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Patient” or “subject” are used interchangeably to refer to a human or a non-human animal (e.g., a mammal). In one embodiment, the patient is human.

“Treat”, “treating”, treatment” and the like refer to a course of action (such as administering expanded stem cells produced by the methods disclosed herein or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

“In need of treatment” as used herein refers to a judgment made by a physician or other caregiver that a subject requires or will benefit from treatment. This judgment is made based on a variety of factors that are in the realm of the physician's or caregiver's expertise.

“Prevent”, “preventing”, “prevention” and the like refer to a course of action (such as administering expanded stem cells produced by the methods disclosed herein or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

“In need of prevention” as used herein refers to a judgment made by a physician or other caregiver that a subject requires or will benefit from preventative care. This judgment is made based on a variety of factors that are in the realm of a physician's or caregiver's expertise.

“Therapeutically effective amount” refers to the administration of expanded stem cells prepared by methods disclosed herein to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like.

“In a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

“Inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, inhibit, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, inhibits, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. An inhibitor can, unless stated otherwise, reduce the activity of the target protein either directly or indirectly by modulating the activity of a member of the signaling pathway in a manner that reduces the activity of the target protein. Direct inhibition can be obtained, for instance, by binding to a protein and preventing the protein from interacting with an endogenous molecule, such as an enzyme, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and activates, the target protein.

“Reduce,” “reduction” or “decrease” or “inhibit” typically means the activity of the target protein observed in its active state is reduced by at least about 10%, 20%, 25%, 30%, 35%, 40%, 45%, 50%, 55%, 60%, 65%, 70%, 75%, 80%, 85%.

“Modulate” means to change or induce an alteration in a particular biological activity of a target protein either directly or indirectly. Modulation includes, but is not limited to, stimulating or inhibiting an activity (e.g., by activating a receptor so as to initiate a signal transduction cascade, by inhibiting a receptor so as to prevent or decrease a signal transduction cascade, by activating an endogenous inhibitor that attenuates a biological activity, or by inhibiting the activity of a protein that inhibits a particular biological function).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

“Enriched” when used in the context of cell population refers to a cell population selected based on the presence of one or more cell surface markers, for example, CD34+.

“CD34+ cells” refers to cells that express on their surface CD34 marker. CD34+ cells can be detected and counted using for example flow cytometry and fluorescently labeled anti-CD34 antibodies.

“Enriched in CD34+ cells” means that a cell population has been selected based on the presence of CD34 marker. Accordingly, the percentage of CD34+ cells in the cell population after selection method is higher than the percentage of CD34+ cells in the initial cell population before selecting step based on CD34 markers. For example, CD34+ cells may represent at least 50%, 60%, 70%, 80% or at least 90% of the cells in a cell population enriched in CD34+ cells.

Medium refers to culture medium comprising nutritive substances.

“Alkyl”, by itself or as part of another substituent, means, unless otherwise stated, a saturated straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “deuteroalkyl”, by itself or as part of another substituent, refers to an alkyl group wherein from one to five hydrogen atoms have been replaced by deuterium. An example of a “deuteroalkyl” group is —CD₃.

“Alkylene” refers to a divalent alkyl group as defined herein. Examples of alkylene include methylene, ethylene, and the like.

“Alkenyl”, by itself or as part of another substituent, means, unless otherwise stated, a straight or branched chain hydrocarbon radical containing one or two double bonds and having the number of carbon atoms designated (i.e. C₂₋₆ means two to six carbons). Examples of alkenyl groups include ethenyl, n-propenyl, isopropenyl, n-butenyl, and the like.

“Alkynyl”, by itself or as part of another substituent, means, unless otherwise stated, a straight or branched chain hydrocarbon radical containing a triple and having the number of carbon atoms designated (i.e. C₂₋₆ means two to six carbons). Examples of alkynyl groups include ethynyl, propynyl, and the like.

“Alkoxy,” “alkylamino” and “alkylthio” (or thioalkoxy) are used in their conventional sense and refer to those alkyl groups attached to the remainder of the molecule via an oxygen atom, an amino group, or a sulfur atom, respectively. The terms “deuteroalkoxy is used in its conventional sense, and refer to deuteroalkyl, as defined herein, that is attached to the remainder of the molecule via an oxygen atom.

“Aryl” means, unless otherwise stated, a polyunsaturated, typically aromatic, hydrocarbon group which can be a single ring or multiple rings (up to three rings) which are fused together or linked covalently. Non-limiting examples of aryl groups include phenyl, naphthyl and biphenyl.

“Cycloalkyl” refers to hydrocarbon rings having the indicated number of ring atoms (e.g., C₃₋₆ cycloalkyl) and being fully saturated or having no more than one double bond between ring vertices.

“Heterocycloalkyl” refers to a ring having the indicated number of vertices (C₃₋₇ refers to a 3- to 7-membered ring) and having from one to five heteroatoms selected from N, O, and S, which replace one to five of the carbon vertices, and wherein the nitrogen and sulfur atoms are optionally oxidized, and the nitrogen atom(s) are optionally quaternized. More specifically, the subscript refers to the total number of ring vertices including the carbon and heteroatom ring vertices. The heterocycloalkyl may be a monocyclic, a bicyclic or a polycylic ring system. Non-limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom.

As used herein, a wavy line, “ [symbol] ”, that intersects a single, double or triple bond in any chemical structure depicted herein, represent the point attachment of the single, double, or triple bond to the remainder of the molecule. Additionally, a bond extending to the center of a ring (e.g., a phenyl ring) is meant to indicate attachment at any of the available ring vertices. One of skill in the art will understand that multiple substituents shown as being attached to a ring will occupy ring vertices that provide stable compounds and are otherwise sterically compatible. For a divalent component, a representation is meant to include either orientation (forward or reverse). For example, the group “—C(O)NH—” is meant to include a linkage in either orientation: —C(O)NH— or —NHC(O)—, and similarly, “—O—CH₂CH₂—” is meant to include both —O—CH₂CH₂— and —CH₂CH₂—O—.

“Halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

“Heteroaryl” refers to a 5- to 10-membered aromatic ring (or fused ring system) that contains from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

As used herein, the term “heteroatom” is meant to include oxygen (O), nitrogen (N), sulfur (S) and silicon (Si).

“Pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of the present invention may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (2H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

“Pharmaceutical composition” refers to the active agent in combination with a pharmaceutically acceptable carrier e.g. a carrier commonly used in the pharmaceutical industry.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Other than in the operating examples, or where otherwise indicated, all numbers expressing quantities of ingredients or reaction conditions used herein should be understood as modified in all instances by the term “about.”

“Comprising” or “comprises” is used in reference to compositions, methods, and respective component(s) thereof, that are essential to the method or composition, yet open to the inclusion of unspecified elements, whether essential or not.

“Consisting of” refers to compositions, methods, and respective components thereof as described herein, which are exclusive of any element not recited in that description of the embodiment.

“Consisting essentially of” refers to those elements required for a given embodiment. The term permits the presence of elements that do not materially affect the basic and novel or functional characteristic(s) of that embodiment.

“About” as used herein means + or −10% deviation from each of the initial value listed unless indicated otherwise.

### METHODS

In one aspect, provided is a method of in vitro or ex-vivo culturing of stem cells and/or lineage committed progenitor cells comprising culturing a population of the stem cells and/or lineage committed progenitor cells in a medium comprising a compound of formula I, II, III or IV:

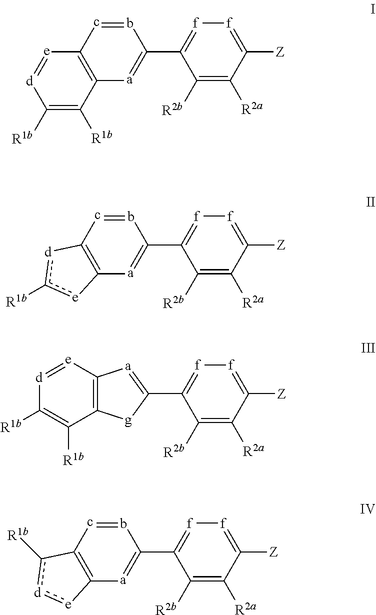

or a pharmaceutically acceptable salt, solvate or hydrate thereof, wherein
one of the dashed bonds is a single bond and the other of the dashed bonds is a double bond;
each of ring vertices a, b and c is independently selected from the group consisting of C(R^{1a}) and N;
each of ring vertices d and e is independently selected from the group consisting of C(R^{1b}) N, NH, N(C₁₋₄ alkyl) and N(C₁₋₄ haloalkyl) provided at least of d and e is other than C(R^{1b});
each ring vertex f is selected from the group consisting of C(R^{2c}), C(R^{2d}) and N;
ring vertex g is selected from the group consisting of O, S and N(R^{1a});
Z is selected from the group consisting of:

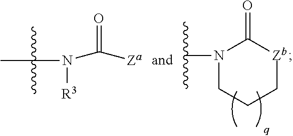

Z^{a} is selected from the group consisting of:
   (i) a 5- or 6-membered heteroaryl group having at least one nitrogen atom as a ring member, which is optionally substituted with from 1 to 4 R⁴;
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
   (iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a},
   —OC(O)NR^{a}R^{b}, —NR^{b}C(O)R^{a}, —NR^{b}C(O)₂R^{c}, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—P(O)(OH)₂, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;

and wherein the aliphatic and cyclic portions of R^{d}, R^{e} and R^{f} are optionally further substituted with from one to three halogen, hydroxy, methyl, amino, methylamino, dimethylamino and carboxylic acid groups;

wherein the compound of formula I, II, III, or IV antagonizes the activity of aryl hydrocarbon receptor.

In a second aspect, provided is a method for producing an expanded population of stem cells and/or lineage committed progenitor cells comprising contacting a population of the stem cells and/or lineage committed progenitor cells with a compound of formula I, II, III or IV, as defined in the first aspect above;
wherein the compound of formula I, II, III or IV antagonizes the activity of aryl hydrocarbon receptor; and
the stem cells and/or progenitor cells are cultured under conditions allowing expansion of the stem cells and/or progenitor cells respectively.

In a first embodiment of the second aspect, the stem cells and/or lineage committed progenitor cells are expanded in vivo by administering to a patient in need thereof a therapeutically effective amount of a compound of formula I, II, III or IV wherein each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R, —NR^{d}C(O)NR^{d}R^{e}, —NR^{d}R^{e}, —OR^{d}, —S(O)₂NR^{d}R^{e}, —X¹—CN, —X¹—CO₂R, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine.

In a second embodiment of the second aspect, the stem cells and/or lineage committed progenitor cells are expanded in vitro or ex vivo. For in vitro or ex vivo expansion, the method comprises culturing a population of the stem cells and/or lineage committed progenitor cells in a medium comprising a compound of formula I, II, III or IV wherein each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine.

In some embodiments, the stem cell population and/or lineage committed progenitor cells increases by about 10%, 15%, 20%, 25%, 30%, 35%, 40%, 45%, 59%, 55%, 60%, 65%, 70%, 75%, 80%, 85%, or 90% from the initial amount or increases by at least about a 2-fold, 3-fold, 4-fold, 5-fold or 10-fold, or greater from an initial amount.

In some embodiments of the first and second aspects and first and second embodiments contained within the second aspect, the stem cells and/or progenitor cells are embryonic stem cells and/or lineage committed embryonic progenitor cells.

In some embodiments of the first and second aspects and first and second embodiments contained with the second aspect, the stem cells and/or progenitor cells are induced pluripotent stem cells and/or lineage committed induced pluripotent progenitor cells.

In some embodiments of the first and second aspects and first and second embodiments contained with the second aspect, the stem cells and/or progenitor cells are hematopoietic stem cells and/or lineage committed hematopoietic progenitor cells.

In some embodiments of the first and second aspects and first and second embodiments contained with the second aspect, the stem cells are hematopoietic stem cells.

In some embodiments of the first and second aspects and first and second embodiments contained with the second aspect, the stem cells and/or progenitor cells are genetically modified hematopoietic stem cells and/or lineage committed genetically modified hematopoietic progenitor cells.

In some embodiments of the first and second aspects and first and second embodiment contained with the second aspect, the stem cells and/or progenitor cells thereof are mesenchymal stem cells and/or lineage committed mesenchymal progenitor cells or genetically modified mesenchymal stem cells and/or lineage committed genetically modified mesenchymal progenitor cells.

Embodiments related to in vitro or ex vivo expansion of hematopoietic stem cells and/or lineage committed hematopoietic progenitor cells, genetically modified hematopoietic stems cells and/or lineage committed genetically modified hematopoietic progenitor cells, mesenchymal stem cells and/or lineage committed mesenchymal progenitor cells, and genetically modified mesenchymal stems cells, and/or lineage committed genetically modified mesenchymal progenitor cells:

In some embodiments, wherein the stem cells and/or progenitor cells are (i) hematopoietic stem cells and/or lineage committed hematopoietic progenitor cells or (ii) genetically modified hematopoietic stems cells and/or lineage committed genetically modified hematopoietic progenitor cells, the method further includes culturing the hematopoietic stem cells and/or lineage committed hematopoietic progenitor cells or genetically modified hematopoietic stems cells and/or lineage committed genetically modified hematopoietic progenitor cells in the presence of one or more agent(s) selected from a Notch agonist, an agent that inhibits TGFβ signaling, an agent that reduces the activity of an ikaros family transcription factor, an agent that inhibits histone demethylation, an agent that inhibits p38 signaling, an agent that inhibits histone deacetylation, and an agent that inhibits a protein that promotes β-catenin degradation.

Representative examples of Notch agonists include those disclosed in, e.g., in US 2014/0369973 and in U.S. Pat. No. 7,399,633 and methods for expanding hematopoietic stems cells using AhR antagonists and Notch agonists are described in PCT application publication No. WO2013/086436, the disclosures of each of which are incorporated herein by reference. In some embodiments the Notch agonist is an extracellular domain of a Delta protein or a Jagged/Serrate protein or a Notch-binding portion of any Delta protein or a Jagged protein optionally fused to a fusion partner such as Fc region of an IgG. In some embodiments, the Notch agonist is Delta-^{ext-IgG}.

Agents that can be used to inhibit TGF signaling include, for example, 2-[3-(6-methyl-2-pyridinyl)-1H-pyrazol-4-yl]-1,5-naphthyridine (ALK5 inhibitor II, also known as E-616452), 4-[3-(2-pyridinyl)-1H-pyrazol-4-yl]-quinoline (LY36494, also known as ALK5 Inhibitor I), 3-(6-methyl-2-pyridinyl)-N-phenyl-4-(4-quinolinyl)-1H-pyrazole-1-carbothioamide (also known as A83-01), (4-(5-benzol[1,3]dioxol-5-yl-4-pyrldin-2-yl-1H-imidazol-2-yl)-benzamide hydrate (also known as SB431542), 4-[4-(1,3-benzodioxol-5-yl)-5-(2-pyridinyl)-1H-imidazol-2-yl]-benzamide hydrate, 4-[4-(3,4-methylene-dioxyphenyl)-5-(2-pyridyl)-1H-imidazol-2-yl]-benzamide hydrate, an Alk5 inhibitor), Galunisertib (LY2157299, an Alk5 inhibitor), 4-[2-[4-(2-pyridin-2-yl-5,6-dihydro-4H pyrrolo[1,2-b]pyrazol-3-yl)quinolin-7-yl]oxyethyl]morpholine (also known as LY2109761an Alk5/TGF RII inhibitor), 6-[2-tert-butyl-5-(6-methylpyridin-2-yl)-1H-imidazol-4-yl]quinoxaline (also known as SB525334, an Alk5 inhibitor), N-(oxan-4-yl)-4-[4-(5-pyridin-2-yl-1H-pyrazol-4-yl)pyridin-2-yl]benzamide (also known as GW788388, an Alk5 inhibitor), 3-[6-amino-5-(3,4,5-trimethoxyphenyl)pyridin-3-yl]phenol (also known as K02288, an Alk4/Alk5 inhibitor), 2-(5-chloro-2-fluorophenyl)-N-pyridin-4-ylpteridin-4-amine (also known as SD-208, an Alk5 inhibitor), N-((4-([1,2,4]triazolo[1,5-a]pyridin-6-yl)-5-(6-methylpyridin-2-yl)-1H-imidazol-2-yl)methyl)-2-fluoroaniline (also known as EW-7197, an Alk4/Alk5 inhibitor), and 5-[6-[4-(1-piperazinyl)phenyl]-pyrazolo[1,5-a]pyrimidin-3-yl]-quinoline (also known as LDN-212854, an Alk4/Alk5 inhibitor).

Additional examples of agents that inhibit TGF signaling include, but are not limited to, SM16, a small molecule inhibitor of TGF receptor ALK5, (see Fu et al. Arteriosclerosis, Thrombosis and Vascular Biology 28:665 (2008)), SB-505124 (an Alk4/Alk5 inhibitor, disclosed in Dacosta Byfield et al. Molecular Pharmacology 65:744 (2004)), and 6-bromo-indirubin-3′-oxime (disclosed in U.S. Pat. No. 8,298,825), the disclosures of each of which are incorporated herein by reference. Additional examples of inhibitors of TGFβ signaling are described in, e.g., Callahan et al. Journal of Medicinal Chemistry 45:999 (2002); Sawyer et al. Journal of Medicinal Chemistry 46:3953 (2003); Gellibert et al. Journal of Medicinal Chemistry 47:4494 (2004); Tojo et al. Cancer Science 96:791 (2005); Petersen et al. Kidney International 73:705 (2008); Yingling et al. Nature Reviews Drug Discovery 3:1011 (2004); Byfield et al. Molecular Pharmacology 65:744 (2004); Dumont et al. Cancer Cell 3:531 (2003); WO 2002/094833; WO 2004/026865; WO 2004/067530; WO 2009/032667; WO 2004/013135; WO 2003/097639; WO 2007/048857; WO 2007/018818; WO 2006/018967; WO2005/039570; WO 2000/031135; WO 1999/058128; U.S. Pat. Nos. 6,509,318; 6,090,383; 6,419,928; 7,223,766; 6,476,031; 6,419,928; 7,030,125; 6,943,191; US 2005/0245520; US 2004/0147574; US 2007/0066632; US 2003/0028905; US 2005/0032835; US 2008/0108656; US 2004/015781; US 2004/0204431; US 2006/0003929; US 2007/0155722; US 2004/0138188; and US 2009/0036382, the disclosures of each which are incorporated herein by reference.

Additional agents that can be used to inhibit TGFβ signaling include modulators of bone morphogenetic protein (BMP) signaling. BMP is a member of the TGFβ superfamily of ligands, and modulators of BMP signaling, such as inhibitors of Alk2, Alk3, and Alk6, can be used e.g., to expand hematopoietic stem cells. Exemplary BMP inhibitors include 4-[6-(4-isopropoxyphenyl)-pyrazolo[1,5-a]pyrimidin-3-yl]quinoline (also known as DMH1), 4-[6-[4-(1-methylethoxy)-phenyl]pyrazolo[1,5-a]pyrimidin-3-yl]-quinoline), 3-(6-amino-5-(3,4,5-trimethoxyphenyl)pyridin-3-yl)phenol (also known as K02288), 5-[6-[4-(1-piperazinyl)-phenyl]pyrazolo[1,5-a]pyrimidin-3-yl]-quinoline (also known as LDN-212854), 4-[6-[4-(1-piperazinyl)-phenyl]pyrazolo[1,5-a]pyrimidin-3-yl]-quinoline (also known as LDN-193189), 1-(4-(6-methyl-5-(3,4,5-trimethoxy-phenyl)pyridin-3-yl)phenyl)piperazine (also known as LDN-214117), and (5-[6-(4-methoxy-phenyl)-pyrazolo[1,5-a]pyrimidin-3-yl]quinoline (also known as ML347). Exemplary TGFβ antibodies include and Lerdelimumab and GC-1008. Additional agents that inhibit TGFβ signaling are disclosed in WO2016210292, the disclosure of which are incorporated herein by reference.

Agents that can be used to inhibit histone demethylation include 2-(1R,2S)-2-(4-(benzyloxy)phenyl)cyclopropyl-amino)-1-(4-methylpiperazin-1-yl)ethanone, HCl (LSD1 inhibitor IV RN-1), 2-(2-(benzyloxy)-3,5-difluorophenyl)-cyclopropan-1-amine (LSD1 inhibitor II S2101), (1S,2S)—N-(1-(2,3-dihydrobenzo[b][1,4]dioxin-6-yl)ethyl)-2-phenylcyclopropan-1-amine (LSD1 inhibitor LSD1-C76), methyl-3-(4-(4-carbamimidoyl-benzoyl)-piperazine-1-carbonyl)-5-((4-carbamimidoylpiperazin-1-yl)methyl)benzoate (LSD1 inhibitor III CBB1007), (E,E)-1,1′-((propane-1,3-diylbis(azanediyl))bis(propane-3,1-diyl))bis(2,3-dimethylguanidine) (LSD1 inhibitor I), Tranylcypromine, phenelzine, propargylamine, and derivatives thereof described in US 2013/0095067, tranylcypromine derivatives (described in US 2014/0163041), BIX 01294 (2-(hexahydro-4-methyl-1H-1,4-diazepin-1-yl)-6,7-dimethoxy-N-[1-(phenylmethyl)-4-piperidinyl]-4-quinazolinamine trihydrochloride, described in WO 2014/057997); UNC 0638 (2-cyclohexyl-6-methoxy-N-[1-(1-methylethyl)-4-piperidinyl]-7-[3-(1-pyrrolidinyl)propoxy]-4-quinazolinamine, described in WO 2013/050422), and CARMI inhibitor (3,5-bis(3-bromo-4-hydroxy-benzylidene)-1-benzylpiperidin-4-one, a histone arginine methyltransferase inhibitor), the disclosures of each of which are incorporated herein by reference.

Agents that can be used to inhibit histone deacetylation include Trichostatin A ((N-hydroxy-N′-phenyl-octanediamide), valproic acid, butyrylhydroxamic acid, isotadax, Panobinostat, MS-275, M344, SAHA, N-(7-trifluoroacetylpropionyl)aniline, 2-[(4-acetylaminophenyl)-carbonylamino]aniline, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, RGFP966, Rocilinostat (ACY-1215), CI994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

Examples of p38 inhibitor include 4-(4-fluorophenyl)-2-(4-methylsulfinylphenyl)-5-(4-pyridyl)-1H-imidazole (also known as SB203580), 4(4-fluorophenyl)-2-(4-hydroxyphenyl)-5-(4-pyridyl) 1H-imidazole (also known as SB202 1 90), SB203580, BIRB796 (doramapimod), VX702, SB202190, LY2228820, VX745, Vinorelbine (Navelbine), PH797804, pamapimod, CMPD-1, E01428, JX401, ML3403, RWJ67657, SB239063, SCI0469 hydrochloride, SKF86002 dihydrochloride, SX01 1, TAK715, e.g., as described in US 2014/0127231, the disclosure of which is incorporated herein by reference, and pexmetinib (ARRY-614), PH-797804 (3-(3-bromo-4-((2,4-difluorobenzyl)oxy)-6-methyl-2-oxopyridin-1(2H)-yl)-N,4-dimethylbenzamide), Losmapimod (GW856553X), and Skepinone-L.

Exemplary compounds that inhibit a protein that inhibits β-catenin degradation include GSK3 inhibitor CHIR99021, AR-A014418, and BIO (2′Z,3′E)-6-bromoindirubin-3′-oxime), lithium chloride, and FGF2.

Examples of agents that reduces the activity of an ikaros family member transcription factor include pomalidomide, lenalidomide, and thalidoamide. Additional agents that inhibit ikaros family members are disclosed in WO2017161001, the disclosure of which is incorporated herein by reference. Inhibition of ikaros activity up-regulates express of a Notch gene.

Other classes of molecules that can be used as to reduces the activity of an ikaros family transcription factor, inhibit histone demethylation, p38 signaling, histone deacetylation, TGFβ signaling, or a protein that promotes β-catenin degradation include iRNAs, miRNAs, peptides, and antibodies. For example, endogenous proteins that modulate signal transduction events may be used to attenuate these events, in vitro and ex vivo and utilizing the protein sequence of the above targets, antibodies, mRNA and iRNA can be readily prepared by methods well known in the art. For instance, a variety of proteins that antagonize the TGF signaling cascade can be used, including Decorin, an extracellular matrix proteoglycan that negatively regulates TGF activity, as well as Lefty1, Lefty2, Follistatin, Noggin, Chordin, Cerberus, Germlin, Inhibin, Cystatin C, Recombinant Mouse Lefty-I (an ACVR2B inhibitor), as well as the Smad proteins Smad6 and Smad7, which serve to prevent the phosphorylation of the R-Smad proteins or recruit ubiquitin ligases to the TGF receptor type I so as to promote the degradation of the receptor. These proteins are described in detail in U.S. Pat. No. 8,298,825, the disclosure of which is incorporated herein by reference in its entirety.

In some embodiments, wherein the stem cells and/or progenitor cells are (i) hematopoietic stem cells and/or lineage committed hematopoietic progenitor cells or (ii) genetically modified hematopoietic stems cells and/or lineage committed genetically modified hematopoietic progenitor cells, the method further comprises culturing the hematopoietic stem cells and/or lineage committed hematopoietic progenitor cells or genetically modified hematopoietic stems cells and/or lineage committed genetically modified hematopoietic progenitor cells in the presence of cytokines and growth factors, generally known in the art for hematopoietic stem cell and/or lineage committed hematopoietic progenitor cell expansion. Such cytokines and growth factors include without limitation IL-1, IL-3, IL-6, IL-11, G-CSF, GM-CSF, SCF, FlT3-L, thrombopoietin (TPO), erythropoietin, and analogs thereof “Analogs” refers to any structural variants of the cytokines and growth factors having the biological activity as the naturally occurring forms, including without limitation, variants with enhanced or decreased biological activity when compared to the naturally occurring forms or cytokine receptor agonists such as an agonist antibody against the TPO receptor (for example, VB22B sc(Fv)2 as detailed in patent publication WO 2007/145227, and the like). Cytokine and growth factor combinations are chosen to expand HSC and/or progenitor cells thereof while limiting the production of terminally differentiated cells. In some embodiments, one or more cytokines and growth factors are selected from the group consisting of SCF, Flt3-L and TPO. In some embodiments, at least TPO is used in a serum-free medium under suitable conditions for expansion of HSC and/or progenitor cells thereof.

In some embodiments, wherein the stem cells and/or progenitor cells are (i) mesenchymal stem cells and/or lineage committed mesenchymal progenitor cells or (ii) genetically modified mesenchymal stems cells and/or lineage committed genetically modified mesenchymal progenitor cells, the method further comprises culturing the cells the presence of nicotinamide.

In some embodiments, wherein the stem cells and/or progenitor cells are (i) mesenchymal stem cells and/or lineage committed mesenchymal progenitor cells or (ii) genetically modified mesenchymal stems cells and/or lineage committed genetically modified mesenchymal progenitor cells, the method further comprises culturing the cells the presence of FGF-4. Additional methods of expanding MSCs are disclosed in US 20140023623, the disclosure of which is incorporated herein by reference in its entirety.

When more than one agent is used in any of the methods above, the stem cells and/or progenitor cells can be contacted with the one or more agents simultaneously. Alternatively, the stem cells and/or progenitor cells can be contacted with said one or more agents at different times (e.g., sequentially).

In some embodiments, in any of the in vitro or ex vivo methods above, the stem cells and/or lineage committed progenitor cells are cultured with from about 1 pm to about 100 uM or from about 100 pm to about 10 um of a compound of formula I.

In some embodiments, in any of the in vitro or ex vivo methods above, the stem cells and/or lineage committed progenitor cells are cultured in the presence of a compound of formula I from about 1 day to about 90 days or from 3 days to 90 days, or from about 2 to about 35 days or from about 7 to about 35 days or 5 days to 21 days.

In some embodiments, in any of the in vitro or ex vivo methods above, the stem cells and/or lineage committed progenitor cells are cultured in the presence of a compound of Formula I from about 1 day to 3 days.

In some embodiments, in any of the in vitro or ex vivo methods above, the stem cells and/or lineage committed progenitor cells are cultured in the presence of a compound of Formula I during a time sufficient for a 10 to 50,000-fold expansion or 100 to 10,000-fold expansion of the stem cells and/or lineage committed progenitor cells thereof as compared to a population of stem cells cultured under the same conditions in the absence of a compound of Formula I.

### Embodiments Related to Compounds of Formulae I, II, III, and IV in Above Methods

Embodiment 1: In embodiment 1, in any of the methods above, the compounds of formulae I, II, III, and IV are wherein one of ring vertices a, b, c, d and e is N. In certain selected embodiments, each ring vertex f is CH.

Embodiment 2: In embodiment 2, in any of the methods above, the compounds of formulae I, II, III, and IV are wherein two of ring vertices a, b, c, d and e is N. In certain selected embodiments, each ring vertex f is CH.

Embodiment 3: In embodiment 3, in any of the methods above, the compounds of formulae I, II, III, and IV are wherein three of ring vertices a, b, c, d and e is N. In certain selected embodiments, each ring vertex f is CH.

Embodiment 4: In embodiment 4, in any of the methods above, the compounds of formulae I, II, III, and IV are those wherein Z is:

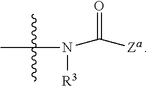

Embodiment 5: In embodiment 5, in any of the methods and Embodiments 1 to 4 above, Z^{a} is selected from the group consisting of pyrazole, imidazole, oxazole, isoxazole, 1,2,3-triazole, 1,2,4-triazole, pyridine, pyrimidine, pyridazine and pyrazine, each of which is optionally substituted with from 1 to 2 R⁴. In other embodiments, Z^{a} is selected from the group consisting of tetrahydropyran, tetrahydrothiopyran, morpholine, piperidine and piperazine.

Embodiment 6: In embodiment 6, in any of the methods above, the compounds are those of Formula I and are represented by formulae Ia, Ib, Ic, Id, Ie, If, Ig, and Ih:

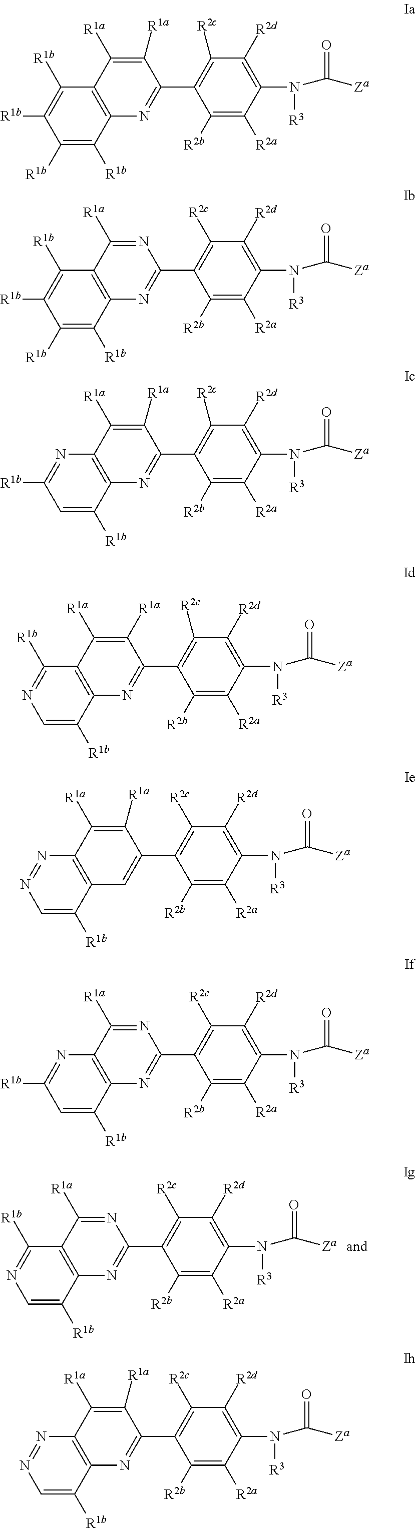

In a first subembodiment of Embodiment 6, in any of the methods above, the compounds are those of formula Ia, wherein Z^{a} is pyrazole or pyridine, each of which is optionally substituted with from 1 to 3 R⁴. In a second subembodiment of Embodiment 6, in any of the methods above, the compounds are those of formula Ib, wherein Z^{a} is pyrazole or pyridine, each of which is optionally substituted with from 1 to 3 R⁴. In a third subembodiment of Embodiment 6, in any of the methods above, the compounds are those of formula Ic, wherein Z^{a} is pyrazole or pyridine, each of which is optionally substituted with from 1 to 3 R⁴. In a fourth subembodiment of Embodiment 6, in any of the methods above, the compounds are those of formula Id, wherein Z^{a} is pyrazole or pyridine, each of which is optionally substituted with from 1 to 3 R⁴. In a fifth subembodiment of Embodiment 6, in any of the methods above, the compounds are those of formula If, wherein Z^{a} is pyrazole or pyridine, each of which is optionally substituted with from 1 to 3 R⁴. In a sixth subembodiment of Embodiment 6, in any of the methods above, the compounds are those of formula Ig, wherein Z^{a} is pyrazole or pyridine, each of which is optionally substituted with from 1 to 3 R⁴.

Embodiment 7: In embodiment 7, in any of the methods above, the compounds have formula Ia1:

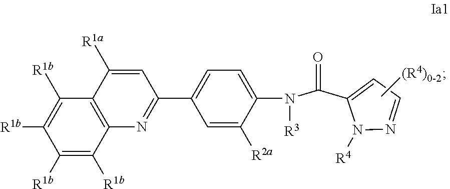

wherein:
each R^{1a} and R^{1b} is independently selected from the group consisting of H, halogen,

—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};

R^{2a} is selected from the group consisting of H, F and CH₃;
each R⁴ is independently selected from the group consisting of halogen, —CN, —R^{f},

—CO₂R^{d}, —CONR^{d}R^{e}, —NR^{e}C(O)₂R^{f}, —NR^{d}C(O)NR^{d}R^{e}, —NR^{d}R^{e}, —OR^{d}, —X¹—CN,

—X¹—CO₂R^{d}, —X¹—CONR^{d}R^{e}, —X¹—NR^{d}R^{e}, —X¹—OR^{d} and —X¹—Y.

Embodiment 8: In embodiment 8, in any of the methods above, the compounds have formula Ib1:

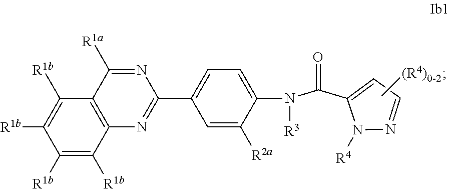

wherein
each R^{1a} and R^{1b} is independently selected from the group consisting of H, halogen,

—R^{c}, —OC(O)NR^{a}R^{b}, —NR^{b}C(O)R^{a}, —NR^{b}C(O)₂R^{c}, —N^{a}C(O)NR^{a}R^{b}, —NR^{a}R^{b} and —OR^{a};

R^{2a} is selected from the group consisting of H, F and CH₃;
each R⁴ is independently selected from the group consisting of halogen, —CN, —R^{f},

—CO₂R^{d}, —CONR^{d}R^{e}, —NR^{e}C(O)₂R^{f}, —NR^{d}C(O)NR^{d}R^{e}, —NR^{d}R^{e}, —OR^{d}, —X¹—CN,

—X¹—CO₂R, —X¹—CONR^{d}R^{e}, —X¹—NR^{d}R^{e}, —X¹—OR^{d} and —X¹—Y.

Embodiment 9: In embodiment 9, in any of the methods above, the compounds have formula If1:

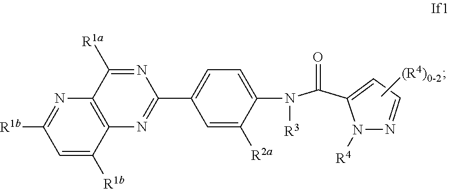

wherein
each R^{1a} and R^{1b} is independently selected from the group consisting of H, halogen,

—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};

R^{2a} is selected from the group consisting of H, F and CH₃;
each R⁴ is independently selected from the group consisting of halogen, —CN, —R^{e},

—CO₂R^{d}, —CONR^{d}R^{e}, —NR^{e}C(O)₂R^{f}, —NR^{d}C(O)NR^{d}R^{e}, —NR^{d}R^{e}, —OR^{d}, —X¹—CN,

—X¹—CO₂R^{d}, —X¹—CONR^{d}R^{e}, —X¹—NR^{d}R^{e}, —X¹—OR^{d} and —X¹—Y.

Embodiment 10: In embodiment 10, in any of the methods above, the compounds have formula Ig1:

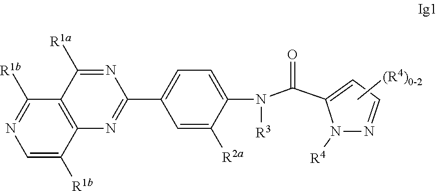

wherein
each R^{1a} and R^{1b} is independently selected from the group consisting of H, halogen,

—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};

R^{2a} is selected from the group consisting of H, F and CH₃;
each R⁴ is independently selected from the group consisting of halogen, —CN, —R^{f},

—CO₂R^{d}, —CONR^{d}R^{e}, —NR^{e}C(O)₂R^{f}, —NR^{d}C(O)NR^{d}R^{e}, —NR^{d}R^{e}, —OR^{d}, —X¹—CN,

—X¹—CO₂R^{d}, —X¹—CONR^{d}R^{e}, —X¹—NR^{d}R^{e}, —X¹—OR^{d} and —X¹—Y.

Embodiment 11: In embodiment 11, in any of the methods above, the compounds have formula I:

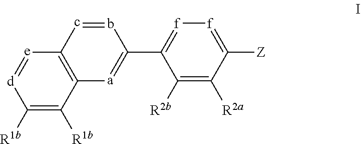

wherein d is selected from the group consisting of C(CN), C(C₁₋₄ haloalkyl) and C(C₁₋₄ haloalkoxy), and in still further embodiments, d is C(CF₃).

In a first subembodiment of Embodiment 11, the compounds are represented by a formula selected from the group consisting of:

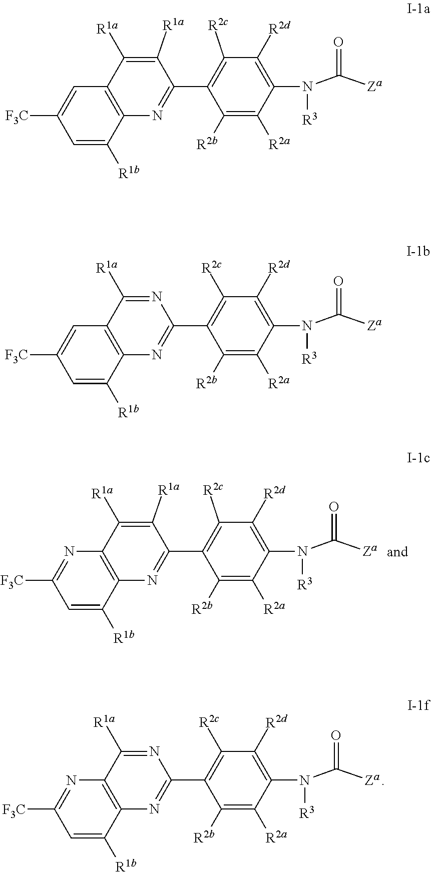

Within the first subembodiment of Embodiment 11, in some embodiments of formulae I-1a, I-1b, I-1c and I-1f, Z^{a} is selected from the group consisting of pyrazolyl, imidazolyl, oxazolyl, isoxazolyl, 1,2,3-triazolyl, 1,2,4-triazolyl, pyridinyl, pyrimidinyl, pyridazinyl and pyrazinyl, each of which is substituted with from 0 to 2 R⁴.

Within the first subembodiment of Embodiment 11, in some embodiments compounds herein are represented by formula I-1a, wherein Z^{a} is pyrazolyl or pyridinyl, each of which is substituted with 0 to 3 R⁴. In other selected embodiments, compounds herein are represented by formula I-1b, wherein Z^{a} is pyrazolyl or pyridinyl, each of which is substituted with 0 to 3 R⁴. In yet further selected embodiments, compounds herein are represented by formula I-1c, wherein Z^{a} is pyrazolyl or pyridinyl, each of which is substituted with 0 to 3 R⁴. In other further selected embodiments, compounds herein are represented by formula I-1f, wherein Z^{a} is pyrazolyl or pyridinyl, each of which is substituted with 0 to 3 R⁴.

Within Embodiment 11, embodiments of formula I, and the embodiments above, provided as formulae I-1a, I-1b, I-1c and I-1f, and embodiments contained therein, in some selected embodiments, each R⁴ is independently selected from the group consisting of halogen, —CN, —R^{f}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; each R^{d} and R^{e} is independently selected from hydrogen or C₁₋₈ alkyl; and each R is independently selected from the group consisting of C₁₋₈ alkyl.

Within Embodiment 11, embodiments of formula I, and the embodiments above, provided as formulae I-1a, I-1b, I-1c and I-1f, and embodiments contained therein, in some selected embodiments, each R^{1a} is hydrogen. In other selected embodiments, only one of R^{2a}, R^{2b}, R^{2c} and R^{2d} is other than hydrogen. In still other selected embodiments, only one of R^{2a}, R^{2b}, R^{2c} and R^{2d} is other than hydrogen, and is selected from the group consisting of F and CH₃. In other selected embodiments, Z^{a} is pyrazolyl or pyridinyl, each of which is substituted with 0 to 3 R⁴; each R^{1a} is hydrogen; one of R^{2a}, R^{2b}, R^{2c} and R^{2d} is other than hydrogen; and R³ is hydrogen or methyl.

Embodiment 12: Within Embodiment 12, in any of the methods above, compounds provided herein are represented by the formula I:

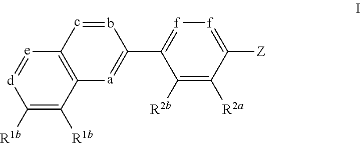

or a pharmaceutically acceptable salt, solvate or hydrate thereof, wherein:
each of ring vertices a, b and c is independently selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
is:

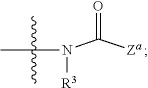

Z^{a} is a 5- or 6-membered heteroaryl group having at least one nitrogen atom as a ring member, which is substituted with from 0 to 4 R⁴;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f};
each R⁴ is independently selected from the group consisting of halogen, —CN, —R^{e}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{b}C(O)₂R^{f}, —X¹—NR^{d}C(O)NR^{d}R^{e}, —X¹—N^{d}R^{e}, —X¹—OR^{d}, —X¹—P(O)(OH)₂, —X¹—Y and —X¹—S(O)₂NR^{d}R^{e}; wherein each X¹ is independently C₁₋₆ alkylene and Y is selected from the group consisting of pyrazolyl, imidazolyl, 1,2,3-triazolyl, 1,2,4-triazolyl, tetrazolyl, piperidinyl, pyrrolidinyl, tetrahydrofuranyl, tetrahydropyranyl and morpholinyl;
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, phenyl and 5- or 6-membered heteroaryl;
wherein the aliphatic and cyclic portions of R^{d}, R^{e} and R^{f} are optionally further substituted with from one to three halogen, hydroxy, methyl, amino, methylamino, dimethylamino and carboxylic acid groups.

Within embodiment 12, in one selected group of embodiments, at least one R^{1b} is selected from the group consisting of C₁₋₄ haloalkyl, C₁₋₄ haloalkoxy and —CN. Within embodiment 12, in another selected group of embodiments, each R^{1a} is hydrogen. Within embodiment 12, in still another selected group of embodiments, each of R^{2a}, R^{2b}, R^{2c} and R^{2d} is selected from the group consisting of hydrogen, F and CH₃.

In a first subembodiment of Embodiment 12, compounds provided herein are represented by a formula selected from the group consisting of:

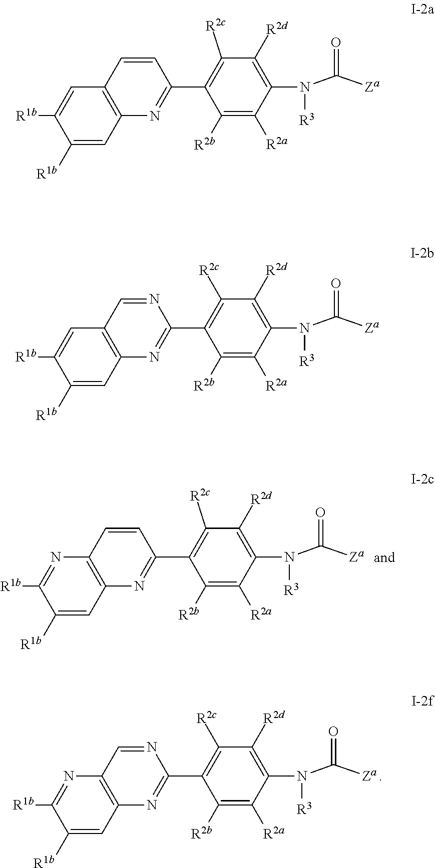

Within the first subembodiment of Embodiment 12, in some embodiments, the compound has formula I-2a, wherein one R^{1b} is CF₃. In other embodiments, the compound has formula I-2b, wherein one R^{1b} is CF₃. In still other embodiments, the compound has formula I-2c, wherein one R^{1b} is CF₃. In yet other embodiments, the compound has formula I-2f, wherein one R^{1b} is CF₃.

Within the first subembodiment of Embodiment 12 and embodiments contained therein, in some embodiments of formulae I-2a, I-2b, I-2c and I-2f, compounds provided herein are those wherein Z^{a} is pyrazolyl and is substituted with 0 to 2 R⁴; one or two of R^{2a}, R^{2b}, R^{2c} and R^{2d} is selected from the group consisting of F and CH₃; and one R^{1b} is selected from the group consisting of C₁₋₄ haloalkyl and C₁₋₄ haloalkoxy.

Embodiment 13: Within Embodiment 13, in any of the methods above, provided herein are compounds of formula:

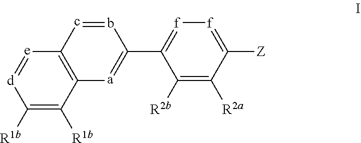

or a pharmaceutically acceptable salt, solvate or hydrate thereof, wherein;
each of ring vertices a, b and c is independently selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
is:

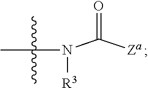

Z^{a} is a 5- or 6-membered heteroaryl group having at least one nitrogen atom as a ring member, which is substituted with from 0 to 4 R⁴;
each R^{1a} and R^{1b} is independently selected from the group consisting of hydrogen, deuterium, halogen, —CN, —NO₂, —R^{c}, —CO₂R^{a}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{c}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups; and at least one R^{1a} and R^{1b} is other than hydrogen;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f};
each R⁴ is independently selected from the group consisting of halogen, —CN, —R^{e}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{b}C(O)₂R^{f}, —X¹—NR^{d}C(O)NR^{d}R^{e}, —X¹—N^{d}R^{e}, —X¹—OR^{d}, —X¹—P(O)(OH)₂, —X¹—Y and —X¹—S(O)₂NR^{d}R^{e}; wherein each X¹ is independently C₁₋₆alkylene and Y is selected from the group consisting of pyrazolyl, imidazolyl, 1,2,3-triazolyl, 1,2,4-triazolyl, tetrazolyl, piperidinyl, pyrrolidinyl, tetrahydrofuranyl, tetrahydropyranyl and morpholinyl;
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, and 5- or 6-membered heteroaryl;
wherein the aliphatic and cyclic portions of R^{d}, R^{e} and R^{f} are optionally further substituted with from one to three halogen, hydroxy, methyl, amino, methylamino, dimethylamino and carboxylic acid groups.

In a first subembodiment of Embodiment 13, further selected embodiments are those wherein each R^{1a} is hydrogen and at least one R^{1b} is other than hydrogen. In a second of subembodiment of Embodiment 13, are further selected embodiments wherein the compound has a formula selected from the group consisting of:

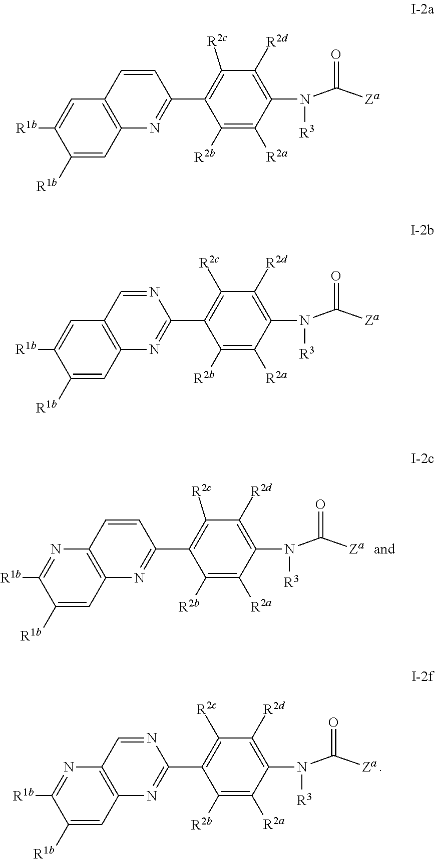

In some selected embodiments within Embodiment 13 and and further embodiments contained therein, Z^{a} is selected from the group consisting of pyrazolyl, imidazolyl, oxazolyl, isoxazolyl, 1,2,3-triazolyl, 1,2,4-triazolyl, pyridinyl, pyrimidinyl, pyridazinyl and pyrazinyl, each of which is substituted with from 0 to 2 R⁴. In other selected embodiments, only one of R², R^{2b}, R^{2c} and R^{2d} is other than hydrogen. In yet other selected embodiments, Z^{a} is pyrazolyl or pyridinyl and is substituted with 0 to 2 R⁴.

With reference to those embodiments just described as having formula I-2a, I-2b, I-2c or I-2f, further selected embodiments are those wherein one R^{1b} is selected from the group consisting of C₁₋₄ haloalkyl, C₁₋₄ haloalkoxy and —CN. In other selected embodiments, R³ is hydrogen or CH₃. In still other selected embodiments, only one of R^{2a}, R^{2b}, R^{2c} and R^{2d} is other than hydrogen; one R^{1b} is selected from the group consisting of C₁₋₄ haloalkyl, C₁₋₄ haloalkoxy and —CN; and R³ is hydrogen or CH₃.

With reference to the Embodiments 12 and 13, embodiments of formula I, or the embodiments above, provided as formulae I-2a, I-2b, I-2c and I-2f, in some selected embodiments, each R⁴ is independently selected from the group consisting of halogen, —CN, —R^{f}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y; wherein each X¹ is independently C₁₋₆ alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R^{f} is independently selected from the group consisting of C₁₋₈ alkyl.

Embodiment 14: Within Embodiment 14, in any of the methods above, provided herein are compounds of formula:

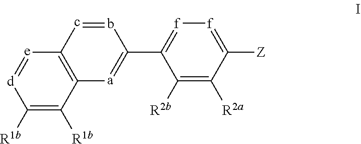

or a pharmaceutically acceptable salt, solvate or hydrate thereof, wherein;
each of ring vertices a, b and c is independently selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
is:

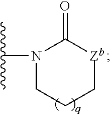

Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f};
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, phenyl and 5- or 6-membered heteroaryl;
wherein the aliphatic and cyclic portions of R^{d}, R^{e} and R^{f} are optionally further substituted with from one to three halogen, hydroxy, methyl, amino, methylamino, dimethylamino and carboxylic acid groups.

In a first subembodiment of Embodiment 14, in some selected embodiments compounds of Formula I are those wherein at least one R^{1b} is selected from the group consisting of C₁₋₄ haloalkyl, C₁₋₄ haloalkoxy and —CN. In a second subembodiment of Embodiment 14, in other selected embodiments compounds of Formula I are selected from the group consisting of:

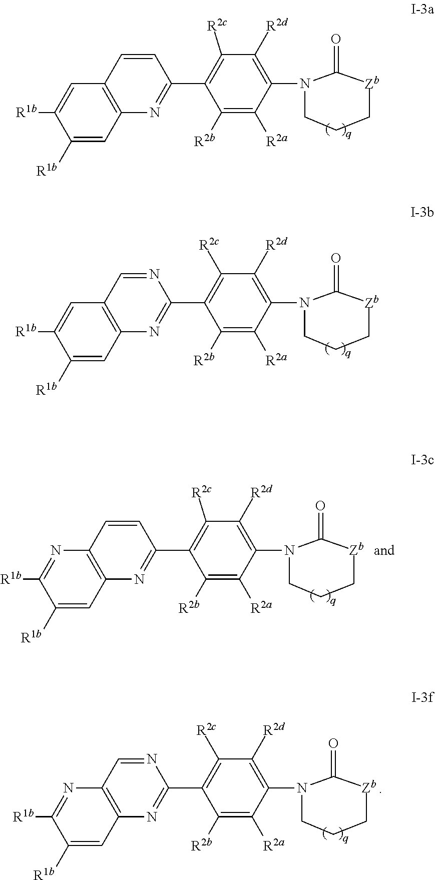

Within this group of embodiments and the selected embodiments having formula I-3a, I-3b, I-3c or I-3f, further selected embodiments are those wherein Z^{b} is selected from the group consisting of O, NH, N(CH₃) and CH₂. In still other selected embodiments, q is 0 or 1. In this other selected embodiments, only one of R^{2a}, R^{2b}, R^{2c} and R^{2d} is other than hydrogen. In yet other selected embodiments, one R^{1b} is CF₃. In one particular group of embodiments, Z^{b} is selected from the group consisting of O, NH, N(CH₃) and CH₂; q is 0 or 1; only one of R^{2a}, R^{2b}, R^{2c} and R^{2d} is other than hydrogen; and one R^{1b} is CF₃.

Embodiment 15: Within Embodiment 15, in any of the methods above, compounds provided herein have the formula:

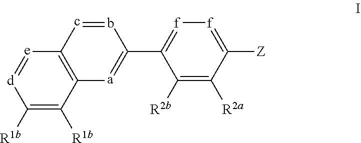

or a pharmaceutically acceptable salt, solvate or hydrate thereof, wherein;
each of ring vertices a, b and c is independently selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
is:

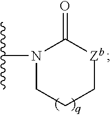

Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R, —NR^{a}C(O)NR^{a}R^{b}, —N^{a}R^{b}, —OR^{c}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups; and at least one R^{1b} is other than hydrogen;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f};
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, phenyl and 5- or 6-membered heteroaryl;
wherein the aliphatic and cyclic portions of R^{d}, R^{e} and R^{f} are optionally further substituted with from one to three halogen, hydroxy, methyl, amino, methylamino, dimethylamino and carboxylic acid groups.

In a first subembodiment of Embodiment 15, in some selected embodiments compounds of formula I are those wherein at least one R^{1b} is selected from the group consisting of C₁₋₄ haloalkyl, C₁₋₄ haloalkoxy and —CN. In a second subembodiment of Embodiment 15, in another selected embodiments compounds of Formula I are selected from the group consisting of:

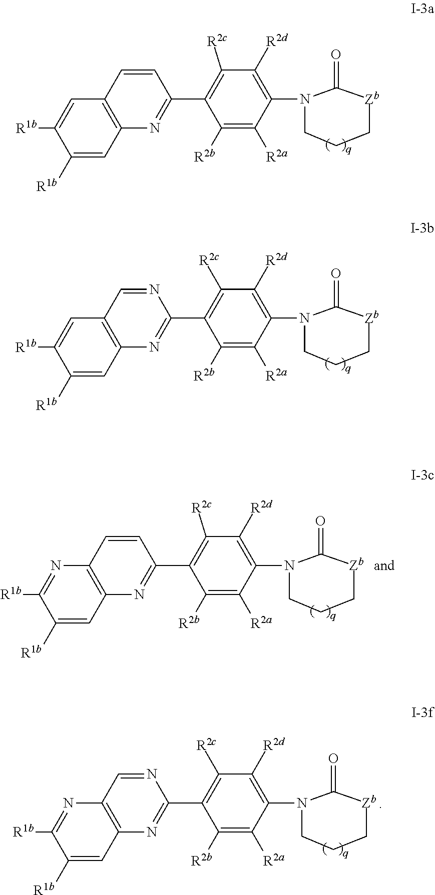

Within this group of embodiments and the selected embodiments having formula I-3a, I-3b, I-3c or I-3f, further selected embodiments are those wherein Z^{b} is selected from the group consisting of O, NH, N(CH₃) and CH₂. In still other selected embodiments, q is 0 or 1. In this other selected embodiments, only one of R^{2a}, R^{2b}, R^{2c} and R^{2d} is other than hydrogen. In yet other selected embodiments, one R^{1b} is CF₃. In one particular group of embodiments, Z^{b} is selected from the group consisting of O, NH, N(CH₃) and CH₂; q is 0 or 1; only one of R^{2a}, R^{2b}, R^{2c} and R^{2d} is other than hydrogen; and one R^{1b} is CF₃.

Embodiment 16: Within embodiment 16, in any of the methods above, in another group of embodiments, compounds provided herein have the formula:

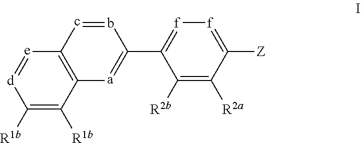

or a pharmaceutically acceptable salt, solvate or hydrate thereof, wherein:
each of ring vertices a, b and c is independently selected from the group consisting of C(R^{1a}) and N;
each of ring vertices d and e is selected from the group consisting of C(R^{1b}) and N;
each ring vertex f is selected from the group consisting of C(R^{2c}), C(R^{2d}) and N;
is:

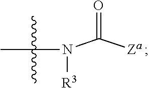

Z^{a} is pyrazolyl, which is substituted with from 0 to 4 R⁴;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f};
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—N^{d}R^{e}, —X¹—OR^{d}, —X¹—P(O)(OH)₂, —X¹—Y and —X¹—S(O)₂NR^{d}R^{e}; wherein each X¹ is independently C₁₋₆alkylene and Y is selected from the group consisting of pyrazolyl, imidazolyl, 1,2,3-triazolyl, 1,2,4-triazolyl, tetrazolyl, piperidinyl, pyrrolidinyl, tetrahydrofuranyl, tetrahydropyranyl and morpholinyl;
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, and 5- or 6-membered heteroaryl;
wherein the aliphatic and cyclic portions of R^{d}, R^{e} and R^{f} are optionally further substituted with from one to three halogen, hydroxy, methyl, amino, methylamino, dimethylamino and carboxylic acid groups.

Within Embodiment 16, some selected embodiments are those compounds wherein Z^{a} is substituted with 1 or 2 R⁴. In still other selected embodiments, compounds are provided having a formula selected from the group consisting of

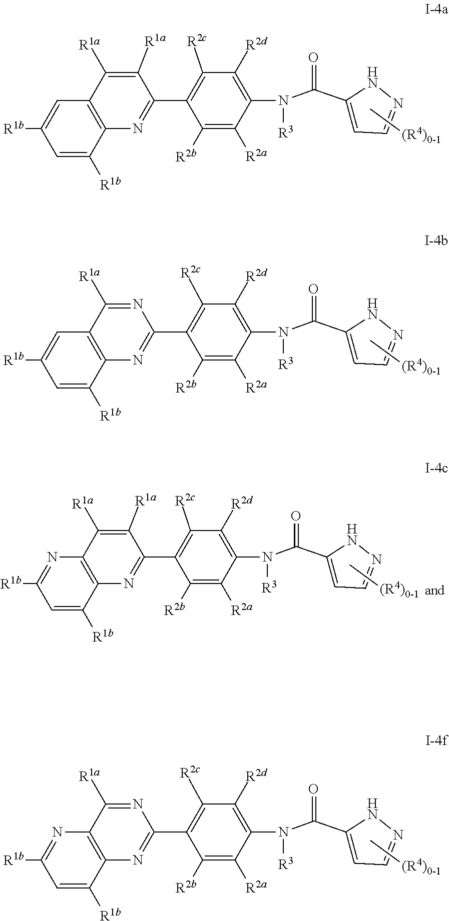

For those embodiments provided as having formula I-4a, I-4b, I-4c or I-4f, certain selected embodiments are those wherein R^{2a}, R^{2b}, R^{2c} and R^{2d} are each independently selected from the group consisting of H, F and CH₃. In some selected embodiments, the compounds are represented by formula I-4a. In other selected embodiments, the compounds are represented by formula I-4b. In still other selected embodiments, the compounds are represented by formula I-4c. In yet other selected embodiments, the compounds are represented by formula I-4f.

For those embodiments described as having formula I-4a, I-4b, I-4c or I-4f, some further embodiments are those wherein only one of R^{2a}, R^{2b}, R^{2c} and R^{2d} is other than hydrogen. In still further embodiments, one R^{1b} is selected from the group consisting of C₁₋₄ haloalkyl and C₁₋₄ haloalkoxy. In yet further embodiments, each R^{1a} is hydrogen. In further embodiments, R³ is selected from the group consisting of H and CH₃. In some selected further embodiments, the compounds described as having formula I-4a, I-4b, I-4c or I-4f, are those wherein each R^{1a} is hydrogen; one R^{1b} is selected from the group consisting of C₁₋₄ haloalkyl and C₁₋₄ haloalkoxy; one or two of R^{2a}, R^{2b}, R^{2c} and R^{2d} is other than hydrogen; and R³ is selected from the group consisting of hydrogen, C₁₋₃ alkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}.

With reference to Embodiment 16 of formula I, or the embodiments above, provided as formulae I-4a, I-4b, I-4c and I-4f, in some selected embodiments, each R⁴ is independently selected from the group consisting of halogen, —CN, —R^{f}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y; wherein each X¹ is independently C₁₋₆ alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R^{f} is independently selected from the group consisting of C₁₋₈ alkyl.

Embodiment 17: In another group of embodiments, in any of the method above, compounds provided herein have the formula II:

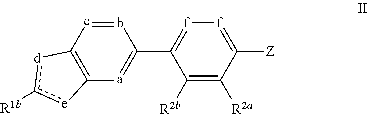

or a pharmaceutically acceptable salt, solvate or hydrate thereof, wherein
one of the two dashed bonds is a single bond and the other of the two dashed bonds is a double bond;
each of ring vertices a, b and c is independently selected from the group consisting of C(R^{1a}) and N;
each of ring vertices d and e is independently selected from the group consisting of C(R^{1b}) N, NH, N(C₁₋₄ alkyl) and N(C₁₋₄ haloalkyl) provided at least of d and e is other than C(R^{1b});
each ring vertex f is selected from the group consisting of C(R^{2c}), C(R^{2d}) and N;
is selected from the group consisting of:

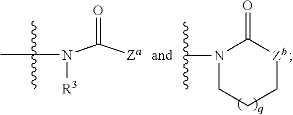

Z^{a} is selected from the group consisting of:
   (i) a 5- or 6-membered heteroaryl group having at least one nitrogen atom as a ring member, which is optionally substituted with from 1 to 4 R⁴;
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
   (iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —NO₂, —R^{c}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b};
wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, and C₂₋₆ alkynyl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;
   and wherein the aliphatic and cyclic portions of R^{d}, R^{e} and R^{f} are optionally further substituted with from one to three halogen, hydroxy, methyl, amino, methylamino, dimethylamino and carboxylic acid groups.

Embodiment 18: Within Embodiment 18, in any of the methods above, in another group of embodiments, compounds provided herein have the formula II:

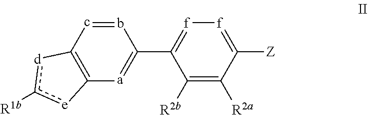

or a pharmaceutically acceptable salt, solvate or hydrate thereof, wherein
one of the dashed bonds is a single bond and the other of the dashed bonds is a double bond;
each of ring vertices a, b and c is independently selected from the group consisting of C(R^{1a}) and N;
each of ring vertices d and e is independently selected from the group consisting of C(R^{1b}) N, NH, N(C₁₋₄ alkyl) and N(C₁₋₄ haloalkyl) provided at least of d and e is other than C(R^{1b});
each ring vertex f is selected from the group consisting of C(R^{2c}), C(R^{2d}) and N;
Z is selected from the group consisting of:

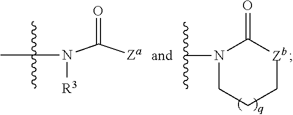

Z^{a} is selected from the group consisting of:
   (i) a 5- or 6-membered heteroaryl group having at least one nitrogen atom as a ring member, which is optionally substituted with from 1 to 4 R⁴; and
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —N^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, phenyl and heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;

and wherein the aliphatic and cyclic portions of R^{d}, R^{e} and R^{f} are optionally further substituted with from one to three halogen, hydroxy, methyl, amino, methylamino, dimethylamino and carboxylic acid groups.

In a first subembodiment of Embodiment 18, compounds of formula II are represented by a formula selected from the group consisting of II-1a to II-1h:

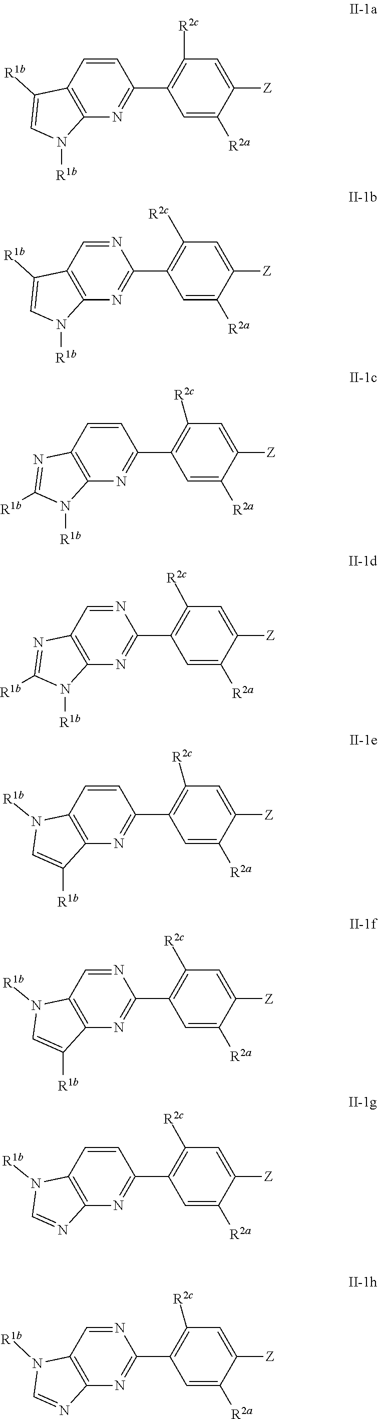

In certain of these embodiments, the compound has formula II-1a. In other of these embodiments, the compound has formula II-1b. In still other of these embodiments, the compound has formula II-1c. In yet other of these embodiments, the compound has formula II-1d. In certain of these embodiments, the compound has formula II-1e. In certain of these embodiments, the compound has formula II-1f. In certain of these embodiments, the compound has formula II-1g. In certain of these embodiments, the compound has formula II-1h.

(i) Within Embodiments 17 and 18, and embodiments and subembodiments contained therein, in one selected group of embodiments, Z is:

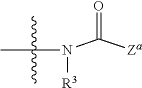

wherein Z^{a} is a 5- or 6-membered heteroaryl group having at least one nitrogen atom as a ring member, which is optionally substituted with from 1 to 4 R⁴.

Within embodiments in group (i), in one selected group of embodiments, compounds are provided wherein Z^{a} is selected from the group consisting of pyrazolyl, imidazolyl, oxazolyl, isoxazolyl, 1,2,3-triazolyl, 1,2,4-triazolyl, pyridinyl, pyrimidinyl, pyridazinyl and pyrazinyl, each of which is substituted with from 0 to 2 R⁴.

Within embodiments in group (i), in another selected group of embodiments, compounds are provided wherein Z^{a} is pyrazolyl or pyridinyl, each of which is substituted with 0 to 3 R⁴.

Within embodiments in group (i), in still another selected group of embodiments compounds are provided wherein Z^{a} is pyrazolyl substituted with 0 to 3 R⁴. With this group of embodiments in further embodiments compounds are provided wherein Z^{a} is pyrazol-5-yl substituted with 1 or 2 R⁴.

Within embodiments in group (i), in still another further selected group of embodiments compounds are provided wherein Z^{a} is pyridinyl substituted with 0 to 3 R⁴. With this group of embodiments in further embodiments compounds are provided wherein Z^{a} is pyridin-3yl or pyridin-4-yl substituted with 1 or 2 R⁴. Within this group of embodiments in still further embodiments compounds are provided wherein Z^{a} is pyridin-3-yl or pyridin-4-yl substituted with 1 or 2 R⁴ wherein at least 1 R⁴ is located carbon at the 2-position of the pyridin-3-yl or pyridin-4-yl ring.

Within embodiments in group (i) and further embodiments contained therein, in further selected group of embodiments compounds are provided wherein R³ is selected from the group consisting of hydrogen, C₁₋₃ alkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}. In a further selected group of embodiments, compounds are provided wherein R³ is hydrogen or methyl.

Within embodiments in group (i) and further embodiments contained therein, in further selected group of embodiments compounds are provided each R⁴ is independently selected from the group consisting of halogen, —CN, —R^{f}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y; wherein each X¹ is independently C₁₋₆ alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R is independently selected from the group consisting of C₁₋₈ alkyl.

(ii) Within Embodiments 16 and 17, and embodiments and subembodiments contained therein, in another selected group of embodiments, Z is:

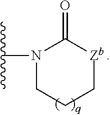

Within embodiments in group (ii), in a further selected group of embodiments compounds are provided wherein Z^{b} is C(R^{z})₂, wherein each R^{z} is independently selected from the group consisting of H, C₁₋₄ alkyl, C₁₋₄ haloalkyl and C₁₋₄ alkoxy. In still other selected embodiments, q is 1 or 2.

Within embodiments in group (ii), in another further selected group of embodiments compounds are provided wherein Z^{b} is N(R^{z}), wherein each R^{z} is independently selected from the group consisting of H, C₁₋₄ alkyl, C₁₋₄ haloalkyl and C₁₋₄ alkoxy. In still other selected embodiments, q is 1 or 2.

Within embodiments in group (ii), in further embodiments, compounds are provided wherein Z^{b} is selected from the group consisting of NH, N(CH₃) and CH₂. In still other selected embodiments, q is 1 or 2.

Within embodiments in group (ii), in yet another further selected group of embodiments compounds are provided wherein Z^{a} is O. In still other selected embodiments, q is 1 or 2.

(iii) Within Embodiments 16 and 17, and groups of embodiments contained therein above (i.e., (i) and (ii) and embodiments therein), in some selected embodiments, each R^{1a} is hydrogen.

(iv) Within Embodiments 16 and 17, and groups of embodiments contained therein above (i.e., (i), (ii), and (iii) and embodiments therein), in other selected embodiments, only one or two of R^{2a}, R^{2b}, R^{2c} and R^{2d} are other than hydrogen. In still other selected embodiments, only one or two of R^{2a}, R^{2b}, R^{2c} and R^{2d} is other than hydrogen, and are selected from the group consisting of F and CH₃. In still other selected embodiments, only R^{2a} is other than hydrogen, and is selected from the group consisting of F and CH₃.

(v) Within Embodiments 16 and 17, and groups of embodiments contained therein, in another selected group of embodiments (i.e., (i), (ii), (iii), and (iv) and embodiments therein), at least one R^{1b} is selected from the group consisting of C₁₋₄ haloalkyl or C₁₋₄ haloalkoxy.

(vi) Within Embodiments 16 and 17, and groups of embodiments contained therein above (i.e., (i), (ii), (iii), (iv), and (v) and embodiments therein), in another selected group of embodiments, one of R^{1b} is other than hydrogen and is C₁₋₄ haloalkyl (e.g. CF₃) or C₁₋₄ haloalkoxy (e.g., trifluoromethoxy), each R^{1a} is hydrogen, one of R^{2a}, R^{2b}, R^{2c} and R^{2d} are other than hydrogen and are independently selected from the group consisting of F and CH₃. In a further group of embodiments, R^{2a} is methyl.

Embodiment 19: In embodiment 19, in any of the methods disclosed above, in another group of embodiments, compounds provided herein have the formula III:

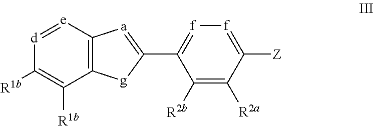

or a pharmaceutically acceptable salt, solvate or hydrate thereof, wherein:
ring vertex a is selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
ring vertex g is selected from the group consisting of O, S and N(R^{1a});
Z is selected from the group consisting of:

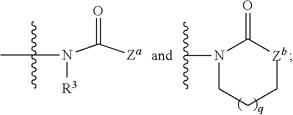

Z^{a} is selected from the group consisting of:
   (i) a 5- or 6-membered heteroaryl group having at least one nitrogen atom as a ring member, which is optionally substituted with from 1 to 4 R⁴;
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
   (iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —R^{c}, —OC(O)NR^{a}R^{b}, —NR^{b}C(O)₂R^{c}, —NR^{a}C(O)NR^{a}R^{b}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, and C₂₋₆ alkynyl, and wherein the aliphatic and cyclic portions of R^{a}, R^{b} and R are optionally further substituted with from one to three halogen, hydroxy, methyl, amino, methylamino, and dimethylamino groups; and at least one of R^{1b} is other than hydrogen;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{d}C(O)NR^{d}R^{e}, —NR^{d}R^{e}, —OR^{d}, —S(O)₂NR^{d}R^{e}, —X¹—CN, —X¹—CO₂R, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;
and wherein the aliphatic and cyclic portions of R^{d}, R^{e} and R^{f} are optionally further substituted with from one to three halogen, hydroxy, methyl, methylamino, and carboxylic acid groups

Embodiment 20: Within Embodiment 20, in any of the methods above, compounds provided herein have the Formula III:

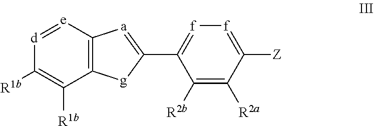

or a pharmaceutically acceptable salt, solvate or hydrate thereof, wherein:
ring vertex a is selected from the group consisting of C(R^{1a}) and N;
each of ring vertices d and e is independently selected from the group consisting of C(R^{1b}) and N;
each ring vertex f is selected from the group consisting of C(R^{2c}), C(R^{2d}) and N;
ring vertex g is selected from the group consisting of O, S and N(R^{1a});
Z is selected from the group consisting of:

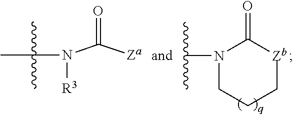

Z^{a} is a 5- or 6-membered heteroaryl group having at least one nitrogen atom as a ring member, which is optionally substituted with from 1 to 4 R⁴;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —R^{c}, —CONR^{a}R^{b}, —C(O)R^{a}, —OC(O)NR^{a}R^{b}, —NR^{b}C(O)₂R^{c}, —NR^{a}C(O)NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and heteroaryl, and wherein the aliphatic and cyclic portions of R^{a}, R^{b} and R are optionally further substituted with from one to three halogen, hydroxy, methyl, amino, methylamino, and dimethylamino groups and at least one of R^{1b} is other than hydrogen;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy; R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;
   and wherein the aliphatic and cyclic portions of R^{d}, R^{e} and R^{f} are optionally further substituted with from one to three halogen, hydroxy, methyl, amino, methylamino, dimethylamino and carboxylic acid groups.

In a first subembodiment of Embodiment 20, compounds of formula III are represented by a formula selected from the group consisting of III-1a to III-1d:

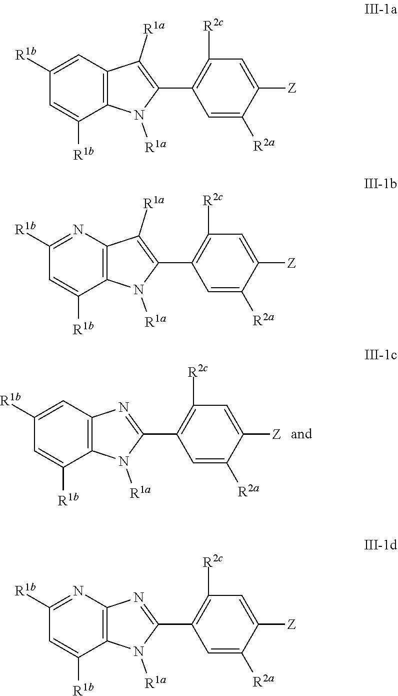

In certain of these embodiments, the compound has formula III-1a. In other of these embodiments, the compound has formula III-1b. In still other of these embodiments, the compound has formula III-1c. In yet other of these embodiments, the compound has formula III-1d.

In a second subembodiment of Embodiment 20, compounds of formula III are represented by a formula selected from the group consisting of III-2a to III-2h:

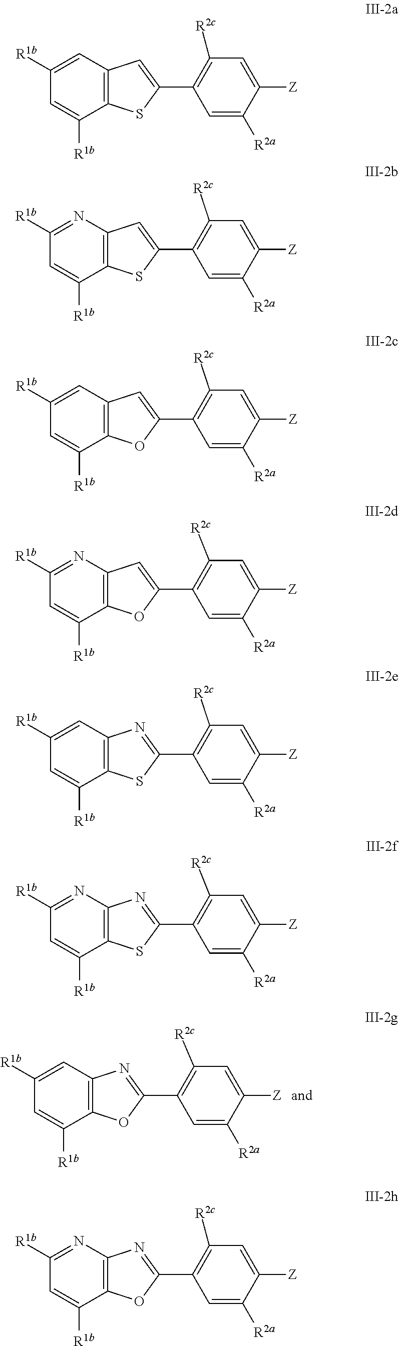

In certain of these embodiments, the compound has formula III-2a. In other of these embodiments, the compound has formula III-2b. In still other of these embodiments, the compound has formula III-2c. In yet other of these embodiments, the compound has formula III-2d. In yet other of these embodiments, the compound has formula III-2e. In yet other of these embodiments, the compound has formula III-2f. In yet other of these embodiments, the compound has formula III-2g. In yet other of these embodiments, the compound has formula III-2h.

(i) Within Embodiments 19 and 20 and groups of embodiments and subembodiments contained within, in one selected group of embodiments, Z is:

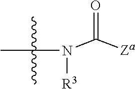

wherein Z^{a} is a 5- or 6-membered heteroaryl group having at least one nitrogen atom as a ring member, which is optionally substituted with from 1 to 4 R⁴.

Within embodiments in group (i), in one selected group of embodiments compounds are provided wherein Z^{a} is selected from the group consisting of pyrazolyl, imidazolyl, oxazolyl, isoxazolyl, 1,2,3-triazolyl, 1,2,4-triazolyl, pyridinyl, pyrimidinyl, pyridazinyl and pyrazinyl, each of which is substituted with from 0 to 2 R⁴.

Within embodiments in group (i), in another selected group of embodiments compounds are provided wherein Z^{a} is pyrazolyl or pyridinyl, each of which is substituted with 0 to 3 R⁴.

Within embodiments in group (i), in still another selected group of embodiments compounds are provided wherein Z^{a} is pyrazolyl substituted with 0 to 3 R⁴. With this group of embodiments in further embodiments compounds are provided wherein Z^{a} is pyrazol-5-yl substituted with 1 or 2 R⁴.

Within embodiments in group (i), in still another further selected group of embodiments compounds are provided wherein Z^{a} is pyridinyl substituted with 0 to 3 R⁴. With this group of embodiments in further embodiments compounds are provided wherein Z^{a} is pyridin-3yl or pyridin-4-yl substituted with 1 or 2 R⁴. With this group of embodiments in still further embodiments compounds are provided wherein Z^{a} is pyridin-3yl or pyridin-4-yl substituted with 1 or 2 R⁴ wherein at least 1 R⁴ is located carbon at the 2-position of the pyridin-3yl or pyridin-4-yl ring.

Within embodiments in group (i) and further embodiments contained therein, in further selected group of embodiments compounds are provided wherein R³ is selected from the group consisting of hydrogen, C₁₋₃ alkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}. In one selected group of embodiments compounds are provided wherein R³ is hydrogen or methyl.

Within embodiments in group (i) and further embodiments contained therein, in further selected group of embodiments compounds are provided each R⁴ is independently selected from the group consisting of halogen, —CN, —R^{f}, —CO₂R^{d}, —CONR^{d}R^{e}, —OR^{d}, —X¹—CN, —X¹—CO₂R, —X¹—CONR^{d}R^{e}, —X¹—OR^{d}, and —X¹—Y; wherein each X¹ is independently C₁₋₆ alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R^{f} is independently selected from the group consisting of C₁₋₈ alkyl.

(ii) Within Embodiments 19 and 20 and groups of embodiments and subembodiments contained within, in another selected group of embodiments, Z is:

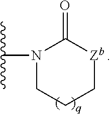

Within group (ii) embodiments and further embodiments contained therein, in further selected group of embodiments compounds are provided wherein Z^{a} is C(R^{z})₂, wherein each R^{z} is independently selected from the group consisting of H, C₁₋₄ alkyl, C₁₋₄ haloalkyl and C₁₋₄ alkoxy. In still other selected embodiments, q is 1 or 2.

Within group (ii) embodiments and further embodiments contained therein, in another further selected group of embodiments compounds are provided wherein Z^{a} is N(R^{z}), wherein each R^{z} is independently selected from the group consisting of H, C₁₋₄ alkyl, C₁₋₄ haloalkyl and C₁₋₄ alkoxy. In still other selected embodiments, q is 1 or 2.

Within group (ii) embodiments and further embodiments contained therein, in further embodiments, compounds are provided wherein Z^{b} is selected from the group consisting of NH, N(CH₃) and CH₂. In still other selected embodiments, q is 1 or 2.

Within group (ii) embodiments and further embodiments contained therein, in yet another further selected group of embodiments compounds are provided wherein Z^{a} is O. In still other selected embodiments, q is 1 or 2.

(iii) Within Embodiments 19 and 20 and groups of embodiments and subembodiments contained therein above (i.e., (i) and (ii) and embodiments therein), in some selected embodiments, only one or two of R^{2a}, R^{2b}, R^{2c} and R^{2d} is other than hydrogen.

(iv) Within Embodiments 19 and 20 and groups of embodiments and subembodiments contained therein above (i.e., (i) and (ii) and embodiments therein), in some selected embodiments, only one or two of R^{2a}, R^{2b}, R^{2c} and R^{2d} is other than hydrogen, and are independently selected from the group consisting of F and CH₃.

(v) Within Embodiments 19 and 20 and groups of embodiments and subembodiments contained therein (i.e., (i), (ii), (iii), and (iv) and embodiments therein), in some selected embodiments, at least one R^{1b} is selected from the group consisting of halo, C₁₋₄ haloalkyl, or C₁₋₄ haloalkoxy.

(vi) Within Embodiments 19 and 20 and groups of embodiments and subembodiments contained therein (i.e., (i), (ii), (iii), (iv) and (v) and embodiments therein), in another selected group of embodiments, one or two of R^{1b} are other than hydrogen and are independently selected from hydrogen, halo, C₁₋₄ haloalkyl (e.g. CF₃) and C₁₋₄ haloalkoxy (e.g., trifluoromethoxy), each R^{1a} is hydrogen or methyl, one or two of R^{2a}, R^{2b}, R^{2c} and R^{2d} are other than hydrogen and are independently selected from the group consisting of F and CH₃. In some selected embodiments, R^{2a} is methyl.

Embodiment 21: Within Embodiment 21, in any of the methods above compounds provided herein have the formula IV:

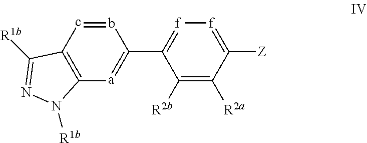

or a pharmaceutically acceptable salt, solvate or hydrate thereof, wherein:
each of ring vertices a, b and c is independently selected from the group consisting of C(R^{1a}) and N;
each ring vertex f is selected from the group consisting of C(R^{2c}), C(R^{2d}) and N;
Z is selected from the group consisting of:

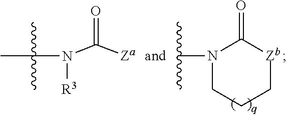

Z^{a} is selected from the group consisting of:
   (i) a 5- or 6-membered heteroaryl group having at least one nitrogen atom as a ring member, which is optionally substituted with from 1 to 4 R⁴;
   (ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
   (iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, and C₂₋₆ alkynyl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{b}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;
   and wherein the aliphatic and cyclic portions of R^{d}, R^{e} and R^{f} are optionally further substituted with from one to three halogen, hydroxy, methyl, amino, methylamino, dimethylamino and carboxylic acid groups.

Embodiment 22: Within Embodiment 22, in any of the methods above, compounds provided herein have the formula IV:

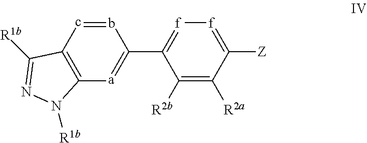

or a pharmaceutically acceptable salt, solvate or hydrate thereof, wherein:
each of ring vertices a, b and c is independently selected from the group consisting of C(R^{1a}) and N;
each ring vertex f is selected from the group consisting of C(R^{2c}), C(R^{2d}) and N;
Z is selected from the group consisting of:

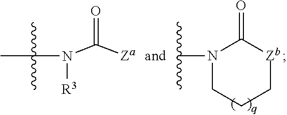

Z^{a} is a 5- or 6-membered heteroaryl group having at least one nitrogen atom as a ring member, which is optionally substituted with from 1 to 4 R⁴;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, and C₂₋₆ alkynyl, and wherein the aliphatic and cyclic portions of R^{a}, R^{b} and R are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{e}C(O)₂R^{f}, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, aryl and heteroaryl;
and wherein the aliphatic and cyclic portions of R^{d}, R^{e} and R^{f} are optionally further substituted with from one to three halogen, hydroxy, methyl, amino, methylamino, dimethylamino and carboxylic acid groups.

In one subembodiment of Embodiment 22, compounds of formula IV are represented by a formula selected from the group consisting of IV-1a and IV-1b:

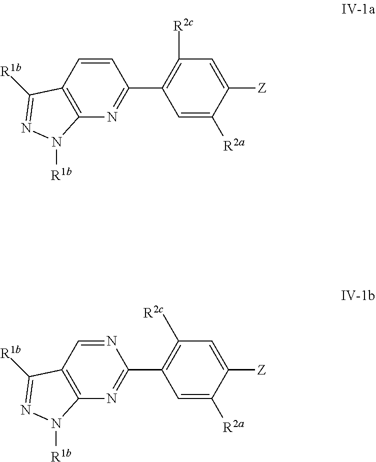

In certain of these embodiments, the compound has formula IV-1a. In other of these embodiments, the compound has formula IV-1b.

(i) Within Embodiments 21 and 22 and groups of embodiments contained therein, in one selected group of embodiments, Z is:

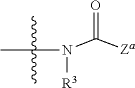

wherein Z^{a} is a 5- or 6-membered heteroaryl group having at least one nitrogen atom as a ring member, which is optionally substituted with from 1 to 4 R⁴.

Within embodiments in group (i) of embodiments, in another selected group of embodiments compounds are provided wherein Z^{a} is selected from the group consisting of pyrazolyl, imidazolyl, oxazolyl, isoxazolyl, 1,2,3-triazolyl, 1,2,4-triazolyl, pyridinyl, pyrimidinyl, pyridazinyl and pyrazinyl, each of which is substituted with from 0 to 2 R⁴.

Within embodiments in group (i) of embodiments, in another selected group of embodiments compounds are provided wherein Z^{a} is pyrazolyl or pyridinyl, each of which is substituted with 0 to 3 R⁴.

Within embodiments in group (i) of embodiments, in still another selected group of embodiments compounds are provided wherein Z^{a} is pyrazolyl substituted with 0 to 3 R⁴. With this group of embodiments in further embodiments compounds are provided wherein Z^{a} is pyrazol-5-yl substituted with 1 or 2 R⁴.

Within embodiments in group (i) of embodiments, in still another further selected group of embodiments compounds are provided wherein Z^{a} is pyridinyl substituted with 0 to 3 R⁴. With this group of embodiments in further embodiments compounds are provided wherein Z^{a} is pyridin-3yl or pyridin-4-yl substituted with 1 or 2 R⁴. With this group of embodiments in still further embodiments compounds are provided wherein Z^{a} is pyridin-3yl or pyridin-4-yl substituted with 1 or 2 R⁴ wherein at least 1 R⁴ is located carbon at the 2-position of the pyridin-3-yl or pyridin-4-yl ring.

Within embodiments in group (i) and further embodiments contained therein, in further selected group of embodiments compounds are provided wherein R³ is selected from the group consisting of hydrogen, C₁₋₃ alkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}. In one selected group of embodiments compounds are provided wherein R³ is hydrogen or methyl.

Within embodiments in group (i) and further embodiments contained therein, in another further selected group of embodiments compounds are provided each R⁴ is independently selected from the group consisting of halogen, —CN, —R^{f}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y;

wherein each X¹ is independently C1.6 alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R is independently selected from the group consisting of C₁₋₈ alkyl.

(ii) Within Embodiments 21 and 22 and groups of embodiments and subembodiments contained therein (i.e., (i), (ii), (iii), and (iv) and embodiments therein), in another selected group of embodiments, Z is:

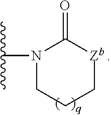

Within embodiments in group (ii), in further selected group of embodiments compounds are provided wherein Z^{a} is C(R^{z})₂, wherein each R^{z} is independently selected from the group consisting of H, C₁₋₄ alkyl, C₁₋₄ haloalkyl and C₁₋₄ alkoxy. In still other selected embodiments, q is 1 or 2.

Within embodiments in group (ii), in another further selected group of embodiments compounds are provided wherein Z^{a} is N(R^{z}), wherein each R^{z} is independently selected from the group consisting of H, C₁₋₄ alkyl, C₁₋₄ haloalkyl and C₁₋₄ alkoxy. In still other selected embodiments, q is 1 or 2.

Within embodiments in group (ii), in further embodiments, compounds are provided wherein Z^{b} is selected from the group consisting of NH, N(CH₃) and CH₂. In still other selected embodiments, q is 1 or 2.

Within embodiments in group (ii), in yet another further selected group of embodiments compounds are provided wherein Z^{a} is O. In still other selected embodiments, q is 1 or 2.

(iii) Within Embodiments 21 and 22 and groups of embodiments and subembodiments contained therein above (i.e., (i) and (ii) and embodiments therein), in some selected embodiments, each R^{1a} is hydrogen.

(iv) Within Embodiments 21 and 22 and groups of embodiments and subembodiments contained therein above (i.e., (i) and (ii) and embodiments therein), only one or two of R^{2a}, R^{2b}, R^{2c} and R^{2d} are other than hydrogen. In still other selected embodiments, only one or two of R^{2a}, R^{2b}, R^{2c} and R^{2d} are other than hydrogen, and is selected from the group consisting of F and CH₃.

(v) Within Embodiments 21 and 22 and groups of embodiments and subembodiments contained therein above (i.e., (i) and (ii) and embodiments therein), in another selected group of embodiments, at least one R^{1b} is selected from the group consisting of C₁₋₄ alkyl, C₁₋₄ haloalkyl or C₁₋₄ haloalkoxy.

(vi) Within Embodiments 21 and 22 and groups of embodiments and subembodiments contained therein above (i.e., (i), (ii), (iii), (iv) and (v) and embodiments therein), in another selected group of embodiments, one of R^{1b} is other than hydrogen and is C₁₋₄ alkyl (e.g., methyl), C₁₋₄ haloalkyl (e.g. CF₃) or C₁₋₄ haloalkoxy (e.g., trifluoromethoxy), each R^{1a} is hydrogen, one or two of R^{2a}, R^{2b}, R^{2c} and R^{2d} are other than hydrogen and are independently selected from the group consisting of F and CH₃. In a further group of embodiments, R^{2a} is methyl.

With reference to the Embodiments 17, 18, 19, 20, 21 and 22, and the embodiments and subembodiments discussed therein, selected embodiments are those wherein each R⁴, when present, is independently selected from the group consisting of halogen, —CN, —R^{e}, —CO₂R^{d}, —CONR^{d}R^{e}, —OR^{d}, —X¹—CN, —X¹—CO₂R^{d}, —X¹—CONR^{d}R^{e}, —X¹—OR^{d}, and —X¹—Y; wherein each X¹ is independently C₁₋₆ alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and each R^{d} and R^{e} is independently selected from hydrogen or C₁₋₈ alkyl; and each R^{f} is independently selected from the group consisting of C₁₋₈ alkyl.

In some selected embodiments, in any of the methods above, the compounds are those in Table 1 having +++ or ++++ activity.

### Stem Cell Sources for In Vitro or Ex Vivo Expansion:

The starting stem cells and/or lineage committed progenitor cells population for use in the in vitro and ex vivo methods disclosed herein are either commercially available or can be isolated from various tissues and/or organs of a mammal or can be prepared by method well known in the art.

For example, embryonic stem cells (ESCs) are derived from the inner cell mass of an embryo (see Thomson et. al, Science. 1998 Nov. 6; 282(5391):1145-7) whereas induced pluripotent stem cells (iPSCs) are derived from somatic cells (see Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20).

Hematopoietic stem cells and/or lineage committed progenitor cells thereof can be isolated from bone marrow, umbilical cord, peripheral blood, liver, thymus, lymph, and spleen. Mesenchymal stem cells and/or lineage committed progenitor cells thereof can be isolated from bone marrow, umbilical cord, adipose tissue, molar cells, and amniotic fluid. In some embodiments, the stem cells are isolated from a patient in need of stem cell therapy. Stems cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

The aforementioned crude or un-fractionated sources containing the desired stem cells can be used in the methods disclosed herein directly after isolation or after purification and/or enrichment for a specific type of stem cell. The purification and/or enrichment can be based on presence or absence of specific cellular marker(s). Methods for enriching stem cells are known to those of skill in the art e.g., flow cytometry. In some embodiments, the starting population of hematopoietic stem cells are enriched in Endothelial Protein C Receptor (EPCR+) and/or CD34+, CD38+, CD90+, CD45RA+, CD133 and/or CD49f+ cells. In some embodiments, the hematopoietic stem cells are enriched in CD34+ cells and/or EPCR+ cells. In some embodiments, the hematopoietic stem cells are enriched in CD34+ cells. Enriched as used above means at least about 10%, 20% 30%, 50%, 60%, 70%, 80% or 90% of HSCs express one or more of above listed markers. In some embodiment, the starting population of cells for expansion for use in the methods disclosed herein consist essentially of CD34+ cells. With respect to MSCs, in some embodiments, the starting MSCs are positive for one or more of CD105, CD90, CD44, CD73, CD29, CD13, CDE34, CD146, CD106, CD54 and CD166 markers. In some embodiments, the MSCs are at least over 30%, 50%, 60%, 70%, 80% or 90% are CD106+ and preferably CD45− cells. In some embodiments, an expanded stem cell and/or lineage committed progenitor cell population accordingly to the methods disclosed herein can also serve as a starting population for further expansion according to the methods disclosed herein.

“Genetically modified stem cell(s)” refers to a naturally occurring stem cell that has exogenous nucleic acid artificially introduced inside the cell and/or one or more endogenous gene(s) artificially, either completely or partially, deleted, replaced, and/or one or more endogenous gene(s) mutated. Genetically modified stem cells are prepared from naturally occurring stem cells by methods well known in the art. For example, exogenous nucleic acid can be introduced into the target stem cell by viral or non-viral gene transfer, including but not limited to, electroporation, lipofection, sonoporation, gene gun, plasmid transfer, phage integrase, transposons, AdV, AAV, and Lentivirus transfection, and ZNF, TALENs, and CRISPR/Cas, (preferably Cas9) methodologies. ZNF, TALENs, and CRISPR/Cas9 methodologies can also be used to partially or fully delete and/or replace a gene(s) from a target cell (see for example PCT Applications publication nos. WO 2015/057976 and WO2016/182959). In some embodiments, the genetically modified stem cells are made after culturing of naturally occurring stem cells with a compound of Formula I. In some embodiments, the genetically modified stem cells are made prior to culturing of naturally occurring stem cells with a compound of Formula I. In some embodiments, the genetically modified stem cells are made after expansion of naturally occurring stem cells according to the methods disclosed herein. In some embodiments, the genetically modified stem cells are made prior to expansion of naturally occurring stem cells according to the methods disclosed herein. In some embodiments, the exogenous nucleic acid is integrated (covalently linked) into the genome of the modified cells. In some embodiments, the exogenous nucleic acid is not integrated (covalently linked) into the genome of the modified cells. In some embodiments, the exogenous nucleic acid comprises one or more genes and is integrated into the genome of the modified cell. In some embodiments, the exogenous nucleic acid can be a complete gene(s) or a fragment thereof. In some embodiments, the exogeneous nucleic acid encodes a molecule selected from the group consisting of a protein and RNA (e.g., mRNA, siRNA, miRNA, or shRNA).

In some embodiments, one or more endogenous genes that is a member of a pathway whose activity is inhibited (e.g., TGFβ pathway, histone demethylase pathway) in the methods disclosed herein is fully or partially deleted. In some embodiments, the exogenous nucleic acid comprises one or more genes that expresses a product (e.g., protein, RNAi, mRNA) having a therapeutic utility. For example, a nucleic acid that expresses RNAi or mRNA that can silence the expression of an endogenous gene involved in a disease pathway is inserted. In some embodiments, the exogenous nucleic acid, preferably a gene is introduced and or an endogenous gene is deleted or replaced using ZNF, TALENs, and/or CRISPR/Cas9 methodology.

In yet another aspect, provided is a method of making a genetically modified stem cell comprising culturing the naturally occurring stem cell with a compound of Formula I (or any embodiment thereof disclosed herein). In some embodiment, the cell is modified after it is cultured with a compound of Formula I (or any embodiment thereof disclosed herein). In some embodiment, the cell is modified before it is contacted with a compound of Formula I (or any embodiment thereof disclosed herein). In any of the above embodiments, the stem cell that is HSC. In any of the above embodiments, the cell is modified using viral transfection, ZNF, TALENs, and/or CRISPR/Cas (preferably CRISPR/Cas9) methodology. In any of the above embodiments, the cell is modified for transplantation into a patient in need thereof.

### ADDITIONAL EMBODIMENTS

Embodiment 23: Embodiment 23 is directed to the method of the first aspect described above. Within embodiment 23, in a first subembodiment, the method of embodiment 23 is wherein the stem cells and/or lineage committed progenitor cells, are cultured under conditions that maintain functional potential of the stem cells and/or lineage committed progenitor cells.

Embodiment 24: Embodiment 24 is directed a method for producing an expanded population of stem cells and/or lineage committed progenitor cells in vitro or ex vivo comprising culturing a population of the stem cells and/or lineage committed progenitor cells in a medium comprising a compound of Formula I, II, III or IV (and subembodiments thereof contained herein above), wherein the compound of Formula I, II, III or IV (and subembodiments thereof contained herein above) antagonizes the activity of aryl hydrocarbon receptor; and the stem cells and/or progenitor cells are cultured under conditions allowing expansion of the stem cells and/or progenitor cells. Within embodiment 24, in a first subembodiment, the method of embodiment 24 further comprises differentiating the expanded stem cells to lineage committed progenitor cells thereof under conditions that cause differentiation of the expanded stem cells to lineage committed progenitor cells thereof.

Embodiment 25: In embodiment 25, the method of any one of embodiments 23 and 24, and subembodiments contained therein, is wherein the method is carried out ex vivo.

Embodiment 26: In embodiment 26, the method of any one of embodiments 23, 24, 25, and any subembodiments contained therein, is wherein the stem cells and lineage committed progenitor cells are human cells.

Embodiment 27: In embodiment 27, the method of any one of embodiments 23 to 26, and any subembodiments contained therein, is wherein the stem cells and/or lineage committed progenitor cells are hematopoietic stem cells and/or lineage committed hematopoietic progenitor cells. In a subembodiment of embodiment 27, the stem cells and/or lineage committed progenitor cells are hematopoietic stem cells and lineage committed hematopoietic progenitor cells thereof.

Embodiment 28: In embodiment 28, the method of any one of embodiments 23 to 26, and any subembodiments contained therein, is wherein the stem cells and/or lineage committed progenitor cells are genetically modified hematopoietic stem cells and/or lineage committed progenitor cells thereof. In one subembodiment of embodiment 28, the genetically modified hematopoietic stem cells and/or lineage committed genetically modified hematopoietic progenitor cells thereof comprise an exogenous nucleic acid. In a second subembodiment of embodiment 28, the genetically modified hematopoietic stem cells and/or lineage committed genetically modified hematopoietic progenitor cells thereof comprise an exogenous nucleic acid that is integrated in the genome of the modified cells.

Embodiment 29: In embodiment 29, the method of any one of embodiments 27 or 28, and any subembodiments contained therein, further comprises culturing the (i) hematopoietic stem cells and/or lineage committed hematopoietic progenitor cells or (ii) genetically modified hematopoietic stem cells and/or lineage committed genetically modified hematopoietic progenitor cells in the presence of a Notch agonist. Within embodiment 29, in a first subembodiment, the Notch agonist is an extracellular domain of a Delta protein or a Jagged protein or a Notch-binding portion of extracellular domain of a Delta protein or a Jagged protein optionally fused to Fc region of an IgG. Within embodiment 29, in a second subembodiment, the Notch agonist is Delta-^{ext-IgG}.

Embodiment 30: In embodiment 30, the method of any one of embodiments 27 to 29, and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits TGFβ signaling. Within embodiment 30, in a first subembodiment, the agent is a TGFβ receptor inhibitor. Within embodiment 30, in a second subembodiment, the agent is a TGFβ receptor inhibitor selected from the group consisting of 2-[3-(6-methyl-2-pyridinyl)-1H-pyrazol-4-yl]-1,5-naphthyridine, 4-[3-(2-pyridinyl)-1H-pyrazol-4-yl]-quinoline, 4-[6-(4-isopropoxyphenyl)-pyrazolo[1,5-a]pyrimidin-3-yl]quinoline, and 3-(6-methyl-2-pyridinyl)-N-phenyl-4-(4-quinolinyl)-1H-pyrazole-1-carbothioamide. Within embodiment 30, in a third subembodiment, the TGFβ receptor inhibitor is 3-(6-methyl-2-pyridinyl)-N-phenyl-4-(4-quinolinyl)-1H-pyrazole-1-carbothioamide.

Embodiment 31: In embodiment 31, the method of any one of embodiments 27 to 30 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits histone demethylation. Within embodiment 31, in a subembodiment the agent is a histone demethylase inhibitor. Within embodiment 31, in a first subembodiment, the histone demethylase inhibitor is a LSD1 inhibitor. Within embodiment 31, in a second subembodiment, the histone demethylase inhibitor is selected from the group consisting of 2-(1R,2S)-2-(4-(benzyloxy)phenyl)cyclopropylamino)-1-(4-methylpiperazin-1-yl)ethanone, HCl (LSD1 inhibitor IV RN-1), 2-(2-(benzyloxy)-3,5-difluorophenyl)-cyclopropan-1-amine (LSD1 inhibitor II S2101), (1S,2S)—N-(1-(2,3-dihydrobenzo[b][1,4]-dioxin-6-yl)ethyl)-2-phenylcyclopropan-1-amine (LSDI inhibitor LSD1-C76), methyl-3-(4-(4-carbamimidoylbenzoyl)piperazine-1-carbonyl)-5-((4-carbamimidoylpiperazin-1-yl)methyl)-benzoate (LSDI inhibitor III Cnn1007), (E,E)-1,1′-((propane-1,3-diylbis(azanediyl))-bis(propane-3,1-diyl))bis(2,3-dimethylguanidine) (LSD1 inhibitor I), and Tranylcypromine. Within embodiment 31, in a third subembodiment, the histone demethylase inhibitor is Tranylcypromine.

Embodiment 32: In embodiment 32, the method of any one of embodiments 27 to 31 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits histone acetylation. Within embodiment 32, in a subembodiment, the agent is a histone deacetylase inhibitor. Within embodiment 32, in a first subembodiment, the histone deacetylase inhibitor is selected from the group consisting of Trichostatin A, valproic acid, butyrylhydroxamic acid, and istodax. Within embodiment 32, in a second subembodiment, the histone deacetylase inhibitor is Trichostatin A.

Embodiment 33: In embodiment 33, the method of any one of embodiments 27 to 32 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits p38 signaling and/or an agent that inhibits a protein that promotes β-catenin degradation. Within embodiment 33, in a first subembodiment, the p38 inhibitor is 4-(4-fluorophenyl)-2-(4-methylsulfinylphenyl)-5-(4-pyridyl)-1H-imidazole (SB203580). Within embodiment 33, in a second embodiment, the agent that inhibits β-catenin degradation is 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR99021), LiCl, BIO, or FGF2.

Embodiment 34: In embodiment 34, the method of any one of embodiments 27 to 33, and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that reduces the activity of an ikaros family member transcription factor. With embodiment 34, in a first subembodiment, the agent that reduces the activity of an ikaros family member transcription factor is pomalidomide, lenalidomide, or thalidoamide.

Embodiment 35: In embodiment 35, the method of any one of embodiments 27 to 34 and any subembodiments contained therein, further comprises culturing the population of (i) hematopoietic stem cells and/or lineage committed hematopoietic progenitor cells or (ii) genetically modified hematopoietic stems cells and/or lineage committed genetically modified hematopoietic progenitor cells under conditions that maintain the functional potential of the hematopoietic stem cells and/lineage committed hematopoietic progenitor cells or genetically modified hematopoietic stems cells, and/or lineage committed genetically modified hematopoietic progenitor cells.

Embodiment 36: In embodiment 36, the method of any one of embodiments 27 to 35, and any subembodiments contained therein, is wherein the hematopoietic stem cells (including those used for producing genetically modified hematopoietic stem cells) and/or lineage committed hematopoietic progenitor cells are from bone marrow, umbilical cord blood, or mobilized peripheral blood.

Embodiment 37: In embodiment 37, the method of any one of embodiments 27 to 35, and any subembodiments contained therein, is wherein the hematopoietic stem cells (including those used for producing genetically modified hematopoietic stem cells) and/or lineage committed hematopoietic progenitor cells are from bone marrow, umbilical cord blood, or mobilized peripheral blood. Within embodiment 37, in one subembodiment the hematopoietic stem cells, including those for producing genetically modified hematopoietic stem cells, are enriched in Endothelial Protein C Receptor (EPCR+) and/or CD34+, CD38+, CD90+, CD45RA+, CD133 and/or CD49f+. Within embodiment 37, in a second subembodiment, the hematopoietic stem cells, including those for producing genetically modified hematopoietic stem cells, are enriched in CD34+ cells and/or EPCR+. Within embodiment 37, in a third subembodiment, the hematopoietic stem cells, including those for producing genetically modified hematopoietic stem cells, are enriched in CD34+ cells.

Embodiment 38: In embodiment 38, the method of any one of embodiments 27 to 37, and any subembodiments contained therein, is wherein the hematopoietic stem cells, including those for producing genetically modified hematopoietic stem cells, consist essentially of CD34+ cells.

Embodiment 39: In embodiment 39, the method of any one of embodiments 27 to 38, and any subembodiments contained therein, further comprising culturing the (i) hematopoietic and/or lineage committed hematopoietic progenitor cells or (ii) genetically modified hematopoietic stem cells and/or lineage committed genetically modified hematopoietic progenitor cells in the presence of a sufficient amount of one or more of IL6, Flt-3-L, TPO, and SCF. Within embodiment 39, in a first subembodiment, the cells are cultured in the presence of IL6, Flt-3-L, TPO, and SCF.

Embodiment 40: In embodiment 40, the method of any one of embodiments 27 to 39, and any subembodiments contained therein, is wherein the amount of the compound of Formula I, II, III, or IV in the cell culture medium is from about 1 μm to about 100 uM. Within embodiment 40, in a first subembodiment, the amount of the compound of Formula I, II, III, or IV in the cell culture is from about 100 μm to about 10 um.

Embodiment 41: In embodiment 41, the method of any one of embodiments 27 to 40, and any subembodiments contained therein, is wherein the stem cells and/or lineage committed progenitor cells are cultured in the presence of a compound of Formula I, II, III, or IV (and subembodiments thereof contained herein above) from about 1 day to about 90 days. Within embodiment 41, in a first subembodiment, the stem cells and/or lineage committed progenitor cells are cultured from about 2 to about 35 days. Within embodiment 41, in a second subembodiment, the stem cells and/or lineage committed progenitor cells are cultured from about 2 to about 35 days. Within embodiment 41, in a third subembodiment, the stem cells and/or lineage committed progenitor cells are cultured from about 3 days to about 90 days. Within embodiment 41, in a fourth subembodiment, the stem cells and/or lineage committed progenitor cells are cultured from about 7 to about 35 days. Within embodiment 41, in a fourth subembodiment, the stem cells and/or lineage committed progenitor cells are cultured from about 7 to about 21 days. Within embodiment 41, in a fourth subembodiment, the stem cells and/or lineage committed progenitor cells are cultured from about 1 to about 3 days.

Embodiment 42: In embodiment 42, the method of any one of embodiments 27 to 41, and any subembodiments contained therein, is wherein the hematopoietic stem cells and/or lineage committed hematopoietic progenitor cells are cultured in the presence of a compound of Formula I, II, III, or IV (and subembodiments thereof contained herein above) during a time sufficient for about 2- to 50,000-fold expansion of hematopoietic cells, preferably CD34+ cells, and/or lineage committed hematopoietic progenitor cells as compared to a population of hematopoietic and/or lineage committed hematopoietic progenitor cells in the absence of a compound of Formula I, II, III, or IV (and subembodiments thereof contained herein above) respectively.

Embodiment 43: In embodiment 43, the method of any one of embodiments 27 to 42, and any subembodiments contained therein, is wherein the hematopoietic stem cells and/or lineage committed hematopoietic progenitor cells or genetically modified hematopoietic stem cells and/or lineage committed genetically modified hematopoietic progenitor cells are contacted with said one or more agents in embodiments 29 to 34 and 39 simultaneously.

Embodiment 44: In embodiment 44, the method of any one of embodiments 27 to 42, and any subembodiments contained therein, is wherein the hematopoietic stem cells and/or lineage committed hematopoietic progenitor cells or genetically modified hematopoietic stem cells and/or lineage committed genetically modified hematopoietic progenitor cells thereof are contacted with said one or more agents in embodiments 29 to 34 and 39 at different times.

Embodiment 45: In embodiment 45, the method of any one of embodiments 27 to 44 and any subembodiments contained therein, is wherein the hematopoietic stem cells and/or lineage committed hematopoietic progenitor cells, including those used to prepared genetically modified hematopoietic stem cells, are originally within a mononuclear cell fraction prior to treatment with said one or more agents.

Embodiment 46: In embodiment 46, the method of any one of embodiments 27 to 44 and any subembodiments contained therein, is wherein the hematopoietic stem cells, including those used to prepared genetically modified hematopoietic stem cells, are originally within a CD34+, CD34+CD38−, CD34+CD38−CD90+, CD34+CD38−CD90+CD45RA−, or CD34+CD38−CD90+CD45RA−CD49F+ enriched cell fraction prior to contacting said one or more agents.

Embodiment 47: In embodiment 47, the method of any one of embodiments 27 to 44 and any subembodiments contained therein, is wherein the hematopoietic stem cells and/or lineage committed progenitor cells, including those used to prepared genetically modified hematopoietic stem cells, are originally within an un-enriched cell fraction prior to contacting said one or more agents.

Embodiment 48: In embodiment 48, the method of any one of embodiments 1 to 47 and any subembodiments contained therein, is wherein the compound of Formula I, II, III, or IV is wherein one of ring vertices a, b, c, d and e is N.

Embodiment 49: In embodiment 49, the method of embodiment 48 and any subembodiments contained therein, wherein the compound of Formula I, II, III, or IV is wherein each ring vertex f is CH.

Embodiment 50: In embodiment 50, the method of any one of embodiments 1 to 47 and any subembodiments contained therein, is wherein the compound of Formula I, II, III, or IV is wherein two of ring vertices a, b, c, d and e are N.

Embodiment 51: In embodiment 51, the method of embodiments 50 and any subembodiments contained therein, is wherein the compound of Formula I, II, III, or IV is wherein each ring vertex f is CH.

Embodiment 52: In embodiment 52, the method of any one of embodiments 1 to 47 and any subembodiments contained therein, is wherein the compound of Formula I, II, III, or IV is wherein three of ring vertices a, b, c, d and e are N.

Embodiment 53: In embodiment 53, the method of embodiment 52 and any subembodiments contained therein, is wherein the compound of Formula I, II, III, or IV is wherein each ring vertex f is CH.

Embodiment 54: In embodiment 54, the method of any one of embodiments 1 to 53 and any subembodiments contained therein, is wherein the compound of I, II, III, or IV is wherein Z is

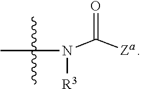

Embodiment 55: In embodiment 55, the method of embodiment 54 and any subembodiments contained therein, is wherein the compound of I, II, III, or IV is wherein Z^{a} is selected from the group consisting of pyrazole, imidazole, oxazole, isoxazole, 1,2,3-triazole, 1,2,4-triazole, pyridine, pyrimidine, pyridazine and pyrazine, each of which is optionally substituted with from 1 to 2 R⁴.

Embodiment 56: In embodiment 56, the method of any one of embodiments 1 to 47 and any subembodiments contained therein, is wherein the compound is a compound of Formula I is represented by formulae Ia, Ib, Ic, Id, Ie, If, Ig, and Ih:

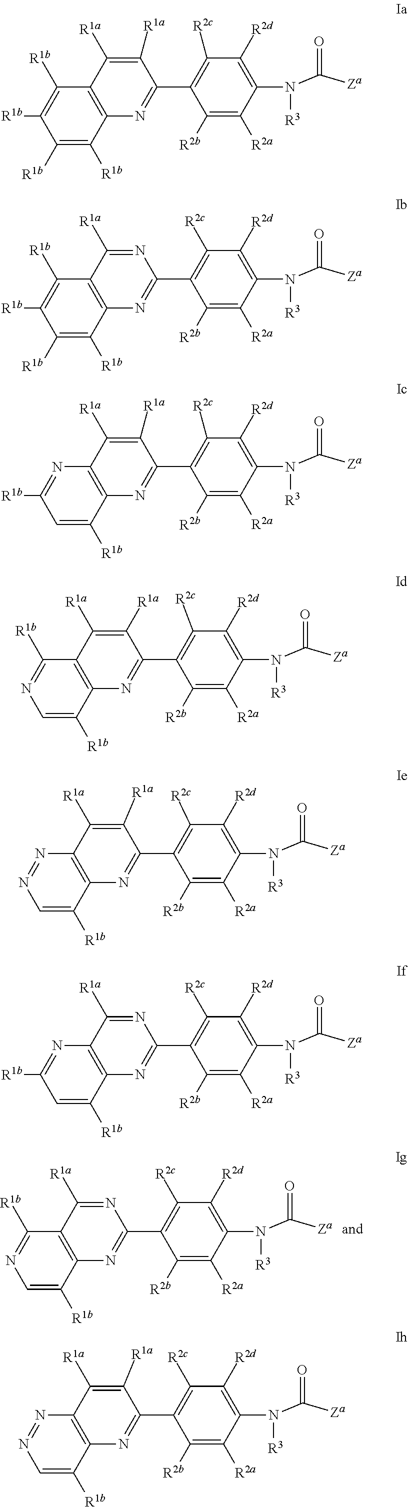

Embodiment 57: In embodiment 57, the method of embodiment 56 and any subembodiments contained therein, is wherein the compound is a compound of formula Ia wherein Z^{a} is pyrazole or pyridine, each of which is optionally substituted with from 1 to 3 R⁴.

Embodiment 58: In embodiment 58, the method of embodiment 56 and any subembodiments contained therein, is wherein the compound is a compound of formula Ib wherein Z^{a} is pyrazole or pyridine, each of which is optionally substituted with from 1 to 3 R⁴.

Embodiment 59: In embodiment 59, the method of embodiment 56 and any subembodiments contained therein, is wherein the compound is a compound of formula Ic wherein Z^{a} is pyrazole or pyridine, each of which is optionally substituted with from 1 to 3 R⁴.

Embodiment 60: In embodiment 60, the method of embodiment 56 and any subembodiments contained therein, is wherein the compound is a compound of formula Id wherein Z^{a} is pyrazole or pyridine, each of which is optionally substituted with from 1 to 3 R⁴.

Embodiment 61: In embodiment 61, the method of embodiment 56 and any subembodiments contained therein, is wherein the compound is a compound of formula Ie wherein Z^{a} is pyrazole or pyridine, each of which is optionally substituted with from 1 to 3 R⁴.

Embodiment 62: In embodiment 62, the method of embodiment 56 and any subembodiments contained therein, is wherein the compound is a compound of formula If wherein Z^{a} is pyrazole or pyridine, each of which is optionally substituted with from 1 to 3 R⁴.

Embodiment 63: In embodiment 63, the method of embodiment 56 and any subembodiments contained therein, is wherein the compound is a compound of formula Ig wherein Z^{a} is pyrazole or pyridine, each of which is optionally substituted with from 1 to 3 R⁴.

Embodiment 64: In embodiment 64, the method of embodiment 57 and any subembodiments contained therein, is wherein the compound of formula Ia has the structure:

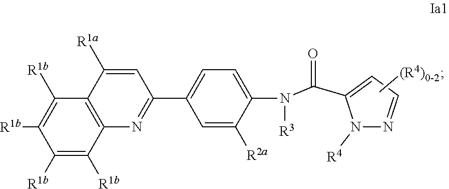

wherein
each R^{1a} and R^{1b} is independently selected from the group consisting of H, halogen,
—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
   R^{2a} is selected from the group consisting of H, F and CH₃;
   each R⁴ is independently selected from the group consisting of hydrogen,
halogen, —CN, —R^{f}, —CO₂R^{d}, —CONR^{d}R^{e}, —NR^{e}C(O)₂R^{f}, —NR^{d}C(O)NR^{d}R^{e},
—NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

Embodiment 65: In embodiment 65, the method of embodiment 58 and any subembodiments contained therein, is wherein the compound of formula Ib has the structure:

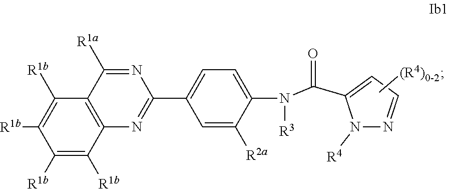

wherein
each R^{1a} and R^{1b} is independently selected from the group consisting of H, halogen,
—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
   R^{2a} is selected from the group consisting of H, F and CH₃;
   each R⁴ is independently selected from the group consisting of hydrogen,
halogen, —CN, —R^{f}, —CO₂R^{d}, —CONR^{d}R^{e}, —NR^{e}C(O)₂R^{f}, —NR^{d}C(O)NR^{d}R^{e},
—NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

Embodiment 66: In embodiment 66, the method of embodiment 62 and any subembodiments contained therein, is wherein the compound of formula If has the structure:

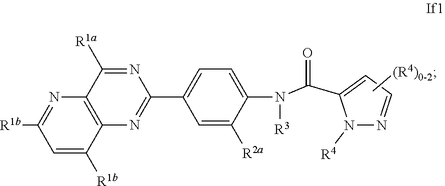

wherein
each R^{1a} and R^{1b} is independently selected from the group consisting of H, halogen,
—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
   R^{2a} is selected from the group consisting of H, F and CH₃;
   each R⁴ is independently selected from the group consisting of hydrogen,
halogen, —CN, —R^{f}, —CO₂R^{d}, —CONR^{d}R^{e}, —NR^{e}C(O)₂R^{f}, —NR^{d}C(O)NR^{d}R^{e},
—NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

Embodiment 67: In embodiment 67, the method of embodiment 63 and any subembodiments contained therein, is wherein the compound of formula Ig has the structure:

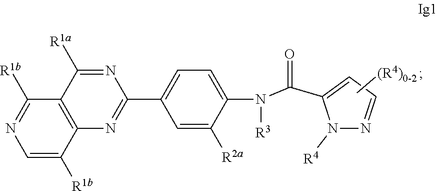

wherein
each R^{1a} and R^{1b} is independently selected from the group consisting of H, halogen,
—R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
   R^{2a} is selected from the group consisting of H, F and CH₃;
   each R⁴ is independently selected from the group consisting of hydrogen,
halogen, —CN, —R^{f}, —CO₂R^{d}, —CONR^{d}R^{e}, —NR^{e}C(O)₂R^{f}, —NR^{d}C(O)NR^{d}R^{e},
—NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

Embodiment 68: In embodiment 46, the method of any one of embodiments 1 to 47 and any subembodiments contained therein, is wherein the compound is selected from Table 1.

Embodiment 69: In embodiment 47, the method of any one of embodiments 1 to 47 and any subembodiments contained therein, is wherein the compound is selected from Table 1 and having +++ or ++++ activity.

Embodiment 70: In embodiment 48, the method of any one of embodiments 1 to 47 and any subembodiments contained therein, wherein the compound is selected from Table 1 and having ++++ activity.

Embodiment 71: An ex vivo or in vitro composition comprising a cell population of expanded stem cells and/or lineage committed progenitor cells thereof and a compound of Formula I, II, III, IV, Ia, Ib, Ic, Id, Ie, If, Ig, Ih, Ia1, Ib1, If1, Ig1, or a compound disclosed in Table 1. Within embodiment 71, in a first subembodiment the expanded stem cells are hematopoietic stem cells. Within embodiment 71, in a second subembodiment the expanded cells are hematopoietic stem cells and/or lineage committed hematopoietic progenitor cells. Within embodiment 71, in a third subembodiment the expanded cells are hematopoietic stem cells and lineage committed progenitor cells thereof. Within embodiment 71, in a fourth subembodiment the expanded cells are genetically modified hematopoietic stem cells and/or lineage committed genetically modified hematopoietic progenitor cells thereof.

Embodiment 72: A composition comprising a cell population of expanded stem cells and/or lineage committed progenitor cells obtained or obtainable by culturing ex vivo a starting population of the stem cells and/or lineage committed progenitor cells thereof with a compound of Formula I, II, III, or IV, Ia, Ib, Ic, Id, Ie, If, Ig, Ih, Ia1, Ib1, If1, Ig1, or a compound disclosed in Table 1. Within embodiment 72, in a first subembodiment the expanded cells are hematopoietic stem cells and/or lineage committed progenitor cells. Within embodiment 729, in a second subembodiment the expanded cells are hematopoietic stem cells and lineage committed progenitor cells thereof. Within embodiment 72, in a third subembodiment the expanded cells are genetically modified hematopoietic stem cells and/or lineage committed genetically modified hematopoietic progenitor cells thereof.

Embodiment 73: A composition comprising a cell population of expanded hematopoietic stem cells and/or progenitors thereof obtained or obtainable by culturing ex vivo a starting population of hematopoietic stem cells according to the method of any one of the embodiments 24 to 70.

Embodiment 74: In embodiment 74, the composition of any one of embodiments 71 to 73 is wherein the composition further comprises a pharmaceutically acceptable medium. In one subembodiment of embodiment 52, the composition comprising the stem cells is substantially free of a compound of Formula I, II, III, or IV and/or any other component of the cell culture. “Substantially free” as used herein means the composition comprising the stem cells contains less than about 10%, 20%, 30%, 40%, or 50%, preferably less than about 10% or 20% of formula a compound of Formula I, II, II, or IV and/or any other component of the cell culture, more preferably less than about 10% of a compound of Formula I, II, III, or IV and/or any other component of the cell culture medium.

Embodiment 75: In embodiment 75, the composition of any one of embodiments 71 to 73 is wherein the composition is suspended in a pharmaceutically acceptable medium suitable for transplantation into a patient, preferably the patient is a human. In a first subembodiment of embodiment 75, the patient is administered a therapeutically effective amount of stem cells. In a second subembodiment of embodiment 75, the composition comprising the stem cells is substantially free of a compound of Formula I, II, III, or IV and/or any other component of the cell culture. “Substantially free” as used herein means the composition comprising the stem cells contains less than about 10%, 20%, 30%, 40%, or 50%, preferably less than about 10% or 20% of formula a compound of Formula I, II, III, or IV and/or any other component of the cell culture, more preferably less than about 10% of a compound of I, II, III, or IV and/or any other component of the cell culture.

Embodiment 76: Embodiment 76 is directed to a method of treating a disease treatable by hematopoietic stem cell therapy (i.e., naturally occurring or genetically modified hematopoietic stem cells and/or lineage committed progenitor cells) comprising administering to a patient in need thereof a composition of any one of embodiments 71 to 75.

Embodiment 77: In embodiment 77, the method of embodiment 76 is wherein the disease is an immunodeficient disease, an autoimmune disorder, or a hematopoietic disorder.

Embodiment 78: In embodiment 78, the method of embodiment 76 is wherein the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

Embodiment 79: In embodiment 79, the method of embodiment 76 is wherein the disease is Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perform mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E).

Embodiment 80: An ex vivo or in vitro composition comprising a cell population of stem cells and/or lineage committed progenitor cells and a compound of Formula I, II, III, or IV (and any embodiments thereof disclosed in embodiments 48 to 70. Within embodiment 80, in a first subembodiment the stem cells are hematopoietic stem cells and/or lineage committed hematopoietic progenitor cells. Within embodiment 80, in a second subembodiment the stem cells are hematopoietic stem cells and lineage committed hematopoietic progenitor cells thereof. Within embodiment 80, in a third subembodiment the stem cells are hematopoietic stem cells.

Embodiment 81: A catheter comprising a cell population of expanded hematopoietic stem cells and/or lineage committed hematopoietic progenitor cells thereof obtained or obtainable by culturing ex vivo a starting population of cells comprising hematopoietic stem cells and/or lineage committed progenitor cells thereof and a compound of Formula I according to the method of any one of embodiments 24 to 70.

Embodiment 82: A syringe comprising a cell population of expanded hematopoietic stem cells and/or lineage committed progenitor cells thereof obtained or obtainable by culturing ex vivo a starting population of cells comprising hematopoietic stem cells and/or lineage committed progenitor cells thereof and a compound of Formula I according to the method of any one of embodiments 24 to 70.

Embodiment 83: Embodiment 83 is directed to a method of preparing genetically modified stem cells and/or lineage committed progenitor cells thereof comprising culturing naturally occurring stem cells and/or lineage committed progenitor cells thereof and a compound of formula I, II, III, or IV (and any embodiments thereof disclosed in embodiments 26 to 70). Within embodiment 83, in a first subembodiment the stem cells are hematopoietic stem cells and/or lineage committed progenitor cells thereof.

Embodiment 84: Embodiment 84, the method of embodiment 83 comprises culturing the hematopoietic stem cells and/or lineage committed progenitor cells thereof with a compound of formula I, II, III, or IV (and any embodiments thereof disclosed in embodiments 26 to 70) from 1 to 3 days prior to genetically modification the stem cells. In one subembodiment of embodiment 84, the hematopoietic cells are modified using CRISPR/Cas 9 method.

### Compositions and Therapeutic Uses

The expanded stem cells produced by any of the methods disclosed herein can be used for treatment of various diseases treatable by stem cell-based therapy. The expanded stem cells produced by in vitro or ex vivo methods disclosed herein may be used without further purification (i.e., directly after expansion) or after further purification and/or selection step(s). For example, the expanded stem cells may be washed to remove the compound of formula I, II, III, or IV and/or one or more agents used in the expansion methods disclosed herein and then resuspended in an appropriate cell suspension medium for short term use or in a long-term storage medium or in a pharmaceutically acceptable medium suitable for administration to a patient. Accordingly, in one aspect provided is an ex vivo or in vitro composition comprising a cell population of expanded stem cells and/or lineage committed progenitor cells and a compound of formula I, II, III, or IV (or any embodiment thereof disclosed herein). In another aspect, provided is an ex vivo or in vitro composition comprising a cell population of expanded stem cells and/or lineage committed progenitor cells substantively free of one or more agents used for the expansion of the stem cells in the methods disclosed herein (e.g., compound of formula I, II, III, or IV; Notch agonist; an agent that inhibits TGFβ signaling; TPO; etc.). In yet another aspect, provided is a composition comprising a cell population of expanded stem cells and/or lineage committed progenitor cells obtained or obtainable by any of the in vitro or ex vivo expansion methods disclosed herein. In yet another aspect, provided is a composition comprising a cell population of expanded stem cells and/or lineage committed progenitor cells obtained or obtainable by any of the in vitro or ex vivo expansion methods disclosed herein that is substantially free of one or more agents used for the expansion of the stem cells in the methods disclosed herein. Substantially free as used herein means the composition comprises less than about 5%, 10%, 20%, 30%, 40%, or 50%, preferably less than about 5% of one or more agents used in the expansion methods disclosed herein. “About” applies to each of the stated value.

In some embodiments, the expanded stem cells of any of aforementioned compositions are HSCs. In some embodiments, the expanded stem cells of any of aforementioned compositions are MSCs. In some embodiments the expanded stem cells of any of aforementioned compositions are genetically modified HSCs.

In some embodiments, any of the aforementioned compositions comprising expanded stem cells is resuspended in a pharmaceutically acceptable medium suitable for administration to a subject in need of stem cell therapy.

Accordingly, also provided herein are methods of treating diseases treatable by stem cell therapy comprising administering to a patient in need thereof a therapeutically effective amount of expanded stem cells prepared according to the stem cell expansion methods disclosed herein, optionally in a pharmaceutically acceptable medium. In a non-limiting example, the stem cells are transplanted to a subject in need of transplantation of stem cells. As used herein, the term “subject in need of transplantation of stem cells” include, e.g., subjects suffering from a disorder that can be treated by, i.e., can benefit from, transplantation of stem cells. Transplantation of the expanded stem cell population of the present disclosure may be by any suitable means of administration, for example, by subcutaneous, intravenous, intramuscular, or intracisternal injection or infusion techniques (e.g., cell suspensions) in a pharmaceutically acceptable medium. A pharmaceutically acceptable carrier for infusion of a composition comprising cells into a patient may comprise, for example, buffered saline with 5% HSA or supplemented basal medium or medium as known in the art. In other embodiments, transplantation can be facilitated via the use of catheter-based systems, pumps, or syringes.

It will be understood, however, that the therapeutic amount of expanded stem cells and frequency of administration for any particular subject may be varied and will depend upon a variety of factors including the activity and viability of the specific cells employed, and the age, body weight, general health, sex, diet, mode and time of administration, rate of engraftment, drug combination, the severity of the particular condition, and the host undergoing therapy. In some embodiments, a therapeutically effective amount of expanded cells transplanted into a subject is an amount capable of engrafting such transplanted cells in the subject, whereby such cells ameliorate of the symptoms of the disease by eliciting the desired biological response. In certain embodiments, stem cells administered to a subject can be in an amount between 1×10⁶ to 5×10⁸ cells/kg body weight, which can be delivered in single or multiple doses. In other embodiments, stem cells provided to a subject can be in an amount of about 1×10⁶, 2×10⁶, 5×10⁶, 10⁷, 5×10⁷, 10⁸ cells/kg body weight, or an amount between any of the above listed values. The cells may be administered on a regimen of 1 to 5 times per day, 1-3 times per day, once or twice per day, or every other day.

For methods where the stem cells are expanded in vivo, the compounds of formula I, II, III, or IV (and any embodiments thereof) provided herein may be in the form of compositions suitable for administration to a subject. In general, such compositions are “pharmaceutical compositions” comprising a compound of formula I, II, III, or IV (and any embodiments thereof) and one or more pharmaceutically acceptable or physiologically acceptable diluents, carriers or excipients. In certain embodiments, the compound of formula I, II, III, or IV (and any embodiments thereof) is present in a therapeutically acceptable amount.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present invention.

The pharmaceutical compositions containing the active ingredient (e.g., of formula I, II, III, or IV (and any embodiments thereof)) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount a compound of formula I, II, III, or IV (and any embodiments thereof) and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-hydroxyethyl)piperazine-N′-(2-ethanesulfonic acid) (HEPES), 2-(N-morpholino)ethane-sulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver an AhR modulator, including implants (e.g., implantable pumps) and catheter systems, slow injection pumps and devices, all of which are well known to the skilled artisan.

Depot injections, which are generally administered subcutaneously or intramuscularly, may also be utilized to release the compounds disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The present invention contemplates the administration of a compound of formula I, II, III, or IV (and any embodiments thereof) in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compound of formula I, II, III, or IV (and any embodiments thereof) contemplated by the present invention may also be in the form of any other suitable pharmaceutical composition (e.g., sprays for nasal or inhalation use) currently known or developed in the future.

For in vivo expansion, compounds of formula I, II, III, or IV (and any embodiments thereof) and compositions thereof can be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. Particular embodiments of the present invention contemplate oral administration.

Compounds of formula I, II, III, or IV (and any embodiments thereof) may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of formula I, II, III, or IV (and any embodiments thereof) may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, compounds of formula I, II, III, or IV (and any embodiments thereof) contemplated by the present invention may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the desired compound of formula I, II, III, or IV (and any embodiments thereof) is contained in a “unit dosage form”. The phrase “unit dosage form” refers to physically discrete units, each unit containing a predetermined amount of the AhR modulator, either alone or in combination with one or more additional agents, sufficient to produce the desired effect. It will be appreciated that the parameters of a unit dosage form will depend on the particular agent and the effect to be achieved.

The disease to be treated by the expanded stem cells according to the methods disclosed herein, including in vivo expansion, will depend on the specific type of stem cell. For example, expanded HSCs can be used for subjects whose bone marrow was destroyed by chemotherapy or radiation therapy used to treat some cancers. Expanded HSCs can be also used for subjects suffering from impaired hematopoiesis. In some embodiments the patient is suffering from a disease selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome. In some embodiments, the patient is suffering from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease AR I NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E). Stem cells for use in the present disclosure for treatment may be the subject's own cells (autologous transplantation) or those of a donor (allogeneic transplantation). Accordingly, the disclosure further provides expanded HSCs or its composition for use in allogeneic or autologous stem cell transplantation in a subject.

Diseases treatable by MSCs include bone or cartilage disease, a neurodegenerative disease, a cardiac disease, a hepatic disease, cancer, nerve damage, wound healing, autoimmune disease, graft versus host disease, spinal cord injury and tissue regeneration. Bone defects suitable for treatment include, but are not limited to osteogenesis imperfecta, fracture, congenital bone defects, and the like. Further, the expanded mesenchymal stem cells of the present disclosure can be implanted in a subject to provide osseous and connective tissue Support of orthopedic and other (e.g. dental) prosthetic devices. Such as joint replacements and/or tooth implants. The mesenchymal stem cells of the present disclosure can be used to treat CNS diseases.

Representative examples of CNS diseases or disorders include, but are not limited to, a pain disorder, a motion disorder, a dissociative disorder, a mood disorder, an affective disorder, a neurodegenerative disease or disorder and a convulsive disorder. More specific examples of such conditions include, but are not limited to, Parkinson's, ALS, Multiple Sclerosis, Huntingdon's disease, autoimmune encephalomyelitis, diabetic neuropathy, glaucomatous neuropathy, macular degeneration, action tremors and tardive dyskinesia, panic, anxiety, depression, alcoholism, insomnia, manic behavior, Alzheimer's and epilepsy. As mentioned, since MSCs can differentiate into cartilage, the mesenchymal stem cells of the present disclosure may be suitable for the treatment of joint conditions including, but not limited to osteoarthritis, rheumatoid arthritis, inflammatory arthritis, chondromalacia, avascular necrosis, traumatic arthritis and the like.

Bone marrow-derived mesenchymal stem cells (MSCs) are known to interact with hematopoietic stem cells (HSCs) and immune cells and represent potential cellular therapy to enhance allogeneic hematopoietic engraftment and prevent graft-versus-host disease (GVHD).

In any of the methods above, when the stem cells are hematopoietic stems cells and/or progenitors thereof, they may be administered in combination with one or more mobilizing agents selected from the group consisting of a CXCR4 antagonist (e.g., AMD3100), GCSF, and GRO. A “mobilizing agent” is an agent capable of inducing the migration of expanded stem cells from the bone marrow of a subject to the peripheral blood.

### Assays

### Inhibition of AhR and Stem Cell Expansion:

The ability of compounds of formula I, II, III, or IV to antagonize AhR and facilitate expansion of stem cells can be assessed by using, for example, an art-accepted assay or model, examples of which are will be apparent to the skilled artisan. The ability of the compounds described herein to antagonize AhR and to facilitate expansion of hematopoietic stem cells can be assessed by methods set forth in the Experimental section below.

### EXAMPLES

The following examples are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: wt=wildtype; bp=base pair(s); kb=kilobase(s); nt=nucleotides(s); aa=amino acid(s); s or sec=second(s); min=minute(s); h or hr=hour(s); ng=nanogram; g=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; IHC=immunohistochemistry; DMEM=Dulbeco's Modification of Eagle's Medium; EDTA=ethylenediaminetetraacetic acid.

### Example 001: 1-methyl-N-(2-methyl-4-(6-(trifluoromethyl)quinolin-2-yl)phenyl)-1H-pyrazole-5-carboxamide

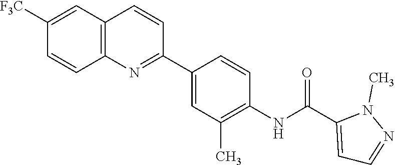 [SMILES: Cc1cc(-c2ccc3cc(C(F)(F)F)ccc3n2)ccc1NC(=O)c1ccnn1C]

A mixture of 2-methyl-4-(6-(trifluoromethyl)quinolin-2-yl)aniline (10 g, 33.08 mmol, 1.00 equiv), 1-methyl-1H-pyrazole-5-carboxylic acid (4.4 g, 34.89 mmol, 1.05 equiv), DIPEA (8.5 g, 65.77 mmol, 2.00 equiv) and HATU (4.4 g, 11.57 mmol, 1.50 equiv) in DMF (200 mL) was stirred overnight at room temperature. The reaction was quenched by the addition of 600 mL of water, extracted three times with 200 mL of ethyl acetate and the combined organic layers washed with 50 mL of water. Volatiles were eliminated under reduced pressure. The crude product was purified by recrystallization with methanol to afford the desired final product as a yellow solid in 76% yield. (ES, m/z): [M+H]⁺ 411; ¹H-NMR (DMSO-d₆, 300 MHz, ppm): δ2.40 (s, 3H), δ4.12 (s, 3H), δ7.12-7.13 (d, 1H), δ7.57-7.61 (m, 2H), δ8.00-8.04 (d, 1H), δ8.19-8.36 (m, 4H), δ8.54 (s, 1H), δ8.66-8.69 (d, 1H), δ10.00 (s, 1H).

### Step 1: ethyl (E)-3-(2-amino-5-(trifluoromethyl)phenyl)acrylate

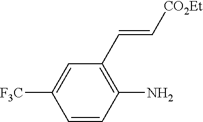 [SMILES: CCOC(=O)/C=C/c1cc(C(F)(F)F)ccc1N]

A mixture of 2-bromo-4-(trifluoromethyl)aniline (40 g, 166.65 mmol, 1.00 equiv), ethyl prop-2-enoate (34 g, 339.61 mmol, 2.00 equiv), DIPEA (64 g, 495.20 mmol, 3.00 equiv), P(O-tol)₃ (10 g, 0.20 equiv) and Pd(OAc)₂ (4 g, 17.82 mmol, 0.10 equiv) in DMF (400 mL) was stirred at 100° C. overnight. The reaction was quenched by the addition of 1200 mL of water and extracted with ethyl acetate (3×200 mL). The organic layers were combined and concentrated under reduced pressure and the resulting residue was purified by silica gel column chromatography with ethyl acetate/petroleum ether (1:10) to afford ethyl (E)-3-(2-amino-5-(trifluoromethyl)phenyl)acrylate as a yellow solid in 86% yield.

### Step 2: 6-(trifluoromethyl)quinolin-2(1H)-one

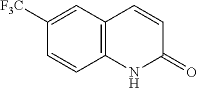 [SMILES: O=c1ccc2cc(C(F)(F)F)ccc2[nH]1]

Over a solution of ethyl (E)-3-(2-amino-5-(trifluoromethyl)phenyl)acrylate (37 g, 142.73 mmol, 1.00 equiv) in dioxane (92.7 mL), HCl (12N, 28 mL) was added. The resulting solution was stirred for 3 h at 100° C. and quenched by the addition of 200 mL of water. The solids were collected by filtration to afford the desired final product as a yellow solid in 89% yield.

### Step 3: 2-chloro-6-(trifluoromethyl)quinoline

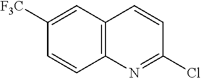 [SMILES: FC(F)(F)c1ccc2nc(Cl)ccc2c1]

Phosphoryl trichloride (135 mL) was added over 6-(trifluoromethyl)-1,2-dihydroquinolin-2-one (27 g, 126.67 mmol, 1.00 equiv). The reaction mixture was stirred for 1 h at 120° C. and concentrated under vacuum. Water (100 mL) was added and the resulting solid was collected by filtration to afford the desired final product in 68% yield.

### Step 4: 2-methyl-4-(6-(trifluoromethyl)quinolin-2-yl)aniline

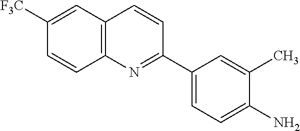 [SMILES: Cc1cc(-c2ccc3cc(C(F)(F)F)ccc3n2)ccc1N]

Over a solution of 2-chloro-6-(trifluoromethyl)quinoline (20 g, 86.36 mmol, 1.00 equiv) and 2-methyl-4-(4,4,5,5-tetramethyl-1,3,2-dioxaborolan-2-yl)aniline (24 g, 102.95 mmol, 1.20 equiv) in ethylene glycol dimethyl ether (400 mL) and water (80 mL), sodium carbonate (27.4 g, 258.51 mmol, 3.00 equiv) and Pd(PPh₃)₄(5 g, 4.33 mmol, 0.05 equiv) were added. The resulting solution was stirred for 3 h at 90° C. and the reaction quenched by addition of 200 mL of water. Extraction with ethyl acetate (3×200 mL) followed by evaporation of volatiles under reduced pressure afforded a residue that was purified by silica gel chromatography with ethyl acetate/petroleum ether (1:5) to afford the desired product as a yellow solid in 67% yield.

### Example 002: N,1-Dimethyl-N-(2-methyl-4-(6-(trifluoromethyl)quinolin-2-yl)phenyl)-1H-pyrazole-5-carboxamide

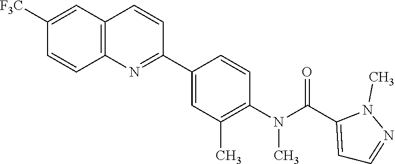 [SMILES: Cc1cc(-c2ccc3cc(C(F)(F)F)ccc3n2)ccc1N(C)C(=O)c1ccnn1C]

Over a solution of 1-methyl-N-(2-methyl-4-(6-(trifluoromethyl)quinolin-2-yl)phenyl)-1H-pyrazole-5-carboxamide (100 mg, 0.24 mmol, 1 equiv) in DMF (1 mL, 0.01 mmol, 0.056 equiv), NaH (9.8 mg, 0.41 mmol) was added. The resulting solution was stirred for 30 min at room temperature. CH₃I (34.6 mg, 0.24 mmol, 1 equiv) was added and the reaction mixture stirred for an additional hour at room temperature. The reaction was quenched with a saturated solution of NH₄Cl (20 mL) and extracted with ethyl acetate (3×20 ml). The organic layers were combined and concentrated under reduced pressure. The resulting residue was purified by preparative TLC with dichloromethane/methanol (40/1) to afford the desired final product as a white solid in 69% yield. LCMS (ES, m/z): [M+H]⁺ 425; ¹H-NMR (400 MHz, DMSO-d₆, ppm): δ 8.70-8.68 (d, 1H), δ8.55 (s, 1H), δ8.34-8.32 (d, 1H), δ8.28-8.25 (d, 2H), δ8.18-8.16 (d, 1H), δ 8.05-8.02 (d, 1H), δ7.50-7.48 (d, 1H), δ7.15 (s, 1H), δ5.55-5.54 (d, 1H), δ4.01 (s, 3H), δ3.33 (s, 3H), δ2.28 (s, 3H)

### Example 003: 1-methyl-N-(4-(quinoxalin-2-yl)phenyl)-1H-pyrazole-5-carboxamide

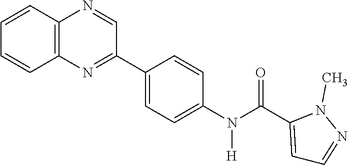 [SMILES: Cn1nccc1C(=O)Nc1ccc(-c2cnc3ccccc3n2)cc1]

A THE solution of 4-(quinoxalin-2-yl)aniline, 1-methyl-1H-pyrazole-5-carboxylic acid, DIPEA and T3P was stirred at room temperature for 15 hours. The reaction was quenched with water and extracted twice with ethyl acetate. The organic layers were combined and washed with a saturated solution of NaHCO₃, water and brine. The resulting solution was dried with anhydrous MgSO₄ and concentrated under reduced pressure to afford a residue that was purified by chromatography in silca gel (hexanes/ethyl acetate from 10% to 100%) to afford the desired final product as a yellow solid in 37% yield. LCMS (ES, m/z): [M+H]⁺ 330; ¹H-NMR (400 MHz, DMSO-d₆, ppm): 9.38 (s, 1H), 8.30 (d, 2H), 8.18 (m, 2H), 7.83 (m, 5H), 7.58 (d, 1H), 6.73 (d, 1H), 4.3 (s, 3H).

### Step 1: 4-(quinoxalin-2-yl)aniline

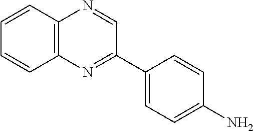 [SMILES: Nc1ccc(-c2cnc3ccccc3n2)cc1]

The title compound was prepared analogously to Example 001, step 4, where 2-bromoquinoxaline was substituted in place of 2-chloro-6-(trifluoromethyl)quinoline and 4-(4,4,5,5-tetramethyl-1,3,2-dioxaborolan-2-yl)aniline was substituted in place of 3-methyl-4-(tetramethyl-1,3,2-dioxaborolan-2-yl)aniline. Yield=68%.

### Example 004: N-Ethyl-1-methyl-N-(2-methyl-4-(6-(trifluoromethyl)quinolin-2-yl)phenyl)-1H-pyrazole-5-carboxamide

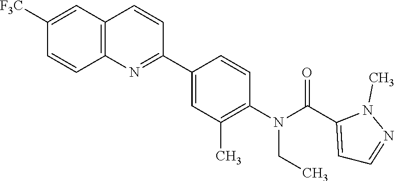 [SMILES: CCN(C(=O)c1ccnn1C)c1ccc(-c2ccc3cc(C(F)(F)F)ccc3n2)cc1C]

The title compound was prepared analogously to Example 002, where ethyl iodide was substituted in place of methyl iodide. LCMS (ES, m/z): [M+H]⁺ 439; ¹H-NMR (400 MHz, DMSO-d₆, ppm): δ 8.70-8.68 (d, 1H), δ8.55 (s, 1H), δ8.34-8.32 (d, 1H), δ8.28-8.25 (d, 2H), δ8.19-8.17 (d, 1H), δ8.05-8.02 (d, 1H), δ7.47-7.45 (d, 1H), δ7.15 (s, 1H), δ5.53 (s, 1H). δ4.11-4.01 (m, 1H), δ3.93 (s, 3H), δ3.69-3.60 (m, 1H), δ2.52 (s, 3H), δ1.24-1.20 (t, 3H).

### Example 005: N-(2-fluoro-3-methyl-4-(6-(trifluoromethyl)quinolin-2-yl)phenyl)-1-methyl-1H-pyrazole-5-carboxamide

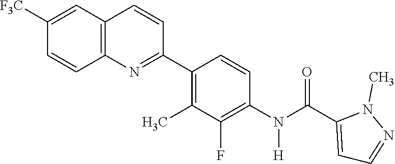 [SMILES: Cc1c(-c2ccc3cc(C(F)(F)F)ccc3n2)ccc(NC(=O)c2ccnn2C)c1F]

Pd(PPh₃)₄ (49.9 mg, 0.04 mmol, 0.1 equiv) was added to a solution of 2-chloro-6-(trifluoromethyl)quinoline (100 mg, 0.43 mmol, 1 equiv), N-(2-fluoro-3-methyl-4-(4,4,5,5-tetramethyl-1,3,2-dioxaborolan-2-yl)phenyl)-1-methyl-1H-pyrazole-5-carboxamide (170.6 mg, 0.47 mmol, 1.1 equiv) and Na₂CO₃ (91.5 mg, 0.86 mmol, 2 equiv) in DME (2 mL) and H₂O (0.5 mL). After stirring at 90° C. for three hours, the reaction was quenched with water (10 ml). The aqueous layer was extracted with EtOAc (2×20 mL) and the combined organic layers concentrated under reduced pressure. The residue was purified by preparative TLC (dichloromethane/MeOH=40:1) to afford the desired product as a white solid in 63% yield. LCMS (ES, m/z): [M+H]⁺ 429; ¹H-NMR (DMSO-d₆, 300 MHz, ppm): δ2.35-2.36 (d, 3H), δ4.11 (s, 3H), δ7.14-7.15 (d, 1H), δ7.44-7.47 (d, 1H), δ7.56-7.57 (d, 1H), δ7.60-7.65 (m, 1H), δ7.91-7.94 (d, 1H), δ8.04-8.08 (m, 1H), δ8.25-8.28 (d, 1H), δ8.61 (s, 1H), δ8.69-8.72 (d, 1H), δ10.21 (s, 1H)

### Step 1: 4-bromo-2-fluoro-3-methylaniline

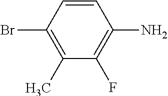

To a stirred solution of 2-fluoro-3-methylaniline (4 g, 31.96 mmol, 1 equiv) in acetonitrile (100 mL), NBS (6.2 g, 35.03 mmol, 1.096 equiv) was added in portions at 10° C. under nitrogen atmosphere. The resulting mixture was stirred at room temperature for 1 hour and concentrated under reduced pressure. The residue was purified by silica gel column chromatography, with ethyl acetate/petroleum ether (1:30) as eluent to afford 4-bromo-2-fluoro-3-methylaniline as a red solid in 86% yield.

### Step 2: N-(4-bromo-2-fluoro-3-methylphenyl)-1-methyl-1H-pyrazole-5-carboxamide

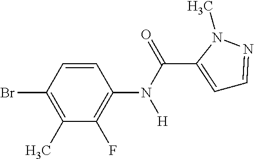 [SMILES: Cc1c(Br)ccc(NC(=O)c2ccnn2C)c1F]

To a solution of 4-bromo-2-fluoro-3-methylaniline (3 g, 14.70 mmol, 1 equiv), 1-methyl-1H-pyrazole-5-carboxylic acid (2.2 g, 17.44 mmol, 1.186 equiv) and DIPEA (3.8 g, 29.3 mmol, 1.994 equiv) in DMF (60.0 mL, 0.44 mmol, 0.028 equiv), HATU (8.4 g, 22.01 mmol, 1.497 equiv) was added. The resulting mixture was stirred at room temperature overnight. The reaction was quenched by the addition of water (180 mL) and the aqueous layer extracted with EtOAc (2×100 mL). The organic layers were combined and concentrated under reduced pressure. The residue was purified by silica gel column chromatography, with EtOAc/PE (1:10) to afford N-(4-bromo-2-fluoro-3-methylphenyl)-1-methyl-1H-pyrazole-5-carboxamide as a yellow solid in 83% yield.

### Step 3: N-(2-fluoro-3-methyl-4-(4,4,5,5-tetramethyl-1,3,2-dioxaborolan-2-yl)phenyl)-1-methyl-1H-pyrazole-5-carboxamide

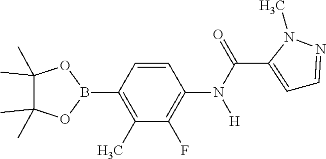 [SMILES: Cc1c(B2OC(C)(C)C(C)(C)O2)ccc(NC(=O)c2ccnn2C)c1F]

To a mixture of N-(4-bromo-2-fluoro-3-methylphenyl)-1-methyl-1H-pyrazole-5-carboxamide (3.8 g, 12.1 mmol, 1 equiv), 4,4,5,5-tetramethyl-2-(tetramethyl-1,3,2-dioxaborolan-2-yl)-1,3,2-dioxaborolane (3.4 g, 13.39 mmol, 1.1 equiv) and KOAc (3.6 g, 36.52 mmol, 3 equiv) in 1,4-dioxane (76 mL), Pd(dppf)Cl₂ (0.9 g, 1.22 mmol, 0.1 equiv) was added. The resulting mixture was stirred at 80° C. for 5 hours followed by elimination of volatiles under reduced pressure. The residue was purified by silica gel column chromatography with EtOAc/petroleum ether (1:15) to afford the desired final product as a yellow solid in 87% yield.

### Example 006: N-(2-fluoro-3-methyl-4-(6-(trifluoromethyl)quinolin-2-yl)phenyl)-N,1-dimethyl-1H-pyrazole-5-carboxamide

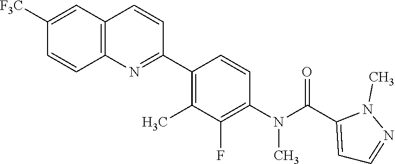 [SMILES: Cc1c(-c2ccc3cc(C(F)(F)F)ccc3n2)ccc(N(C)C(=O)c2ccnn2C)c1F]

The title compound was prepared analogously to Example 002, where N-(2-fluoro-3-methyl-4-(6-(trifluoromethyl)quinolin-2-yl)phenyl)-1-methyl-1H-pyrazole-5-carboxamide was substituted in place of 1-methyl-N-(2-methyl-4-(6-(trifluoromethyl)quinolin-2-yl)phenyl)-1H-pyrazole-5-carboxamide. LCMS (ES, m/z): [M+H]⁺ 443; ¹H-NMR (DMSO-d₆, 300 MHz, ppm): δ2.25-2.26 (d, 3H), δ3.38 (s, 3H), δ3.98 (s, 3H), δ5.85 (s, 1H), δ7.29 (s, 1H), δ7.43-7.8 (m, 2H), 67.87-7.90 (d, 1H), δ8.04-8.08 (d, 1H), δ8.24-827 (d, 1H), δ8.61 (s, 1H), δ8.69-8.72 (d, 1H)

### Example 007: N-(2-fluoro-6-methyl-4-(6-(trifluoromethyl)quinolin-2-yl)phenyl)-N,1-dimethyl-1H-pyrazole-5-carboxamide

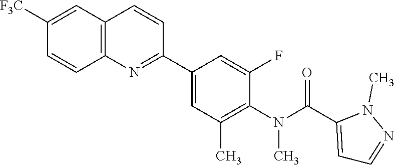 [SMILES: Cc1cc(-c2ccc3cc(C(F)(F)F)ccc3n2)cc(F)c1N(C)C(=O)c1ccnn1C]

The title compound was prepared analogously to Example 002, where N-(2-fluoro-6-methyl-4-(6-(trifluoromethyl)quinolin-2-yl)phenyl)-1-methyl-1H-pyrazole-5-carboxamide was substituted in place of N,1-Dimethyl-N-(2-methyl-4-(6-(trifluoromethyl)quinolin-2-yl)phenyl)-1H-pyrazole-5-carboxamide. LCMS (ES, m/z): [M+H]⁺ 443; ¹H-NMR (400 MHz, DMSO-d₆, ppm): δ 8.73-8.70 (d, 1H), δ8.56 (s, 1H), δ8.37-8.35 (d, 1H), δ8.29-8.27 (d, 1H), δ8.17 (s, 1H), δ8.07-8.04 (m, 2H), 67.21-7.20 (d, 1H), δ5.70-5.69 (d, 1H), δ3.99-3.95 (d, 3H), δ3.36-3.31 (d, 3H), δ2.41 (s, 3H)

### Example 008: N-(2-fluoro-6-methyl-4-(6-(trifluoromethyl)quinolin-2-yl)phenyl)-1-methyl-1H-pyrazole-5-carboxamide

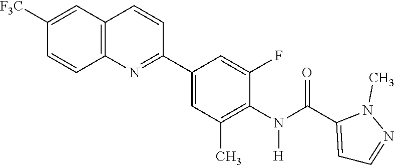 [SMILES: Cc1cc(-c2ccc3cc(C(F)(F)F)ccc3n2)cc(F)c1NC(=O)c1ccnn1C]

The title compound was prepared analogously to Example 001, where N-(2-fluoro-6-methyl-4-(6-(trifluoromethyl)quinolin-2-yl)phenyl)-1-methyl-1H-pyrazole-5-carboxamide was substituted in place of 1-methyl-N-(2-methyl-4-(6-(trifluoromethyl)quinolin-2-yl)phenyl)-1H-pyrazole-5-carboxamide. LCMS (ES, m/z): [M+H]+ 429; ¹H-NMR (300 MHz, DMSO-d₆, ppm): δ2.40 (s, 3H), δ4.11 (s, 3H), δ7.14 (s, 1H), δ7.58-7.59 (d, 1H), δ8.03-8.18 (m, 3H), 68.29-8.32 (d, 1H), δ8.39-8.42 (d, 1H), δ8.57 (s, 1H), δ8.71-8.74 (d, 1H), δ10.09 (s, 1H) Step 1: 4-bromo-2-fluoro-6-methylaniline

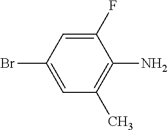

The title compound was prepared analogously to Example 005, step 1, where 2-fluoro-6-methylaniline was substituted in place of 2-fluoro-3-methylaniline. Yield=98%.

### Step 2: 2-fluoro-6-methyl-4-(4,4,5,5-tetramethyl-1,3,2-dioxaborolan-2-yl)aniline

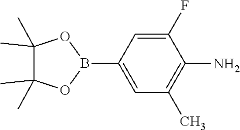 [SMILES: Cc1cc(B2OC(C)(C)C(C)(C)O2)cc(F)c1N]

The title compound was prepared analogously to Example 005, step 2, where 4-bromo-2-fluoro-6-methylaniline was substituted in place of 4-bromo-2-fluoro-3-methylaniline. Yield=85%.

### Example 009: tert-butyl methyl(2-methyl-4-(6-(trifluoromethyl)quinazolin-2-yl)phenyl)carbamate

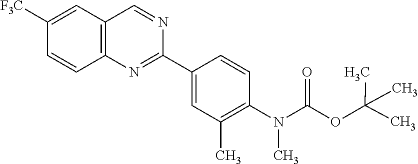 [SMILES: Cc1cc(-c2ncc3cc(C(F)(F)F)ccc3n2)ccc1N(C)C(=O)OC(C)(C)C]

To a solution of 2-chloro-6-(trifluoromethyl)quinazoline (4 g, 17.20 mmol, 1 equiv) and tert-butyl N-methyl-N-[2-methyl-4-(tetramethyl-1,3,2-dioxaborolan-2-yl)phenyl]carbamate (5.97 g, 17.20 mmol, 1 equiv) in toluene (80 mL) and EtOH (40 mL), K₂CO₃ (7.13 g, 51.59 mmol, 3 equiv) and Pd(PPh₃)₄(1.99 g, 1.72 mmol, 0.1 equiv) were added. The resulting mixture was stirred at 100° C. overnight. The reaction was quenched by the addition of water (200 mL) and the resulting mixture was extracted with EtOAc (2×100 mL). The combined organic layers were concentrated under reduced pressure and the residue purified by silica gel column chromatography with EtOAc/petroleum ether (1/20) as eluent to afford the desired product as a white solid in 70% yield. LCMS (ES, m/z): [M+H]⁺ 418; ¹H-NMR (300 MHz, CDCl₃, ppm): δ1.36-1.55 (m, 9H), δ2.36 (s, 3H), δ3.21 (s, 3H), δ7.29 (s, 1H), δ8.06-8.10 (q, 1H), δ8.21-8.26 (t, 2H), δ8.46-8.49 (d, 1H), δ8.52 (s, 1H), δ9.56 (s, 1H)

### Step 1: tert-butyl methyl(2-methyl-4-(4,4,5,5-tetramethyl-1,3,2-dioxaborolan-2-yl)phenyl)carbamate

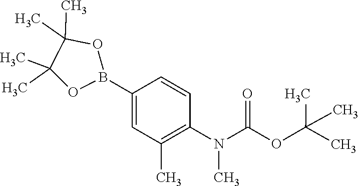 [SMILES: Cc1cc(B2OC(C)(C)C(C)(C)O2)ccc1N(C)C(=O)OC(C)(C)C]

The title compound was prepared analogously to Example 005, step 3, where tert-butyl (4-bromo-2-methylphenyl)(methyl)carbamate was substituted in place of N-(4-bromo-2-fluoro-3-methylphenyl)-1-methyl-1H-pyrazole-5-carboxamide. Yield=62%.

### Example 010: N,1-dimethyl-N-(2-methyl-4-(6-(trifluoromethoxy)quinolin-2-yl)phenyl)-1H-pyrazole-5-carboxamide

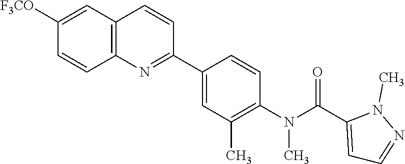 [SMILES: Cc1cc(-c2ccc3cc(OC(F)(F)F)ccc3n2)ccc1N(C)C(=O)c1ccnn1C]

The title compound was prepared analogously to Example 002 where 1-methyl-N-(2-methyl-4-(6-(trifluoromethoxy)quinolin-2-yl)phenyl)-1H-pyrazole-5-carboxamide was substituted in place of N,1-dimethyl-N-(2-methyl-4-(6-(trifluoromethyl)quinolin-2-yl)phenyl)-1H-pyrazole-5-carboxamide. Yield=14%. LCMS (ES, m/z): [M+H]⁺ 441; ¹H-NMR (400 MHz, CDCl₃, ppm): δ 8.30-8.28 (d, 2H), δ8.12 (s, 1H), δ8.04-8.02 (d, 1H), δ7.96-7.94 (d, 1H), δ7.70 (s, 1H), δ7.65-7.62 (d, 1H), δ7.35-7.32 (d, 1H), δ7.09 (s, 1H), δ5.53 (s, 1H), δ4.20 (s, 3H), δ3.43 (s, 3H), δ2.31 (s, 3H)

### Example 011: 1-methyl-N-(2-methyl-4-(6-(trifluoromethoxy)quinolin-2-yl)phenyl)-1H-pyrazole-5-carboxamide

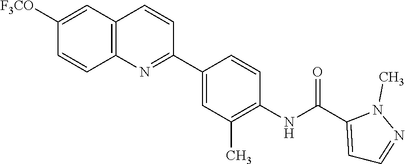 [SMILES: Cc1cc(-c2ccc3cc(OC(F)(F)F)ccc3n2)ccc1NC(=O)c1ccnn1C]

The title compound was prepared analogously to Example 005 where 2-chloro-6-(trifluoromethoxy)quinoline was substituted in place of 2-chloro-6-(trifluoromethyl)quinoline. Yield=53%. LCMS (ES, m/z): [M+1]⁺ 427; ¹H-NMR: (400 MHz, DMSO-d₆, ppm): δ 9.97 (s, 1H), δ 8.58-8.56 (d, 1H), δ8.30-8.15 (m, 4H), δ8.08 (s, 1H), δ7.79-7.76 (m, 1H), δ7.60-7.56 (d, 2H), δ7.12-7.11 (d, 1H), δ4.11 (s, 3H), δ2.39 (s, 3H).

### Step 1: Ethyl (E)-3-(2-amino-5-(trifluoromethoxy)phenyl)acrylate

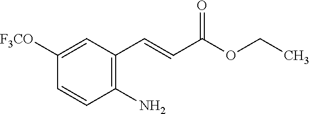 [SMILES: CCOC(=O)/C=C/c1cc(OC(F)(F)F)ccc1N]

The title compound was prepared analogously to Example 001, step 1, where 2-bromo-4-(trifluoromethoxy)aniline was substituted in place of 2-bromo-4-(trifluoromethyl)aniline. Yield=90%.

### Step 2: 6-(Trifluoromethoxy)quinolin-2(1H)-one

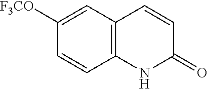 [SMILES: O=c1ccc2cc(OC(F)(F)F)ccc2[nH]1]

The title compound was prepared analogously to Example 001, step 2, where Ethyl (E)-3-(2-amino-5-(trifluoromethoxy)phenyl)acrylate was substituted in place of ethyl (E)-3-(2-amino-5-(trifluoromethyl)phenyl)acrylate. Yield=98%.

### Step 3: 2-Chloro-6-(trifluoromethoxy)quinoline

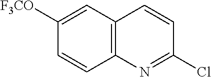 [SMILES: FC(F)(F)Oc1ccc2nc(Cl)ccc2c1]

The title compound was prepared analogously to Example 001, step 3, where 6-(trifluoromethoxy)quinolin-2(1H)-one was substituted in place of 6-(trifluoromethyl)-1,2-dihydroquinolin-2-one. Yield=98%.

### Example 012: N-(4-(8-chloroquinolin-2-yl)-2-methylphenyl)-1-methyl-1H-pyrazole-5-carboxamide

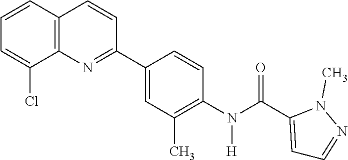 [SMILES: Cc1cc(-c2ccc3cccc(Cl)c3n2)ccc1NC(=O)c1ccnn1C]

The title compound was prepared analogously to Example 003, step 1, where 4-(8-chloroquinolin-2-yl)-2-methylaniline was substituted in place of 4-(quinoxalin-2-yl)aniline. Yield=28%. LCMS (ES, m/z): [M+H]⁺ 377; ¹H-NMR (400 MHz, CDCl₃, ppm): 8.29 (s, 1H), 8.28 (d, 1H), 8.20 (d, 1H), 8.13 (dd, 1H), 8.00 (d, 1H), 7.88 (dd, 1H), 7.77 (dd, 1H), 7.70 (s, 1H), 7.56 (d, 1H), 7.46 (m, 1H), 6.73 (d, 1H), 4.30 (s, 3H), 2.45 (s, 3H).

### Step 1: 4-(8-chloroquinolin-2-yl)-2-methylaniline

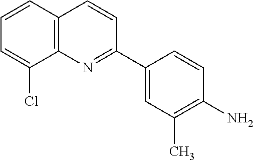 [SMILES: Cc1cc(-c2ccc3cccc(Cl)c3n2)ccc1N]

The title compound was prepared analogously to Example 014, step 1, where 2,8-chloroquinoline was substituted in place of 2-chloro-6-fluoroquinoline and 2-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=98%.

### Example 013: 1-methyl-N-(2-methyl-4-(1,6-naphthyridin-2-yl)phenyl)-1H-pyrazole-5-carboxamide

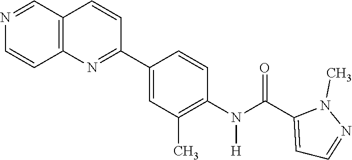 [SMILES: Cc1cc(-c2ccc3cnccc3n2)ccc1NC(=O)c1ccnn1C]

The title compound was prepared analogously to Example 003, where 2-Methyl-4-(1,6-naphthyridin-2-yl)aniline was substituted in place of 4-(quinoxalin-2-yl)aniline. LCMS (ES, m/z): [M+1]⁺ 344; ¹H-NMR (400 MHz, CDCl₃, ppm): 9.28 (s, 1H). 8.75 (d, 1H), 8.36 (d, 1H), 8.24 (d, 1H), 8.17 (s, 1H), 8.04 (m, 3H), 7.69 (s, 1H), 7.53 (s, 1H), 6.69 (s, 1H), 4.25 (s, 3H), 2.47 (s, 3H).

### Step 1: 2-Methyl-4-(1,6-naphthyridin-2-yl)aniline

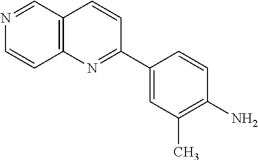 [SMILES: Cc1cc(-c2ccc3cnccc3n2)ccc1N]

The title compound was prepared analogously to Example 014, where 2-chloro-1,6-naphthyridine was substituted in place of 2-chloro-6-fluoroquinoline and 2-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=89%.

### Example 014: N-(4-(6-fluoroquinolin-2-yl)-2-methylphenyl)-1-methyl-1H-pyrazole-5-carboxamide

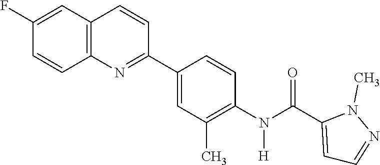 [SMILES: Cc1cc(-c2ccc3cc(F)ccc3n2)ccc1NC(=O)c1ccnn1C]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline. Yield=88%. LCMS (ES, m/z): [M+H]⁺ 361; ¹H-NMR (300 MHz, CDCl₃, ppm): δ2.47 (s, 3H), δ4.27 (s, 3H), δ6.70-6.71 (d, 1H), δ7.44-7.56 (m, 3H), δ7.69 (s, 1H), δ7.90-7.93 (d, 1H), δ8.00-8.03 (q, 1H), δ8.15-8.22 (m, 4H)

### Step 1: tert-butyl (4-(6-fluoroquinolin-2-yl)-2-methylphenyl)carbamate

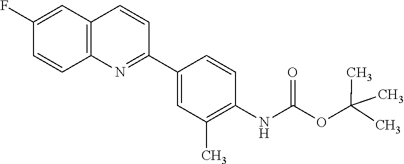 [SMILES: Cc1cc(-c2ccc3cc(F)ccc3n2)ccc1NC(=O)OC(C)(C)C]

A solution of 2-chloro-6-fluoroquinoline (81 mg, 0.45 mmol), tert-butyl (2-methyl-4-(4,4,5,5-tetramethyl-1,3,2-dioxaborolan-2-yl)phenyl)carbamate (164 mg, 0.49 mmol), bis(triphenylphosphine)palladium (II) chloride (16 mg, 0.022 mmol) and K₂CO₃ (123 mg, 0.89 mmol) in ethanol (4 mL) and water (1 mL), was heated at 80 degrees for 4 hours. The reaction was cooled down to room temperature and diluted with water, extracted with ethyl acetate three times, dried with MgSO₄, filtered and concentrated under reduced pressure. Purification of the resulting crude material by silica gel chromatography, afforded the desired final product as a white solid in 88% yield.

### Step 2: 4-(6-fluoroquinolin-2-yl)-2-methylaniline

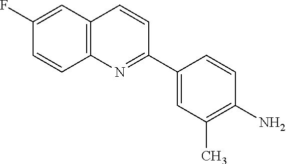 [SMILES: Cc1cc(-c2ccc3cc(F)ccc3n2)ccc1N]

TFA (0.15 mL) was added over a solution of tert-butyl (4-(6-fluoroquinolin-2-yl)-2-methylphenyl)carbamate (139 mg, 0.39 mmol) in dichloromethane (5 mL) at room temperature. The mixture was stirred for 6 hours and the reaction stopped by addition of water. The pH of the mixture was brought to neutral by addition of 1M NaOH and extracted three times with CH₂Cl₂. The combined organic layers were washed with brine, dried over MgSO₄ and concentrated under reduced pressure to afford a crude material that was purified by silica gel chromatography to afford the desired final product as a yellow solid in 81% yield.

### Example 015: tert-butyl (2-methyl-4-(4-methylquinolin-2-yl)phenyl)carbamate

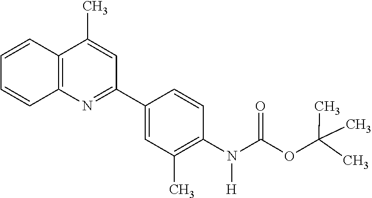 [SMILES: Cc1cc(-c2cc(C)c3ccccc3n2)ccc1NC(=O)OC(C)(C)C]

The title compound was prepared analogously to Example 014, step 1, where 2-chloro-4-methylquinoline was substituted in place 2-chloro-6-fluoroquinoline. Yield=31%. LCMS (ES, m/z): [M+H]⁺ 349; ¹H-NMR (300 MHz, CDCl₃, ppm): 8.19 (d, 1H), 8.06 (m, 2H), 7.96 (m, 2H), 7.70 (m, 2H), 7.53 (m, 1H), 6.41 (s, 1H), 2.76 (s, 3H), 2.38 (s, 3H), 1.55 (s, 9H).

### Example 016: 1-Methyl-N-(2-methyl-4-(4-methylquinolin-2-yl)phenyl)-1H-pyrazole-5-carboxamide

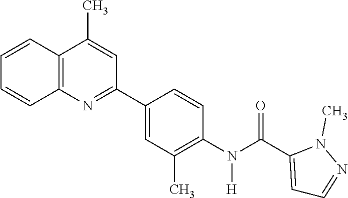 [SMILES: Cc1cc(-c2cc(C)c3ccccc3n2)ccc1NC(=O)c1ccnn1C]

The title compound was prepared analogously to Example 003, where 2-methyl-4-(4-methylquinolin-2-yl)aniline was substituted in place of 4-(quinoxalin-2-yl)aniline. Yield=41%. LCMS (ES, m/z): [M+H]⁺ 357; ¹H-NMR (300 MHz, CDCl₃, ppm): 8.20 (m, 2H), 8.01 (d, 2H), 7.82-7.47 (m, 5H), 7.26 (s, 1H), 6.70 (s, 1H), 4.25 (s, 3H), 2.81 (s, 3H), 2.46 (s, 3H)

### Step 1: 2-methyl-4-(4-methylquinolin-2-yl)aniline

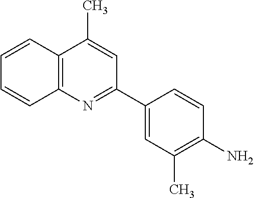 [SMILES: Cc1cc(-c2cc(C)c3ccccc3n2)ccc1N]

The title compound was prepared analogously to Example 014, step 2, where tert-butyl (2-methyl-4-(4-methylquinolin-2-yl)phenyl)carbamate was substituted in place of tert-butyl (4-(6-fluoroquinolin-2-yl)-2-methylphenyl)carbamate. Yield=84%.

### Example 017: tert-butyl (2-methyl-4-(8-methylquinolin-2-yl)phenyl)carbamate

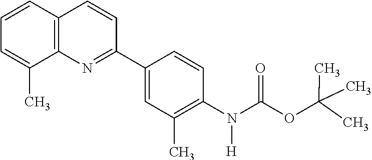 [SMILES: Cc1cc(-c2ccc3cccc(C)c3n2)ccc1NC(=O)OC(C)(C)C]

The title compound was prepared analogously to Example 014, step 1, where 2-chloro-8-methylquinoline was substituted in place 2-chloro-6-fluoroquinoline. Yield=26%. LCMS (ES, m/z): [M+H]⁺ 349; ¹H-NMR (300 MHz, CDCl₃, ppm): 8.16 (d, 1H), 8.12 (s, 1H), 8.04 (s, 2H), 7.87 (d, 1H), 7.64 (d, 1H), 7.56 (d, 1H), 7.39 (m, 1H), 6.40 (s, 1H), 2.91 (3H), 2.39 (s, 3H), 1.56 (s, 9H)

### Example 018: N-(4-(8-fluoroquinolin-2-yl)-2-methylphenyl)-1-methyl-1H-pyrazole-5-carboxamide

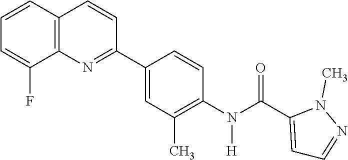 [SMILES: Cc1cc(-c2ccc3cccc(F)c3n2)ccc1NC(=O)c1ccnn1C]

The title compound was prepared analogously to Example 003, where 4-(8-fluoroquinolin-2-yl)-2-methylaniline was substituted in place 4-(quinoxalin-2-yl)aniline. Yield=33%. LCMS (ES, m/z): [M+H]⁺ 361; ¹H-NMR (CDCl₃, 400 MHz, ppm): 2.50 (s, 3H), 4.30 (s, 3H), 6.7 (s, 1H), 7.47 (m, 2H), 7.56 (d, 1H), 7.68 (m, 2H), 7.95 (d, 1H), 8.06 (dd, 1H), 8.25 (m, 3H).

### Step 1: tert-butyl (4-(8-fluoroquinolin-2-yl)-2-methylphenyl)carbamate

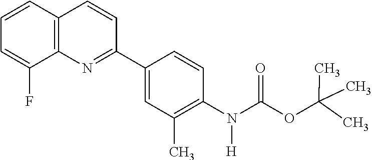 [SMILES: Cc1cc(-c2ccc3cccc(F)c3n2)ccc1NC(=O)OC(C)(C)C]

The title compound was prepared analogously to Example 014, step 1, where 2-chloro-8-fluoroquinoline was substituted in place 2-chloro-6-fluoroquinoline. Yield=28%

### Step 2: 4-(8-fluoroquinolin-2-yl)-2-methylaniline

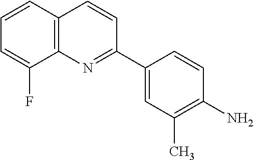 [SMILES: Cc1cc(-c2ccc3cccc(F)c3n2)ccc1N]

The title compound was prepared analogously to Example 014, step 2, where tert-butyl (4-(8-fluoroquinolin-2-yl)-2-methylphenyl)carbamate was substituted in place of tert-butyl (4-(6-fluoroquinolin-2-yl)-2-methylphenyl)carbamate. Yield=33%.

### Example 019: N-(4-(7-fluoroquinolin-2-yl)-2-methylphenyl)-1-methyl-1H-pyrazole-5-carboxamide

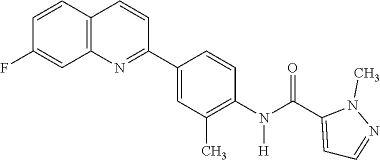 [SMILES: Cc1cc(-c2ccc3ccc(F)cc3n2)ccc1NC(=O)c1ccnn1C]

The title compound was prepared analogously to Example 003, where 4-(7-fluoroquinolin-2-yl)-2-methylaniline was substituted in place 4-(quinoxalin-2-yl)aniline. Yield=46%. LCMS (ES, m/z): [M+H]⁺ 361; ¹H-NMR (CDCl₃, 400 MHz, ppm): 8.21 (m, 3H), 8.01 (dd, 1H), 7.85 (m, 3H), 7.67 (s, 1H), 7.54 (d, 1H), 7.33 (m, 1H), 6.69 (d, 1H), 4.25 (s, 3H), 2.46 (s, 3H).

### Step 1: tert-butyl (4-(7-fluoroquinolin-2-yl)-2-methylphenyl)carbamate

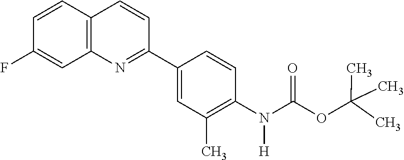 [SMILES: Cc1cc(-c2ccc3ccc(F)cc3n2)ccc1NC(=O)OC(C)(C)C]

The title compound was prepared analogously to Example 014, step 1, where 2-chloro-7-fluoroquinoline was substituted in place 2-chloro-6-fluoroquinoline. Yield=65%

### Step 2: 4-(7-fluoroquinolin-2-yl)-2-methylaniline

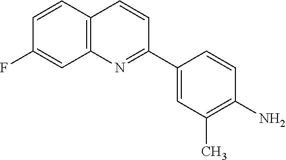 [SMILES: Cc1cc(-c2ccc3ccc(F)cc3n2)ccc1N]

The title compound was prepared analogously to Example 014, step 2, where tert-butyl (4-(7-fluoroquinolin-2-yl)-2-methylphenyl)carbamate was substituted in place of tert-butyl (4-(6-fluoroquinolin-2-yl)-2-methylphenyl)carbamate. Yield=79%.

### Example 020: 1-Methyl-N-(2-methyl-4-(8-methylquinolin-2-yl)phenyl)-1H-pyrazole-5-carboxamide

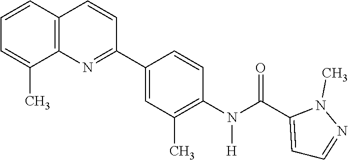 [SMILES: Cc1cc(-c2ccc3cccc(C)c3n2)ccc1NC(=O)c1ccnn1C]

The title compound was prepared analogously to Example 003, where 2-Methyl-4-(8-methylquinolin-2-yl)aniline was substituted in place of 4-(quinoxalin-2-yl)aniline. Yield=22%. LCMS (ES, m/z): [M+H]⁺ 357; ¹H-NMR (CDCl₃, 400 MHz, ppm): 2.37 (s, 3H), 2.85 (s, 3H), 4.18 (s, 3H), 6.6 (d, 1H), 7.18 (s, 1H), 7.32 (m, 1H), 7.45 (d, 1H), 7.50 (d, 1H), 7.57 (m, 1H), 7.81 (d, 1H), 8.00-8.15 (m, 4H).

### Step 1: tert-butyl (2-methyl-4-(8-methylquinolin-2-yl)phenyl)carbamate

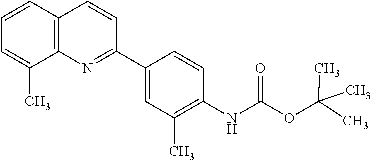 [SMILES: Cc1cc(-c2ccc3cccc(C)c3n2)ccc1NC(=O)OC(C)(C)C]

The title compound was prepared analogously to Example 014, step 1, where tert-butyl (2-methyl-4-(8-methylquinolin-2-yl)phenyl)carbamate was substituted in place of tert-butyl (4-(6-fluoroquinolin-2-yl)-2-methylphenyl)carbamate. Yield=82%

### Step 2: 2-Methyl-4-(8-methylquinolin-2-yl)aniline

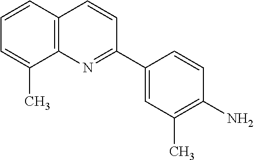 [SMILES: Cc1cc(-c2ccc3cccc(C)c3n2)ccc1N]

The title compound was prepared analogously to Example 014, step 2, where tert-butyl (2-methyl-4-(8-methylquinolin-2-yl)phenyl)carbamate was substituted in place of tert-butyl (4-(6-fluoroquinolin-2-yl)-2-methylphenyl)carbamate. Yield=82%

### Example 021: N-(4-(Isoquinolin-6-yl)-2-methylphenyl)-1-methyl-1H-pyrazole-5-carboxamide

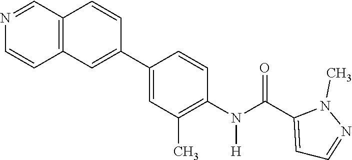 [SMILES: Cc1cc(-c2ccc3cnccc3c2)ccc1NC(=O)c1ccnn1C]

The title compound was prepared analogously to Example 003, where 4-(isoquinolin-6-yl)-2-methylaniline was substituted in place of 4-(quinoxalin-2-yl)aniline. Yield=7%. LCMS (ES, m/z): [M+H]⁺ 343; ¹H-NMR (CDCl₃, 400 MHz, ppm): 2.48 (s, 3H), 4.28 (s, 3H), 6.75 (d, 1H), 7.58 (d, 1H), 7.7 (m, 3H), 7.95 (d, 1H), 8.05 (dd, 1H), 8.15 (m, 3H), 8.55 (d, 1H), 9.37 (s, 1H).

### Step 1: 4-(isoquinolin-6-yl)-2-methylaniline

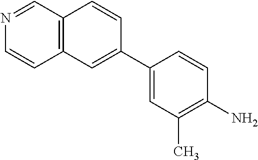 [SMILES: Cc1cc(-c2ccc3cnccc3c2)ccc1N]

The title compound was prepared analogously to Example 014, step 1, where 6-bromoisoquinoline was substituted in place 2-chloro-6-fluoroquinoline and 2-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=46%

### Example 022: tert-butyl (4-(8-fluoroquinolin-2-yl)-2-methylphenyl)carbamate

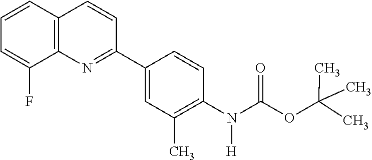 [SMILES: Cc1cc(-c2ccc3cccc(F)c3n2)ccc1NC(=O)OC(C)(C)C]

The title compound was prepared analogously to Example 014, step 1, where 2-chloro-8-fluoroquinoline was substituted in place 2-chloro-6-fluoroquinoline. Yield=28%. LCMS (ES, m/z): [M+H]⁺ 353; ¹H-NMR (CDCl₃, 400 MHz, ppm): 8.21 (dd, 1H), 8.13 (d, 1H), 8.08 (d, 1H), 7.95 (m, 2H), 7.59 (m, 1H), 7.42 (m, 2H), 6.43 (s, 1H), 2.38 (s, 3H), 1.55 (s, 9H)

### Example 023: N-(4-(6-fluoroquinolin-2-yl)-2-methylphenyl)benzamide

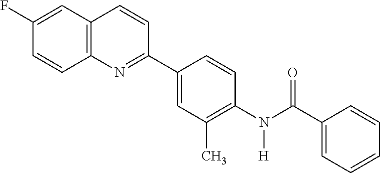 [SMILES: Cc1cc(-c2ccc3cc(F)ccc3n2)ccc1NC(=O)c1ccccc1]

To a solution of 4-(6-fluoroquinolin-2-yl)-2-methylaniline (100 mg, 0.40 mmol, 1 equiv) and benzoyl chloride (61.3 mg, 0.44 mmol, 1.1 equiv) in CH₂Cl₂(1 mL) at room temperature, triethylamine (80.2 mg, 0.79 mmol, 2 equiv) was added. The reaction mixture was stirred at room temperature for 3 hours and quenched with water (10 mL). The resulting mixture was extracted with dichloromethane (3×20 mL) and concentrated under reduced pressure. The residue was purified by recrystallization from dichloromethane/hexane=1:10 to afford the desired product as a white solid in 92% yield. LCMS (ES, m/z): [M+H]⁺ 357; ¹H-NMR (DMSO-d₆, 400 MHz, ppm): δ3.01 (s, 3H), δ7.54-7.61 (m, 4H), δ7.62-7.73 (m, 1H), δ7.81-7.84 (m, 1H), δ8.01-8.03 (t, 2H), δ8.12-8.17 (m, 2H), δ8.21-8.24 (m, 2H), δ8.45-8.47 (d, 1H), δ9.98 (s, 1H)

### Example 024: methyl (4-(6-fluoroquinolin-2-yl)-2-methylphenyl)carbamate

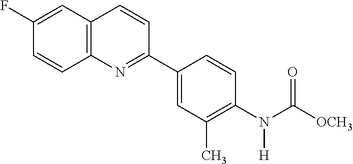 [SMILES: COC(=O)Nc1ccc(-c2ccc3cc(F)ccc3n2)cc1C]

The title compound was prepared analogously to Example 005 where 2-chloro-6-fluoroquinoline was substituted in place of 2-chloro-6-(trifluoromethyl)quinoline and (4-((methoxycarbonyl)amino)-3-methylphenyl)boronic acid was substituted in place of N-(2-fluoro-3-methyl-4-(4,4,5,5-tetramethyl-1,3,2-dioxaborolan-2-yl)phenyl)-1-methyl-1H-pyrazole-5-carboxamide. Yield=15%. LCMS (ES, m/z): [M+H]⁺ 311; ¹H-NMR (CD₃OD, 300 MHz, ppm): δ2.38 (s, 3H), δ3.78 (s, 3H), δ7.54-7.62 (m, 2H), δ7.69-7.71 (d, 1H), δ7.93-7.96 (m, 1H), δ7.99-8.02 (m, 2H), δ8.10-8.15 (m, 1H), δ8.33-8.35 (d, 1H)

### Example 025: N-(4-(6-fluoroquinolin-2-yl)-2-methylphenyl)tetrahydro-2H-pyran-4-carboxamide

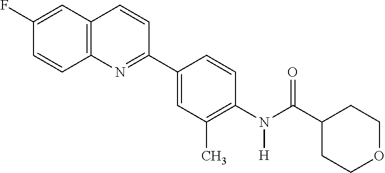 [SMILES: Cc1cc(-c2ccc3cc(F)ccc3n2)ccc1NC(=O)C1CCOCC1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and tetrahydro-2H-pyran-4-carboxylic acid was substituted in place of 1-methyl-1H-pyrazole-5-carboxylic acid. Yield=68%. LCMS (ES, m/z): [M+H]⁺ 365; ¹H-NMR (300 MHz, CD₃OD, ppm): 8.36-8.34 (d, 1H), 8.18-8.13 (m, 1H), 8.07-7.97 (m, 3H), 7.65-7.56 (m, 3H), 4.08-4.04 (m, 2H), 3.59-3.51 (m, 2H), 2.84-2.74 (m, 1H), 2.40 (s, 3H), 2.00-1.87 (m, 4H)

### Example 026: N-(4-(6-fluoroquinolin-2-yl)-2-methylphenyl)picolinamide

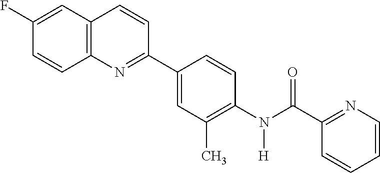 [SMILES: Cc1cc(-c2ccc3cc(F)ccc3n2)ccc1NC(=O)c1ccccn1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and picolinic acid was substituted in place of 1-methyl-1H-pyrazole-5-carboxylic acid. Yield=87%. LCMS (ES, m/z): [M+H]⁺ 358; ¹H-NMR (300 MHz, CDCl₃, ppm): 10.34 (s, 1H), 8.68-8.67 (t, 1H), 8.61-8.58 (d, 1H), 8.38-8.35 (m, 1H), 8.21-8.18 (m, 3H), 8.06-8.00 (m, 1H), 7.99-7.94 (m, 2H), 7.63-7.31 (m, 3H), 2.60 (s, 3H)

### Example 027: N-(4-(6-fluoroquinolin-2-yl)-2-methylphenyl)nicotinamide

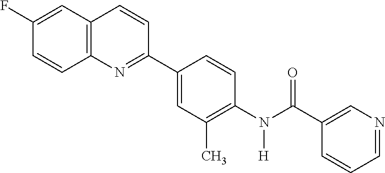 [SMILES: Cc1cc(-c2ccc3cc(F)ccc3n2)ccc1NC(=O)c1cccnc1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and nicotinic acid was substituted in place of 1-methyl-1H-pyrazole-5-carboxylic acid. Yield=55%. LCMS (ES, m/z): [M+H]⁺ 358; ¹H-NMR (300 MHz, CDCl₃, ppm): 9.19 (s, 1H), 8.85-8.34 (d, 1H), 8.32-8.8.18 (m, 5H), 8.06-8.02 (m, 1H), 7.95-7.89 (m, 2H), 7.57-7.46 (m, 3H), 2.52 (s, 3H)

### Example 028: N-(4-(6-fluoroquinolin-2-yl)-2-methylphenyl)isonicotinamide

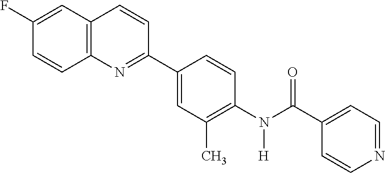 [SMILES: Cc1cc(-c2ccc3cc(F)ccc3n2)ccc1NC(=O)c1ccncc1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and pyridine-4-carboxylic acid was substituted in place of 1-methyl-1H-pyrazole-5-carboxylic acid. Yield=59%. LCMS (ES, m/z): [M+H]⁺ 358; ¹H-NMR (300 MHz, CDCl₃, ppm): 8.88-8.86 (d, 2H), 8.23-8.16 (m, 4H), 8.05-8.02 (d, 1H), 7.94-7.88 (m, 2H), 7.79-7.77 (d, 2H), 7.57-7.45 (m, 2H), 2.51 (s, 3H).

### Example 029: N-(4-(6-fluoroquinolin-2-yl)-2-methylphenyl)pyridazine-4-carboxamide

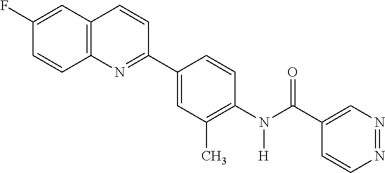 [SMILES: Cc1cc(-c2ccc3cc(F)ccc3n2)ccc1NC(=O)c1ccnnc1]

4-(6-fluoroquinolin-2-yl)-2-methylaniline (100 mg, 0.40 mmol, 1 equiv) and pyridazine-4-carboxylic acid (73.8 mg, 0.59 mmol, 1.5 equiv) were dissolved in dichloromethane (5 mL) and diisopropylethylamine (102.5 mg, 0.79 mmol, 2 equiv) and HATU (226.1 mg, 0.59 mmol, 1.5 equiv) were added next. The resulting solution was stirred at room temperature overnight and quenched by the addition of 5 mL of water. Extraction with dichloromethane (2×5 ml) and elimination of volatiles under reduced pressure, afford a residue that was purified by preparative TLC with dichloromethane/methanol (25:1) to afford the desired product as a white solid in 58% yield. LCMS (ES, m/z): [M+H]⁺ 359; ¹H-NMR (300 MHz, CDCl₃, ppm): δ2.25 (s, 3H), δ7.63-7.66 (m, 1H), δ7.68-7.73 (m, 1H), δ7.81-7.84 (m, 1H), δ8.13-8.17 (m, 3H), δ8.22-8.24 (m, 2H), δ8.46-8.48 (d, 1H), δ9.53-9.54 (d, 1H), δ9.70 (s, 1H), δ10.47 (s, 1H)

### Example 030: N-(4-(6-fluoroquinolin-2-yl)-2-methylphenyl)pyrimidine-5-carboxamide

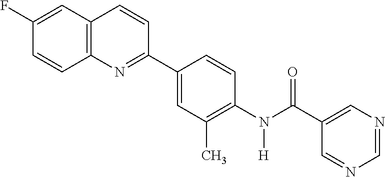 [SMILES: Cc1cc(-c2ccc3cc(F)ccc3n2)ccc1NC(=O)c1cncnc1]

The title compound was prepared analogously to Example 029 where pyrimidine-5-carboxylic acid was substituted in place of pyridazine-4-carboxylic acid. Yield=38%. LCMS (ES, m/z): [M+H]⁺ 359; ¹H-NMR (400 MHz, DMSO-d₆, ppm): δ2.42 (s, 3H), δ7.64-7.81 (m, 2H), δ7.81-7.84 (m, 1H), δ8.14-8.17 (m, 2H), δ8.23-8.25 (m, 2H), δ8.46-8.48 (d, 1H), δ9.33 (s, 2H), δ9.40 (s, 1H), δ10.33 (s, 1H).

### Example 031: N-(4-(6-fluoroquinolin-2-yl)-2-methylphenyl)pyridazine-3-carboxamide

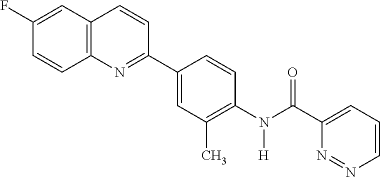 [SMILES: Cc1cc(-c2ccc3cc(F)ccc3n2)ccc1NC(=O)c1cccnn1]

The title compound was prepared analogously to Example 029 where pyridazine-3-carboxylic acid was substituted in place of pyridazine-4-carboxylic acid. Yield=42%. LCMS (ES, m/z): [M+H]⁺ 359; ¹H-NMR (300 MHz, DMSO-d₆, ppm): δ2.47 (s, 3H), δ7.67-7.74 (m, 1H), 57.80-7.84 (m, 1H), δ7.93-8.00 (m, 1H), δ8.01-8.04 (m, 1H), δ8.13-8.18 (m, 2H), δ8.22-8.24 (m, 2H), δ8.36-8.39 (m, 1H), δ8.45-8.51 (m, 1H), δ9.50-9.56 (m, 1H), δ10.73 (s, 1H).

### Example 032: N-(4-(6-fluoroquinolin-2-yl)-2-methylphenyl)pyrazine-2-carboxamide

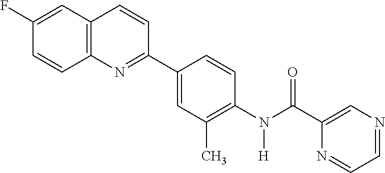 [SMILES: Cc1cc(-c2ccc3cc(F)ccc3n2)ccc1NC(=O)c1cnccn1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and pyrazine-2-carboxylic acid was substituted in place of 1-methyl-1H-pyrazole-5-carboxylic acid. Yield=85%. LCMS (ES, m/z): [M+H]⁺ 359; ¹H-NMR (400 MHz, DMSO-d₆, ppm): δ2.45 (s, 3H), δ7.67-7.73 (m, 1H), δ7.78-7.83 (m, 1H), δ7.96-8.03 (m, 1H), δ8.12-8.18 (m, 2H), δ8.21-8.23 (m, 2H), δ8.45-8.47 (d, 1H), δ8.86 (s, 1H), δ8.98-8.99 (d, 1H), δ9.35 (s, 1H), δ10.36 (s, 1H).

### Example 033: N-(4-(6-fluoroquinolin-2-yl)-2-methylphenyl)-1-methyl-1H-imidazole-2-carboxamide

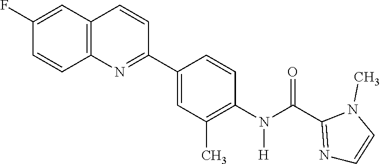 [SMILES: Cc1cc(-c2ccc3cc(F)ccc3n2)ccc1NC(=O)c1nccn1C]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 1-methyl-1H-imidazole-2-carboxylic acid was substituted in place of 1-methyl-1H-pyrazole-5-carboxylic acid. Yield=39%. LCMS (ES, m/z): [M+H]⁺ 361; ¹H-NMR (300 MHz, DMSO-d₆, ppm): δ9.84 (s, 1H), δ8.46-8.43 (d, 1H), δ8.22-8.19 (m, 2H), δ8.16-8.11 (m, 2H), δ7.99-7.96 (d, 1H), δ7.83-7.82 (m, 1H), δ7.72-7.69 (m, 1H), δ7.49 (s, 1H), δ7.12 (s, 1H), δ4.03 (s, 3H), δ2.08 (s, 3H)

### Example 034: N-(4-(6-fluoroquinolin-2-yl)-2-methylphenyl)-1-methyl-1H-imidazole-5-carboxamide

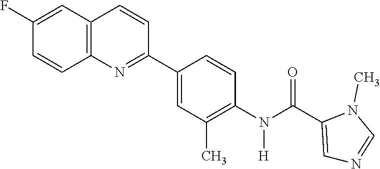 [SMILES: Cc1cc(-c2ccc3cc(F)ccc3n2)ccc1NC(=O)c1cncn1C]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 1-methyl-1H-imidazole-5-carboxylic acid was substituted in place of 1-methyl-1H-pyrazole-5-carboxylic acid. LCMS (ES, m/z): [M+1]⁺ 361; ¹H-NMR (300 MHz, CD₃OD, ppm): δ8.40-8.37 (d, 1H), δ8.19-8.17 (m, 1H), δ8.16-8.08 (m, 1H), δ8.05-8.02 (t, 2H), δ7.85-7.83 (d, 2H), δ7.66-7.57 (m, 3H), δ3.99 (s, 3H), δ2.46 (s, 3H)

### Example 035: N-(4-(6-fluoroquinolin-2-yl)-2-methylphenyl)-1-methyl-1H-imidazole-4-carboxamide

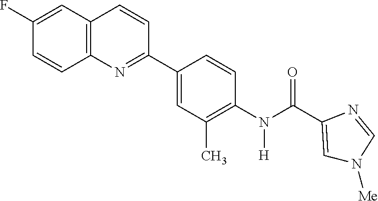 [SMILES: Cc1cc(-c2ccc3cc(F)ccc3n2)ccc1NC(=O)c1cn(C)cn1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and pyrimidine-5-carboxylic acid was substituted in place of 1-methyl-1H-pyrazole-5-carboxylic acid. Yield=39%. LCMS (ES, m/z): [M+H]⁺ 361; ¹H-NMR (300 MHz, DMSO-d₆, ppm): δ9.41 (s, 1H), δ8.45-8.42 (d, 1H), δ8.21-8.11 (m, 5H), δ7.87 (s, 1H), δ7.83-7.79 (m, 2H), δ7.72-7.65 (m, 1H), δ3.76 (s, 3H), δ2.28 (s, 3H)

### Example 036: N-(4-(6-fluoroquinolin-2-yl)-2-methylphenyl)oxazole-4-carboxamide

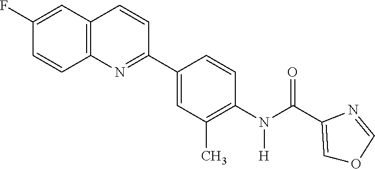 [SMILES: Cc1cc(-c2ccc3cc(F)ccc3n2)ccc1NC(=O)c1cocn1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 1,3-oxazole-4-carboxylic acid was substituted in place of 1-methyl-1H-pyrazole-5-carboxylic acid. Yield=33%. LCMS (ES, m/z): [M+H]⁺ 348; ¹H-NMR (300 MHz, DMSO-d₆, ppm): δ2.40 (s, 3H), δ7.66-7.73 (m, 1H), δ7.79-7.84 (m, 2H), δ8.12-8.16 (m, 2H), δ8.19-8.23 (m, 2H), δ8.44-8.47 (d, 1H), δ8.65-8.66 (d, 1H), δ8.84-8.85 (d, 1H), δ9.71 (s, 1H)

### Example 037: N-(4-(6-fluoroquinolin-2-yl)-2-methylphenyl)isoxazole-5-carboxamide

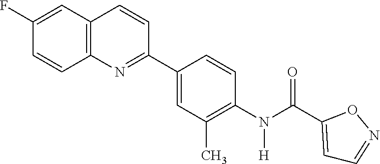 [SMILES: Cc1cc(-c2ccc3cc(F)ccc3n2)ccc1NC(=O)c1ccno1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 1,2-oxazole-5-carboxylic acid was substituted in place of 1-methyl-1H-pyrazole-5-carboxylic acid. Yield=34%. LCMS (ES, m/z): [M+H]⁺ 348; ¹H-NMR (300 MHz, DMSO-d₆, ppm): δ2.38 (s, 3H), δ7.28-7.29 (d, 1H), δ7.55-7.57 (d, 1H), δ7.68-7.73 (m, 1H), δ7.81-7.84 (m, 1H), δ8.13-8.17 (m, 2H), δ8.22-8.24 (m, 2H), δ8.46-48 (d, 1H), δ8.84-8.85 (d, 1H), δ10.51 (s, 1H)

### Example 038: N-(4-(6-fluoroquinolin-2-yl)-2-methylphenyl)-5-methylisoxazole-4-carboxamide

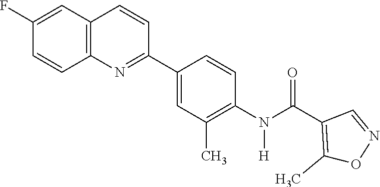 [SMILES: Cc1cc(-c2ccc3cc(F)ccc3n2)ccc1NC(=O)c1cnoc1C]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 5-methyl-1,2-oxazole-4-carboxylic acid was substituted in place of 1-methyl-1H-pyrazole-5-carboxylic acid. Yield=16%. LCMS (ES, m/z): [M+H]⁺ 362; ¹H-NMR (300 MHz, DMSO-d₆, ppm): δ2.38 (s, 3H), δ2.70-2.73 (d, 3H), δ7.55-7.58 (m, 1H), δ7.66-7.73 (m, 1H), δ7.80-7.84 (m, 1H), δ8.12-8.17 (m, 2H), δ8.21-8.24 (m, 2H), δ8.44-8.47 (d, 1H), δ9.08 (s, 1H), δ9.81 (s, 1H)

### Example 039: N-(4-(6-fluoroquinolin-2-yl)-2-methylphenyl)oxazole-2-carboxamide

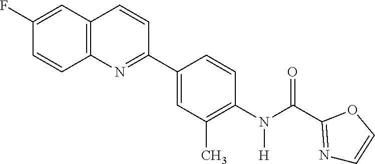 [SMILES: Cc1cc(-c2ccc3cc(F)ccc3n2)ccc1NC(=O)c1ncco1]

The title compound was prepared analogously to Example 001 where 4-(6-fluoroquinolin-2-yl)-2-methylaniline was substituted in place of 2-methyl-4-(6-(trifluoromethyl)quinolin-2-yl)aniline and 1,3-oxazole-2-carboxylic acid was substituted in place of 1-methyl-1H-pyrazole-5-carboxylic acid. Yield=14%. LCMS (ES, m/z): [M+H]⁺ 348; ¹H-NMR (300 MHz, DMSO-d₆, ppm): δ2.40 (s, 3H), δ7.58 (s, 1H), δ7.65-7.73 (m, 2H), δ7.81-7.84 (m, 1H), δ8.12-8.17 (m, 2H), δ8.21-8.23 (m, 2H), δ8.44-8.48 (m, 2H), δ10.38 (s, 1H)

### Example 040: N-(4-(6-fluoroquinolin-2-yl)-2-methylphenyl)acetamide

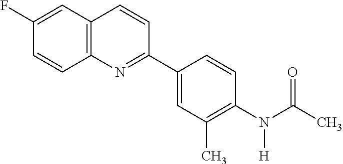 [SMILES: CC(=O)Nc1ccc(-c2ccc3cc(F)ccc3n2)cc1C]

Triethylamine (80.2 mg, 0.79 mmol, 2.000 equiv) was added over a solution of 4-(6-fluoroquinolin-2-yl)-2-methylaniline (100 mg, 0.40 mmol, 1 equiv) and acetyl chloride (34.2 mg, 0.44 mmol, 1.099 equiv) in CH₂Cl₂ (1 mL) at room temperature. The reaction mixture was stirred at room temperature for 3 hours and quenched by addition of water (10 mL). The mixture was extracted with CH₂Cl₂ (3×20 mL) and the organic layers combined and concentrated under reduced pressure. The residue was purified by recrystallization from dichloromethane/hexane=1/10 to afford the desired final product as a white solid in 77% yield. LCMS (ES, m/z): [M+H]⁺ 295; ¹H-NMR (DMSO-d₆, 400 MHz, ppm): δ2.11 (s, 3H), δ2.34 (s, 3H), δ7.66-7.71 (m, 2H), δ7.79-7.82 (m, 1H), δ8.04-8.06 (m, 1H), δ8.10-8.14 (m, 2H), δ8.16-8.19 (d, 1H), δ8.42-8.44 (d, 1H), δ9.37 (s, 1H).

### Example 041: N-(4-(6-fluoroquinolin-2-yl)-2-methylphenyl)morpholine-4-carboxamide

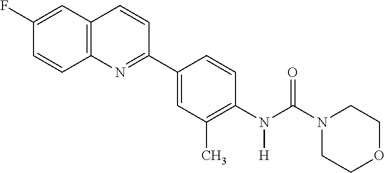 [SMILES: Cc1cc(-c2ccc3cc(F)ccc3n2)ccc1NC(=O)N1CCOCC1]

To a −15° C. solution of 4-(6-fluoroquinolin-2-yl)-2-methylaniline (100 mg, 0.40 mmol, 1 equiv), in dichloromethane (3 mL) was added triphosgene (46.9 mg, 0.16 mmol, 0.4 equiv), followed by triethylamine (100.3 mg, 0.99 mmol, 2.501 equiv). The reaction mixture was stirred at 0° C. for 30 minutes and morpholine (38.0 mg, 0.44 mmol, 1.1 equiv) was added. The reaction mixture was stirred at room temperature for one additional hour and quenched by the slow addition of 10 mL of water. The resulting solution was extracted with CH₂Cl₂ (3×20 mL), the organic layers were combined and concentrated under reduced pressure to afford a residue that was purified by recrystallization from dichloromethane and hexane. The desired final product was isolated as a yellow solid in 80% yield. LCMS (ES, m/z): [M+H]⁺ 366; ¹H-NMR (DMSO-d₆, 400 MHz, ppm): δ2.31 (s, 3H), δ3.44-3.46 (t, 4H), δ3.62-3.65 (t, 4H), δ7.44-7.46 (d, 1H), δ7.66-7.70 (m, 1H), δ7.78-7.81 (m, 1H), δ8.03-8.05 (m, 1H), δ8.10-8.14 (m, 2H), δ8.16-8.18 (d, 2H), δ8.41-8.43 (d, 1H

### Example 042: N-(5-(6-fluoroquinolin-2-yl)-3-methylpyridin-2-yl)-1-methyl-1H-pyrazole-5-carboxamide

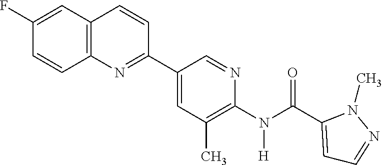 [SMILES: Cc1cc(-c2ccc3cc(F)ccc3n2)cnc1NC(=O)c1ccnn1C]

The title compound was prepared analogously to Example 001, where 5-(6-fluoroquinolin-2-yl)-3-methylpyridin-2-amine was substituted in place of 2-methyl-4-(6-(trifluoromethyl)quinolin-2-yl)aniline. Yield=31%. LCMS (ES, m/z): [M+H]⁺ 362; ¹H-NMR (DMSO-d₆, 300 MHz, ppm): δ2.35 (s, 3H), δ4.08-4.11 (d, 3H), δ7.15-7.16 (d, 1H), δ7.55-7.56 d, 1H), δ7.70-7.77 (m, 1H), δ7.84-8.88 (m, 1H), δ8.16-8.21 (m, 1H), δ8.29-8.32 (d, 1H), δ8.51-8.54 (d, 1H), δ8.59-8.60 (d, 1H), δ9.18-9.19 (d, 1H), δ10.72 (s, 1H)

### Step 1: 5-(6-fluoroquinolin-2-yl)-3-methylpyridin-2-amine

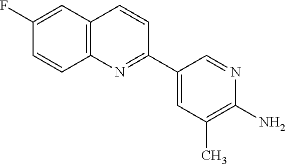 [SMILES: Cc1cc(-c2ccc3cc(F)ccc3n2)cnc1N]

To a solution of 2-chloro-6-fluoroquinoline (200 mg, 1.1 mmol, 1 equiv), 3-methyl-5-(4,4,5,5-tetramethyl-1,3,2-dioxaborolan-2-yl)pyridin-2-amine (387 mg, 1.7 mmol, 1.501 equiv), Na₂CO₃ (351 mg, 3.3 mmol, 2.998 equiv) in DME (4 ml) and H₂O (0.8 ml), Pd(PPh₃)₄ (127 mg, 0.01 mmol, 0.100 equiv) was added. The reaction was heated at 90° C. for 3 hours, and quenched with water (10 mL). The aqueous layer was extracted with EtOAc (3×20 mL), the organic layers were combined and concentrated under reduced pressure. The residue was purified by preparative TLC with dichloromethane/MeOH=(20:1) to afford the desired product as a yellow solid in 64% yield.

### Example 043: N-(5-(6-fluoroquinolin-2-yl)-4-methylpyridin-2-yl)-1-methyl-1H-pyrazole-5-carboxamide

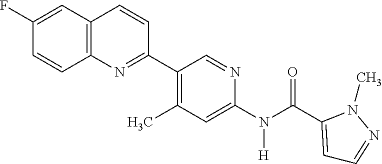 [SMILES: Cc1cc(NC(=O)c2ccnn2C)ncc1-c1ccc2cc(F)ccc2n1]

The title compound was prepared analogously to Example 001 where 5-(6-fluoroquinolin-2-yl)-4-methylpyridin-2-amine was substituted in place of 2-methyl-4-(6-(trifluoromethyl)quinolin-2-yl)aniline. Yield=30%. LCMS (ES, m/z): [M+H]⁺ 362; ¹H-NMR (DMSO-d₆, 400 MHz, ppm): δ2.49 (s, 3H), δ4.14 (s, 3H), δ7.34-7.35 (d, 1H), δ7.54-7.55 (d, 1H), δ7.71-7.76 (m, 1H), δ7.86-7.89 (t, 2H), δ8.13-8.17 (m, 1H), δ8.20 (s, 1H), δ8.48-8.50 (d, 1H), δ8.65 (s, 1H), δ10.94 (s, 1H)

### Step 1: 4-methyl-5-(4,4,5,5-tetramethyl-1,3,2-dioxaborolan-2-yl)pyridin-2-amine

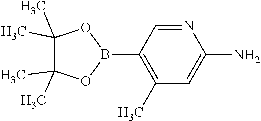 [SMILES: Cc1cc(N)ncc1B1OC(C)(C)C(C)(C)O1]

A solution of 5-bromo-4-methylpyridin-2-amine (10 g, 53.47 mmol, 1 equiv), 4,4,5,5-tetramethyl-2-(tetramethyl-1,3,2-dioxaborolan-2-yl)-1,3,2-dioxaborolane (14.9 g, 58.68 mmol, 1.097 equiv), KOAc (15.7 g, 159.97 mmol, 2.992 equiv) and Pd(dppf)Cl₂ (3.9 g, 5.33 mmol, 0.100 equiv) in 1,4-dioxane (250 ml), was stirred at 115° C. under nitrogen atmosphere overnight. The mixture was allowed to cool down to room temperature and filtered. The filtrate was concentrated under vacuum and the residue was diluted with 100 mL of petroleum ether. The solids were collected by filtration and washed with petroleum ether affording the desired product as a yellow solid in 19% yield.

### Step 2: 5-(6-fluoroquinolin-2-yl)-4-methylpyridin-2-amine

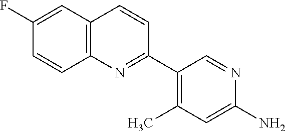 [SMILES: Cc1cc(N)ncc1-c1ccc2cc(F)ccc2n1]

The title compound was prepared analogously to Example 042, step 1 where 4-methyl-5-(4,4,5,5-tetramethyl-1,3,2-dioxaborolan-2-yl)pyridin-2-amine was substituted in place of 3-methyl-5-(4,4,5,5-tetramethyl-1,3,2-dioxaborolan-2-yl)pyridin-2-amine. Yield=75%.

### Example 044: N-(5-(6-fluoroquinolin-2-yl)-6-methylpyridin-2-yl)-1-methyl-1H-pyrazole-5-carboxamide

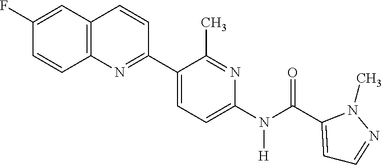 [SMILES: Cc1nc(NC(=O)c2ccnn2C)ccc1-c1ccc2cc(F)ccc2n1]

The title compound was prepared analogously to Example 001 where 5-(6-fluoroquinolin-2-yl)-6-methylpyridin-2-amine was substituted in place of 2-methyl-4-(6-(trifluoromethyl)quinolin-2-yl)aniline. Yield=27%. LCMS (ES, m/z): [M+H]⁺ 362; ¹H-NMR (400 MHz, DMSO-d₆, ppm): δ2.62 (s, 3H), δ4.14 (s, 3H), δ7.37-7.38 (d, 1H), δ7.54-7.55 (d, 1H), 57.71-7.76 (m, 1H), δ7.85-7.89 (m, 2H), δ8.03-8.05 (d, 1H), δ8.12-8.18 (m, 2H), δ8.48-8.50 (d, 1H), δ10.96 (s, 1H).

### Step 1: 6-methyl-5-(4,4,5,5-tetramethyl-1,3,2-dioxaborolan-2-yl)pyridin-2-amine

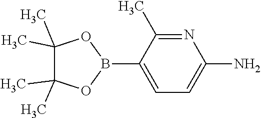 [SMILES: Cc1nc(N)ccc1B1OC(C)(C)C(C)(C)O1]

The title compound was prepared analogously to Example 043, step 1 where 5-bromo-6-methylpyridin-2-amine was substituted in place of 5-bromo-4-methylpyridin-2-amine. Yield=36%.

### Step 2: 5-(6-fluoroquinolin-2-yl)-6-methylpyridin-2-amine

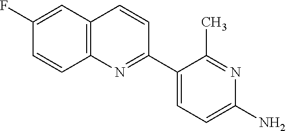 [SMILES: Cc1nc(N)ccc1-c1ccc2cc(F)ccc2n1]

The title compound was prepared analogously to Example 042, step 1, where 6-methyl-5-(4,4,5,5-tetramethyl-1,3,2-dioxaborolan-2-yl)pyridin-2-amine was substituted in place of 3-methyl-5-(4,4,5,5-tetramethyl-1,3,2-dioxaborolan-2-yl)pyridin-2-amine. Yield=90%

### Example 045: N-(6-(6-fluoroquinolin-2-yl)-2-methylpyridin-3-yl)-1-methyl-1H-pyrazole-5-carboxamide

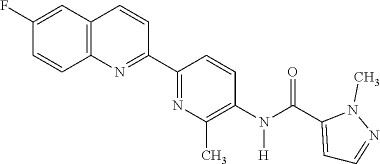 [SMILES: Cc1nc(-c2ccc3cc(F)ccc3n2)ccc1NC(=O)c1ccnn1C]

The title compound was prepared analogously to Example 001 where 6-(6-fluoroquinolin-2-yl)-2-methylpyridin-3-amine was substituted in place of 2-methyl-4-(6-(trifluoromethyl)quinolin-2-yl)aniline. Yield=17%. LCMS (ES, m/z): [M+H]⁺ 362; ¹H-NMR (400 MHz, CDCl₃, ppm): 8.64-8.61 (d, 2H), 8.58-8.54 (t, 1H), 8.34 (s, 1H), 8.27-8.25 (d, 1H), 7.80 (s, 1H), 7.55-7.53 (m, 2H), 7.52-7.47 (m, 1H), 6.77 (s, 1H), 4.25 (s, 3H), 2.73 (s, 3H)

### Step 1: 2-methyl-6-(tributylstannyl)pyridin-3-amine

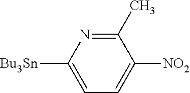

Over a solution of 6-bromo-2-methyl-3-nitropyridine (400 mg, 1.84 mmol, 1.00 equiv), Pd(dppf)Cl₂ (150 mg, 0.21 mmol, 0.10 equiv), and KOAc (2 g, 20.38 mmol, 3.00 equiv) in toluene (6 mL), bis(tributyltin) (3.2 g, 7.83 mmol, 3.00 equiv) was added. The resulting solution was stirred for 12 hours at 110° C. The reaction was cooled down to room temperature and stopped by the addition of water. Extraction with ethyl acetate three times followed by evaporation of volatiles under reduced pressure afforded a crude material that was purified by neutral alumina gel with ethyl acetate/petroleum ether (1:100) to afford the desired product as colorless oil in 25% yield.

### Step 2: 6-fluoro-2-(6-methyl-5-nitropyridin-2-yl)quinoline

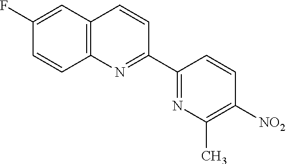 [SMILES: Cc1nc(-c2ccc3cc(F)ccc3n2)ccc1[N+](=O)[O-]]

Pd(PPh₃)₄(54 mg, 0.05 mmol, 0.10 equiv) was added over a solution of 2-chloro-6-fluoroquinoline (84 mg, 0.46 mmol, 1.00 equiv) and 2-methyl-3-nitro-6-(tributylstannyl)pyridine (200 g, 468.20 mmol, 1.00 equiv) in toluene (2 mL). The resulting mixture was stirred for 12 h at 110° C. The reaction was cooled down to room temperature, quenched with water and extracted with ethyl acetate three times. The combined organic layers were dried with MgSO₄ and concentrated under reduced pressure. The resulting residue was purified by silica gel chromatography with ethyl acetate/petroleum ether (1:2) to afford the desired final product as a colorless oil in 91% yield.

### Step 3: 6-(6-fluoroquinolin-2-yl)-2-methylpyridin-3-amine

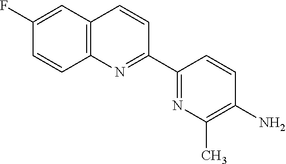 [SMILES: Cc1nc(-c2ccc3cc(F)ccc3n2)ccc1N]

Over a mixture of 6-fluoro-2-(6-methyl-5-nitropyridin-2-yl)quinoline (140 mg, 0.49 mmol, 1.00 equiv) in water (2 mL), Fe (28 mg, 3.00 equiv) and NH₄Cl (133 mg, 2.49 mmol, 5.00 equiv) were added. The resulting solution was stirred for 2 h at 100° C. and extracted with ethyl acetate three times. The organic layers were combined, dried with MgSO₄ and the volatiles evaporated under reduced pressure. The residue was purified by TLC with dichloromethane/methanol (20:1) to afford the desired product as a white solid in 58% yield.

### Example 046: N-(6-(6-fluoroquinolin-2-yl)-5-methylpyridin-3-yl)-1-methyl-1H-pyrazole-5-carboxamide

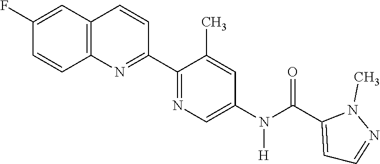 [SMILES: Cc1cc(NC(=O)c2ccnn2C)cnc1-c1ccc2cc(F)ccc2n1]

The title compound was prepared analogously to Example 001 where 6-(6-fluoroquinolin-2-yl)-5-methylpyridin-3-amine was substituted in place of 2-methyl-4-(6-(trifluoromethyl)quinolin-2-yl)aniline. Yield=39%. LCMS (ES, m/z): [M+H]⁺ 362; ¹H-NMR (400 MHz, CDCl₃, ppm): δ2.76 (s, 3H), δ4.26 (s, 3H), δ7.20-7.26 (m, 1H), δ7.49-7.53 (m, 3H), δ8.05-8.07 (d, 1H), δ8.18-8.20 (m, 1H), δ8.26-8.38 (d, 1H), δ8.75 (s, 1H), δ9.20 (s, 2H)

### Step 1: 3-methyl-5-nitro-2-(tributylstannyl)pyridine

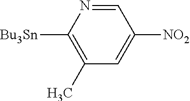

The title compound was prepared analogously to Example 045, step 1, where 2-bromo-3-methyl-5-nitropyridine was substituted in place of 6-bromo-2-methyl-3-nitropyridine. Yield=36%.

### Step 2: 6-fluoro-2-(3-methyl-5-nitropyridin-2-yl)quinoline

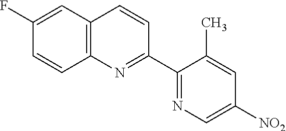 [SMILES: Cc1cc([N+](=O)[O-])cnc1-c1ccc2cc(F)ccc2n1]

The title compound was prepared analogously to Example 045, step 2, where 3-methyl-5-nitro-2-(tributylstannyl)pyridine was substituted in place of 2-methyl-3-nitro-6-(tributylstannyl)pyridine. Yield=30%.

### Step 3: 6-(6-fluoroquinolin-2-yl)-5-methylpyridin-3-amine

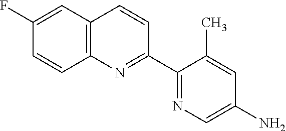 [SMILES: Cc1cc(N)cnc1-c1ccc2cc(F)ccc2n1]

The title compound was prepared analogously to Example 045, step 3, where 6-fluoro-2-(3-methyl-5-nitropyridin-2-yl)quinoline was substituted in place of 6-fluoro-2-(6-methyl-5-nitropyridin-2-yl)quinoline. Yield=61%.

### Example 047: N-(4-(6-fluoroquinolin-2-yl)-2-(trifluoromethoxy)phenyl)-1-methyl-1H-pyrazole-5-carboxamide

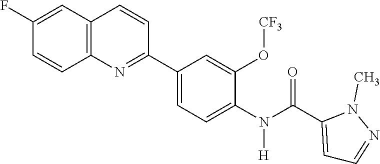 [SMILES: Cn1nccc1C(=O)Nc1ccc(-c2ccc3cc(F)ccc3n2)cc1OC(F)(F)F]

The title compound was prepared analogously to Example 003, where 4-(6-fluoroquinolin-2-yl)-2-(trifluoromethoxy)aniline was substituted in place of 4-(quinoxalin-2-yl)aniline. Yield=33%. LCMS (ES, m/z): [M+H]⁺ 431; ¹H-NMR (400 MHz, CDCl₃, ppm): 8.66 (d, 1H), 8.29-8.08 (m, 5H), 7.89 (d, 1H), 7.58-7.43 (m, 3H), 6.69 (d, 1H), 4.27 (s, 3H).

### Step 1: 4-(6-fluoroquinolin-2-yl)-2-(trifluoromethoxy)aniline

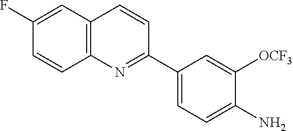 [SMILES: Nc1ccc(-c2ccc3cc(F)ccc3n2)cc1OC(F)(F)F]

The title compound was prepared analogously to Example 014, step 1, where 4-(4,4,5,5-tetramethyl-1,3,2-dioxaborolan-2-yl)-2-(trifluoromethoxy)aniline was substituted in place of tert-butyl (2-methyl-4-(4,4,5,5-tetramethyl-1,3,2-dioxaborolan-2-yl)phenyl)carbamate. Yield=34%.

### Example 048: N-(4-(6-fluoroquinolin-2-yl)-2-(trifluoromethyl)phenyl)-1-methyl-1H-pyrazole-5-carboxamide

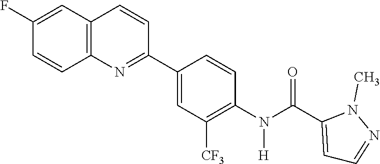 [SMILES: Cn1nccc1C(=O)Nc1ccc(-c2ccc3cc(F)ccc3n2)cc1C(F)(F)F]

The title compound was prepared analogously to Example 003, where 4-(6-fluoroquinolin-2-yl)-2-(trifluoromethyl)aniline was substituted in place of 4-(quinoxalin-2-yl)aniline. Yield=24%. LCMS (ES, m/z): [M+H]⁺ 415; ¹H-NMR (400 MHz, CDCl₃, ppm): 8.61 (m, 2H), 8.39 (dd, 1H), 8.23 (m, 3H), 7.94 (d, 1H), 7.54 (m, 3H), 6.71 (s, 1H), 4.30 (s, 3H)

### Step 1: 4-(6-fluoroquinolin-2-yl)-2-(trifluoromethyl)aniline

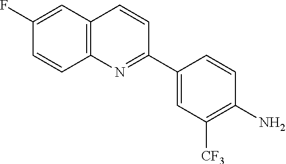 [SMILES: Nc1ccc(-c2ccc3cc(F)ccc3n2)cc1C(F)(F)F]

The title compound was prepared analogously to Example 014, step 1, where 4-(4,4,5,5-tetramethyl-1,3,2-dioxaborolan-2-yl)-2-(trifluoromethyl)aniline was substituted in place of tert-butyl (2-methyl-4-(4,4,5,5-tetramethyl-1,3,2-dioxaborolan-2-yl)phenyl)carbamate. Yield=98%.

### Example 049: N-(4-(8-chloro-6-fluoroquinolin-2-yl)-2-methylphenyl)-1-methyl-1H-pyrazole-5-carboxamide

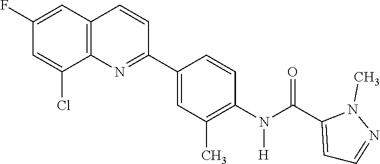 [SMILES: Cc1cc(-c2ccc3cc(F)cc(Cl)c3n2)ccc1NC(=O)c1ccnn1C]

The title compound was prepared analogously to Example 001 where 4-(8-chloro-6-fluoroquinolin-2-yl)-2-methylaniline was substituted in place of 2-methyl-4-(6-(trifluoromethyl)quinolin-2-yl)aniline. Yield=42%. LCMS (ES, m/z): [M+H]⁺ 395; ¹H-NMR (400 MHz, CDCl₃, ppm): δ8.10-8.06 (m, 2H), δ7.64-7.57 (m, 3H), δ7.56-7.54 (m, 2H), δ7.44-7.43 (d, 2H), δ6.68 (s, 1H), δ4.26 (s, 3H), δ2.42 (s, 3H).

### Step 1: N-(2-bromo-4-fluorophenyl)cinnamamide

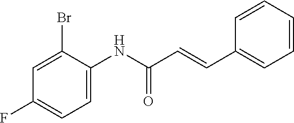 [SMILES: O=C(/C=C/c1ccccc1)Nc1ccc(F)cc1Br]

Pyridine (10.39 g, 131.35 mmol, 1.00 equiv) was added over a solution of 4-dimethylaminopyridine (1.618 g, 13.24 mmol, 0.10 equiv) in dichloromethane (80 mL). The reaction mixture was cooled down to 0° C. and a solution of cinnamoyl chloride (21.85 g, 131.15 mmol, 1.00 equiv) in dichloromethane (66 mL) was added. The resulting solution was stirred for 15 min at 0° C. and a solution of 2-bromo-4-fluoroaniline (25.0 g, 131.57 mmol, 1.00 equiv) in dichloromethane (125 mL) was added dropwise. The cooling bath was removed and the mixture was allowed to react at room temperature for one additional hour. After quenching with 100 mL of H₂O, the solids were collected by filtration to afford the desired product as yellow solid in 88% yield.

### Step 2: 8-bromo-6-fluoroquinolin-2(1H)-one

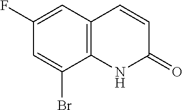 [SMILES: O=c1ccc2cc(F)cc(Br)c2[nH]1]

AlCl₃ (44.89 g, 336.66 mmol, 3.00 equiv) was added over N-(2-bromo-4-fluorophenyl)cinnamamide (36 g, 112.45 mmol, 1.00 equiv) and the resulting mixture was stirred for 1 h at 100° C. The reaction was quenched by the addition of 500 mL of ice/water and the solids collected by filtration to afford the desired product as a white solid in 91% yield

### Step 3: 8-bromo-2-chloro-6-fluoroquinoline

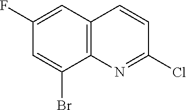 [SMILES: Fc1cc(Br)c2nc(Cl)ccc2c1]

The title compound was prepared analogously to Example 001, step 3, where 8-bromo-6-fluoroquinolin-2(1H)-one was substituted in place of 6-(trifluoromethyl)quinolin-2(1H)-one. Yield=84%.

### Step 4: 8-bromo-6-fluoro-2-(3-methyl-4-nitrophenyl)quinoline

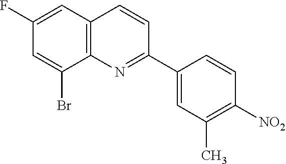 [SMILES: Cc1cc(-c2ccc3cc(F)cc(Br)c3n2)ccc1[N+](=O)[O-]]

The title compound was prepared analogously to Example 001, step 4, where 8-bromo-2-chloro-6-fluoroquinoline was substituted in place of 2-chloro-6-(trifluoromethyl)quinoline and 4,4,5,5-tetramethyl-2-(3-methyl-4-nitrophenyl)-1,3,2-dioxaborolane was substituted in place of 2-methyl-4-(4,4,5,5-tetramethyl-1,3,2-dioxaborolan-2-yl)aniline. Yield=96%.

### Step 5: 4-(8-chloro-6-fluoroquinolin-2-yl)-2-methylaniline

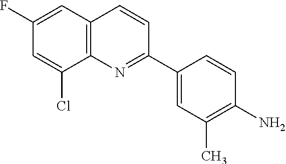 [SMILES: Cc1cc(-c2ccc3cc(F)cc(Cl)c3n2)ccc1N]

The title compound was prepared analogously to Example 045, step 3, where 8-bromo-6-fluoro-2-(3-methyl-4-nitrophenyl)quinoline was substituted in place of 6-fluoro-2-(6-methyl-5-nitropyridin-2-yl)quinoline.

### Example 050: N-(4-(6-fluoroquinolin-2-yl)-3-methylphenyl)-1-methyl-1H-pyrazole-5-carboxamide

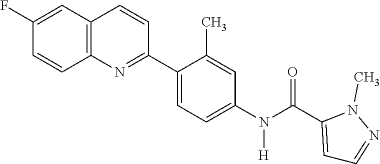 [SMILES: Cc1cc(NC(=O)c2ccnn2C)ccc1-c1ccc2cc(F)ccc2n1]

The title compound was prepared analogously to Example 003, where 4-(6-fluoroquinolin-2-yl)-3-methylaniline was substituted in place of 4-(quinoxalin-2-yl)aniline. Yield=16%; LCMS (ES, m/z): [M+H]⁺ 361; ¹H-NMR (400 MHz, CDCl₃, ppm): 8.19 (d, 2H), 7.80 (s, 1H), 7.54 (m, 7H), 6.72 (s, 1H), 4.24 (s, 3H), 2.46 (s, 3H).

### Step 1: 6-fluoro-2-(2-methyl-4-nitrophenyl)quinoline

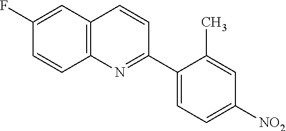 [SMILES: Cc1cc([N+](=O)[O-])ccc1-c1ccc2cc(F)ccc2n1]

The title compound was prepared analogously to Example 014, step 1, where 4,4,5,5-tetramethyl-2-(3-methyl-4-nitrophenyl)-1,3,2-dioxaborolane was substituted in place of tert-butyl (2-methyl-4-(4,4,5,5-tetramethyl-1,3,2-dioxaborolan-2-yl)phenyl)carbamate. Yield=65%.

### Step 2: 4-(6-fluoroquinolin-2-yl)-3-methylaniline

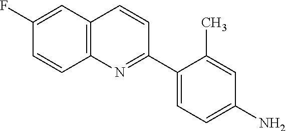 [SMILES: Cc1cc(N)ccc1-c1ccc2cc(F)ccc2n1]

6-fluoro-2-(2-methyl-4-nitrophenyl)quinoline (308 mg, 1.15 mmol) was dissolved in MeOH (15 mL) at room temperature. Zn dust (226 mg, 3.46 mmol) and ammonium chloride (185 mg, 3.46 mmol) were added and the resulting mixture stirred at room temperature for 18 hours. The reaction mixture was filtered and the volatiles evaporated. The resulting residue was purified by chromatography on silica gel with ethyl acetate in hexanes (0 to 100%) to afford the desired product as a yellow solid in 95% yield.

### Example 051: N-(4-(8-bromo-6-fluoroquinolin-2-yl)-2-methylphenyl)-1-methyl-1H-pyrazole-5-carboxamide

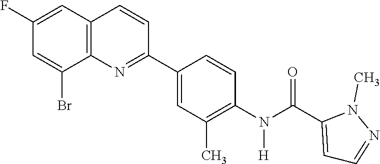 [SMILES: Cc1cc(-c2ccc3cc(F)cc(Br)c3n2)ccc1NC(=O)c1ccnn1C]

The title compound was prepared analogously to Example 001 where 4-(8-bromo-6-fluoroquinolin-2-yl)-2-methylaniline was substituted in place of 2-methyl-4-(6-(trifluoromethyl)quinolin-2-yl)aniline. Yield=63%. LCMS (ES, m/z): [M+H]⁺ 439, 441; ¹H-NMR (400 MHz, CDCl₃, ppm): δ2.47 (s, 3H), δ4.26 (s, 3H), δ6.69-6.70 (d, 1H), δ7.43-7.45 (m, 1H), δ7.54-7.55 (d, 1H), δ7.65 (s, 1H), δ7.87-7.90 (m, 1H), δ7.96-7.98 (d, 1H), δ8.10-8.21 (m, 3H), δ8.25 (s, 1H)

### Step 1: 4-(8-bromo-6-fluoroquinolin-2-yl)-2-methylaniline

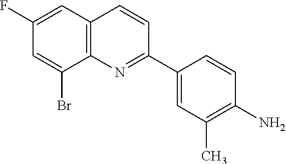 [SMILES: Cc1cc(-c2ccc3cc(F)cc(Br)c3n2)ccc1N]

The title compound was prepared analogously to Example 045, step 3, where 8-bromo-6-fluoro-2-(3-methyl-4-nitrophenyl)quinoline was substituted in place of 6-fluoro-2-(6-methyl-5-nitropyridin-2-yl)quinoline. Yield=38%.

### Example 052: N-(4-(6-fluoro-8-methylquinolin-2-yl)-2-methylphenyl)-1-methyl-1H-pyrazole-5-carboxamide

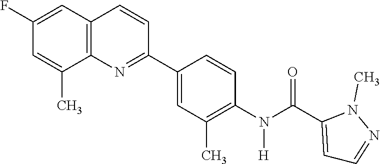 [SMILES: Cc1cc(-c2ccc3cc(F)cc(C)c3n2)ccc1NC(=O)c1ccnn1C]

Pd(dppf)Cl₂ (16.7 mg, 0.02 mmol, 0.1 equiv) was added over a solution of N-(4-(8-bromo-6-fluoroquinolin-2-yl)-2-methylphenyl)-1-methyl-1H-pyrazole-5-carboxamide (100 mg, 0.23 mmol, 1 equiv), methylboronic acid (27.3 mg, 0.46 mmol, 2 equiv) and K₂CO₃ (62.9 mg, 0.46 mmol, 2 equiv) in toluene (2 mL). The resulting solution was stirred at 100° C. overnight and then quenched by the addition of 5 mL of water. The mixture was extracted with ethyl acetate (2×5 mL) and the organic layers combined and concentrated under reduced pressure. The residue was purified by silica gel chromatography with dichloromethane/methanol (30/1) to afford the desired final product as a white solid in 48% yield. LCMS (ES, m/z): [M+H]⁺ 375; ¹H-NMR (400 MHz, DMSO-d₆, ppm): δ2.37 (s, 3H), δ2.85 (s, 3H), δ4.11 (s, 3H), δ7.10-7.11 (d, 1H), δ7.53-7.64 (m, 4H), δ8.17-8.23 (m, 3H), δ8.41-8.43 (d, 1H), δ9.97 (s, 1H)

### Example 053: N-(4-(8-cyclopropyl-6-fluoroquinolin-2-yl)-2-methylphenyl)-1-methyl-1H-pyrazole-5-carboxamide

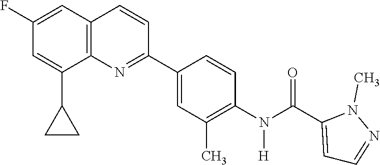 [SMILES: Cc1cc(-c2ccc3cc(F)cc(C4CC4)c3n2)ccc1NC(=O)c1ccnn1C]

The title compound was prepared analogously to Example 052, where cyclopropylboronic acid was substituted in place of methylboronic acid. Yield=42%. LCMS (ES, m/z): [M+H]⁺ 401; ¹H-NMR (400 MHz, CDCl₃, ppm): δ0.90-0.94 (m, 2H), δ1.26-1.29 (m, 2H), δ2.46 (s, 3H), δ3.47-3.56 (m, 1H), δ4.26 (s, 3H), δ6.68-6.69 (d, 1H), δ6.91-6.94 (m, 1H), δ7.20-7.23 (m, 1H), δ7.54 (s, 1H), δ7.63 (s, 1H), δ7.91-7.93 (d, 1H), δ8.08-8.18 (m, 4H)

### Example 054: N-(4-(8-cyano-6-fluoroquinolin-2-yl)-2-methylphenyl)-1-methyl-1H-pyrazole-5-carboxamide

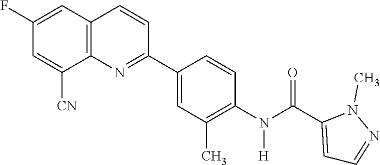 [SMILES: Cc1cc(-c2ccc3cc(F)cc(C#N)c3n2)ccc1NC(=O)c1ccnn1C]

Over a solution of N-[4-(8-bromo-6-fluoroquinolin-2-yl)-2-methylphenyl]-1-methyl-1H-pyrazole-5-carboxamide (100 mg, 0.23 mmol, 1 equiv) in DMSO (2 mL), CuCN (81.6 mg, 0.91 mmol, 4.002 equiv) was added. The resulting solution was stirred for 4 hr at 140° C. and then quenched by the addition of 6 mL of water. The mixture was extracted with ethyl acetate and the combined organic layers (2×4 ml) concentrated. The residue was purified by preparative TLC with dichloromethane/methanol (20/1) to afford the desired product as a white solid in 41% yield. LCMS (ES, m/z): [M+H]⁺ 386; H-NMR (400 MHz, DMSO-d₆, ppm): δ2.39 (s, 3H), δ4.11 (s, 3H), δ7.12 (s, 1H), δ7.57-7.63 (m, 2H), δ8.23-8.29 (m, 3H), δ8.40-8.47 (m, 2H), δ8.60-8.62 (d, 1H)

### Example 055: N-(4-(6-fluoro-8-isopropylquinolin-2-yl)-2-methylphenyl)-1-methyl-1H-pyrazole-5-carboxamide

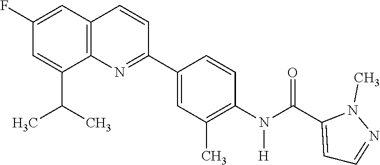 [SMILES: Cc1cc(-c2ccc3cc(F)cc(C(C)C)c3n2)ccc1NC(=O)c1ccnn1C]

A solution of N-(4-(6-fluoro-8-(prop-1-en-2-yl)quinolin-2-yl)-2-methylphenyl)-1-methyl-1H-pyrazole-5-carboxamide (45 mg, 0.11 mmol, 1 equiv) and 10% Pd on carbon (10 mg) in MeOH (5 mL) and THE (5 mL) was hydrogenated at atmospheric pressure and room temperature for 2 hours. The solids were filtered out and the organic solution concentrated. The residue was purified by preparative TLC with dichloromethane/methanol (30/1) to afford the desired product as a white solid in 22% yield. LCMS (ES, m/z): [M+H]⁺ 403; ¹H-NMR (300 MHz, DMSO-d₆, ppm): δ1.29-1.40 (d, 6H), δ2.36 (s, 3H), δ4.09 (s, 3H), δ4.36-4.42 (m, 1H), δ7.09 (s, 1H), δ7.50-7.62 (m, 4H), δ8.14-8.21 (m, 3H), δ8.39-8.42 (d, 1H), δ9.95 (s, 1H)

### Step 1: N-(4-(6-fluoro-8-(prop-1-en-2-yl)quinolin-2-yl)-2-methylphenyl)-1-methyl-1H-pyrazole-5-carboxamide

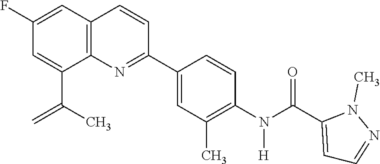 [SMILES: C=C(C)c1cc(F)cc2ccc(-c3ccc(NC(=O)c4ccnn4C)c(C)c3)nc12]

The title compound was prepared analogously to Example 052, where 4,4,5,5-tetramethyl-2-(prop-1-en-2-yl)-1,3,2-dioxaborolane acid was substituted in place of methylboronic acid. Yield=38%.

### Example 056: N-(4-(8-methoxyquinolin-2-yl)-2-methylphenyl)-1-methyl-1H-pyrazole-5-carboxamide

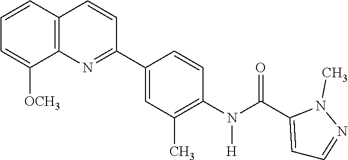 [SMILES: COc1cccc2ccc(-c3ccc(NC(=O)c4ccnn4C)c(C)c3)nc12]

The title compound was prepared analogously to Example 001, where 4-(8-methoxyquinolin-2-yl)-2-methylaniline was substituted in place of 2-methyl-4-(6-(trifluoromethyl)quinolin-2-yl)aniline. Yield=74%. LCMS (ES, m/z): [M+H]⁺ 373; ¹H-NMR (300 MHz, CDCl₃, ppm): δ2.44 (s, 3H), δ4.11 (s, 3H), δ4.25 (s, 3H), δ6.68 (s, 1H), δ7.07-7.09 (d, 1H), δ7.39-7.48 (m, 2H), δ7.52-7.53 (d, 1H), δ7.57 (s, 1H), δ7.90-7.92 (d, 1H), δ7.98-8.01 (d, 1H), δ8.11-8.20 (m, 3H).

### Step 1: 8-methoxy-2-(3-methyl-4-nitrophenyl)quinoline

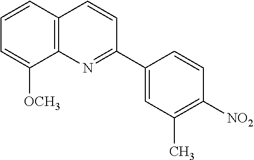 [SMILES: COc1cccc2ccc(-c3ccc([N+](=O)[O-])c(C)c3)nc12]

The title compound was prepared analogously to Example 001, step 4 where 2-chloro-8-(methoxy)quinoline was substituted in place of 2-chloro-6-(trifluoromethyl)quinoline. Yield=73%.

### Step 2: 4-(8-methoxyquinolin-2-yl)-2-methylaniline

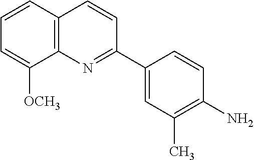 [SMILES: COc1cccc2ccc(-c3ccc(N)c(C)c3)nc12]

The title compound was prepared analogously to Example 045, step 3 where 8-methoxy-2-(3-methyl-4-nitrophenyl)quinoline was substituted in place of 6-fluoro-2-(6-methyl-5-nitropyridin-2-yl)quinoline. Yield=99%.

### Example 057: 1-methyl-N-(2-methyl-4-(5-(trifluoromethyl)quinolin-2-yl)phenyl)-1H-pyrazole-5-carboxamide

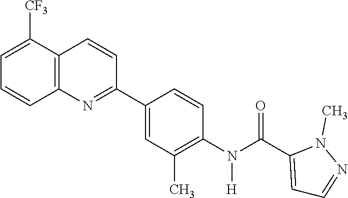 [SMILES: Cc1cc(-c2ccc3c(C(F)(F)F)cccc3n2)ccc1NC(=O)c1ccnn1C]

The title compound was prepared analogously to Example 001, step 4, where 2-chloro-5-(trifluoromethyl)quinoline was substituted in place of 2-chloro-6-(trifluoromethyl)quinoline and 1-methyl-N-[2-methyl-4-(tetramethyl-1,3,2-dioxaborolan-2-yl)phenyl]-1H-pyrazole-5-carboxamide was substituted in place of 2-methyl-4-(4,4,5,5-tetramethyl-1,3,2-dioxaborolan-2-yl)aniline. Yield=63%. LCMS: (ES, m/z): [M+H]⁺ 411; ¹H-NMR: (400 MHz, DMSO-d₆, ppm): δ9.99 (s, 1H), δ8.57-8.55 (d, 1H), δ8.41-8.38 (d, 2H), δ8.26 (s, 1H), δ8.19-8.17 (d, 1H), δ8.09-8.07 (d, 1H), δ7.96-7.92 (t, 1H), δ7.61-7.56 (m, 2H), δ7.12-7.11 (d, 1H), δ4.11 (s, 3H), δ2.08 (s, 3H).

### Step 1: ethyl (E)-3-(2-amino-6-(trifluoromethyl)phenyl)acrylate

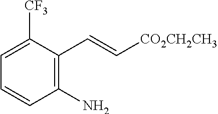

The title compound was prepared analogously to Example 001, step 1, where 2-bromo-3-(trifluoromethyl)aniline was substituted in place of 2-bromo-4-(trifluoromethyl)aniline. Yield=69%.

### Step 2: 5-(trifluoromethyl)quinolin-2(1H)-one

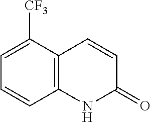 [SMILES: O=c1ccc2c(C(F)(F)F)cccc2[nH]1]

The title compound was prepare analogously to Example 001, step 2, where ethyl (E)-3-(2-amino-6-(trifluoromethyl)phenyl)acrylate was substituted in place of (E)-3-(2-amino-5-(trifluoromethyl)phenyl)acrylate. Yield=35%.

### Step 3: 2-chloro-5-(trifluoromethyl)quinoline

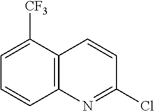 [SMILES: FC(F)(F)c1cccc2nc(Cl)ccc12]

The title compound was prepared analogously to Example 001, step 3, where 5-(trifluoromethyl)quinolin-2(1H)-one was substituted in place of 6-(trifluoromethyl)-1,2-dihydroquinolin-2-one. Yield=52%.

### Example 058: N-(4-(6-fluoro-8-(trifluoromethyl)quinolin-2-yl)-2-methylphenyl)-1-methyl-1H-pyrazole-5-carboxamide

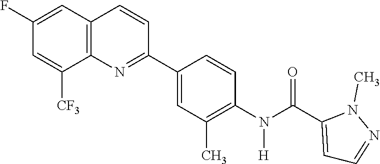 [SMILES: Cc1cc(-c2ccc3cc(F)cc(C(F)(F)F)c3n2)ccc1NC(=O)c1ccnn1C]

The title compound was prepared analogously to Example 001 where 4-(6-fluoro-8-(trifluoromethyl) quinolin-2-yl)-2-methylaniline was substituted in place of 2-methyl-4-(6-(trifluoromethyl)quinolin-2-yl)aniline. Yield=51%. LCMS (ES, m/z): [M+H]⁺ 429; ¹H-NMR (400 MHz, CDCl₃, ppm): δ2.46 (s, 3H), δ4.26 (s, 3H), δ6.68-6.69 (d, 1H), δ7.54 (s, 1H), δ7.61-7.64 (m, 2H), δ7.86-7.88 (m, 1H), δ8.02-8.04 (d, 1H), δ8.10-8.11 (d, 1H), δ8.12-8.22 (m, 3H)

### Step 1: ethyl (E)-3-(2-amino-5-fluoro-3-(trifluoromethyl)phenyl)acrylate

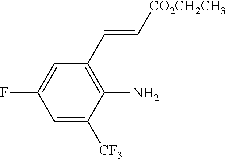

The title compound was prepared analogously to Example 001, step 1, where 2-bromo-4-fluoro-6-(trifluoromethyl)aniline was substituted in place of 2-bromo-4-(trifluoromethyl)aniline. Yield=23%.

### Step 2: 6-fluoro-8-(trifluoromethyl)quinolin-2(1H)-one

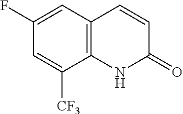 [SMILES: O=c1ccc2cc(F)cc(C(F)(F)F)c2[nH]1]

The title compound was prepared analogously to Example 001, step 2, where ethyl (E)-3-(2-amino-5-fluoro-3-(trifluoromethyl)phenyl)acrylate was substituted in place of (E)-3-(2-amino-5-(trifluoromethyl)phenyl)acrylate. Yield=81%.

### Step 3: 2-chloro-6-fluoro-8-(trifluoromethyl)quinoline

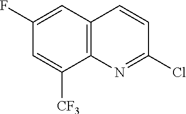 [SMILES: Fc1cc(C(F)(F)F)c2nc(Cl)ccc2c1]

The title compound was prepared analogously to Example 001 step 3, where 6-fluoro-8-(trifluoromethyl)quinolin-2(1H)-one was substituted in place of 6-(trifluoromethyl)-1,2-dihydroquinolin-2-one. Yield=34%.

### Step 4: 4-(6-fluoro-8-(trifluoromethyl)quinolin-2-yl)-2-methylaniline

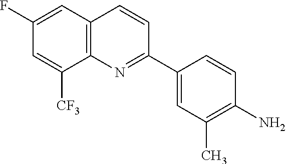 [SMILES: Cc1cc(-c2ccc3cc(F)cc(C(F)(F)F)c3n2)ccc1N]

The title compound was prepared analogously to Example 001, step 4, where 2-chloro-6-fluoro-8-(trifluoromethyl)quinoline was substituted in place 2-chloro-6-(trifluoromethyl)quinoline. Yield=88%.

### Example 059: 1-methyl-N-(2-methyl-4-(8-methyl-1,6-naphthyridin-2-yl)phenyl)-1H-pyrazole-5-carboxamide

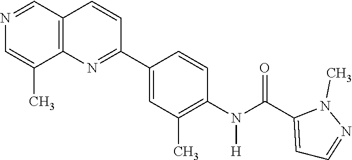 [SMILES: Cc1cc(-c2ccc3cncc(C)c3n2)ccc1NC(=O)c1ccnn1C]

The title compound was prepared analogously to Example 001, where 2-methyl-4-(8-methyl-1,6-naphthyridin-2-yl)aniline was substituted in place of 2-methyl-4-(6-(trifluoromethyl)quinolin-2-yl)aniline. Yield=20%. LCMS (ES, m/z): [M+H]⁺ 358; ¹H-NMR (DMSO-d₆, 300 MHz, ppm): δ2.27 (s, 3H), δ2.83 (s, 3H), δ4.20 (s, 3H), δ7.12 (s, 1H), δ7.56-7.64 (m, 2H), δ8.23-8.26 (d, 1H), δ8.30 (s, 1H), δ8.34-8.37 (d, 1H), δ8.61-8.64 (d, 2H), δ9.24 (s, 1H), δ10.00 (s, 1H).

### Step 1: ethyl (E)-3-(4-amino-5-methylpyridin-3-yl)acrylate

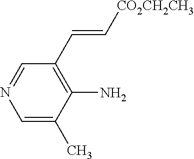

The title compound was prepared analogously to Example 001, step 1, where 3-bromo-5-methylpyridin-4-amine was substituted in place of 2-bromo-4-(trifluoromethyl)aniline. Yield=52%.

### Step 2: 8-methyl-1,6-naphthyridin-2(1H)-one

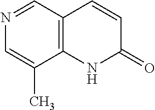

EtONa (2.9 g, 4.00 equiv) was added over a solution of ethyl 3-(4-amino-5-methylpyridin-3-yl)acrylate (2.2 g, 10.67 mmol, 1.00 equiv) in ethanol (66 mL). The resulting solution was stirred for 2 h at 80° C. The reaction was then quenched by the addition of 100 mL of saturated NH₄Cl and extracted with ethyl acetate (3×50 mL). The organic layers were combined and concentrated under reduced pressure to afford the desired final product as a white solid in 9% yield.

### Step 3: 2-chloro-8-methyl-1,6-naphthyridine

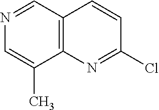

Over a solution of 8-methyl-1,2-dihydro-1,6-naphthyridin-2-one (130 mg, 0.81 mmol, 1.00 equiv) in toluene (5 mL), PPh₃ (638.8 mg, 2.44 mmol, 3.00 equiv) and Cl₃CCN (116 mg, 1.00 equiv) were added. The solution was stirred at 110° C. for 3 hours. The pH value of the solution was adjusted to 7 with 2N HCl and extracted with ethyl acetate (3×20 mL). The organic layers were combined and concentrated under reduced pressure. The resulting residue was purified by preparative TLC with dichloromethane/methanol (25:1) as eluent to afford the final product as a yellow solid in 21% yield.

### Step 4: 2-methyl-4-(8-methyl-1,6-naphthyridin-2-yl)aniline

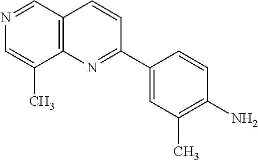 [SMILES: Cc1cc(-c2ccc3cncc(C)c3n2)ccc1N]

The title compound was prepared analogously to Example 001, step 4, where 2-chloro-8-methyl-1,6-naphthyridine was substituted in place of 2-chloro-6-(trifluoromethyl)quinoline and 2-methyl-4-(4,4,5,5-tetramethyl-1,3,2-dioxaborolan-2-yl)aniline was substituted in place of 3-methyl-4-(tetramethyl-1,3,2-dioxaborolan-2-yl)aniline. Yield=72%.

### Example 060: N-(4-(4-chloroisoquinolin-6-yl)-2-methylphenyl)-1-methyl-1H-pyrazole-5-carboxamide

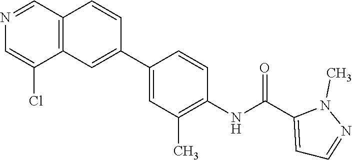 [SMILES: Cc1cc(-c2ccc3cncc(Cl)c3c2)ccc1NC(=O)c1ccnn1C]

The title compound was prepared analogously to Example 001, where 4-(4-chloroisoquinolin-6-yl)-2-methylaniline was substituted in place of 2-methyl-4-(6-(trifluoromethyl)quinolin-2-yl)aniline. Yield=89%. LCMS (ES, m/z): [M+H]⁺ 377; 1H-NMR: (300 MHz, DMSO-d₆, ppm): δ2.37 (s, 3H), δ4.10 (s, 3H), δ7.09-7.10 (d, 1H), δ7.54-7.56 (m, 2H), δ7.75-7.84 (m, 2H), δ8.18-8.37 (m, 3H), δ8.68 (s, 1H), δ9.35 (s, 1H), δ9.98 (s, 1H)

### Step 1: 4-chloro-6-(3-methyl-4-nitrophenyl)isoquinoline

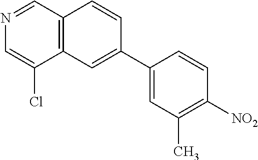 [SMILES: Cc1cc(-c2ccc3cncc(Cl)c3c2)ccc1[N+](=O)[O-]]

Na₂CO₃(524.5 mg, 4.95 mmol, 2 equiv) and Pd(dppf)Cl₂ (181.0 mg, 0.25 mmol, 0.1 equiv) were added to a solution of 6-bromo-4-chloroisoquinoline (600 mg, 2.47 mmol, 1 equiv) and 4,4,5,5-tetramethyl-2-(3-methyl-4-nitrophenyl)-1,3,2-dioxaborolane (976.4 mg, 3.71 mmol, 1.5 equiv) in DME (6 ml) and water (1.5 mL). The mixture was stirred at 50° C. for four hours. Volatiles were removed under reduced pressure and the residue was purified by silica gel column chromatography with petroleum ether/ethyl acetate (10:1) to afford the desired final product as a brown solid (700 mg, 95%)

### Step 2: 4-(4-chloroisoquinolin-6-yl)-2-methylaniline

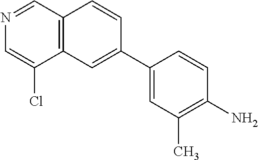 [SMILES: Cc1cc(-c2ccc3cncc(Cl)c3c2)ccc1N]

The title compound was prepared analogously to Example 045, step 3 where 4-chloro-6-(3-methyl-4-nitrophenyl)isoquinoline was substituted in place of 6-fluoro-2-(6-methyl-5-nitropyridin-2-yl)quinoline. Yield=58%.

### Example 061: 1-methyl-N-(2-methyl-4-(4-methylisoquinolin-6-yl)phenyl)-1H-pyrazole-5-carboxamide

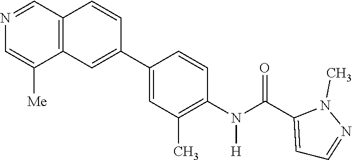 [SMILES: Cc1cc(-c2ccc3cncc(C)c3c2)ccc1NC(=O)c1ccnn1C]

To a solution of N-[4-(4-chloroisoquinolin-6-yl)-2-methylphenyl]-1-methyl-1H-pyrazole-5-carboxamide (300 mg, 0.80 mmol, 1 equiv) in DMF (3 mL), methylboronic acid (143.0 mg, 2.39 mmol, 3 equiv) was added, followed by KOAc (234.4 mg, 2.39 mmol, 3 equiv) and Pd(dppf)Cl₂ (58.3 mg, 0.08 mmol, 0.1 equiv). The resulting mixture was heated at 140 degree ° C. overnight. The reaction was quenched with water, extracted with CH₂Cl₂ (2×5 mL) and the organic layers combined and concentrated under vacuum. The residue was purified by Prep-TLC with dichloromethane/MeOH (20:1) to afford the desired product as a white solid in 47% yield. LCMS (ES, m/z): [M+H]⁺ 357; ¹H-NMR (400 MHz, DMSO-d₆, ppm): δ2.37 (s, 3H), δ2.69 (s, 3H), δ4.10 (s, 3H), δ7.09-7.10 (d, 1H), δ7.55-7.56 (d, 1H), δ7.60 (s, 1H), δ7.77-7.81 (m, 1H), δ7.89 (s, 1H), δ8.06-8.08 (m, 1H), δ8.20-8.25 (m, 2H), δ8.34 (s, 1H), δ9.19 (s, 1H), δ9.98 (s, 1H)

### Example 062: 1-methyl-N-(2-methyl-4-(4-methylcinnolin-6-yl)phenyl)-1H-pyrazole-5-carboxamide

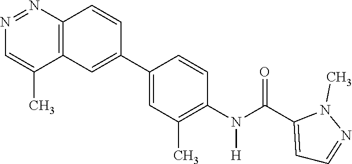 [SMILES: Cc1cc(-c2ccc3nncc(C)c3c2)ccc1NC(=O)c1ccnn1C]

The title compound was prepared analogously to Example 001, where 2-methyl-4-(4-methylcinnolin-6-yl)aniline was substituted in place of 2-methyl-4-(6-(trifluoromethyl)quinolin-2-yl)aniline. Yield=31%. LCMS (ES, m/z): [M+H]⁺ 358; ¹H-NMR (300 MHz, DMSO-d₆, ppm): δ2.38 (s, 3H), δ2.78 (s, 3H), δ4.11 (s, 3H), δ7.11 (s, 1H), δ7.53-7.56 (d, 2H), δ7.81-7.84 (d, 1H), δ7.91 (s, 1H), δ8.30-8.33 (d, 1H), δ8.39 (s, 1H), δ8.49-8.52 (d, 1H), δ9.26 (s, 1H), δ9.98 (s, 1H)

### Step 1: 4-(4-chlorocinnolin-6-yl)-2-methylaniline

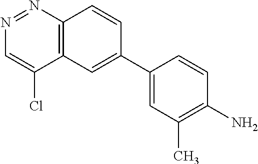 [SMILES: Cc1cc(-c2ccc3nncc(Cl)c3c2)ccc1N]

The title compound was prepared analogously to Example 042, step 1 where 2-methyl-4-(4,4,5,5-tetramethyl-1,3,2-dioxaborolan-2-yl)aniline was substituted in place of 3-methyl-5-(tetramethyl-1,3,2-dioxaborolan-2-yl)pyridin-2-amine and 6-bromo-4-chlorocinnoline was substituted in place of 2-chloro-6-fluoroquinoline. Yield=58%.

### Step 2: 2-methyl-4-(4-methylcinnolin-6-yl)aniline

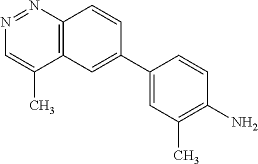 [SMILES: Cc1cc(-c2ccc3nncc(C)c3c2)ccc1N]

K₂CO₃ (276.7 mg, 2.00 mmol, 3 equiv) and Pd(dppf)Cl₂ (48.8 mg, 0.07 mmol, 0.1 equiv) were added to a solution of 4-(4-chlorocinnolin-6-yl)-2-methylaniline (180 mg, 0.67 mmol, 1 equiv) and methylboronic acid (119.8 mg, 2.00 mmol, 3 equiv) in DMF (2 mL). The resulting mixture was stirred under nitrogen at 140° C. overnight. The reaction was quenched by the addition of water (20 mL). The resulting mixture was extracted with ethyl acetate (2×20 mL) and the combined organic layers were combined and concentrated under reduced pressure. The residue was purified by preparative TLC (CH₂Cl₂/MeOH=20/1) to afford the desired product as a yellow solid in 30% yield

### Example 063: N-(4-(6-(1H-tetrazol-5-yl)quinolin-2-yl)-2-methylphenyl)-1-methyl-1H-pyrazole-5-carboxamide

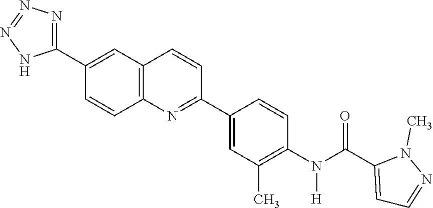 [SMILES: Cc1cc(-c2ccc3cc(-c4nnn[nH]4)ccc3n2)ccc1NC(=O)c1ccnn1C]

To a solution of N-[4-(6-cyanoquinolin-2-yl)-2-methylphenyl]-1-methyl-1H-pyrazole-5-carboxamide (100 mg, 0.27 mmol, 1 equiv) in DMF (5 mL, 0.07 mmol, 0.251 equiv), NH₄Cl (145.6 mg, 2.72 mmol, 10 equiv) and NaN₃ (88.5 mg, 1.36 mmol, 5 equiv) were added. The resulting solution was stirred overnight at 120° C. The reaction was quenched by the addition of 20 mL of water, extracted with ethyl acetate (3×20 ml). The organic layers were combined and concentrated under reduced pressure. The crude product was purified by preparative HPLC (Column: XBridge C18 OBD, 100 Å, 5 μm, 19 mm×250 mm; mobile phase=A: Water (10 mmol/L NH₄HCO₃), mobile phase B=acetonitrile; flow rate=25 mL/min; gradient: 5% B to 55% B in 7 min; Detector=254/220 nm; retention time=5.78 minutes) to afford the desired product as a yellow solid in 33% yield. LCMS (ES, m/z): [M+H]⁺ 411; ¹H-NMR (300 MHz, DMSO-d₆, ppm): δ2.40 (s, 3H), δ4.12 (s, 3H), δ7.11-7.12 (d, 1H), δ7.57-7.60 (t, 2H), δ8.14-8.41 (m, 5H), δ8.63-8.66 (d, 1H), δ8.73-8.74 (d, 1H), δ9.99 (s, 1H)

### Example 064: N-(4-(6-bromoquinolin-2-yl)-2-methylphenyl)-1-methyl-1H-pyrazole-5-carboxamide

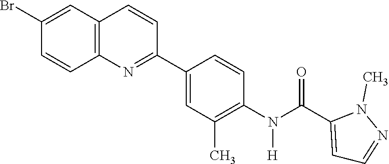 [SMILES: Cc1cc(-c2ccc3cc(Br)ccc3n2)ccc1NC(=O)c1ccnn1C]

The title compound was prepared analogously to Example 001, where 4-(6-bromoquinolin-2-yl)-2-methylaniline was substituted in place of 2-methyl-4-(6-(trifluoromethyl)quinolin-2-yl)aniline. Yield=52%.

### Step 1: 4-(6-bromoquinolin-2-yl)-2-methylaniline

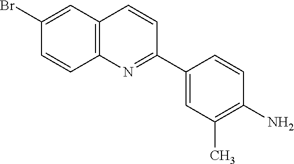 [SMILES: Cc1cc(-c2ccc3cc(Br)ccc3n2)ccc1N]

The title compound was prepared analogously to Example 001, step 4, where 6-bromo-2-chloroquinoline was substituted in place of 2-chloro-6-(trifluoromethyl)quinoline. Yield=70%.

### Example 065: N-(4-(6-Chloroquinolin-2-yl)-2-methylphenyl)-1-methyl-1H-pyrazole-5-carboxamide

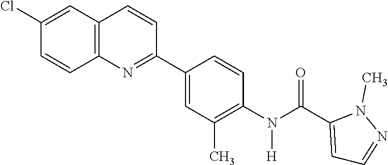 [SMILES: Cc1cc(-c2ccc3cc(Cl)ccc3n2)ccc1NC(=O)c1ccnn1C]

The title compound was prepared analogously to Example 003, where 4-(6-Chloroquinolin-2-yl)-2-methylaniline was substituted in place of 4-(quinoxalin-2-yl)aniline. Yield=35%. LCMS (ES, m/z): [M+H]⁺ 377; ¹H-NMR (400 MHz, CDCl₃, ppm): 8.20 (m, 4H), 8.01 (dd, 1H), 7.92 (d, 1H), 7.84 (d, 1H), 7.68 (m, 2H), 7.54 (d, 1H), 6.69 (d, 1H), 4.26 (s, 3H), 2.47 (s, 3H)

### Step 1: 4-(6-Chloroquinolin-2-yl)-2-methylaniline

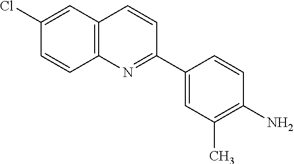 [SMILES: Cc1cc(-c2ccc3cc(Cl)ccc3n2)ccc1N]

The title compound was prepared analogously to Example 014, step 1, where 2,6-dichloroquinoline was substituted in place of 2-chloro-6-fluoroquinoline and 2-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=57%

### Example 066: N-(2-fluoro-4-(6-fluoroquinolin-2-yl)phenyl)-1-methyl-1H-pyrazole-5-carboxamide

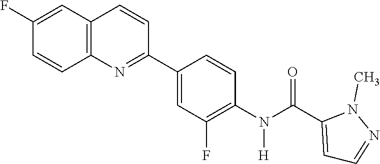 [SMILES: Cn1nccc1C(=O)Nc1ccc(-c2ccc3cc(F)ccc3n2)cc1F]

The title compound was prepared analogously to Example 003, where 2-fluoro-4-(6-fluoroquinolin-2-yl)aniline was substituted in place of 4-(quinoxalin-2-yl)aniline. Yield=20%. LCMS (ES, m/z): [M+H]⁺ 365; ¹H-NMR (400 MHz, CDCl₃, ppm): 8.56 (dd, 1H), 8.15 (m, 3H), 7.96 (m, 2H), 7.89 (d, 1H), 7.48 (m, 3H), 6.73 (d, 1H), 4.25 (s, 3H)

### Step 1: 2-Fluoro-4-(6-fluoroquinolin-2-yl)aniline

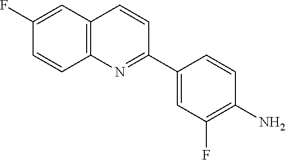 [SMILES: Nc1ccc(-c2ccc3cc(F)ccc3n2)cc1F]

The title compound was prepared analogously to Example 014, step 1, where 2-chloro-6-fluoroquinoline was substituted in place of 1-methyl-N-(2-methyl-4-(6-(trifluoromethyl)quinolin-2-yl)phenyl)-1H-pyrazole-5-carboxamide and 2-fluoro-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=79%

### Example 067: N-(5-fluoro-4-(6-fluoroquinolin-2-yl)-2-methylphenyl)-1-methyl-1H-pyrazole-5-carboxamide

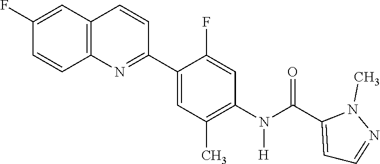 [SMILES: Cc1cc(-c2ccc3cc(F)ccc3n2)c(F)cc1NC(=O)c1ccnn1C]

The title compound was prepared analogously to Example 003, where 2-fluoro-4-(6-fluoroquinolin-2-yl)-5-methylaniline was substituted in place of 4-(quinoxalin-2-yl)aniline. Yield=32%. LCMS (ES, m/z): [M+H]⁺ 379; ¹H-NMR (400 MHz, CDCl₃, ppm): 8.4-7.92 (m, 5H), 7.67 (s, 1H), 7.50 (m, 3H), 6.69 (d, 1H), 4.26 (s, 3H), 2.42 (s, 3H)

### Step 1: 5-fluoro-4-(6-fluoroquinolin-2-yl)-2-methylaniline

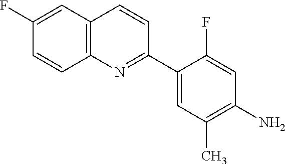 [SMILES: Cc1cc(-c2ccc3cc(F)ccc3n2)c(F)cc1N]

The title compound was prepared analogously to Example 014, step 1, where 2-fluoro-5-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=43%

### Example 068: N-(4-(6-cyclopropylquinolin-2-yl)-2-methylphenyl)-1-methyl-1H-pyrazole-5-carboxamide

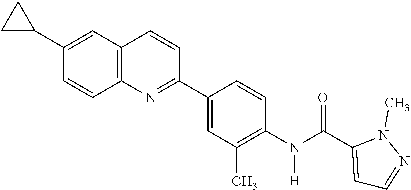 [SMILES: Cc1cc(-c2ccc3cc(C4CC4)ccc3n2)ccc1NC(=O)c1ccnn1C]

The title compound was prepared analogously to Example 052, where N-(4-(6-bromoquinolin-2-yl)-2-methylphenyl)-1-methyl-1H-pyrazole-5-carboxamide was substituted in place of N-(4-(8-bromo-6-fluoroquinolin-2-yl)-2-methylphenyl)-1-methyl-1H-pyrazole-5-carboxamide and cyclopropylboronic acid was substituted in place of methylboronic acid. Yield=53%; LCMS (ES, m/z): [M+H]⁺ 383; ¹H-NMR (400 MHz, CDCl₃, ppm): δ0.86-0.89 (m, 2H), δ1.08-1.19 (m, 2H), δ2.09-2.15 (m, 1H), δ2.47 (s, 3H), δ4.28 (s, 3H), δ6.70 (s, 1H), δ7.45-7.48 (m, 1H), δ7.52-7.56 (m, 2H), δ7.65 (s, 1H), δ7.84-7.86 (d, 1H), δ8.00-8.08 (m, 1H), δ8.10-8.19 (m, 4H).

### Example 069: N-(4-(6-cyclopropylquinolin-2-yl)-2-methylphenyl)-N,1-dimethyl-1H-pyrazole-5-carboxamide

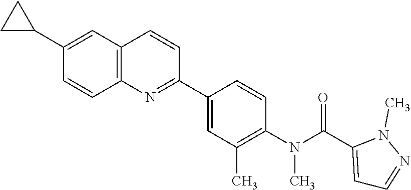 [SMILES: Cc1cc(-c2ccc3cc(C4CC4)ccc3n2)ccc1N(C)C(=O)c1ccnn1C]

The title compound was prepared analogously to Example 002, where N-(4-(6-cyclopropylquinolin-2-yl)-2-methylphenyl)-1-methyl-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=62%. LCMS (ES, m/z): [M+H]⁺ 397; ¹H-NMR (DMSO-d₆, 400 MHz, ppm): δ0.84-8.86 (m, 2H), δ1.06-1.09 (m, 2H), δ2.09-2.19 (m, 1H), δ2.25 (s, 3H), δ3.33 (s, 3H), δ4.00 (s, 3H), δ5.53-5.54 (d, 1H), δ7.14-7.15 (d, 1H), δ7.39-7.48 (d, 1H), δ7.49-7.55 (d, 1H), δ7.68-7.69 (d, 1H), δ7.94-7.96 (d, 1H), δ8.07-8.11 (m, 2H), δ8.18 (s, 1H), δ8.33-8.35 (d, 1H).

### Example 070: 1-methyl-N-(2-methyl-4-(quinolin-2-yl)phenyl)-1H-pyrazole-5-carboxamide

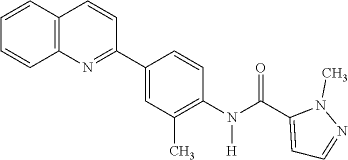 [SMILES: Cc1cc(-c2ccc3ccccc3n2)ccc1NC(=O)c1ccnn1C]

N-[4-(6-bromoquinolin-2-yl)-2-methylphenyl]-1-methyl-1H-pyrazole-5-carboxamide (70 mg, 0.17 mmol, 1 equiv) and 10% Pd on carbon (14 mg) were dissolved in ethyl acetate (5 mL). The mixture was hydrogenated at atmospheric pressure overnight. The solids were filtered out and the solution concentrated. The crude product was purified by preparative TLC (dichloromethane/MeOH=20/1) to afford the desired product as a white solid in 56% yield. LCMS (ES, m/z): [M+H]⁺ 343; ¹H-NMR: (DMSO-d₆, 300 MHz, ppm): δ2.38 (s, 3H), δ4.11 (s, 3H), δ7.10-7.11 (d, 1H), δ7.54-7.63 (m, 3H), δ7.76-7.81 (m, 1H), δ8.00-8.02 (d, 1H), δ8.07-8.10 (d, 1H), δ8.14-8.17 (d, 2H), δ8.20-8.23 (d, 1H), δ8.45-8.48 (d, 1H), δ9.97 (s, 1H).

### Example 071: 1-methyl-N-(2-methyl-4-(6-morpholinoquinolin-2-yl)phenyl)-1H-pyrazole-5-carboxamide

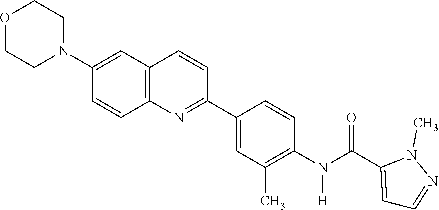 [SMILES: Cc1cc(-c2ccc3cc(N4CCOCC4)ccc3n2)ccc1NC(=O)c1ccnn1C]

A mixture of N-[4-(6-bromoquinolin-2-yl)-2-methylphenyl]-1-methyl-1H-pyrazole-5-carboxamide (150 mg, 0.36 mmol, 1 equiv), morpholine (155.1 mg, 1.78 mmol, 5 equiv), t-BuONa (102.7 mg, 1.07 mmol, 3.001 equiv), Xantphos (41.2 mg, 0.07 mmol, 0.2 equiv), Pd₂(dba)₃.CHCl₃ (36.9 mg, 0.04 mmol, 0.1 equiv) in dioxane (3 mL) was stirred at 100° C. for 4 h. The reaction was then quenched by the addition of 10 mL of water and the resulting solution was extracted with ethyl acetate (3×20 mL) and the combined organic layers concentrated. The residue was purified by preparative TLC with dichloromethane/methanol (20:1) to afford the desired product as a white solid. Yield=26%. LCMS (ES, m/z): [M+H]⁺ 428; ¹H-NMR (DMSO-d₆, 300 MHz, ppm): δ2.36 (s, 3H), 53.27-3.32 (m, 4H), 53.79-3.82 (t, 4H), δ4.10 (s, 3H), δ7.10 (s, 1H), δ7.23-7.24 (d, 1H), δ7.49-7.52 (d, 1H), δ7.55-7.56 (d, 1H), δ7.63-7.67 (t, 1H), δ7.92-7.95 (d, 1H), δ8.03-8.10 (m, 2H), δ8.16 (s, 1H), δ8.22-8.25 (d, 1H), δ9.94 (s, 1H).

### Example 072: N-(4-(6-ethynylquinolin-2-yl)-2-methylphenyl)-1-methyl-1H-pyrazole-5-carboxamide

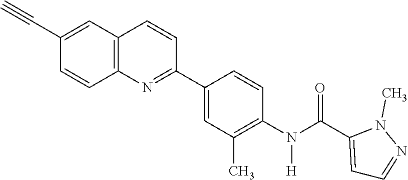 [SMILES: C#Cc1ccc2nc(-c3ccc(NC(=O)c4ccnn4C)c(C)c3)ccc2c1]

To a stirred mixture of 1-methyl-N-(2-methyl-4-(6-((trimethylsilyl)ethynyl)quinolin-2-yl)phenyl)-1H-pyrazole-5-carboxamide (100 mg, 0.23 mmol, 1 equiv) in MeOH (4 mL) was added K₂CO₃ (3.2 mg, 0.02 mmol, 0.1 equiv). The resulting mixture was stirred at room temperature for 1 hour. The reaction was quenched by the addition of water (20 mL) and the resulting mixture was extracted with EtOAc (2×10 mL). The combined organic layers were concentrated under reduced pressure. The crude product was recrystallized with MeOH (5 mL) to afford the desired product as a yellow solid in 98% yield. LCMS: (ES, m/z): [M+H]⁺ 367; ¹H-NMR: (300 MHz, DMSO-d₆, ppm): δ2.39 (s, 3H), δ4.11 (s, 3H), δ4.39 (s, 1H), δ7.11-7.12 (d, 1H), δ7.55-7.58 (q, 2H), δ7.78-7.81 (q, 1H), δ8.05-8.08 (d, 1H), δ8.15-8.25 (m, 4H), δ8.46-8.49 (d, 1H), δ9.98 (s, 1H).

### Step 1: 1-methyl-N-(2-methyl-4-(6-((trimethylsilyl)ethynyl)quinolin-2-yl)phenyl)-1H-pyrazole-5-carboxamide

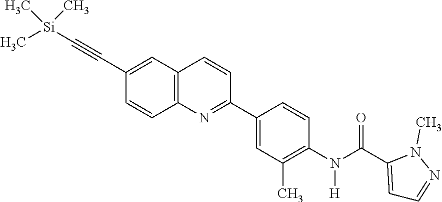 [SMILES: Cc1cc(-c2ccc3cc(C#C[Si](C)(C)C)ccc3n2)ccc1NC(=O)c1ccnn1C]

Et₃N (108.1 mg, 1.07 mmol, 3 equiv), CuI (6.8 mg, 0.04 mmol, 0.1 equiv) and Pd(PPh₃)₂Cl₂ (25.0 mg, 0.04 mmol, 0.1 equiv) were added to a solution of N-[4-(6-bromoquinolin-2-yl)-2-methylphenyl]-1-methyl-1H-pyrazole-5-carboxamide (150 mg, 0.36 mmol, 1 equiv) and ethynyltrimethylsilane (174.9 mg, 1.78 mmol, 5 equiv) in 1,4-dioxane (1.5 mL). The resulting mixture was stirred under nitrogen at 120° C. for 5 hours. The reaction was quenched by the addition of water (20 mL) and extracted with EtOAc (2×20 mL). The combined organic layers were concentrated under reduced pressure and the residue purified by preparative TLC (dichloromethane/MeOH=35/1) to afford the desired product as a white solid in 77% yield.

### Example 073: N-(4-(6-cyanoquinolin-2-yl)-2-methylphenyl)-1-methyl-1H-pyrazole-5-carboxamide

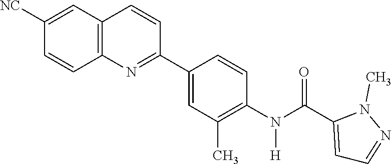 [SMILES: Cc1cc(-c2ccc3cc(C#N)ccc3n2)ccc1NC(=O)c1ccnn1C]

The title compound was prepared analogously to Example 054, where N-[4-(6-bromoquinolin-2-yl)-2-methylphenyl]-1-methyl-1H-pyrazole-5-carboxamide was substituted in place of N-[4-(8-bromo-6-fluoroquinolin-2-yl)-2-methylphenyl]-1-methyl-1H-pyrazole-5-carboxamide. Yield=58%. LCMS: (ES, m/z): [M+H]⁺ 368; ¹H-NMR (400 MHz, CDCl₃, ppm): δ2.47 (s, 3H), δ4.26 (s, 3H), δ6.69-6.70 (d, 1H), δ7.54-7.55 (d, 1H), δ7.67 (s, 1H), δ7.85-7.88 (q, 1H), δ8.00-8.02 (d, 1H), δ8.05-8.07 (q, 1H), δ8.19 (s, 1H), δ8.23-8.28 (m, 4H).

### Example 074: 2-(3-methyl-4-(1-methyl-1H-pyrazole-5-carboxamido)phenyl)quinoline-6-carboxamide

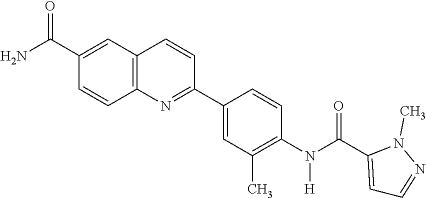 [SMILES: Cc1cc(-c2ccc3cc(C(N)=O)ccc3n2)ccc1NC(=O)c1ccnn1C]

To a solution of N-[4-(6-cyanoquinolin-2-yl)-2-methylphenyl]-1-methyl-1H-pyrazole-5-carboxamide (90 mg, 0.24 mmol, 1 equiv) in MeOH (10 mL) was added NaOH (19.6 mg, 0.49 mmol, 2 equiv) dissolved in 1 mL of water and H₂O₂ (0.5 mL). The resulting mixture was stirred under nitrogen at room temperature overnight. The precipitated solids were collected by filtration and washed with water (3×100 mL). The resulting solid was dried to afford the desired product as a yellow solid in 78% yield. LCMS (ES, m/z): [M+H]⁺ 386; ¹H-NMR (300 MHz, DMSO-d₆, ppm): δ2.39 (s, 3H), δ4.11 (s, 3H), δ7.11 (s, 1H), δ7.50-7.71 (m, 3H), δ8.14-8.26 (m, 6H), δ8.56-8.70 (m, 2H), δ9.99 (s, 1H)

### Example 075: N-(4-(6-aminoquinolin-2-yl)-2-methylphenyl)-1-methyl-1H-pyrazole-5-carboxamide

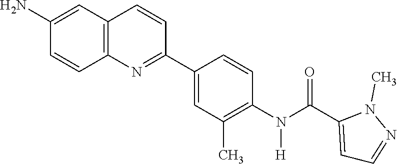 [SMILES: Cc1cc(-c2ccc3cc(N)ccc3n2)ccc1NC(=O)c1ccnn1C]

A mixture of 1-methyl-N-[2-methyl-4-(6-nitroquinolin-2-yl)phenyl]-1H-pyrazole-5-carboxamide (100 mg, 0.26 mmol, 1.00 equiv) and 10% palladium on carbon (40 mg) in methanol (8 mL) and tetrahydrofuran (4 mL) was hydrogenated for 2 hours at 60° C. The solids were filtered out and the solution concentrated under reduced pressure. The residue was purified by preparative TLC with ethyl acetate/petroleum ether (1:5) to afford the desired product as a white solid in 76% yield. LCMS (ES, m/z): [M+H]⁺ 358; ¹H-NMR (DMSO-d₆, 300 MHz ppm): δ2.35 (s, 3H), δ4.11 (s, 3H), δ5.66 (s, 2H), δ6.82-6.83 (s, 1H), δ7.10 (s, 1H), δ7.19-7.20 (d, 1H), δ7.46-7.49 (d, 1H), δ7.55-7.56 (s, 1H), δ7.76-7.78 (d, 1H), δ7.90-7.93 (d, 1H), δ8.01-8.04 (d, 2H), δ8.11 (s, 1H), δ9.93 (s, 1H)

### Example 076: 1-methyl-N-(2-methyl-4-(6-nitroquinolin-2-yl)phenyl)-1H-pyrazole-5-carboxamide

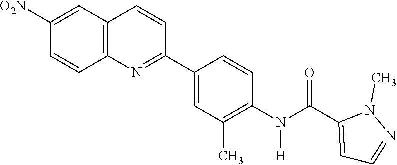 [SMILES: Cc1cc(-c2ccc3cc([N+](=O)[O-])ccc3n2)ccc1NC(=O)c1ccnn1C]

The title compound was prepared analogously to Example 001 where 2-methyl-4-(6-nitroquinolin-2-yl)aniline was substituted in place of 2-methyl-4-(6-(trifluoromethyl)quinolin-2-yl)aniline. Yield=65%. LCMS (ES, m/z): [M+H]⁺ 388; ¹H-NMR (DMSO-d₆, 300 MHz ppm): 2.40 (s, 3H), δ4.12 (s, 3H), δ7.11-7.12 (d, 1H), δ7.57-7.63 (m, 2H), δ8.21-8.30 (m, 3H), δ8.38-8.41 (d, 1H), δ8.41-8.46 (d, 1H), δ8.78-8.81 (d, 1H), δ9.08-9.09 (s, 1H), δ10.00 (s, 1H).

### Step 1: Ethyl (E)-3-(2-amino-5-nitrophenyl)acrylate

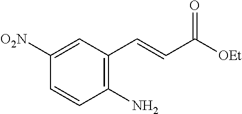 [SMILES: CCOC(=O)/C=C/c1cc([N+](=O)[O-])ccc1N]

The title compound was prepared analogously to Example 001, step 1, where 2-bromo-4-nitroaniline was substituted in place of 2-bromo-4-(trifluoromethyl)aniline. Yield=64%.

### Step 2: 6-nitroquinolin-2(1H)-one

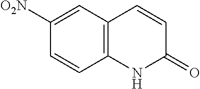 [SMILES: O=c1ccc2cc([N+](=O)[O-])ccc2[nH]1]

The title compound was prepared analogously to Example 001, step 2, where ethyl (E)-3-(2-amino-5-nitrophenyl)acrylate was substituted in place of 2-bromo-4-(trifluoromethyl)aniline. Yield=99%.

### Step 3: 2-chloro-6-nitroquinoline

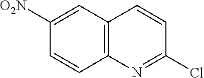 [SMILES: O=[N+]([O-])c1ccc2nc(Cl)ccc2c1]

The title compound was prepared analogously to Example 001, step 3, where 6-nitroquinolin-2(1H)-one was substituted in place of 6-(trifluoromethyl)quinolin-2(1H)-one. Yield=93%.

### Step 4: 2-methyl-4-(6-nitroquinolin-2-yl)aniline

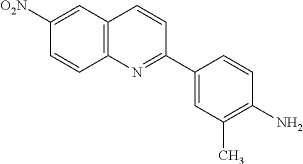 [SMILES: Cc1cc(-c2ccc3cc([N+](=O)[O-])ccc3n2)ccc1N]

The title compound was prepared analogously to Example 001, step 4, where 2-chloro-6-nitroquinoline was substituted in place of 2-chloro-6-(trifluoromethyl)quinoline. Yield=54%.

### Example 077: N-(2-fluoro-4-(6-(trifluoromethyl)quinolin-2-yl)phenyl)-1-methyl-1H-pyrazole-5-carboxamide

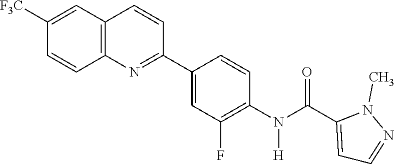 [SMILES: Cn1nccc1C(=O)Nc1ccc(-c2ccc3cc(C(F)(F)F)ccc3n2)cc1F]

The title compound was prepared analogously to Example 003, where 2-fluoro-4-(6-(trifluoromethyl)quinolin-2-yl)aniline was substituted in place of 4-(quinoxalin-2-yl)aniline. Yield=37%. LCMS (ES, m/z): [M+H]⁺ 415; ¹H-NMR (400 MHz, CDCl₃, ppm): 8.61 (dd, 1H), 8.35 (d, 2H), 8.19 (m, 2H), 8.01 (m, 3H), 7.92 (dd, 1H), 7.55 (d, 1H), 6.75 (d, 1H), 4.27 (s, 3H)

### Step 1: 2-fluoro-4-(6-(trifluoromethyl)quinolin-2-yl)aniline

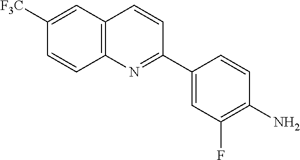 [SMILES: Nc1ccc(-c2ccc3cc(C(F)(F)F)ccc3n2)cc1F]

The title compound was prepare analogously to Example 014, where 2-chloro-6-(trifluoromethyl)quinoline was substituted in place of 2-chloro-6-fluoroquinoline and (4-amino-3-fluorophenyl)boronic acid was substituted in place of tert-butyl (2-methyl-4-(4,4,5,5-tetramethyl-1,3,2-dioxaborolan-2-yl)phenyl)carbamate. Yield=94%.

### Example 078: 1-methyl-N-(2-methyl-4-(7-(trifluoromethyl)quinolin-2-yl)phenyl)-1H-pyrazole-5-carboxamide

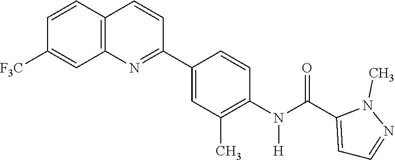 [SMILES: Cc1cc(-c2ccc3ccc(C(F)(F)F)cc3n2)ccc1NC(=O)c1ccnn1C]

The title compound was prepared analogously to Example 001, where 2-methyl-4-(7-(trifluoromethyl)quinolin-2-yl)aniline was substituted in place of 2-methyl-4-(6-(trifluoromethyl)quinolin-2-yl)aniline. Yield=68%. LCMS (ES, m/z): [M+H]⁺ 411; ¹H-NMR (400 MHz, DMSO-d₆, ppm): δ9.98 (s, 1H), δ8.64-8.61 (d, 1H), δ8.42 (s, 1H), δ8.39-8.36 (d, 1H), δ8.28-8.26 (d, 2H), δ8.21-8.18 (d, 1H), δ7.88-7.86 (m, 1H), δ7.60-7.56 (m, 2H), δ7.11 (s, 1H), δ4.11 (s, 3H), δ2.33 (s, 3H).

### Step 1: Ethyl (E)-3-(2-amino-4-(trifluoromethyl)phenyl)acrylate

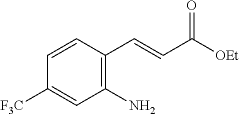 [SMILES: CCOC(=O)/C=C/c1ccc(C(F)(F)F)cc1N]

The title compound was prepared analogously to Example 001, step 1, where 2-bromo-5-(trifluoromethyl)aniline was substituted in place of 2-bromo-4-(trifluoromethyl)aniline. Yield=93%.

### Step 2: 7-(trifluoromethyl)quinolin-2(1H)-one

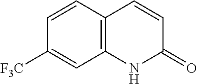 [SMILES: O=c1ccc2ccc(C(F)(F)F)cc2[nH]1]

The title compound was prepared analogously to Example 001, step 2, where Ethyl (E)-3-(2-amino-4-(trifluoromethyl)phenyl)acrylate was substituted in place of (E)-3-(2-amino-5-(trifluoromethyl)phenyl)acrylate. Yield=99%.

### Step 3: 2-chloro-7-(trifluoromethyl)quinoline

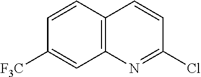 [SMILES: FC(F)(F)c1ccc2ccc(Cl)nc2c1]

The title compound was prepared analogously to Example 001, step 3, where 7-(trifluoromethyl)quinolin-2(1H)-one was substituted in place of 6-(trifluoromethyl)-1,2-dihydroquinolin-2-one. Yield=85%.

### Step 4: 2-methyl-4-(7-(trifluoromethyl)quinolin-2-yl)aniline

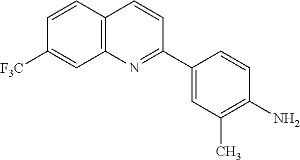 [SMILES: Cc1cc(-c2ccc3ccc(C(F)(F)F)cc3n2)ccc1N]

The title compound was prepared analogously to Example 001, step 4, where 2-chloro-7-(trifluoromethyl)quinoline was substituted in place of 2-chloro-6-(trifluoromethyl)quinoline. Yield=58%.

### Example 079: 1-methyl-N-(2-methyl-4-(6-methylquinolin-2-yl)phenyl)-1H-pyrazole-5-carboxamide

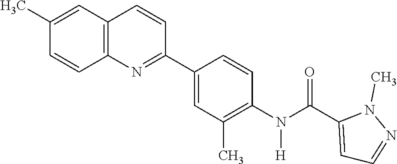 [SMILES: Cc1ccc2nc(-c3ccc(NC(=O)c4ccnn4C)c(C)c3)ccc2c1]

The title compound was prepared analogously to Example 001, where 2-methyl-4-(6-methylquinolin-2-yl)aniline was substituted in place of 2-methyl-4-(6-(trifluoromethyl)quinolin-2-yl)aniline. Yield=48%. LCMS (ES, m/z): [M+H]⁺ 357; ¹H-NMR (300 MHz, CDCl₃, ppm): δ2.46 (s, 3H), δ2.58 (s, 3H), δ4.27 (s, 3H), δ6.70-6.71 (d, 1H), δ7.55-7.61 (m, 3H), δ7.68 (s, 1H), δ7.84-7.87 (d, 1H), δ7.99-8.03 (q, 1H), δ8.09-8.19 (m, 4H)

### Step 1: 2-methyl-4-(6-methylquinolin-2-yl)aniline

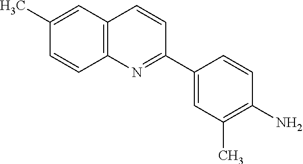 [SMILES: Cc1ccc2nc(-c3ccc(N)c(C)c3)ccc2c1]

The title compound was prepared analogously to Example 001, step 4, where 2-chloro-6-methylquinoline was substituted in place of 2-chloro-6-(trifluoromethyl)quinoline. Yield=72%.

### Example 080: 2,2,2-trifluoro-N-(2-methyl-4-(6-(trifluoromethyl)quinolin-2-yl)phenyl)acetamide

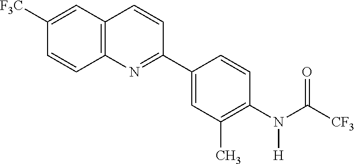 [SMILES: Cc1cc(-c2ccc3cc(C(F)(F)F)ccc3n2)ccc1NC(=O)C(F)(F)F]

To a solution of (Z)-2,2,2-trifluoro-N-(2-methyl-4-(6-(trifluoromethyl)quinolin-2-yl)phenyl)acetimidoyl chloride in THE was added an excess of a concentrated NH₄OH solution. The mixture was stirred at room temperature for 5 minutes. The reaction mixture was diluted with water, extracted with ethyl acetate and dried over MgSO₄. The volatiles were removed under reduced pressure to afford the desired product as a white solid in 93% yield. LCMS (ES, m/z): [M+H]⁺ 399; ¹H-NMR (400 MHz, DMSO-d₆, ppm): 11.12 (s, 1H), 8.70 (d, 1H), 8.55 (s, 1H), 8.40-8.15 (m, 4H), 8.03 (dd, 1H), 7.50 (d, 1H), 3.31 (s, 3H)

### Step 1: (Z)-2,2,2-Trifluoro-N-(2-methyl-4-(6-(trifluoromethyl)quinolin-2-yl)phenyl)acetimidoyl chloride

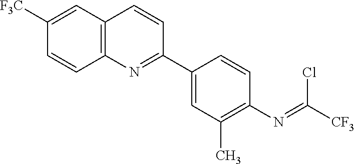 [SMILES: Cc1cc(-c2ccc3cc(C(F)(F)F)ccc3n2)ccc1/N=C(\Cl)C(F)(F)F]

To a solution of Ph₃P in CCl₄ at 0° C. was added Et₃N and stirred for 10 min. Trifluoroacetic acid was added and stirred for another 10 min at 0 degree. Then a solution of 2-methyl-4-(6-(trifluoromethyl)quinolin-2-yl)aniline in CCl₄ was added dropwise. The mixture was heated under reflux for 1 hour and the sovent removed in vacuo. The residue was chromatographed eluting with 0-100% EtOAc-Hexanes to obtain the desired product as a light yellow powder in 18% yield.

### Example 081: 2,2,2-Trifluoro-N-methyl-N-(2-methyl-4-(6-(trifluoromethyl)quinolin-2-yl)phenyl)acetamide

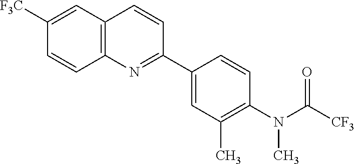 [SMILES: Cc1cc(-c2ccc3cc(C(F)(F)F)ccc3n2)ccc1N(C)C(=O)C(F)(F)F]

The title compound was prepared analogously to Example 002, where 2,2,2-trifluoro-N-(2-methyl-4-(6-(trifluoromethyl)quinolin-2-yl)phenyl)acetamide was substituted in place of 1-methyl-N-(2-methyl-4-(6-(trifluoromethyl)quinolin-2-yl)phenyl)-1H-pyrazole-5-carboxamide. Yield=46%. LCMS (ES, m/z): [M+H]⁺ 413; ¹H-NMR (400 MHz, CDCl₃, ppm): 8.40 (m, 2H), 8.21 (s, 2H), 8.10-7.90 (m, 3H), 7.37 (d, 1H), 3.36 (s, 3H), 2.43 (s, 3H)

### Example 082: 1-Methyl-N-(2-methyl-4-(1-methyl-1H-indol-6-yl)phenyl)-1H-pyrazole-5-carboxamide

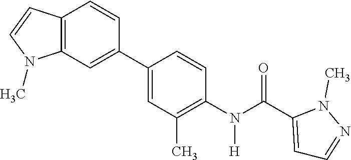 [SMILES: Cc1cc(-c2ccc3ccn(C)c3c2)ccc1NC(=O)c1ccnn1C]

The title compound was prepared analogously to Example 003, where 2-Methyl-4-(1-methyl-1H-indol-6-yl)aniline was substituted in place of 4-(quinoxalin-2-yl)aniline. Yield=32%. LCMS (ES, m/z): [M+H]⁺ 345; ¹H-NMR (400 MHz, CDCl₃, ppm): 7.95 (d, 1H), 7.68 (d, 1H), 7.62-7.47 (m, 5H), 7.37 (dd, 1H), 7.10 (d, 1H), 6.67 (s, 1H), 6.50 (d, 1H), 4.26 (s, 3H), 3.85 (s, 3H), 2.40 (s, 3H)

### Step 1: 1-methyl-6-(3-methyl-4-nitrophenyl)-1H-indole

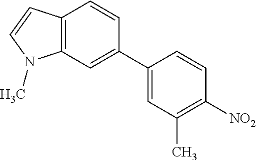 [SMILES: Cc1cc(-c2ccc3ccn(C)c3c2)ccc1[N+](=O)[O-]]

The title compound was prepared analogously to Example 014, step 1, where 6-bromo-1-methyl-1H-indole was substituted in place of 2-chloro-6-fluoroquinoline and 4,4,5,5-tetramethyl-2-(3-methyl-4-nitrophenyl)-1,3,2-dioxaborolane was substituted in place of tert-butyl (2-methyl-4-(4,4,5,5-tetramethyl-1,3,2-dioxaborolan-2-yl)phenyl)carbamate. Yield=74%

### Step 2: 2-Methyl-4-(1-methyl-1H-indol-6-yl)aniline

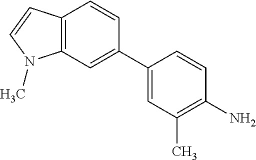 [SMILES: Cc1cc(-c2ccc3ccn(C)c3c2)ccc1N]

The title compound was prepared analogously to Example 050, step 2, where 1-methyl-6-(3-methyl-4-nitrophenyl)-1H-indole was substituted in place of 6-fluoro-2-(2-methyl-4-nitrophenyl)quinoline. Yield=95%.

### Example 083: 1-methyl-N-(2-methyl-4-(1-methyl-1H-indol-5-yl)phenyl)-1H-pyrazole-5-carboxamide

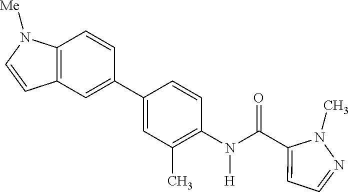 [SMILES: Cc1cc(-c2ccc3c(ccn3C)c2)ccc1NC(=O)c1ccnn1C]

The title compound was prepared analogously to Example 001, where 2-methyl-4-(1-methyl-1H-indol-5-yl)aniline was substituted in place of 2-methyl-4-(6-(trifluoromethyl)quinolin-2-yl)aniline. LCMS (ES, m/z): [M+H]⁺ 345; ¹H-NMR (300 MHz, DMSO-d₆, ppm): 9.89 (s, 1H), 7.85 (s, 1H), 7.61-7.47 (m, 5H), 7.39-7.36 (m, 2H), 7.08 (s, 1H), 6.50-6.49 (d, 1H), 4.10 (s, 3H), 3.83 (s, 3H), 2.31 (s, 3H).

### Step 1: 2-methyl-4-(1-methyl-1H-indol-5-yl)aniline

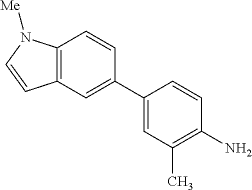 [SMILES: Cc1cc(-c2ccc3c(ccn3C)c2)ccc1N]

The title compound was prepared analogously to Example 001, step 4, where 5-bromo-1-methyl-1H-indole was substituted in place of 2-chloro-6-(trifluoromethyl)quinoline. Yield=9%.

### Example 084: 1-methyl-N-(2-methyl-4-(1-(2,2,2-trifluoroethyl)-1H-pyrrolo[3,2-b]pyridin-5-yl)phenyl)-1H-pyrazole-5-carboxamide

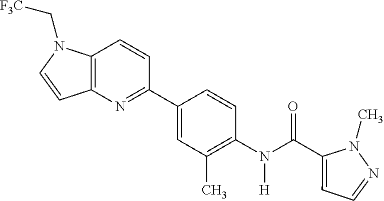 [SMILES: Cc1cc(-c2ccc3c(ccn3CC(F)(F)F)n2)ccc1NC(=O)c1ccnn1C]

The title compound was prepared analogously to Example 001 where 2-methyl-4-(1-(2,2,2-trifluoroethyl)-1H-pyrrolo[3,2-b]pyridin-5-yl)aniline was substituted in place of 2-methyl-4-(6-(trifluoromethyl)quinolin-2-yl)aniline. Yield=55%. LCMS (ES, m/z): [M+H]⁺ 414; ¹H-NMR (400 MHz, CDCl₃, ppm): δ2.41 (s, 3H), δ4.25 (s, 3H), δ4.65-4.71 (q, 2H), δ6.67-6.68 (d, 1H), δ6.89-6.90 (d, 1H), δ7.37-7.38 (d, 1H), δ7.51-7.53 (d, 1H), δ7.63-7.66 (d, 2H), δ7.74-7.76 (m, 1H), δ7.83-7.86 (m, 1H), δ7.98 (s, 1H), δ8.03-8.05 (d, 1H)

### Step 1: 5-chloro-1-(2,2,2-trifluoroethyl)-1H-pyrrolo[3,2-b]pyridine

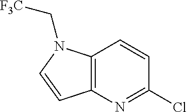 [SMILES: FC(F)(F)Cn1ccc2nc(Cl)ccc21]

A mixture of 5-chloro-1H-pyrrolo[3,2-b]pyridine (1 g, 6.55 mmol, 1 equiv), 1,1,1-trifluoro-2-iodoethane (1.4 g, 6.55 mmol, 1 equiv) and Cs₂CO₃ (2.1 g, 6.55 mmol, 1 equiv) in 10 mL of DMF was stirred for 4 hours at 60° C. The reaction was then quenched by the addition of 30 mL of water, extracted with 10 mL of ethyl acetate and the combined organic layers concentrated under reduced pressure. The residue was purified by silica gel chromatography with ethyl acetate/petroleum ether (1/10) to afford the desired final product as a colorless oil in 42% yield.

### Step 2: 2-methyl-4-(1-(2,2,2-trifluoroethyl)-1H-pyrrolo[3,2-b]pyridin-5-yl)aniline

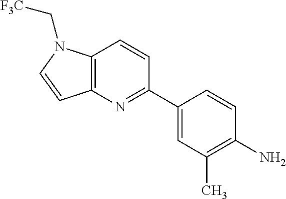 [SMILES: Cc1cc(-c2ccc3c(ccn3CC(F)(F)F)n2)ccc1N]

The title compound was prepared analogously to Example 001, step 4, where 5-chloro-1-(2,2,2-trifluoroethyl)-1H-pyrrolo[3,2-b]pyridine was substituted in place of 2-chloro-6-(trifluoromethyl)quinoline. Yield=11%.

### Example 085: 1-methyl-N-(2-methyl-4-(1-methyl-1H-pyrrolo[3,2-b]pyridin-5-yl)phenyl)-1H-pyrazole-5-carboxamide

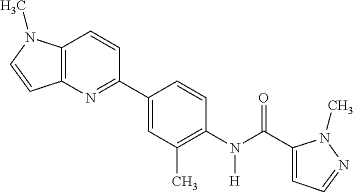 [SMILES: Cc1cc(-c2ccc3c(ccn3C)n2)ccc1NC(=O)c1ccnn1C]

The title compound was prepared analogously to Example 001 where 2-methyl-4-(1-methyl-1H-pyrrolo[3,2-b]pyridin-5-yl)aniline was substituted in place of 2-methyl-4-(6-(trifluoromethyl)quinolin-2-yl)aniline. Yield=51%. LCMS (ES, m/z): [M+H]⁺ 346; ¹H-NMR (DMSO-d₆, 300 MHz, ppm): δ2.28 (s, 3H), δ3.99 (s, 3H), δ4.10 (s, 3H), δ6.78-6.79 (d, 1H), δ7.10-7.11 (d, 1H), δ7.57-7.62 (m, 2H), δ7.90-7.93 (d, 1H), δ7.98-8.05 (m, 3H), δ8.54-8.57 (d, 1H), δ10.02 (s, 1H)

### Step 1: 5-chloro-1-methyl-1H-pyrrolo[3,2-b]pyridine

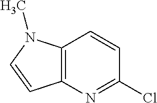

A mixture of 5-chloro-1H-pyrrolo[3,2-b]pyridine (450 mg, 2.95 mmol, 1 equiv), Cs₂CO₃ (960.9 mg, 2.95 mmol, 1 equiv) and iodomethane (418.6 mg, 2.95 mmol, 1.000 equiv) in DMF (11.2 mL), was stirred for 4 h at 60° C. The reaction was quenched by the addition of 30 mL of water, extracted with 10 ml of ethyl acetate and the combined organic layers concentrated under reduced pressure. The residue was purified by silica gel chromatography with ethyl acetate/petroleum ether (1:10) as eluent to afford the desired final product as a yellow oil in 81% yield.

### Step 2: 2-methyl-4-(1-methyl-1H-pyrrolo[3,2-b]pyridin-5-yl)aniline

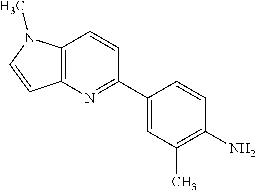 [SMILES: Cc1cc(-c2ccc3c(ccn3C)n2)ccc1N]

The title compound was prepared analogously to Example 001, step 4, where 5-chloro-1-methyl-1H-pyrrolo[3,2-b]pyridine was substituted in place of 2-chloro-6-(trifluoromethyl)quinoline. Yield=18%.

### Example 086: 1-methyl-N-(2-methyl-4-(1-methyl-3-(trifluoromethyl)-1H-indol-6-yl)phenyl)-1H-pyrazole-5-carboxamide

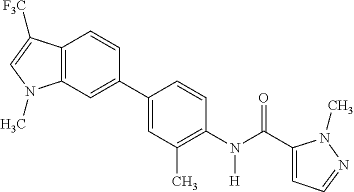 [SMILES: Cc1cc(-c2ccc3c(C(F)(F)F)cn(C)c3c2)ccc1NC(=O)c1ccnn1C]

A mixture of 6-chloro-1-methyl-3-(trifluoromethyl)-1H-indole (300 mg, 1.28 mmol, 1 equiv), 1-methyl-N-[2-methyl-4-(tetramethyl-1,3,2-dioxaborolan-2-yl)phenyl]-1H-pyrazole-5-carboxamide (438.2 mg, 1.28 mmol, 1 equiv), K₃PO₄(545.2 mg, 2.57 mmol, 2 equiv) and Pd-XPhos-G2 (100.9 mg, 0.13 mmol, 0.1 equiv) was dissolved in DMF (3.0 mL). The reaction mixture was irradiated in a microwave reactor for 1 h at 140° C. After dilution with water (20 mL), the aqueous layer was extracted with EtOAc (2×20 mL) and the combined organic layers concentrated under reduced pressure. The residue was purified by preparative TLC (dichloromethane/MeOH=30:1) to afford the desired final product as a white solid in 6% yield. LCMS (ES, m/z): [M+H]⁺ 413; ¹H-NMR (400 MHz, DMSO-d₆, ppm): δ2.34 (s, 3H), δ3.94 (s, 3H), δ4.11 (s, 3H), δ7.09 (s, 1H), δ7.43-7.45 (d, 1H), δ7.55-7.59 (m, 2H), δ7.62-7.71 (m, 2H), δ7.72 (s, 1H), δ7.91 (s, 1H), δ8.03 (s, 1H), δ9.91 (s, 1H)

### Step 1: 6-chloro-3-iodo-1H-indole

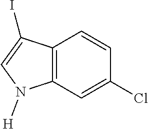 [SMILES: Clc1ccc2c(I)c[nH]c2c1]

Over a solution of 6-chloro-1H-indole (5 g, 32.98 mmol, 1.00 equiv) in DMF (135 mL), potassium hydroxide (4.64 g, 82.69 mmol, 2.50 equiv) was added. A solution of I₂ (8.42 g, 1.00 equiv) in DMF (60 mL) was added to this solution and the resulting mixture stirred for 1 hour at room temperature. After dilution with 400 mL of H₂O, the aqueous layer was extracted with ethyl acetate (2×200 mL) and the organic layers combined and concentrated. The residue was purified by silica gel chromatography with ethyl acetate/petroleum ether (1:20) as eluent to afford the desired product as a white solid. Yield=79%.

### Step 2: 6-chloro-3-iodo-1-methyl-1H-indole

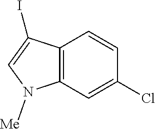 [SMILES: Cn1cc(I)c2ccc(Cl)cc21]

The title compound was prepared analogously to Example 088, step 2, where 6-chloro-3-iodo-1H-indole was substituted in place of 5-(3-methyl-4-nitrophenyl)-1H-indole and methyl iodide was substituted in place of 2,2,2-trifluoroethyl trifluoromethanesulfonate. Yield=99%.

### Step 3: 6-chloro-1-methyl-3-(trifluoromethyl)-1H-indole

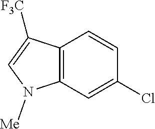 [SMILES: Cn1cc(C(F)(F)F)c2ccc(Cl)cc21]

A mixture of 6-chloro-3-iodo-1-methyl-1H-indole (1 g, 3.43 mmol, lequiv), CuI (3266.5 mg, 17.15 mmol, 5 equiv), KF (996.5 mg, 17.15 mmol, 5 equiv), and TMSCF₃ (2264.0 mg, 17.15 mmol, 5 equiv) in NMP (20 mL) was stirred overnight at 100° C. The reaction was quenched by the addition of 60 mL of water, extracted with ethyl acetate (2×20 mL) and the organic layers combined and concentrated. The residue was purified by silica gel chromatography with ethyl acetate/petroleum ether (1:100) as eluent to afford the desired product as a colorless oil in 99% yield.

### Example 087: N,1-dimethyl-N-(2-methyl-4-(1-methyl-3-(trifluoromethyl)-1H-indol-6-yl)phenyl)-1H-pyrazole-5-carboxamide

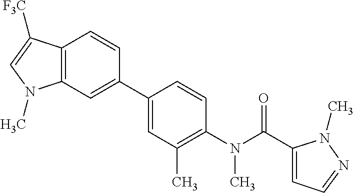 [SMILES: Cc1cc(-c2ccc3c(C(F)(F)F)cn(C)c3c2)ccc1N(C)C(=O)c1ccnn1C]

The title compound was prepared analogously to Example 002, where 1-methyl-N-(2-methyl-4-(1-methyl-3-(trifluoromethyl)-1H-indol-6-yl)phenyl)-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=23%. LCMS (ES, m/z): [M+H]⁺ 427; ¹H-NMR (400 MHz, CDCl₃, ppm): δ2.27 (s, 3H), δ3.44 (s, 3H), δ3.91 (s, 3H), δ4.21 (s, 3H), δ5.52 (s, 1H), δ7.19-7.28 (m, 2H), δ7.46-7.58 (m, 5H), δ7.81-7.83 (d, 1H)

### Example 088: 1-methyl-N-(2-methyl-4-(1-(2,2,2-trifluoroethyl)-1H-indol-5-yl)phenyl)-1H-pyrazole-5-carboxamide

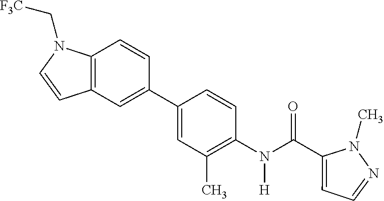 [SMILES: Cc1cc(-c2ccc3c(ccn3CC(F)(F)F)c2)ccc1NC(=O)c1ccnn1C]

The title compound was prepared analogously to Example 001 where 2-methyl-4-(1-(2,2,2-trifluoroethyl)-1H-indol-5-yl)aniline was substituted in place of 2-methyl-4-(6-(trifluoromethyl)quinolin-2-yl)aniline. Yield=31%. LCMS (ES, m/z): [M+H]⁺ 413; ¹H-NMR (400 MHz, CDCl₃, ppm): δ2.40 (s, 3H), δ4.25 (s, 3H), δ4.64-4.70 (m, 2H), δ6.65-6.67 (m, 2H), δ7.14 (s, 1H), δ7.40-7.54 (m, 6H), δ7.83 (s, 1H), δ7.92-7.94 (d, 1H)

### Step 1: 5-(3-methyl-4-nitrophenyl)-1H-indole

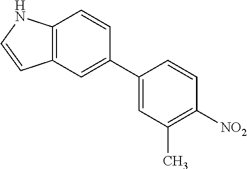 [SMILES: Cc1cc(-c2ccc3[nH]ccc3c2)ccc1[N+](=O)[O-]]

A mixture of 5-(tetramethyl-1,3,2-dioxaborolan-2-yl)-1H-indole (1000 mg, 4.11 mmol, 1 equiv), 4-bromo-2-methyl-1-nitrobenzene (888.6 mg, 4.11 mmol, 1.000 equiv), K₂CO₃ (1137.0 mg, 8.23 mmol, 2 equiv) and Pd(dppf)Cl₂ (301.0 mg, 0.41 mmol, 0.1 equiv) in dioxane (10.0 mL) was stirred at 80° C. for 2 hours. The reaction was concentrated under reduced pressure and the resulting residue was purified by silica gel chromatography with ethyl acetate/petroleum ether (1:10) as eluent to afford the desired product as a yellow solid in 48% yield.

### Step 2: 5-(3-methyl-4-nitrophenyl)-1-(2,2,2-trifluoroethyl)-1H-indole

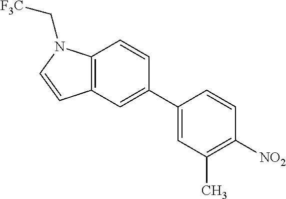 [SMILES: Cc1cc(-c2ccc3c(ccn3CC(F)(F)F)c2)ccc1[N+](=O)[O-]]

5-(3-methyl-4-nitrophenyl)-1H-indole (400 mg, 1.59 mmol, 1 equiv) was dissolved in DMF (5 mL). NaH (63.4 mg, 1.59 mmol, 1 equiv, 60%) was added and the mixture stirred for 0.5 hours. 2,2,2-trifluoroethyl trifluoromethanesulfonate (368.0 mg, 1.59 mmol, 1 equiv) was added and the resulting solution was stirred for 4 additional hours at room temperature. After quenching with 20 mL of saturated NH₄Cl solution, the aqueous layer was extracted with 10 ml of ethyl acetate twice and the combined organic layers concentrated. The residue was purified by silica gel chromatography with ethyl acetate/petroleum ether (1/5) as eluent to afford the desired final product in 62% yield.

### Step 3: 2-methyl-4-(1-(2,2,2-trifluoroethyl)-1H-indol-5-yl)aniline

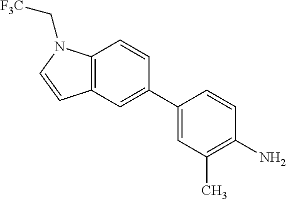 [SMILES: Cc1cc(-c2ccc3c(ccn3CC(F)(F)F)c2)ccc1N]

The title compound was prepared analogously to Example 045, step 3, where 5-(3-methyl-4-nitrophenyl)-1-(2,2,2-trifluoroethyl)-1H-indole was substituted in place of 6-fluoro-2-(6-methyl-5-nitropyridin-2-yl)quinoline. Yield=53%.

### Example 089: 1-Methyl-N-(2-methyl-4-(1-methyl-1H-benzo[d]imidazol-6-yl)phenyl)-1H-pyrazole-5-carboxamide

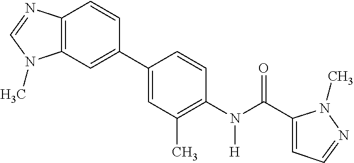 [SMILES: Cc1cc(-c2ccc3ncn(C)c3c2)ccc1NC(=O)c1ccnn1C]

The title compound was prepared analogously to Example 003, where 2-Methyl-4-(1-methyl-1H-benzo[d]imidazol-6-yl)aniline was substituted in place of 4-(quinoxalin-2-yl)aniline. LCMS (ES, m/z): [M+H]⁺ 346; ¹H-NMR (400 MHz, CDCl₃, ppm): 9.90 (s, 1H), 8.21 (s, 1H), 7.90 (s, 1H), 7.75-7.66 (m, 2H), 7.65-7.52 (m, 3H), 7.43 (d, 1H), 7.09 (s, 1H), 4.10 (s, 3H), 3.91 (s, 3H), 2.33 (s, 3H)

### Step 1: 1-Methyl-6-(3-methyl-4-nitrophenyl)-1H-benzo[d]imidazole

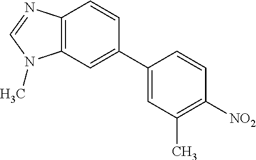 [SMILES: Cc1cc(-c2ccc3ncn(C)c3c2)ccc1[N+](=O)[O-]]

The title compound was prepared analogously to Example 014, step 1, where 6-chloro-1-methyl-1H-benzo[d]imidazole was substituted in place of 2-chloro-6-fluoroquinoline and 4,4,5,5-tetramethyl-2-(3-methyl-4-nitrophenyl)-1,3,2-dioxaborolane was substituted in place of tert-butyl (2-methyl-4-(4,4,5,5-tetramethyl-1,3,2-dioxaborolan-2-yl)phenyl)carbamate. Yield=79%.

### Step 2: 2-Methyl-4-(1-methyl-1H-benzo[d]imidazol-6-yl)aniline

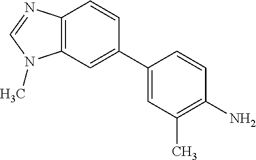 [SMILES: Cc1cc(-c2ccc3ncn(C)c3c2)ccc1N]

The title compound was prepared analogously to Example 050, step 2, where 1-Methyl-6-(3-methyl-4-nitrophenyl)-1H-benzo[d]imidazole was substituted in place of 6-fluoro-2-(2-methyl-4-nitrophenyl)quinoline. Yield=95%.

### Example 090: 1-Methyl-N-(2-methyl-4-(1-methyl-1H-indazol-6-yl)phenyl)-1H-pyrazole-5-carboxamide

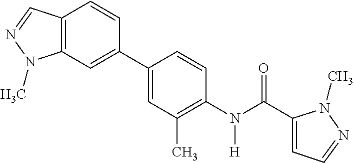 [SMILES: Cc1cc(-c2ccc3cnn(C)c3c2)ccc1NC(=O)c1ccnn1C]

The title compound was prepared analogously to Example 003, 2-Methyl-4-(1-methyl-1H-indazol-6-yl)aniline was substituted in place of 4-(quinoxalin-2-yl)aniline. Yield=18%. LCMS (ES, m/z): [M+H]⁺ 346; ¹H-NMR (400 MHz, CDCl₃, ppm): 8.05-7.96 (m, 2H), 7.78 (d, 1H), 7.63-7.52 (m, 5H), 7.41 (dd, 1H), 6.68 (d, 1H), 4.25 (s, 3H), 4.15 (s, 3H), 2.42 (s, 3H)

### Step 1: 1-Methyl-6-(3-methyl-4-nitrophenyl)-1H-indazole

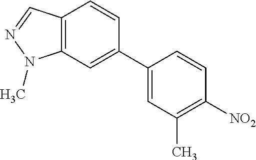 [SMILES: Cc1cc(-c2ccc3cnn(C)c3c2)ccc1[N+](=O)[O-]]

The title compound was prepared analogously to Example 014, step 1, where 6-bromo-1-methyl-1H-indazole was substituted in place of 2-chloro-6-fluoroquinoline and 4,4,5,5-tetramethyl-2-(3-methyl-4-nitrophenyl)-1,3,2-dioxaborolane was substituted in place of tert-butyl (2-methyl-4-(4,4,5,5-tetramethyl-1,3,2-dioxaborolan-2-yl)phenyl)carbamate. Yield=82%.

### Step 2: 2-Methyl-4-(1-methyl-1H-indazol-6-yl)aniline

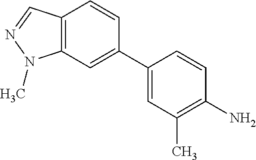 [SMILES: Cc1cc(-c2ccc3cnn(C)c3c2)ccc1N]

The title compound was prepared analogously to Example 050, step 2, where 1-methyl-6-(3-methyl-4-nitrophenyl)-1H-indazole was substituted in place of 6-fluoro-2-(2-methyl-4-nitrophenyl)quinoline. Yield=95%.

### Example 091: 1-Methyl-N-(2-methyl-4-(7-(2,2,2-trifluoroethyl)-7H-pyrrolo[2,3-d]pyrimidin-2-yl)phenyl)-1H-pyrazole-5-carboxamide

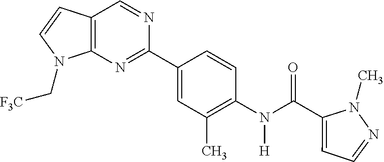 [SMILES: Cc1cc(-c2ncc3ccn(CC(F)(F)F)c3n2)ccc1NC(=O)c1ccnn1C]

The title compound was prepared analogously to Example 003, where 2-Methyl-4-(7-(2,2,2-trifluoroethyl)-7H-pyrrolo[2,3-d]pyrimidin-2-yl)aniline was substituted in place of 4-(quinoxalin-2-yl)aniline. Yield=46%. LCMS (ES, m/z): [M+H]⁺ 415; ¹H-NMR (400 MHz, CDCl₃, ppm): 9.06 (s, 1H), 8.47-8.41 (m, 2H), 8.16 (m, 1H), 7.64 (s, 1H), 7.53 (d, 1H), 7.28 (d, 1H), 6.69-6.65 (m, 2H), 4.96 (q, 2H), 4.26 (s, 3H), 2.45 (s, 3H)

### Step 1: 2-chloro-7-(2,2,2-trifluoroethyl)-7H-pyrrolo[2,3-d]pyrimidine

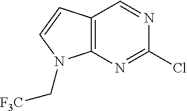 [SMILES: FC(F)(F)Cn1ccc2cnc(Cl)nc21]

The title compound was prepared analogously to Example 088, step 2, where 2-chloro-7H-pyrrolo[2,3-d]pyrimidine was substituted in place of 5-(3-methyl-4-nitrophenyl)-1H-indole and THE was substituted in place of DMF as solvent. Yield=46%.

### Step 2: 2-Methyl-4-(7-(2,2,2-trifluoroethyl)-7H-pyrrolo[2,3-d]pyrimidin-2-yl)aniline

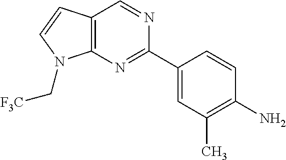 [SMILES: Cc1cc(-c2ncc3ccn(CC(F)(F)F)c3n2)ccc1N]

The title compound was prepared analogously to Example 014, step 1, where 2-chloro-7-(2,2,2-trifluoroethyl)-7H-pyrrolo[2,3-d]pyrimidine was substituted in place of 2-chloro-6-fluoroquinoline and 2-methyl-4-(4,4,5,5-tetramethyl-1,3,2-dioxaborolan-2-yl)aniline was substituted in place of tert-butyl (2-methyl-4-(4,4,5,5-tetramethyl-1,3,2-dioxaborolan-2-yl)phenyl)carbamate. Yield=60%.

### Example 098: N-(4-(6-fluorobenzo[d]thiazol-2-yl)-2-methylphenyl)-1-methyl-1H-pyrazole-5-carboxamide

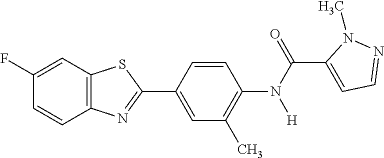 [SMILES: Cc1cc(-c2nc3ccc(F)cc3s2)ccc1NC(=O)c1ccnn1C]

The title compound was prepared analogously to Example 001, where 4-(6-fluorobenzo[d]thiazol-2-yl)-2-methylaniline was substituted in place of 2-methyl-4-(6-(trifluoromethyl)quinolin-2-yl)aniline. Yield=44%. LCMS (ES, m/z): [M+H]⁺ 367; ¹H-NMR (DMSO-d₆, 400 MHz, ppm): δ2.08 (s, 3H), δ4.10 (s, 3H), δ7.10-7.11 (d, 1H), δ7.40-7.46 (m, 1H), δ7.56-7.61 (m, 2H), δ7.93-7.96 (m, 1H), δ8.02 (d, 1H), δ8.07-8.11 (m, 2H), δ10.01 (s, 1H).

### Step 1: 4-(6-fluorobenzo[d]thiazol-2-yl)-2-methylaniline

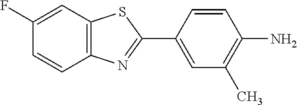 [SMILES: Cc1cc(-c2nc3ccc(F)cc3s2)ccc1N]

The title compound was prepared analogously to Example 001, step 4, where 2-chloro-6-fluorobenzo[d]thiazole was substituted in place of 2-chloro-6-(trifluoromethyl)quinoline. Yield=80%.

### Example 099: N-(4-(5-fluorobenzo[d]thiazol-2-yl)-2-methylphenyl)-1-methyl-1H-pyrazole-5-carboxamide

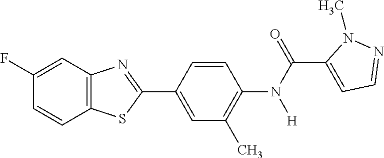 [SMILES: Cc1cc(-c2nc3cc(F)ccc3s2)ccc1NC(=O)c1ccnn1C]

The title compound was prepared analogously to Example 001, where 4-(5-fluorobenzo[d]thiazol-2-yl)-2-methylaniline was substituted in place of 2-methyl-4-(6-(trifluoromethyl)quinolin-2-yl)aniline and 1-methyl-N-(2-methyl-4-(4,4,5,5-tetramethyl-1,3,2-dioxaborolan-2-yl)phenyl)-1H-pyrazole-5-carboxamide was substituted in place of 3-methyl-4-(tetramethyl-1,3,2-dioxaborolan-2-yl)aniline. Yield=23%. LCMS (ES, m/z): [M+H]⁺ 367; ¹H-NMR (CDCl₃, 300 MHz, ppm) δ2.43 (s, 3H), δ4.25 (s, 3H), δ6.67-6.68 (d, 1H), δ7.13-7.19 (m, 1H), δ7.53-7.54 (d, 1H), δ7.66 (s, 1H), δ7.72-7.76 (m, 1H), δ7.80-7.84 (m, 1H), δ7.92-7.94 (d, 1H), δ8.01 (s, 1H), δ8.20-8.23 (d, 1H)

### Example 100: 1-methyl-N-(2-methyl-4-(5-(trifluoromethyl)benzo[d]thiazol-2-yl)phenyl)-1H-pyrazole-5-carboxamide

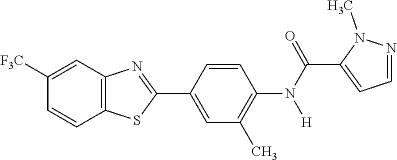 [SMILES: Cc1cc(-c2nc3cc(C(F)(F)F)ccc3s2)ccc1NC(=O)c1ccnn1C]

The title compound was prepared analogously to Example 001, where 2-methyl-4-(5-(trifluoromethyl)benzo[d]thiazol-2-yl)aniline was substituted in place of 2-methyl-4-(6-(trifluoromethyl)quinolin-2-yl)aniline. Yield=56%. LCMS (ES, m/z): [M+H]⁺ 417; ¹H-NMR: (300 MHz, DMSO-d₆, ppm): δ2.39 (s, 3H), δ4.11 (s, 3H), δ7.11-7.12 (d, 1H), δ7.57-7.58 (d, 1H), 57.64-7.67 (d, 1H), δ7.79-7.82 (q, 1H), δ7.99-8.08 (m, 2H), δ8.42-8.45 (d, 2H), δ10.03 (s, 1H)

### Step 1: 2-methyl-4-(5-(trifluoromethyl)benzo[d]thiazol-2-yl)aniline

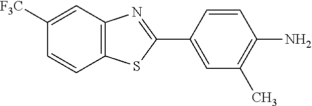 [SMILES: Cc1cc(-c2nc3cc(C(F)(F)F)ccc3s2)ccc1N]

The title compound was prepared analogously to Example 001, step 4, where 2-chloro-5-(trifluoromethyl)-1,3-benzothiazole was substituted in place of 2-chloro-6-(trifluoromethyl)quinoline. Yield=87%.

### Example 101: 1-methyl-N-(2-methyl-4-(6-(trifluoromethyl)benzo[d]thiazol-2-yl)phenyl)-1H-pyrazole-5-carboxamide

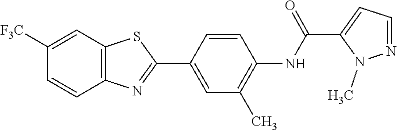 [SMILES: Cc1cc(-c2nc3ccc(C(F)(F)F)cc3s2)ccc1NC(=O)c1ccnn1C]

The title compound was prepared analogously to Example 001, where 2-methyl-4-(6-(trifluoromethyl)benzo[d]thiazol-2-yl)aniline was substituted in place of 2-methyl-4-(6-(trifluoromethyl)quinolin-2-yl)aniline. Yield=50%. LCMS (ES, m/z): [M+H]⁺ 417; ¹H-NMR (300 MHz, CDCl₃, ppm): δ2.46 (s, 3H), δ4.27 (s, 3H), δ6.70-6.71 (d, 1H), δ7.29 (s, 1H), δ7.70-7.77 (m, 2H), δ7.97-8.01 (q, 1H), δ8.06 (s, 1H), δ8.15-8.31 (m, 3H)

### Step 1: 2-methyl-4-(6-(trifluoromethyl)benzo[d]thiazol-2-yl)aniline

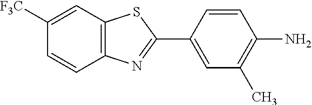 [SMILES: Cc1cc(-c2nc3ccc(C(F)(F)F)cc3s2)ccc1N]

The title compound was prepared analogously to Example 001, step 4, where 2-chloro-6-(trifluoromethyl)-1,3-benzothiazole was substituted in place of 2-chloro-6-(trifluoromethyl)quinoline. Yield=50%.

### Example 102: 1-methyl-N-(2-methyl-4-(6-(trifluoromethyl)-1,5-naphthyridin-2-yl)phenyl)-1H-pyrazole-5-carboxamide

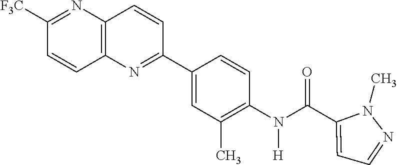 [SMILES: Cc1cc(-c2ccc3nc(C(F)(F)F)ccc3n2)ccc1NC(=O)c1ccnn1C]

The title compound was prepared analogously to Example 001 where 2-methyl-4-(6-(trifluoromethyl)-1,5-naphthyridin-2-yl)aniline was substituted in place of 2-methyl-4-(6-(trifluoromethyl)quinolin-2-yl)aniline. Yield=58%. LCMS (ES, m/z): [M+H]⁺ 412; ¹H-NMR (300 MHz, CDCl₃, ppm): δ2.50 (s, 3H), δ4.28 (s, 3H), δ6.72-6.73 (d, 1H), δ7.56-7.57 (d, 1H), δ7.70 (s, 1H), δ8.00-8.02 (d, 1H), δ8.09-8.13 (q, 1H), δ8.22-8.32 (m, 3H), δ8.57-8.60 (d, 1H), δ8.67-8.70 (d, 1H).

### Step 1: ethyl (E)-3-(3-amino-6-(trifluoromethyl)pyridin-2-yl)acrylate

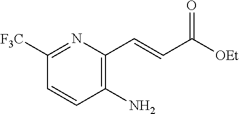 [SMILES: CCOC(=O)/C=C/c1nc(C(F)(F)F)ccc1N]

The title compound was prepared analogously to Example 001, step 1, where 2-bromo-6-(trifluoromethyl)pyridin-3-amine was substituted in place of 2-bromo-4-(trifluoromethyl)aniline. Yield=97%.

### Step 2: 6-(trifluoromethyl)-1,5-naphthyridin-2(1H)-one

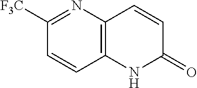 [SMILES: O=c1ccc2nc(C(F)(F)F)ccc2[nH]1]

The title compound was prepared analogously to Example 001, step 2, where (E)-3-(3-amino-6-(trifluoromethyl)pyridin-2-yl)acrylate was substituted in place of (E)-3-(2-amino-5-(trifluoromethyl)phenyl)acrylate. Yield=89%.

### Step 3: 2-chloro-6-(trifluoromethyl)-1,5-naphthyridine

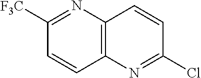 [SMILES: FC(F)(F)c1ccc2nc(Cl)ccc2n1]

The title compound was prepared analogously to Example 001, step 3, 6-(trifluoromethyl)-1,5-naphthyridin-2(1H)-one was substituted in place of 6-(trifluoromethyl)-1,2-dihydroquinolin-2-one. Yield=70%.

### Step 4: 2-methyl-4-(6-(trifluoromethyl)-1,5-naphthyridin-2-yl)aniline

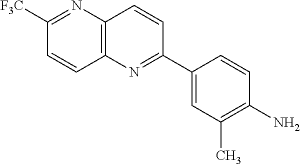 [SMILES: Cc1cc(-c2ccc3nc(C(F)(F)F)ccc3n2)ccc1N]

The title compound was prepared analogously to Example 001, step 4, where 2-chloro-6-(trifluoromethyl)-1,5-naphthyridine was substituted in place of 2-chloro-6-(trifluoromethyl)quinoline. Yield=99%.

### Example 103: N-(2-fluoro-4-(6-(trifluoromethyl)-1,5-naphthyridin-2-yl)phenyl)-1-methyl-1H-pyrazole-5-carboxamide

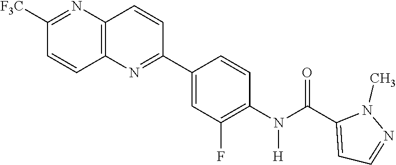 [SMILES: Cn1nccc1C(=O)Nc1ccc(-c2ccc3nc(C(F)(F)F)ccc3n2)cc1F]

The title compound was prepared analogously to Example 001, step 4, where 2-chloro-6-(trifluoromethyl)-1,5-naphthyridine was substituted in place of 2-chloro-6-(trifluoromethyl)quinoline and N-(2-fluoro-4-(4,4,5,5-tetramethyl-1,3,2-dioxaborolan-2-yl)phenyl)-1-methyl-1H-pyrazole-5-carboxamide was substituted in place of 3-methyl-4-(tetramethyl-1,3,2-dioxaborolan-2-yl)aniline. Yield=68%. LCMS (ES, m/z): [M+H]⁺ 416; ¹H-NMR (300 MHz, DMSO-d₆, ppm): 10.30 (s, 1H), 8.79-8.77 (d, 1H), 8.70-8.68 (d, 1H), 8.64-8.62 (d, 1H), 8.34-8.26 (m, 3H), 7.91-7.87 (t, 1H), 7.57 (s, 1H), 7.16 (s, 1H), 4.12 (s, 3H).

### Step 1: N-(4-bromo-2-fluorophenyl)-1-methyl-1H-pyrazole-5-carboxamide

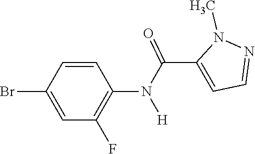 [SMILES: Cn1nccc1C(=O)Nc1ccc(Br)cc1F]

The title compound was prepared analogously to Example 001, where 4-bromo-2-fluoroaniline was substituted in place of 2-methyl-4-(6-(trifluoromethyl)quinolin-2-yl)aniline. Yield=75%.

### Step 2: N-(2-fluoro-4-(4,4,5,5-tetramethyl-1,3,2-dioxaborolan-2-yl)phenyl)-1-methyl-1H-pyrazole-5-carboxamide

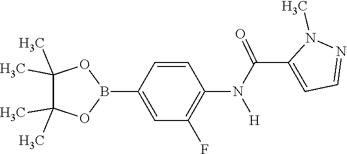 [SMILES: Cn1nccc1C(=O)Nc1ccc(B2OC(C)(C)C(C)(C)O2)cc1F]

The title compound was prepared analogously to Example 005, step 3, where N-(4-bromo-2-fluorophenyl)-1-methyl-1H-pyrazole-5-carboxamide was substituted in place of N-(4-bromo-2-fluoro-3-methylphenyl)-1-methyl-1H-pyrazole-5-carboxamide. Yield=95%.

### Example 104: N-(4-(8-chloro-1,6-naphthyridin-2-yl)-2-methylphenyl)-1-methyl-1H-pyrazole-5-carboxamide

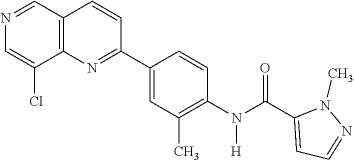 [SMILES: Cc1cc(-c2ccc3cncc(Cl)c3n2)ccc1NC(=O)c1ccnn1C]

The title compound was prepared analogously to Example 001, where 4-(8-chloro-1,6-naphthyridin-2-yl)-2-methylaniline was substituted in place of 2-methyl-4-(6-(trifluoromethyl)quinolin-2-yl)aniline. Yield=42%. LCMS (ES, m/z): [M+H]⁺ 378; ¹H-NMR (400 MHz, CDCl₃, ppm): δ9.15 (s, 1H), δ8.84 (s, 1H), δ8.38-8.35 (d, 1H), δ8.30-8.26 (m, 2H), δ8.16-8.09 (m, 2H), δ7.68 (s, 1H), δ7.55-7.54 (d, 1H), δ6.70-6.69 (d, 1H), δ4.26 (s, 3H), δ2.49 (s, 3H)

### Step 1: ethyl (E)-3-(4-amino-5-chloropyridin-3-yl)acrylate

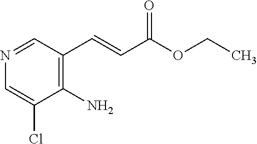

The title compound was prepared analogously to Example 001, step 1, where 3-bromo-5-chloropyridin-4-amine was substituted in place of 2-bromo-4-(trifluoromethyl)aniline. Yield=69%.

### Step 2: 8-chloro-1,6-naphthyridin-2(1H)-one

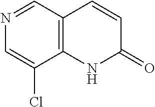 [SMILES: O=c1ccc2cncc(Cl)c2[nH]1]

The title compound was prepared analogously to Example 001, step 2, where ethyl (E)-3-(4-amino-5-chloropyridin-3-yl)acrylate was substituted in place of ethyl (E)-3-(2-amino-5-(trifluoromethyl)phenyl)acrylate. Yield=59%.

### Step 3: 2,8-dichloro-1,6-naphthyridine

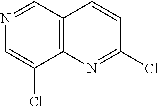 [SMILES: Clc1ccc2cncc(Cl)c2n1]

The title compound was prepared analogously to Example 001, step 3, where 8-chloro-1,6-naphthyridin-2(1H)-one was substituted in place 6-(trifluoromethyl)quinolin-2(1H)-one. Yield=60%.

### Step 4: 4-(8-chloro-1,6-naphthyridin-2-yl)-2-methylaniline

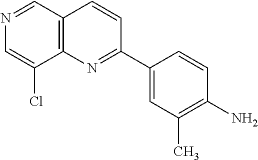 [SMILES: Cc1cc(-c2ccc3cncc(Cl)c3n2)ccc1N]

The title compound was prepared analogously to Example 001, step 4, where 2,8-dichloro-1,6-naphthyridine was substituted in place of 2-chloro-6-(trifluoromethyl)quinoline. Yield=30%.

### Example 105: 1-methyl-N-(2-methyl-4-(8-methyl-6-(trifluoromethyl)-1,5-naphthyridin-2-yl)phenyl)-1H-pyrazole-5-carboxamide

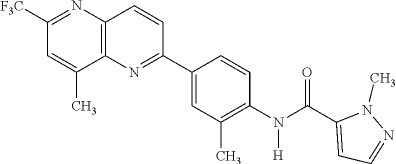 [SMILES: Cc1cc(-c2ccc3nc(C(F)(F)F)cc(C)c3n2)ccc1NC(=O)c1ccnn1C]

The title compound was prepared analogously to Example 005, where 6-chloro-4-methyl-2-(trifluoromethyl)-1,5-naphthyridine was substituted in place of 2-chloro-6-(trifluoromethyl)quinoline and 1-methyl-N-(2-methyl-4-(4,4,5,5-tetramethyl-1,3,2-dioxaborolan-2-yl)phenyl)-1H-pyrazole-5-carboxamide was substituted in place of N-(2-fluoro-3-methyl-4-(4,4,5,5-tetramethyl-1,3,2-dioxaborolan-2-yl)phenyl)-1-methyl-1H-pyrazole-5-carboxamide. Yield=62%. LCMS (ES, m/z): [M+H]⁺ 426; ¹H-NMR (400 MHz, CDCl₃, ppm): δ2.51 (s, 3H), δ3.03 (s, 3H), δ4.29 (s, 3H), δ6.73-6.72 (d, 1H), δ7.57-7.58 (d, 1H), δ7.68 (s, 1H), δ7.84 (s, 1H), δ8.17-8.28 (m, 4H), δ8.53-8.55 (d, 1H)

### Step 1: 2-bromo-4-methyl-6-(trifluoromethyl)pyridin-3-amine

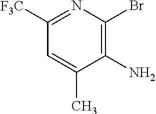

Over a solution of 4-methyl-6-(trifluoromethyl)pyridin-3-amine (2 g, 11.35 mmol, 1 equiv) in dichloromethane (20 mL), NBS (2.0 g, 11.24 mmol, 0.990 equiv) was added. The resulting solution was stirred for 4 hr at room temperature and volatiles evaporated under vacuum. The residue was purified by silica gel chromatography with ethyl acetate/petroleum ether (1:5) as eluent to afford the desired final product as a white solid in 86% yield.

### Step 2: ethyl (E)-3-(3-amino-4-methyl-6-(trifluoromethyl)pyridin-2-yl)acrylate

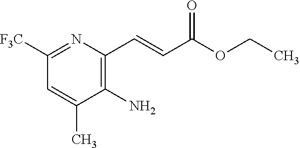 [SMILES: CCOC(=O)/C=C/c1nc(C(F)(F)F)cc(C)c1N]

The title compound was prepared analogously to Example 001, step 1, where 2-bromo-4-methyl-6-(trifluoromethyl)pyridin-3-amine was substituted in place of 2-bromo-4-(trifluoromethyl)aniline. Yield=74%.

### Step 3: 8-methyl-6-(trifluoromethyl)-1,5-naphthyridin-2(1H)-one

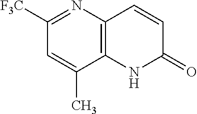 [SMILES: Cc1cc(C(F)(F)F)nc2ccc(=O)[nH]c12]

Into a 50-mL round-bottom flask, was placed ethyl-3-[3-amino-4-methyl-6-(trifluoromethyl)pyridin-2-yl]prop-2-enoate (1 g, 3.65 mmol, 1 equiv), dioxane (5 mL), HCl (6M, 5 mL). The resulting solution was stirred overnight at 100° C. The resulting solution was diluted with 30 mL of water. The solids were collected by filtration. This resulted in 700 mg of 8-methyl-6-(trifluoromethyl)-1,2-dihydro-1,5-naphthyridin-2-one as a grey solid. Yield=84%.

### Step 4: 6-chloro-4-methyl-2-(trifluoromethyl)-1,5-naphthyridine

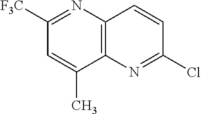 [SMILES: Cc1cc(C(F)(F)F)nc2ccc(Cl)nc12]

The title compound was prepared analogously to Example 001, step 3, where 8-methyl-6-(trifluoromethyl)-1,5-naphthyridin-2(1H)-one was substituted in place of 6-(trifluoromethyl)-1,2-dihydroquinolin-2-one. Yield=59%.

### Example 106: N,1-dimethyl-N-(2-methyl-4-(8-methyl-6-(trifluoromethyl)-1,5-naphthyridin-2-yl)phenyl)-1H-pyrazole-5-carboxamide

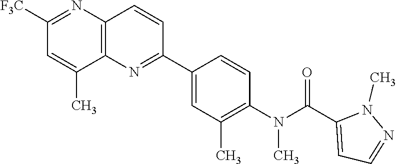 [SMILES: Cc1cc(-c2ccc3nc(C(F)(F)F)cc(C)c3n2)ccc1N(C)C(=O)c1ccnn1C]

The title compound was prepared analogously to Example 002, where 1-methyl-N-(2-methyl-4-(8-methyl-6-(trifluoromethyl)-1,5-naphthyridin-2-yl)phenyl)-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=31%. LCMS (ES, m/z): [M+H]⁺ 440; ¹H-NMR (400 MHz, DMSO-d₆, ppm): δ2.29 (s, 3H), δ2.96 (s, 3H), δ3.33 (s, 3H), δ4.02 (s, 3H), δ5.57-5.58 (d, 1H), δ7.16-7.17 (s, 1H), δ7.50-7.53 (d, 1H), δ8.17 (s, 1H), δ8.24-8.32 (m, 2H), δ8.55-8.65 (m, 2H)

### Example 107: N-(4-(6-fluoroquinazolin-2-yl)-2-methylphenyl)-1-methyl-1H-pyrazole-5-carboxamide

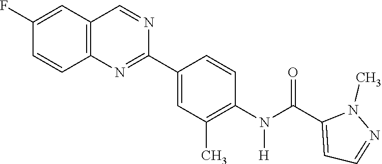 [SMILES: Cc1cc(-c2ncc3cc(F)ccc3n2)ccc1NC(=O)c1ccnn1C]

The title compound was prepared analogously to Example 001, where 4-(6-fluoroquinazolin-2-yl)-2-methylaniline was substituted in place of 2-methyl-4-(6-(trifluoromethyl)quinolin-2-yl)aniline. Yield=32%. LCMS (ES, m/z): [M+H]⁺ 362; ¹H-NMR (400 MHz, DMSO-d₆, ppm): δ2.43 (s, 3H), δ4.11 (s, 3H), δ7.10-7.11 (d, 1H), δ7.55-7.60 (m, 2H), δ7.94-8.02 (m, 2H), δ8.14-8.18 (m, 1H), δ8.39-8.42 (d, 1H), δ8.47-8.48 (d, 1H), δ9.70 (s, 1H), δ9.97 (s, 1H)

### Step 1: 4-(6-fluoroquinazolin-2-yl)-2-methylaniline

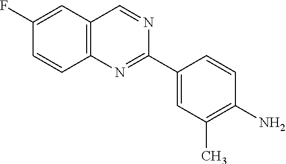 [SMILES: Cc1cc(-c2ncc3cc(F)ccc3n2)ccc1N]

The title compound was prepared analogously to Example 001, step 4, where 2-chloro-6-fluoroquinazoline was substituted in place of 2-chloro-6-(trifluoromethyl)quinoline. Yield=40%.

### Example 108: 1-methyl-N-(2-methyl-4-(7-(trifluoromethyl)quinazolin-2-yl)phenyl)-1H-pyrazole-5-carboxamide

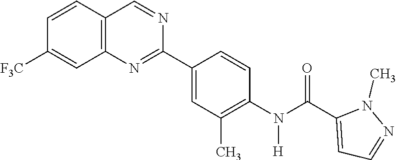 [SMILES: Cc1cc(-c2ncc3ccc(C(F)(F)F)cc3n2)ccc1NC(=O)c1ccnn1C]

The title compound was prepared analogously to Example 001, step 4, where 2-chloro-7-(trifluoromethyl)quinazoline was substituted in place of 2-chloro-6-(trifluoromethyl)quinoline and 1-methyl-N-(2-methyl-4-(4,4,5,5-tetramethyl-1,3,2-dioxaborolan-2-yl)phenyl)-1H-pyrazole-5-carboxamide was substituted in place of 3-methyl-4-(tetramethyl-1,3,2-dioxaborolan-2-yl)aniline. Yield=4%. LCMS (ES, m/z): [M+H]⁺ 412; ¹H-NMR (DMSO-d₆, 300 MHz, ppm): δ2.40 (s, 3H), δ4.11 (s, 3H), δ7.11-7.12 (d, 1H), δ7.56-7.57 (d, 1H), δ7.62-7.64 (d, 1H), δ8.01-8.04 (d, 1H), δ8.44-8.46 (d, 3H), δ8.47-8.53 (d, 1H), δ9.89 (s, 1H), δ10.00 (s, 1H)

### Step 1: 7-(trifluoromethyl)quinazoline-2,4(1H,3H)-dione

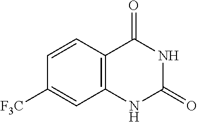 [SMILES: O=c1[nH]c(=O)c2ccc(C(F)(F)F)cc2[nH]1]

A mixture of 2-amino-4-(trifluoromethyl)benzoic acid (5 g, 24.37 mmol, 1.00 equiv) and urea (14.6 g, 243.11 mmol, 10.00 equiv) was stirred for 15 min at 200° C. The resulting solution was allowed to react, with stirring, for an additional 0.5 hours while the temperature was maintained at 100° C. The reaction was then quenched by the addition of 25 mL of water. The solids were collected by filtration to afford the desired final product as a yellow solid in 89% yield.

### Step 2: 2,4-dichloro-7-(trifluoromethyl)quinazoline

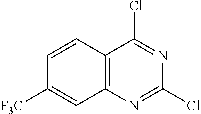 [SMILES: FC(F)(F)c1ccc2c(Cl)nc(Cl)nc2c1]

7-(trifluoromethyl)-1,2,3,4-tetrahydroquinazoline-2,4-dione (3.8 g, 16.51 mmol, 1.00 equiv) in phosphoroyl trichloride (38 mL) was stirred at 120° C. overnight. The resulting mixture was concentrated under vacuum and 100 mL of ice/water added. The pH of the solution was adjusted to pH 7 with sodium bicarbonate. The resulting solution was extracted with dichloromethane (3×300 mL) and the organic layers combined and concentrated under reduced pressure. The resulting residue was purified by silica gel chromatography with ethyl acetate/petroleum ether (1:20) to afford the desired final product as a yellow solid in 41% yield.

### Step 3: 2-chloro-7-(trifluoromethyl)quinazoline

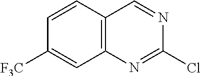 [SMILES: FC(F)(F)c1ccc2cnc(Cl)nc2c1]

Over a solution of 2,4-dichloro-7-(trifluoromethyl)quina (370 mg, 1.3 mmol, 1.00 equiv) in THE (4 mL), Pd(PPh3)₂Cl₂ (97 mg, 0.14 mmol, 0.10 equiv), PPh₃ (363 mg, 1.38 mmol, 1.00 equiv) and Bu₃SnH (809 mg, 2.00 equiv) were added. The resulting solution was stirred for 4 hours at room temperature, quenched with 10 ml of H₂O and extracted with ethyl acetate (3×30 mL). The organic layers were combined and concentrated under reduced pressure. The residue was purified by preparative TLC with ethyl acetate/petroleum ether (1:20) as eluent to afford the desired product as a white solid in 79% yield.

### Example 109: N-(4-(6-fluoro-8-methylquinazolin-2-yl)-2-methylphenyl)-1-methyl-1H-pyrazole-5-carboxamide

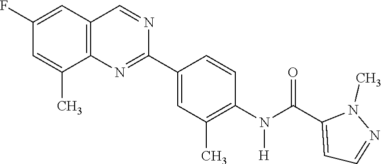 [SMILES: Cc1cc(-c2ncc3cc(F)cc(C)c3n2)ccc1NC(=O)c1ccnn1C]

2-chloro-6-fluoro-8-methylquinazoline (100 mg, 0.51 mmol, 1 equiv), 1-methyl-N-[2-methyl-4-(tetramethyl-1,3,2-dioxaborolan-2-yl)phenyl]-1H-pyrazole-5-carboxamide (173.5 mg, 0.51 mmol, 1 equiv) were dissolved in DME (3 mL) and H₂O (1 mL). Na₂CO₃ (107.8 mg, 1.02 mmol, 2 equiv) and Pd(PPh₃)₄(58.8 mg, 0.05 mmol, 0.1 equiv) were added next. The resulting solution was stirred overnight at 85° C. and quenched by the addition of 5 mL of H₂O. Extraction with ethyl acetate (2×5 mL) and elimination of volatiles under reduced pressure afforded a crude residue that was purified by preparative TLC with dichloromethane/methanol (30/1) as eluent to afford the desired product as a white solid in 32% yield. LCMS (ES, m/z): [M+H]⁺ 376; ¹H-NMR (400 MHz, DMSO-d₆, ppm): δ2.39 (s, 3H), δ2.83 (s, 3H), δ4.11 (s, 3H), δ7.11 (s, 1H), δ7.56-7.60 (m, 2H), δ7.78-7.81 (m, 1H), δ7.84-7.86 (m, 1H), δ8.43-8.45 (m, 1H), δ8.50 (s, 1H), δ9.64 (s, 1H), δ9.98 (s, 1H).

### Step 1: 8-bromo-2-chloro-6-fluoroquinazoline

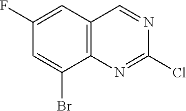 [SMILES: Fc1cc(Br)c2nc(Cl)ncc2c1]

The title compound was prepared analogously to Example 001 step 3, where 8-bromo-6-fluoro-1,2-dihydroquinolin-2-one was substituted in place of 6-(trifluoromethyl)-1,2-dihydroquinolin-2-one. Yield=84%.

### Step 2: 2-chloro-6-fluoro-8-methylquinazoline

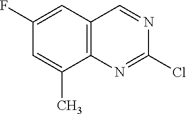

A mixture of 8-bromo-2-chloro-6-fluoroquinazoline (500 mg, 1.91 mmol, 1 equiv), methylboronic acid (114.5 mg, 1.91 mmol, 1 equiv), K₂CO₃ (528.6 mg, 3.82 mmol, 2 equiv), Pd(dppf)Cl₂ (139.9 mg, 0.19 mmol, 0.1 equiv) in 20 mL of DMF was stirred overnight at 120° C. The reaction was then quenched by the addition of 50 mL of water, extracted with ethyl acetate (2×20 mL) and the combined organic layers concentrated. The residue was purified by silica gel chromatography with ethyl acetate/petroleum ether (1:3) to afford the desired product as a yellow solid in 53% yield.

### Example 110: 1-methyl-N-(2-methyl-4-(8-methyl-6-(trifluoromethyl)quinazolin-2-yl)phenyl)-1H-pyrazole-5-carboxamide

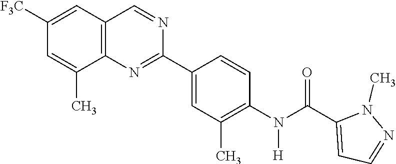 [SMILES: Cc1cc(-c2ncc3cc(C(F)(F)F)cc(C)c3n2)ccc1NC(=O)c1ccnn1C]

A mixture of 2-chloro-8-methyl-6-(trifluoromethyl)quinazoline (100 mg, 0.41 mmol, 1 equiv), 1-methyl-N-(2-methyl-4-(4,4,5,5-tetramethyl-1,3,2-dioxaborolan-2-yl)phenyl)-1H-pyrazole-5-carboxamide (138.4 mg, 0.41 mmol, 1 equiv), Na₂CO₃ (86.0 mg, 0.81 mmol, 2 equiv) and Pd(PPh₃)₄(46.9 mg, 0.04 mmol, 0.1 equiv) in toluene (2 mL) and EtOH (1 mL) was stirred at 80° C. overnight. The resulting solution was diluted with 10 mL of water and extracted with ethyl acetate (2×5 mL). Elimination of volatiles under reduced pressure afforded a residue that was purified by silica gel chromatography with dichloromethane/methanol (30/1) to afford the desired product as a white solid in 35% yield. LCMS (ES, m/z): [M+H]⁺ 426; ¹H-NMR (400 MHz, CDCl₃, ppm): δ2.51 (s, 3H), δ2.94 (s, 3H), δ4.29 (s, 3H), δ6.71-6.72 (d, 1H), δ7.57 (s, 1H), δ7.70 (s, 1H), δ7.93 (s, 1H), δ8.10 (s, 1H), δ8.28-8.32 (m, 1H), δ8.61-8.64 (m, 2H), δ9.53 (s, 1H).

### Step 1: 2-amino-3-methyl-5-(trifluoromethyl)benzonitrile

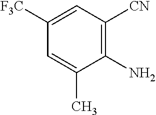

2-bromo-6-methyl-4-(trifluoromethyl)aniline (2.8 g, 11.02 mmol, 1 equiv) and CuCN (1974.3 mg, 22.04 mmol, 2 equiv) were dissolved in DMF (20 mL) and irradiated under microwave conditions for 40 min at 220° C. The reaction was then quenched by the addition of 50 mL of water. The resulting solution was extracted with ethyl acetate (3×50 mL) and the combined organic layers concentrated. The residue was purified by silica gel chromatograpy with dichloromethane/methanol (50/1) to afford the desired product as a yellow solid in 60% yield.

### Step 2: 2,4-dichloro-8-methyl-6-(trifluoromethyl)quinazoline

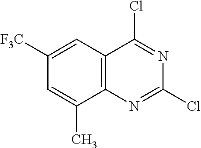 [SMILES: Cc1cc(C(F)(F)F)cc2c(Cl)nc(Cl)nc12]

Over a solution of 2-amino-3-methyl-5-(trifluoromethyl)benzonitrile (300 mg, 1.50 mmol, 1 equiv) in CH₃CN (5 mL, 0.12 mmol, 0.081 equiv), chloro(trichloromethoxy)methanone (444.8 mg, 2.25 mmol, 1.500 equiv) was added. The resulting solution was stirred overnight at 120° C. The reaction was quenched by addition of 30 ml of H₂O, the aqueous layer extracted with ethyl acetate (3×30 mL) and the combined organic layers concentrated under reduced pressure. The resulting residue was purified by preparative-TLC with ethyl acetate/petroleum ether (1/10) as eluent to afford the desired product as a yellow solid in 71% yield.

### Step 3: 2-chloro-8-methyl-6-(trifluoromethyl)quinazoline

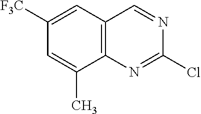 [SMILES: Cc1cc(C(F)(F)F)cc2cnc(Cl)nc12]

A mixture of 2,4-dichloro-8-methyl-6-(trifluoromethyl)quinazoline (200 mg, 0.71 mmol, 1 equiv), PPh₃ (280.0 mg, 1.07 mmol, 1.5 equiv), Bu₃SnH (207.1 mg, 0.71 mmol, 1 equiv) and Pd(PPh₃)₄(82.2 mg, 0.07 mmol, 0.1 equiv) in THE (10 mL) was stirred for 8 hours at room temperature. Elimination of volatiles under reduced pressure afforded a residue that was purified by silica gel chromatography with ethyl acetate/petroleum ether (1:10) to afford the desired final product as a white solid in 45% yield.

### Example 111: N,1-dimethyl-N-(2-methyl-4-(8-methyl-6-(trifluoromethyl)quinazolin-2-yl)phenyl)-1H-pyrazole-5-carboxamide

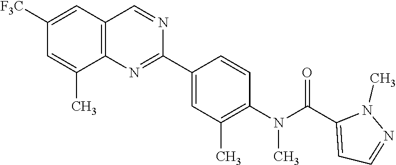 [SMILES: Cc1cc(-c2ncc3cc(C(F)(F)F)cc(C)c3n2)ccc1N(C)C(=O)c1ccnn1C]

The title compound was prepared analogously to Example 002, where 1-methyl-N-(2-methyl-4-(8-methyl-6-(trifluoromethyl)quinazolin-2-yl)phenyl)-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=41%. LCMS (ES, m/z): [M+H]⁺ 440; ¹H-NMR (400 MHz, DMSO-d₆, ppm): δ2.27 (s, 3H), δ2.85 (s, 3H), δ3.33 (s, 3H), δ4.01 (s, 3H), δ5.54-5.55 (d, 1H), δ7.14-7.15 (d, 1H), δ7.49-7.50 (d, 1H), δ8.17 (s, 1H), δ8.40-8.48 (d, 1H), δ8.51-8.52 (d, 2H), δ9.84 (s, 1H)

### Example 112: 1-methyl-N-(2-methyl-4-(6-(trifluoromethyl)quinazolin-2-yl)phenyl)-1H-pyrazole-5-carboxamide

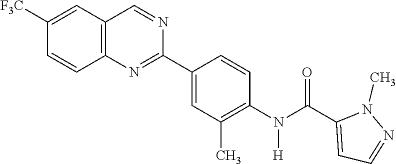 [SMILES: Cc1cc(-c2ncc3cc(C(F)(F)F)ccc3n2)ccc1NC(=O)c1ccnn1C]

The title compound was prepared analogously to Example 014, step 1, where 2-chloro-6-(trifluoromethyl)quinazoline was substituted in place of 2-chloro-6-fluoroquinoline and 1-methyl-N-(2-methyl-4-(4,4,5,5-tetramethyl-1,3,2-dioxaborolan-2-yl)phenyl)-1H-pyrazole-5-carboxamide was substituted in place of tert-butyl (2-methyl-4-(4,4,5,5-tetramethyl-1,3,2-dioxaborolan-2-yl)phenyl)carbamate. Yield=20%. LCMS (ES, m/z): [M+H]⁺ 412; ¹H-NMR (400 MHz, CDCl₃, ppm): 2.40 (s, 3H), 4.18 (s, 3H), 6.60 (d, 1H), 7.46 (d, 1H), 7.61 (s, 1H), 8.00 (dd, 1H), 8.19 (m, 3H), 8.51 (m, 2H), 9.49 (s, 1H).

### Step 1: 2-amino-5-(trifluoromethyl)benzonitrile

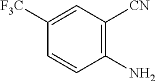 [SMILES: N#Cc1cc(C(F)(F)F)ccc1N]

The title compound was prepared analogously to Example 110, step 1, where 2-bromo-4-(trifluoromethyl)aniline was substituted in place-bromo-6-methyl-4-(trifluoromethyl)aniline. Yield=55%.

### Step 2: 2,4-dichloro-6-(trifluoromethyl)quinazoline

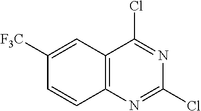 [SMILES: FC(F)(F)c1ccc2nc(Cl)nc(Cl)c2c1]

The title compound was prepared analogously to Example 110, step 2, where 2-amino-5-(trifluoromethyl)benzonitrile was substituted in place 2-amino-3-methyl-5-(trifluoromethyl)benzonitrile. Yield=46%.

### Step 3: 2-chloro-6-(trifluoromethyl)quinazoline

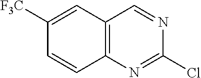 [SMILES: FC(F)(F)c1ccc2nc(Cl)ncc2c1]

The title compound was prepared analogously to Example 110, step 3, 2,4-dichloro-6-(trifluoromethyl)quinazoline was substituted in place of 2,4-dichloro-8-methyl-6-(trifluoromethyl)quinazoline. Yield=50%.

### Example 113: N-(2-fluoro-3-methyl-4-(6-(trifluoromethyl)quinazolin-2-yl)phenyl)-1-methyl-1H-pyrazole-5-carboxamide

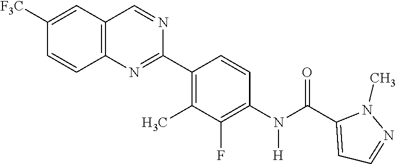 [SMILES: Cc1c(-c2ncc3cc(C(F)(F)F)ccc3n2)ccc(NC(=O)c2ccnn2C)c1F]

The title compound was prepared analogously to Example 110, where 2-chloro-6-(trifluoromethyl)quinazoline was substituted in place of 2-chloro-8-methyl-6-(trifluoromethyl)quinazoline and N-(2-fluoro-3-methyl-4-(4,4,5,5-tetramethyl-1,3,2-dioxaborolan-2-yl)phenyl)-1-methyl-1H-pyrazole-5-carboxamide was substituted in place of 1-methyl-N-(2-methyl-4-(4,4,5,5-tetramethyl-1,3,2-dioxaborolan-2-yl)phenyl)-1H-pyrazole-5-carboxamide. Yield=31%. LCMS (ES, m/z): [M+H]⁺ 430; ¹H-NMR (DMSO-d₆, 300 MHz, ppm):δ2.56-2.57 (d, 3H), δ4.11 (s, 3H), δ7.15-7.16 (d, 2H), δ7.56-7.57 (d, 1H), δ7.66-7.71 (t, 1H), 57.88-7.90 (d, 1H), δ8.25-8.3 (m, 2H), δ8.77 (s, 1H), δ9.92 (s, 1H), δ10.22 (s, 1H)

### Example 114: N-(2-fluoro-3-methyl-4-(6-(trifluoromethyl)quinazolin-2-yl)phenyl)-N,1-dimethyl-1H-pyrazole-5-carboxamide

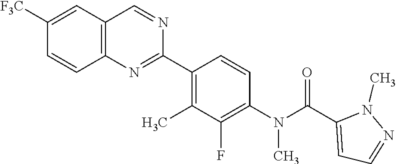 [SMILES: Cc1c(-c2ncc3cc(C(F)(F)F)ccc3n2)ccc(N(C)C(=O)c2ccnn2C)c1F]

The title compound was prepared analogously to Example 002, where N-(2-fluoro-3-methyl-4-(6-(trifluoromethyl)quinazolin-2-yl)phenyl)-1-methyl-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=17%. LCMS (ES, m/z): [M+H]⁺ 444; ¹H-NMR (DMSO-d₆, 300 MHz, ppm):δ2.43 (s, 3H), 53.30-3.33 (d, 3H), δ3.97 (s, 3H), δ5.84 (s, 1H), δ7.26 (s, 1H), δ7.50-7.55 (t, 1H), δ7.80-7.82 (d, 1H), δ8.24-8.34 (m, 2H), δ8.77 (s, 1H), δ9.92 (s, 1H)

### Example 115: N-(2-methyl-4-(6-(trifluoromethyl)quinazolin-2-yl)phenyl)isonicotinamide

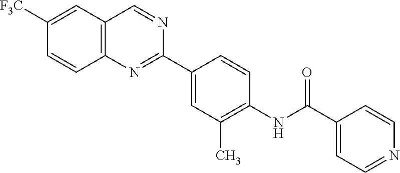 [SMILES: Cc1cc(-c2ncc3cc(C(F)(F)F)ccc3n2)ccc1NC(=O)c1ccncc1]

The title compound was prepared analogously to Example 001, step 4, where 2-methyl-4-(6-(trifluoromethyl)quinazolin-2-yl)aniline was substituted in place of 2-methyl-4-(6-(trifluoromethyl)quinolin-2-yl)aniline and isonicotinic acid was substituted in place of 1-methyl-1H-pyrazole-5-carboxylic acid. Yield=42%. LCMS (ES, m/z): [M+H]⁺ 409; ¹H-NMR (DMSO-d₆, 400 MHz, ppm): δ2.42 (s, 3H), δ7.67-7.70 (t, 1H), δ7.91-7.95 (t, 2H), δ8.24-8.30 (m, 2H), δ8.46-8.49 (t, 1H), δ8.54 (s, 1H), δ8.72 (s, 1H), δ8.81-8.83 (d, 2H), δ9.99 (s, 1H), δ10.29 (s, 1H)

### Step 1: 2-(3-methyl-4-nitrophenyl)-6-(trifluoromethyl)quinazoline

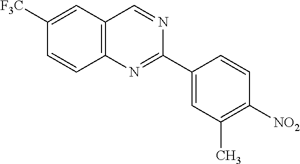 [SMILES: Cc1cc(-c2ncc3cc(C(F)(F)F)ccc3n2)ccc1[N+](=O)[O-]]

The title compound was prepared analogously to Example 136, step 4, where 2-chloro-6-(trifluoromethyl)quinazoline was substituted in place of 2-chloro-6-(trifluoromethyl)quinoline and 4,4,5,5-tetramethyl-2-(3-methyl-4-nitrophenyl)-1,3,2-dioxaborolane was substituted in place of 2-methyl-4-(4,4,5,5-tetramethyl-1,3,2-dioxaborolan-2-yl)aniline. Yield=42%.

### Step 2: 2-methyl-4-(6-(trifluoromethyl)quinazolin-2-yl)aniline

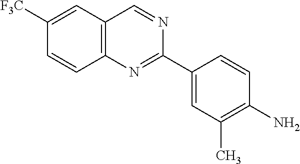 [SMILES: Cc1cc(-c2ncc3cc(C(F)(F)F)ccc3n2)ccc1N]

The title compound was prepared analogously to Example 045, step 3, where 2-(3-methyl-4-nitrophenyl)-6-(trifluoromethyl)quinazoline was substituted in place of 6-fluoro-2-(6-methyl-5-nitropyridin-2-yl)quinoline. Yield=98%.

### Example 116: N-methyl-N-(2-methyl-4-(6-(trifluoromethyl)quinazolin-2-yl)phenyl)isonicotinamide

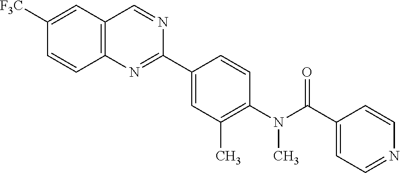 [SMILES: Cc1cc(-c2ncc3cc(C(F)(F)F)ccc3n2)ccc1N(C)C(=O)c1ccncc1]

To a stirred solution of N,2-dimethyl-4-[6-(trifluoromethyl)quinazolin-2-yl]aniline (60 mg, 0.19 mmol, 1 equiv) and isonicotinoyl chloride hydrochloride (40.4 mg, 0.23 mmol, 1.2 equiv) in CH₂Cl₂ (2 mL) were added triethylamine (67 mg, 0.665 mmol, 3.5 equiv). The resulting mixture was stirred at 50° C. overnight. The reaction was quenched with water (10 ml) and the aqueous layer extracted with CH₂Cl₂ (2×20 mL). The combined organic layers were concentrated under vacuum and the residue was purified by Prep-TLC (CH₂Cl₂/MeOH=25:1) to afford the desired product as a white solid in 58% yield. LCMS (ES, m/z): [M+H]⁺ 423; ¹H-NMR (DMSO-d₆, 400 MHz, ppm): δ2.35 (s, 3H), δ3.35 (s, 3H), δ7.24-7.26 (m, 2H), δ7.45-7.47 (d, 1H), δ8.20-8.26 (d, 1H), δ8.27-8.31 (m, 2H), δ8.40-8.56 (m, 3H), δ8.70-8.78 (m, 1H), δ9.85-9.91 (d, 1H)

### Step 1: N,2-dimethyl-4-(6-(trifluoromethyl)quinazolin-2-yl)aniline

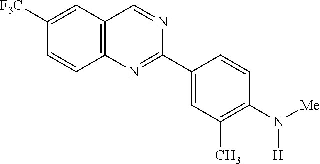 [SMILES: CNc1ccc(-c2ncc3cc(C(F)(F)F)ccc3n2)cc1C]

A solution of tert-butyl N-methyl-N-[2-methyl-4-[6-(trifluoromethyl)quinazolin-2-yl]phenyl] carbamate (5 g, 11.98 mmol, 1 equiv) in HCl (50 mL, 4M dioxane solution) was stirred at room temperature for 2 hours. The resulting mixture was concentrated under reduced pressure and the residue diluted with water (100 mL). The mixture was basified to pH=8 with saturated NaHCO₃ and extracted with EtOAc (2×100 mL). The combined organic layers were concentrated under reduced pressure to afford the desired product as a yellow solid in 82% yield.

### Example 117: 3-methyl-N-(2-methyl-4-(6-(trifluoromethyl)quinazolin-2-yl)phenyl)picolinamide

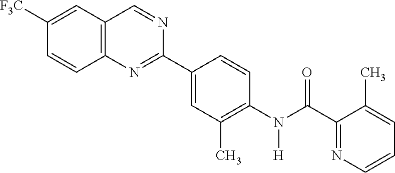 [SMILES: Cc1cc(-c2ncc3cc(C(F)(F)F)ccc3n2)ccc1NC(=O)c1ncccc1C]

The title compound was prepared analogously to Example 001, where 2-methyl-4-(6-(trifluoromethyl)quinazolin-2-yl)aniline was substituted in place of 2-methyl-4-(6-(trifluoromethyl)quinolin-2-yl)aniline and 3-methylpyridine-2-carboxylic acid was substituted in place of 1-methyl-1H-pyrazole-5-carboxylic acid. Yield=46%. LCMS (ES, m/z): [M+H]⁺ 423; ¹H-NMR (300 MHz, CDCl₃, ppm): 10.66 (s, 1H), 9.57 (s, 1H), 8.65-8.61 (m, 3H), 8.53-8.52 (d, 1H), 8.26-8.25 (d, 2H), 8.10-8.07 (m, 1H), 7.72-7.70 (d, 1H), 7.54-7.41 (m, 1H), 2.91 (s, 3H), 2.59 (s, 3H)

### Example 118: N,3-dimethyl-N-(2-methyl-4-(6-(trifluoromethyl)quinazolin-2-yl)phenyl)picolinamide

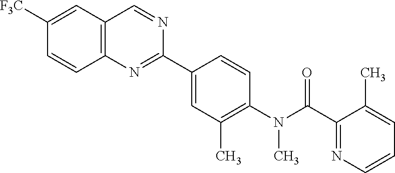 [SMILES: Cc1cc(-c2ncc3cc(C(F)(F)F)ccc3n2)ccc1N(C)C(=O)c1ncccc1C]

The title compound was prepared analogously to Example 002, where 3-methyl-N-(2-methyl-4-(6-(trifluoromethyl)quinazolin-2-yl)phenyl)picolinamide was substituted in place of 1-methyl-N-(2-methyl-4-(6-(trifluoromethyl)quinolin-2-yl)phenyl)-1H-pyrazole-5-carboxamide. Yield=56%. LCMS (ES, m/z): [M+H]⁺ 437; ¹H-NMR (300 MHz, CDCl₃, ppm): 9.61-8.53 (d, 1H), 8.66-8.57 (m, 0.6H), 8.42 (s, 0.8H), 8.27-8.23 (m, 2H), 8.19-8.07 (m, 2.6H), 7.72-7.70 (d, 0.4H), 7.56-7.54 (d, 1H), 7.39-7.37 (d, 0.8H), 7.25-7.23 (d, 0.8H), 3.49 (s, 2.4H), 3.23 (s, 0.6H), 2.57 (s, 3.7H), 2.45 (s, 2.5H).

### Example 119: 2-methyl-N-(2-methyl-4-(6-(trifluoromethyl)quinazolin-2-yl)phenyl)isonicotinamide

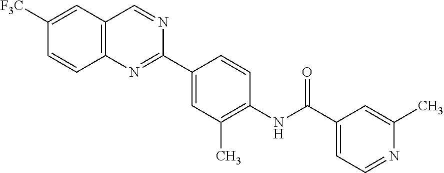 [SMILES: Cc1cc(C(=O)Nc2ccc(-c3ncc4cc(C(F)(F)F)ccc4n3)cc2C)ccn1]

The title compound was prepared analogously to Example 001, where 2-methyl-4-(6-(trifluoromethyl)quinazolin-2-yl)aniline was substituted in place of 2-methyl-4-(6-(trifluoromethyl)quinolin-2-yl)aniline and 2-methylpyridine-4-carboxylic acid was substituted in place of 1-methyl-1H-pyrazole-5-carboxylic acid. Yield=34%. LCMS (ES, m/z): [M+H]⁺ 423; ¹H-NMR (DMSO-d₆, 300 MHz, ppm): δ2.41 (s, 3H), δ2.60 (s, 3H), δ7.65-7.72 (t, 2H), 67.79 (s, 1H), δ8.24-8.31 (t, 2H), δ8.45-8.49 (t, 1H), δ8.53 (s, 1H), δ8.66-8.68 (d, 1H), δ8.72 (s, 1H), δ9.99 (s, 1H), δ10.24 (s, 1H)

### Example 120: N,2-dimethyl-N-(2-methyl-4-(6-(trifluoromethyl)quinazolin-2-yl)phenyl)isonicotinamide

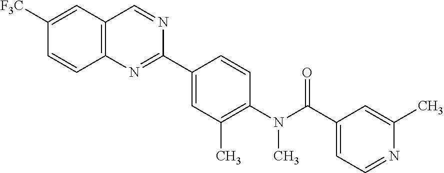 [SMILES: Cc1cc(C(=O)N(C)c2ccc(-c3ncc4cc(C(F)(F)F)ccc4n3)cc2C)ccn1]

The title compound was prepared analogously to Example 002, where 2-methyl-N-(2-methyl-4-(6-(trifluoromethyl)quinazolin-2-yl)phenyl)isonicotinamide was substituted in place of 1-methyl-N-(2-methyl-4-(6-(trifluoromethyl)quinolin-2-yl)phenyl)-1H-pyrazole-5-carboxamide. Yield=21%. LCMS (ES, m/z): [M+H]⁺ 437; ¹H-NMR (DMSO-d₆, 300 MHz, ppm): δ2.35 (s, 6H), δ3.31-3.33 (d, 3H), δ6.96-6.97 (d, 1H), δ7.19 (s, 1H), δ8.45-7.47 (d, 1H), δ8.21-8.8.33 (m, 4H), δ8.41 (s, 1H), δ8.72 (s, 1H), δ9.87 (s, 1H)

### Example 121: 3-methyl-N-(2-methyl-4-(6-(trifluoromethyl)quinazolin-2-yl)phenyl)isonicotinamide

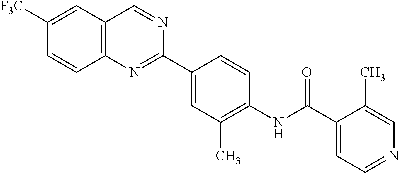 [SMILES: Cc1cc(-c2ncc3cc(C(F)(F)F)ccc3n2)ccc1NC(=O)c1ccncc1C]

The title compound was prepared analogously to Example 001, where 2-methyl-4-(6-(trifluoromethyl)quinazolin-2-yl)aniline was substituted in place of 2-methyl-4-(6-(trifluoromethyl)quinolin-2-yl)aniline and 3-methylpyridine-4-carboxylic acid was substituted in place of 1-methyl-1H-pyrazole-5-carboxylic acid. Yield=43%. LCMS (ES, m/z): [M+H]⁺ 423; ¹H-NMR (300 MHz, DMSO-d₆, ppm): 10.16 (s, 1H), 9.89 (s, 1H), 8.71 (s, 1H), 8.60-8.47 (m, 4H), 8.30-8.27 (m, 2H), 7.99-7.77 (d, 1H), 7.65-7.54 (d, 1H), 2.45-2.44 (d, 6H)

### Example 122: N,3-dimethyl-N-(2-methyl-4-(6-(trifluoromethyl)quinazolin-2-yl)phenyl)isonicotinamide

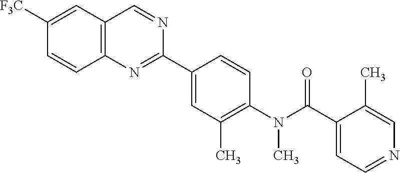 [SMILES: Cc1cnccc1C(=O)N(C)c1ccc(-c2ncc3cc(C(F)(F)F)ccc3n2)cc1C]

To a stirred solution of N,2-dimethyl-4-[6-(trifluoromethyl)quinazolin-2-yl]aniline (60 mg, 0.19 mmol, 1 equiv) and 3-methylpyridine-4-carboxylic acid (25.9 mg, 0.19 mmol, 1 equiv) in DMF (1 mL) were added DIEA (48.9 mg, 0.38 mmol, 2 equiv) and HATU (107.8 mg, 0.28 mmol, 1.5 equiv). The resulting mixture was stirred overnight at 60° C. under nitrogen atmosphere. The mixture was concentrated under reduced pressure and the residue was purified by preparative TLC (CH₂Cl₂/MeOH=20:1) to afford the desired final product as a white solid in 64% yield. LCMS (ES, m/z): [M+H]⁺ 437; ¹H-NMR (300 MHz, CDCl₃, ppm): 9.60-9.55 (d, 1H), 8.68-8.62 (m, 0.6H), 8.02 (s, 0.9H), 8.34 (s, 0.8H), 8.32-8.27 (m, 2H), 8.24-8.18 (m, 1.7H), 8.12-8.10 (m, 1H), 7.46-7.43 (d, 0.2H), 7.35-7.33 (d, 0.2H), 7.15-7.13 (d, 0.8H), 6.91-6.90 (d, 0.8H), 3.49 (s, 2.5H), 3.19 (s, 0.4H), 2.53-2.47 (d, 6H)

### Example 123: 5-methyl-N-(2-methyl-4-(6-(trifluoromethyl)quinazolin-2-yl)phenyl)pyrimidine-4-carboxamide

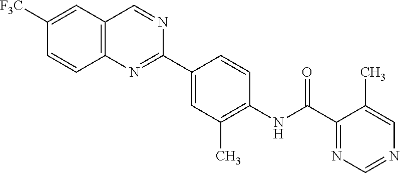 [SMILES: Cc1cc(-c2ncc3cc(C(F)(F)F)ccc3n2)ccc1NC(=O)c1ncncc1C]

A mixture of 5-bromo-N-[2-methyl-4-[6-(trifluoromethyl)quinazolin-2-yl]phenyl]pyrimidine-4-carboxamide (245 mg, 0.50 mmol, 1 equiv), KOAc (98.5 mg, 1.00 mmol, 2 equiv), trimethyl-1,3,5,2,4,6-trioxatriborinane (252.0 mg, 1.00 mmol, 2.000 equiv, 50%), Pd(dppf)Cl₂ (36.7 mg, 0.05 mmol, 0.100 equiv) in dioxane (3.0 mL) was stirred overnight at 100° C. The reaction was quenched by the addition of 15 mL of water, extracted with ethyl acetate (3×5 mL) and the combined organic layers concentrated under reduced pressure. The residue was purified by silica gel chromatography with dichloromethane/methanol (30/1) to afford the desired final product as a white solid in 90% yield. LCMS (ES, m/z): [M+H]⁺ 424; ¹H-NMR (400 MHz, DMSO-d₆, ppm): δ2.47 (s, 3H), δ2.60 (s, 3H), δ8.04-8.06 (d, 1H), δ8.24-8.29 (m, 2H), δ8.48-8.53 (m, 2H), δ8.71 (s, 1H), δ8.95 (s, 1H), δ9.24 (s, 1H), δ9.88 (s, 1H), δ10.46 (s, 1H).

### Step 1: 5-bromo-N-(2-methyl-4-(6-(trifluoromethyl)quinazolin-2-yl)phenyl)pyrimidine-4-carboxamide

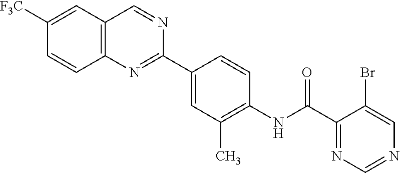 [SMILES: Cc1cc(-c2ncc3cc(C(F)(F)F)ccc3n2)ccc1NC(=O)c1ncncc1Br]

The title compound was prepared analogously to Example 001, where 2-methyl-4-(6-(trifluoromethyl)quinazolin-2-yl)aniline was substituted in place of 2-methyl-4-(6-(trifluoromethyl)quinolin-2-yl)aniline and 5-bromopyrimidine-4-carboxylic acid was substituted in place of 1-methyl-1H-pyrazole-5-carboxylic acid. Yield=79%

### Example 124: N,5-dimethyl-N-(2-methyl-4-(6-(trifluoromethyl)quinazolin-2-yl)phenyl)pyrimidine-4-carboxamide

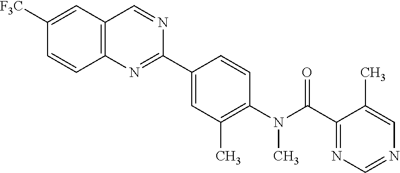 [SMILES: Cc1cc(-c2ncc3cc(C(F)(F)F)ccc3n2)ccc1N(C)C(=O)c1ncncc1C]

The title compound was prepared analogously to Example 002, where 5-methyl-N-(2-methyl-4-(6-(trifluoromethyl)quinazolin-2-yl)phenyl)pyrimidine-4-carboxamide was substituted in place of 1-methyl-N-(2-methyl-4-(6-(trifluoromethyl)quinolin-2-yl)phenyl)-1H-pyrazole-5-carboxamide. Yield=58%. LCMS (ES, m/z): [M+H]⁺ 438; ¹H-NMR (400 MHz, CDCl₃, ppm): δ2.42 (s, 3H), δ2.55 (s, 3H), δ3.49 (s, 3H), δ7.15-7.17 (d, 1H), δ8.06-8.10 (m, 1H), δ8.16-8.22 (m, 1H), δ8.25-8.28 (m, 2H), δ8.45-8.66 (m, 2H), δ8.74 (s, 1H), δ9.52 (s, 1H)

### Example 125: 2,5-dimethyl-N-(2-methyl-4-(6-(trifluoromethyl)quinazolin-2-yl)phenyl)isonicotinamide

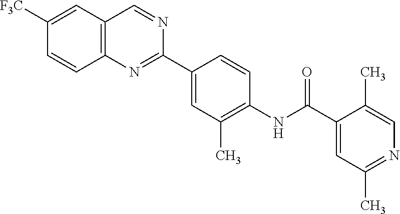 [SMILES: Cc1cc(C(=O)Nc2ccc(-c3ncc4cc(C(F)(F)F)ccc4n3)cc2C)c(C)cn1]

The title compound was prepared analogously to Example 001, where 2-methyl-4-(6-(trifluoromethyl)quinazolin-2-yl)aniline was substituted in place of 2-methyl-4-(6-(trifluoromethyl)quinolin-2-yl)aniline and 2,5-dimethylisonicotinic acid was substituted in place of 1-methyl-1H-pyrazole-5-carboxylic acid. Yield=49%. LCMS (ES, m/z): [M+H]⁺ 437; ¹H-NMR (400 MHz, CDCl₃, ppm): δ2.48 (s, 3H), δ2.53 (s, 3H), δ2.64 (s, 3H), δ7.33 (s, 1H), δ7.53 (s, 1H), δ8.08-8.11 (q, 1H), δ8.21-8.23 (d, 1H), δ8.27 (s, 1H), δ8.34 (brs, 1H), δ8.49 (s, 1H), δ8.58-8.61 (m, 2H), δ9.57 (s, 1H)

### Example 126: N,2,5-trimethyl-N-(2-methyl-4-(6-(trifluoromethyl)quinazolin-2-yl)phenyl)isonicotinamide i

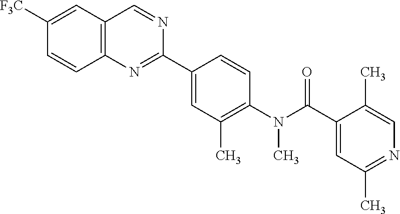 [SMILES: Cc1cc(C(=O)N(C)c2ccc(-c3ncc4cc(C(F)(F)F)ccc4n3)cc2C)c(C)cn1]

To a stirred solution of 2,5-dimethylisonicotinoyl chloride (96 mg, 1.2 equiv) and N,2-dimethyl-4-[6-(trifluoromethyl)quinazolin-2-yl]aniline (150 mg, 1 equiv) in CH₂Cl₂ (2 mL) was added triethylamine (100 mg, 2 equiv). The resulting mixture was stirred for 2 h at room temperature. The reaction was quenched by the addition of water (20 mL). The resulting mixture was extracted with EtOAc (2×20 mL) and the combined organic layers concentrated under reduced pressure. The residue was purified by preparative TLC (CH₂Cl₂/MeOH=30/1) to afford the desired product as a white solid in 46% yield. LCMS: (ES, m/z): [M+H]⁺ 451; ¹H-NMR: (400 MHz, CD₃OD, ppm): δ2.27 (s, 2H), δ2.39 (s, 2H), δ2.43 (s, 1H), δ2.46 (s, 2H), δ2.50 (s, 1H), δ2.60 (s, 1H), δ3.20 (s, 1H), δ3.46 (s, 2H), δ6.96-7.54 (m, 2H), δ8.15-8.67 (m, 6H), δ9.67-9.74 (d, 1H)

### Example 127: 1,3-dimethyl-1-(2-methyl-4-(6-(trifluoromethyl)quinazolin-2-yl)phenyl)urea

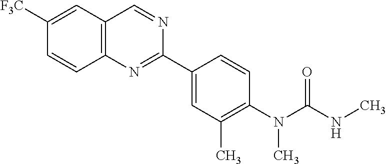 [SMILES: CNC(=O)N(C)c1ccc(-c2ncc3cc(C(F)(F)F)ccc3n2)cc1C]

Over a solution of 4-nitrophenyl methyl(2-methyl-4-(6-(trifluoromethyl)quinazolin-2-yl)phenyl)carbamate in THF, methanamine (2M in THF, 0.4 mL, 4.00 equiv) was added. The resulting solution was stirred overnight at 70° C. The reaction was then diluted by the addition of 20 mL of water and extracted with ethyl acetate (3×50 mL). The organic layers were combined and concentrated under reduced pressure. The residue was purified by preparative TLC with chloroform/methanol (20:1) to afford the desired final product as a white solid in 57% yield. LCMS (ES, m/z): [M+H]⁺ 375; ¹H-NMR (DMSO-d₆, 300 MHz, ppm): δ2.31 (s, 3H), δ2.55 (s, 3H), δ3.12 (s, 3H), δ5.75 (s, 1H), δ7.36-7.39 (d, 1H), δ8.27-8.28 (s, 2H), δ8.43-8.47 (q, 1H), δ8.52 (s, 1H), δ8.72 (s, 1H), δ9.90 (s, 1H)

### Step 1: 4-nitrophenyl methyl(2-methyl-4-(6-(trifluoromethyl)quinazolin-2-yl)phenyl)carbamate

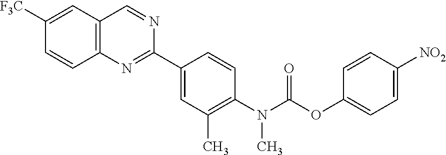 [SMILES: Cc1cc(-c2ncc3cc(C(F)(F)F)ccc3n2)ccc1N(C)C(=O)Oc1ccc([N+](=O)[O-])cc1]

A mixture of N,2-dimethyl-4-[6-(trifluoromethyl)quinazolin-2-yl]aniline (60 mg, 0.19 mmol, 1.00 equiv), triethylamine (38 mg, 0.38 mmol, 2.00 equiv) and 4-nitrophenyl chloroformate (42 mg, 0.21 mmol, 1.10 equiv) in 0.5 mL of THF was stirred for at room temperature 2 hours and used immediately in the next step.

### Example 128: methyl methyl(2-methyl-4-(6-(trifluoromethyl)quinazolin-2-yl)phenyl)carbamate

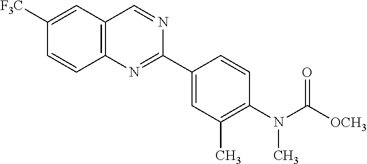 [SMILES: COC(=O)N(C)c1ccc(-c2ncc3cc(C(F)(F)F)ccc3n2)cc1C]

Methyl chloroformate (31.3 mg, 0.33 mmol, 1.5 equiv) was added to a solution of N,2-dimethyl-4-[6-(trifluoromethyl)quinazolin-2-yl]aniline (70 mg, 0.22 mmol, 1 equiv) in pyridine (0.7 mL). The resulting mixture was stirred at 50° C. overnight, and concentrated under reduced pressure. The residue was purified by preparative-TLC (CH₂Cl₂/MeOH=80/1) to afford the desired final product as yellow solid in 32% yield. LCMS (ES, m/z): [M+H]⁺ 376; ¹H-NMR (300 MHz, DMSO-d₆, ppm): δ2.29 (s, 3H), δ3.19 (s, 3H), 53.56-3.72 (d, 3H), δ7.42-7.45 (d, 1H), δ8.27-8.28 (d, 2H), δ8.42-8.45 (q, 1H), δ8.50-8.51 (d, 1H), δ8.71-8.72 (d, 1H), δ9.89 (s, 1H)

### Example 129: 1,1,3-trimethyl-3-(2-methyl-4-(6-(trifluoromethyl)quinazolin-2-yl)phenyl)urea

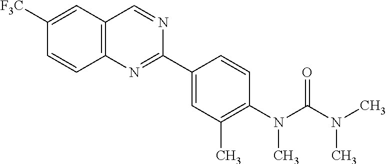 [SMILES: Cc1cc(-c2ncc3cc(C(F)(F)F)ccc3n2)ccc1N(C)C(=O)N(C)C]

A mixture of N, 2-dimethyl-4-[6-(trifluoromethyl)quinazolin-2-yl]aniline (70 mg, 0.22 mmol, 1 equiv), N,N-dimethylcarbamoyl chloride (47.4 mg, 0.44 mmol, 2 equiv) and Et₃N (44.6 mg, 0.44 mmol, 2 equiv) in toluene (2 mL) was stirred at 100° C. overnight. The resulting mixture was concentrated under reduced pressure. The residue was purified by silica gel chromatography with ethyl acetate/hexane (1/10) to afford the desired product as a yellow solid in 44% yield. LCMS (ES, m/z): [M+H]⁺ 389; ¹H-NMR (400 MHz, CDCl₃, ppm): δ9.58 (s, 1H), δ8.56 (s, 1H), δ8.55-8.47 (d, 1H), δ8.28 (s, 1H), δ8.24-8.22 (d, 1H), δ8.12-8.09 (d, 1H), δ7.24-7.22 (d, 1H), δ3.16 (s, 3H), δ2.67 (s, 6H), δ2.43 (s, 3H)

### Example 130: N-methyl-N-(2-methyl-4-(6-(trifluoromethyl)quinazolin-2-yl)phenyl)acetamide

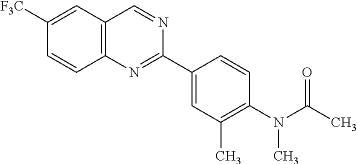 [SMILES: CC(=O)N(C)c1ccc(-c2ncc3cc(C(F)(F)F)ccc3n2)cc1C]

The title compound was prepared analogously to Example 002, where N-(2-methyl-4-(6-(trifluoromethyl)quinazolin-2-yl)phenyl)acetamide was substituted in place of 1-methyl-N-(2-methyl-4-(6-(trifluoromethyl)quinolin-2-yl)phenyl)-1H-pyrazole-5-carboxamide. Yield=59%. LCMS (ES, m/z): [M+H]⁺ 360; ¹H-NMR (300 MHz, CDCl₃, ppm): δ1.87 (s, 3H), δ2.41 (s, 3H), δ3.27 (s, 3H), δ7.33-7.36 (d, 1H), δ8.11-8.14 (m, 1H), δ8.25-8.31 (t, 2H), δ8.54-8.57 (d, 1H), δ8.62 (s, 1H), δ9.61 (s, 1H)

### Step 1: N-(2-methyl-4-(6-(trifluoromethyl)quinazolin-2-yl)phenyl)acetamide

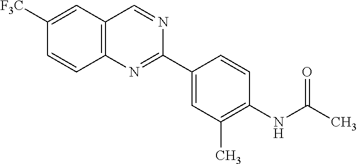 [SMILES: CC(=O)Nc1ccc(-c2ncc3cc(C(F)(F)F)ccc3n2)cc1C]

To a stirred solution of 2-methyl-4-[6-(trifluoromethyl)quinazolin-2-yl]aniline (100 mg, 0.33 mmol, 1 equiv) in CH₂Cl₂ (2 mL) was added triethylamine (50.0 mg, 0.49 mmol, 1.5 equiv) and acetyl chloride (31.1 mg, 0.40 mmol, 1.2 equiv). The resulting mixture was stirred at room temperature for 2 hours. The reaction was quenched by the addition of water (20 mL). The resulting mixture was extracted with CH₂Cl₂ (2×20 mL). The combined organic layers were concentrated under reduced pressure. The residue was purified by preparative TLC (CH₂Cl₂/MeOH=30/1) to afford the desired final product as a yellow solid in 99% yield.

### Example 131: N-methyl-N-(2-methyl-4-(6-(trifluoromethyl)quinazolin-2-yl)phenyl)-1H-imidazole-5-carboxamide

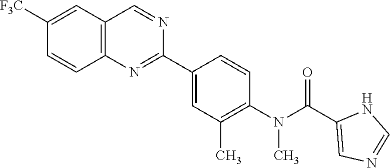 [SMILES: Cc1cc(-c2ncc3cc(C(F)(F)F)ccc3n2)ccc1N(C)C(=O)c1cnc[nH]1]

The title compound was prepared analogously to Example 001, where N,2-dimethyl-4-(6-(trifluoromethyl)quinazolin-2-yl)aniline was substituted in place of 2-methyl-4-(6-(trifluoromethyl)quinolin-2-yl)aniline. Yield=7%. LCMS (ES, m/z): [M+H]⁺ 412; ¹H-NMR (400 MHz, CDCl₃, ppm): δ9.60 (s, 1H), δ8.67 (s, 1H), δ8.63-8.61 (d, 1H), δ8.30 (s, 1H), δ8.25-8.23 (d, 1H), δ8.12-8.10 (m, 1H), δ7.70-7.71 (d, 1H), δ7.40-7.38 (d, 1H), δ5.79 (s, 1H). δ3.43 (s, 3H), δ2.28 (s, 3H

### Example 132: N-methyl-N-(2-methyl-4-(6-(trifluoromethyl)quinazolin-2-yl)phenyl)-1H-imidazole-2-carboxamide

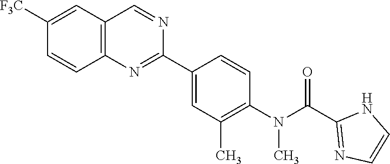 [SMILES: Cc1cc(-c2ncc3cc(C(F)(F)F)ccc3n2)ccc1N(C)C(=O)c1ncc[nH]1]

The title compound was prepared analogously to Example 122, where 1H-imidazole-2-carboxylic acid was substituted in place of 3-methylpyridine-4-carboxylic acid. Yield=9%. LCMS (ES, m/z): [M+H]⁺ 412; ¹H-NMR (400 MHz, DMSO-d₆,ppm): δ10.54-10.21 (d, 1H), δ9.59 (s, 1H), δ8.64-8.55 (m, 2H), δ8.29 (s, 1H), δ8.25-8.22 (t, 1H), δ8.12-8.09 (d, 1H), δ7.42-7.28 (dd, 1.5H), δ7.23 (s, 0.5H), δ7.02-6.89 (d, 1H). δ4.08 (s, 1H), δ3.45 (s, 2H), δ2.98-2.91 (d, 3H)

### Example 133: N-methyl-N-(2-methyl-4-(6-(trifluoromethyl)quinazolin-2-yl)phenyl)-1H-tetrazole-5-carboxamide

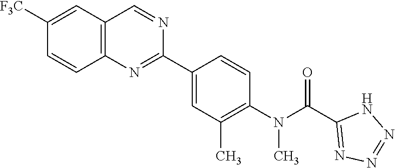 [SMILES: Cc1cc(-c2ncc3cc(C(F)(F)F)ccc3n2)ccc1N(C)C(=O)c1nnn[nH]1]

The title compound was prepared analogously to Example 122, where 1H-1,2,3,4-tetrazole-5-carboxylic acid was substituted in place of 3-methylpyridine-4-carboxylic acid. Yield=15%. LCMS (ES, m/z): [M+H]⁺ 414; ¹H-NMR (400 MHz, CD₃OD, ppm): δ2.34-2.45 (d, 3H), δ3.46-3.53 (d, 3H), δ7.31-7.51 (q, 1H), δ8.15-8.27 (m, 2H), δ8.37-8.66 (m, 3H), δ9.68-9.73 (d, 1H)

### Example 134: N-methyl-N-(2-methyl-4-(6-(trifluoromethyl)quinazolin-2-yl)phenyl)-1H-1,2,4-triazole-5-carboxamide

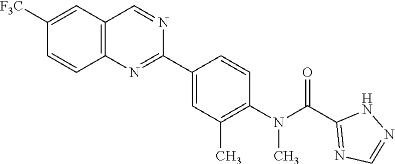 [SMILES: Cc1cc(-c2ncc3cc(C(F)(F)F)ccc3n2)ccc1N(C)C(=O)c1ncn[nH]1]

The title compound was prepared analogously to Example 122, where 1H-1,2,4-triazole-5-carboxylic acid acid was substituted in place of 3-methylpyridine-4-carboxylic acid. Yield=19%. LCMS (ES, m/z): [M+H]⁺ 413; ¹H-NMR (300 MHz, CDCl₃, ppm): δ2.38-2.40 (d, 3H), δ3.48-3.96 (d, 3H), δ5.80 (s, 1H), δ7.32-7.43 (t, 1H), δ7.91 (brs, 1H), δ8.09-8.12 (q, 1H), δ8.21-8.24 (d, 1H), δ8.29 (s, 1H), δ8.48-8.64 (m, 2H), δ9.59 (s, 1H)

### Example 135: 1-methyl-N-(2-methyl-4-(6-(trifluoromethyl)pyrido[3,2-d]pyrimidin-2-yl)phenyl)-1H-pyrazole-5-carboxamide

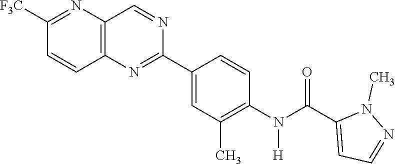 [SMILES: Cc1cc(-c2ncc3nc(C(F)(F)F)ccc3n2)ccc1NC(=O)c1ccnn1C]

DIEA (135.9 mg, 1.05 mmol, 2 equiv) and HATU (299.9 mg, 0.79 mmol, 1.5 equiv) were added to a solution of 2-methyl-4-[6-(trifluoromethyl)pyrido[3,2-d]pyrimidin-2-yl]aniline (160 mg, 0.53 mmol, 1 equiv) and 1-methyl-1H-pyrazole-5-carboxylic acid (99.5 mg, 0.79 mmol, 1.5 equiv) in DMF (2 mL). The resulting solution was stirred overnight at 50° C. The reaction was quenched by the addition of 10 mL of water. The resulting solution was extracted with ethyl acetate (2×3 mL) and concentrated under reduced pressure. The residue was purified by preparative TLC with dichloromethane/methanol (30/1) to afford the desired product as a yellow solid in 24% yield. LCMS (ES, m/z): [M+H]⁺ 413; ¹H-NMR (400 MHz, CDCl₃, ppm): δ2.51 (s, 3H), δ4.29 (s, 3H), δ6.71-6.72 (s, 1H), δ7.58 (s, 1H), δ7.72 (s, 1H), δ8.14-8.16 (d, 1H), δ8.33-8.37 (m, 1H), δ8.59-8.62 (m, 3H), δ9.81 (s, 1H)

### Step 1: 3-amino-6-(trifluoromethyl)picolinonitrile

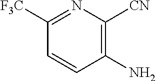 [SMILES: N#Cc1nc(C(F)(F)F)ccc1N]

2-bromo-6-(trifluoromethyl)pyridin-3-amine (10 g, 41.49 mmol, 1 equiv) and CuCN (10.0 g, 111.66 mmol, 2.691 equiv) were dissolved in DMSO (100 mL). The resulting solution was stirred for 4 hours at 120° C. followed by dilution with 400 mL of H₂O. The aqueous solution was extracted with ethyl acetate (2×300 mL) and concentrated under reduced pressure. The residue was purified by silica gel chromatography with ethyl acetate/petroleum ether (1/5) to afford the desired final product as a grey solid in 64% yield.

### Step 2: 3-amino-6-(trifluoromethyl)picolinic acid

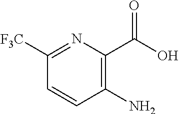 [SMILES: Nc1ccc(C(F)(F)F)nc1C(=O)O]

A solution of 3-amino-6-(trifluoromethyl)pyridine-2-carbonitrile (5 g, 26.72 mmol, 1 equiv) and KOH (50 mL, 2M) in EtOH (25 mL) was refluxed overnight. The ethanol was eliminated under reduced pressure and the pH of the solution was adjusted to 5 with 5% HCl. The solid was collected by filtration to afford the desired product in 78% yield.

### Step 3: 6-(trifluoromethyl)pyrido[3,2-d]pyrimidine-2,4(1H,3H)-dione

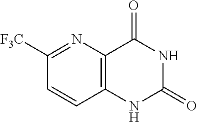 [SMILES: O=c1[nH]c(=O)c2nc(C(F)(F)F)ccc2[nH]1]

3-amino-5-(trifluoromethyl)pyridine-2-carboxylic acid (4.2 g, 20.38 mmol, 1 equiv) and urea (12.2 g, 203.15 mmol, 9.970 equiv) were heated at 200° C. for three hours. The resulting solution was diluted with 150 mL of water and the solid filtrated to afford the desired product in 85% yield.

### Step 4: 2,4-dichloro-6-(trifluoromethyl)pyrido[3,2-d]pyrimidine

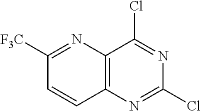 [SMILES: FC(F)(F)c1ccc2nc(Cl)nc(Cl)c2n1]

POCl₃ (20 mL) was added over 6-trifluoromethyl)-1H,2H,3H,4H-pyrido[3,2-d]pyrimidine-2,4-dione (4 g, 17.31 mmol, 1 equiv) and the resulting solution stirred for 3 hours at 100° C. The mixture was concentrated and the residue diluted with 50 mL of saturated NaHCO₃. After extraction with ethyl acetate (2×20 mL), the volatiles were eliminated under reduced pressure to afford a crude material that was purified by silica gel with ethyl acetate/petroleum ether (1/100) to afford the desired product as a white solid in 11% yield.

### Step 5: 2-chloro-6-(trifluoromethyl)pyrido[3,2-d]pyrimidine

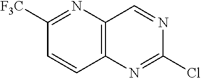 [SMILES: FC(F)(F)c1ccc2nc(Cl)ncc2n1]

Over a solution of 2,4-dichloro-6-(trifluoromethyl)pyrido[3,2-d]pyrimidine (500 mg, 1.87 mmol, 1 equiv) in THE (5 mL), Bu3SnH (544 mg, 1.87 mmol, 1 equiv) and Pd(PPh₃)₄(215.6 mg, 0.19 mmol, 0.1 equiv) were added. The resulting solution was stirred overnight at room temperature and concentrated under reduced pressure. The residue was purified by silica gel with ethyl acetate/petroleum ether (100/1) as eluent to afford the desired product as a white solid in 46% yield.

### Step 6: 2-(3-methyl-4-nitrophenyl)-6-(trifluoromethyl)pyrido[3,2-d]pyrimidine

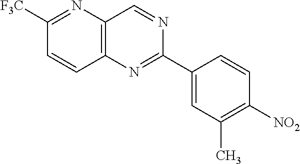 [SMILES: Cc1cc(-c2ncc3nc(C(F)(F)F)ccc3n2)ccc1[N+](=O)[O-]]

A mixture of 2-chloro-6-(trifluoromethyl)pyrido[3,2-d]pyrimidine (400 mg, 1.71 mmol, 1 equiv), 4,4,5,5-tetramethyl-2-(3-methyl-4-nitrophenyl)-1,3,2-dioxaborolane (450.6 mg, 1.71 mmol, 1 equiv), Na₂CO₃ (363.0 mg, 3.42 mmol, 2 equiv), Pd(PPh₃)₄(197.9 mg, 0.17 mmol, 0.1 equiv) in toluene (8 mL) and EtOH (4 mL) was stirred overnight at 100° C. The solids were filtered out and the solution concentrated. The residue was purified by preparative TLC with ethyl acetate/petroleum ether (1/5) as eluent to afford the desired product as a yellow solid in 40% yield.

### Step 7: 2-methyl-4-(6-(trifluoromethyl)pyrido[3,2-d]pyrimidin-2-yl)aniline

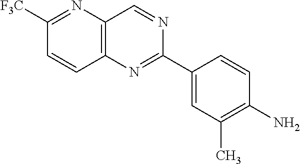 [SMILES: Cc1cc(-c2ncc3nc(C(F)(F)F)ccc3n2)ccc1N]

Over a solution of 2-(3-methyl-4-nitrophenyl)-6-(trifluoromethyl)pyrido[3,2-d]pyrimidine (230 mg, 0.69 mmol, 1 equiv) in 10 mL of ethanol, SnCl₂.2H₂O (621.1 mg, 2.75 mmol, 4 equiv) was added and resulting solution stirred for 2 hours at 80° C. The reaction was quenched by the addition of 20 mL of saturated solution of NaHCO₃, extracted with 3×10 mL of ethyl acetate and concentrated. The residue was purified by preparative TLC with ethyl acetate/petroleum ether (1:2) as eluent to afford the desired final product as a yellow solid in 72% yield.

### Example 136: N,1-dimethyl-N-(2-methyl-4-(6-(trifluoromethyl)pyrido[3,2-d]pyrimidin-2-yl)phenyl)-1H-pyrazole-5-carboxamide

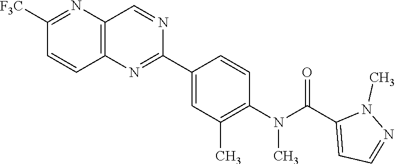 [SMILES: Cc1cc(-c2ncc3nc(C(F)(F)F)ccc3n2)ccc1N(C)C(=O)c1ccnn1C]

NaH (2.1 mg, 0.05 mmol, 1.1 equiv, 60%) was added to a solution of 1-methyl-N-[2-methyl-4-[6-(trifluoromethyl)pyrido[3,2-d]pyrimidin-2-yl]phenyl]-1H-pyrazole-5-carboxamide (20 mg, 0.05 mmol, 1 equiv) in DMF (3 mL) at 0° C. The reaction was stirred for 0.5 h at 0° C. and Mel (7.6 mg, 0.05 mmol, 1.104 equiv) was added. At that point the cooling bath was removed and stirring continued for another 4 hours at room temperature. The reaction was then quenched by the addition of 10 mL of saturated NH₄Cl. The resulting solution was extracted with ethyl acetate (2×3 mL) and concentrated. The residue was purified by preparative TLC with dichloromethane/methanol (30/1) to afford the desired final product as a white solid in 51% yield. LCMS (ES, m/z): [M+H]⁺ 427; ¹H-NMR (400 MHz, DMSO-d₆, ppm): δ2.28 (s, 3H), δ3.34 (s, 3H), δ4.01 (s, 3H), δ5.53-5.54 (d, 1H), δ7.14-7.15 (d, 1H), δ7.51-7.54 (d, 1H), δ8.35-8.50 (m, 3H), δ8.75-8.81 (d, 1H), δ9.96 (s, 1H)

### Example 137: N-(2-fluoro-4-(6-(trifluoromethyl)pyrido[3,2-d]pyrimidin-2-yl)phenyl)-1-methyl-1H-pyrazole-5-carboxamide

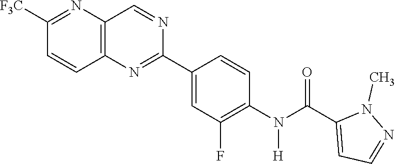 [SMILES: Cn1nccc1C(=O)Nc1ccc(-c2ncc3nc(C(F)(F)F)ccc3n2)cc1F]

The title compound was prepared analogously to Example 122, where 2-fluoro-4-(6-(trifluoromethyl)pyrido[3,2-d]pyrimidin-2-yl)aniline was substituted in place of N,2-dimethyl-4-[6-(trifluoromethyl)quinazolin-2-yl]aniline and 1-methyl-1H-pyrazole-5-carboxylic acid was substituted in place of 3-methylpyridine-4-carboxylic acid. Yield=34%. LCMS (ES, m/z): [M+H]⁺ 417; ¹H-NMR (400 MHz, DMSO-d₆, ppm): δ4.11 (s, 3H), δ7.16-7.17 (d, 1H), δ7.57-7.58 (s, 1H), δ7.92-7.96 (t, 1H), δ8.39-8.43 (q, 1H), δ8.47-8.49 (q, 2H), δ8.80-8.82 (d, 1H), δ9.96 (s, 1H), δ10.35 (s, 1H)

### Step 1: 2-(3-fluoro-4-nitrophenyl)-6-(trifluoromethyl)pyrido[3,2-d]pyrimidine

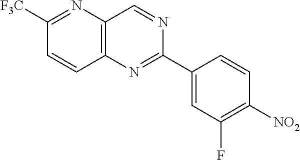 [SMILES: O=[N+]([O-])c1ccc(-c2ncc3nc(C(F)(F)F)ccc3n2)cc1F]

The title compound was prepared analogously to Example 110, where 2-chloro-6-(trifluoromethyl)pyrido[3,2-d]pyrimidine was substituted in place of 2-chloro-8-methyl-6-(trifluoromethyl)quinazoline and 2-(3-fluoro-4-nitrophenyl)-4,4,5,5-tetramethyl-1,3,2-dioxaborolane was substituted in place of 1-methyl-N-(2-methyl-4-(4,4,5,5-tetramethyl-1,3,2-dioxaborolan-2-yl)phenyl)-1H-pyrazole-5-carboxamide. Yield=34%.

### Step 2: 2-fluoro-4-(6-(trifluoromethyl)pyrido[3,2-d]pyrimidin-2-yl)aniline

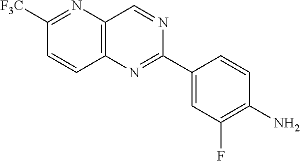 [SMILES: Nc1ccc(-c2ncc3nc(C(F)(F)F)ccc3n2)cc1F]

2-(3-fluoro-4-nitrophenyl)-6-(trifluoromethyl)pyrido[3,2-d]pyrimidine (170 mg, 0.50 mmol, 1 equiv) was dissolved in 10 mL of ethanol and SnCl₂.2H₂O (453.7 mg, 2.01 mmol, 4 equiv) was added. The resulting solution was stirred for 3 hours at 80° C. and quenched by the addition of 20 mL of saturated NaHCO₃. Extraction with ethyl acetate (2×10 mL) and elimination of volatiles under reduced pressure afforded a residue that was purified by silica gel chromatography with ethyl acetate/petroleum ether (1/2). The desired final product was isolated as a yellow solid in 65% yield.

### Example 138: N-(2-fluoro-4-(6-(trifluoromethyl)pyrido[3,2-d]pyrimidin-2-yl)phenyl)-N,1-dimethyl-1H-pyrazole-5-carboxamide

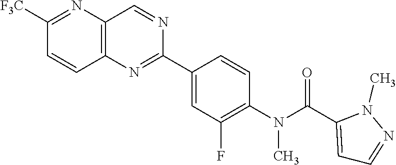 [SMILES: CN(C(=O)c1ccnn1C)c1ccc(-c2ncc3nc(C(F)(F)F)ccc3n2)cc1F]

The title compound was prepared analogously to Example 136, where N-(2-fluoro-4-(6-(trifluoromethyl)pyrido[3,2-d]pyrimidin-2-yl)phenyl)-1-methyl-1H-pyrazole-5-carboxamide was substituted in place of 1-methyl-N-[2-methyl-4-[6-(trifluoromethyl)pyrido[3,2-d]pyrimidin-2-yl]phenyl]-1H-pyrazole-5-carboxamide. Yield=51%. LCMS (ES, m/z): [M+H]⁺ 431; ¹H-NMR (400 MHz, DMSO-d₆, ppm): δ3.42 (s, 3H), δ3.97 (s, 3H), δ5.88 (s, 1H), δ7.22 (s, 1H), δ7.77-7.81 (t, 1H), δ8.28-8.31 (d, 1H), δ8.44-8.51 (m, 2H), δ8.80-8.82 (d, 1H), δ9.97 (s, 1H)

### Example 139: 1-methyl-N-(2-methyl-4-(2-(trifluoromethyl)pyrido[3,2-d]pyrimidin-6-yl)phenyl)-1H-pyrazole-5-carboxamide

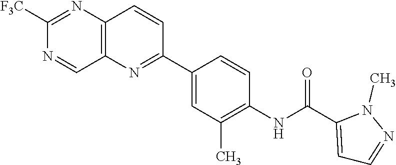 [SMILES: Cc1cc(-c2ccc3nc(C(F)(F)F)ncc3n2)ccc1NC(=O)c1ccnn1C]

The title compound was prepared analogously to Example 109, where 6-chloro-2-(trifluoromethyl)pyrido[3,2-d]pyrimidine was substituted in place of 2-chloro-6-fluoro-8-methylquinazoline. Yield=53%. LCMS (ES, m/z): [M+H]⁺ 413; ¹H-NMR (400 MHz, DMSO-d₆, ppm): δ2.41 (s, 3H), δ4.11 (s, 3H), δ7.11-7.12 (d, 1H), δ7.56-7.57 (d, 1H), δ7.64-7.67 (d, 1H), δ8.25-8.28 (q, 1H), δ8.34-8.35 (d, 1H), 8.72-8.74 (d, 1H), 8.83-8.85 (d, 1H), 9.93 (s, 1H), 10.02 (s, 1H)

### Step 1: 4-bromo-2-(trifluoromethyl)pyrimidin-5-amine

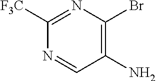 [SMILES: Nc1cnc(C(F)(F)F)nc1Br]

To a solution of 2-(trifluoromethyl)pyrimidin-5-amine (5 g, 30.66 mmol, 1 equiv) in DME (300 mL) was added Br₂ (4.90 g, 30.66 mmol, 1 equiv) and Fe (171.2 mg, 3.07 mmol, 0.1 equiv). The resulting mixture was stirred at 80° C. overnight, quenched by the addition of saturated NaHCO₃ (300 mL) and extracted with CH₂C₂(2×300 mL). The combined organic layers were concentrated under reduced pressure and the residue was purified by silica gel column chromatography with EtOAc/PE (1/10) to afford the desired product as a yellow solid in 20% yield

### Step 2: ethyl (E)-3-(5-amino-2-(trifluoromethyl)pyrimidin-4-yl)acrylate

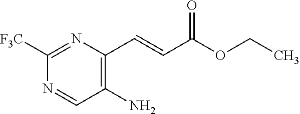 [SMILES: CCOC(=O)/C=C/c1nc(C(F)(F)F)ncc1N]

The title compound was prepared analogously to Example 001, step 1, where 4-bromo-2-(trifluoromethyl)pyrimidin-5-amine was substituted in place 2-bromo-4-(trifluoromethyl)aniline. Yield=76%.

### Step 3: 2-(trifluoromethyl)pyrido[3,2-d]pyrimidin-6(5H)-one

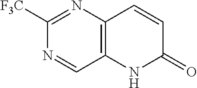 [SMILES: O=c1ccc2nc(C(F)(F)F)ncc2[nH]1]

To a solution of ethyl (E)-3-(5-amino-2-(trifluoromethyl)pyrimidin-4-yl)acrylate (350 mg, 1.34 mmol, 1 equiv) in EtOH (30 mL, 516.41 mmol, 385.387 equiv), EtONa (364.7 mg, 5.36 mmol, 4 equiv) was added. The resulting mixture was stirred at 80° C. for 2 hours. The reaction was quenched by the addition of saturated NH₄Cl (100 mL). The resulting mixture was extracted with EtOAc (2×100 mL). The combined organic layers were concentrated under reduced pressure. The residue was purified by preparative TLC (CH₂C₂/MeOH 20/1) to afford the desired product as a yellow solid in 85% yield.

### Step 4: 6-chloro-2-(trifluoromethyl)pyrido[3,2-d]pyrimidine

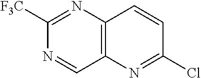 [SMILES: FC(F)(F)c1ncc2nc(Cl)ccc2n1]

The title compound was prepared analogously to Example 001, step 3, where 2-(trifluoromethyl)pyrido[3,2-d]pyrimidin-6(5H)-one was substituted in place of 6-(trifluoromethyl)-1,2-dihydroquinolin-2-one. Yield=41%.

### Example 140: N,1-Dimethyl-N-(2-methyl-4-(6-(trifluoromethyl)quinazolin-2-yl)phenyl)-1H-pyrazole-5-carboxamide

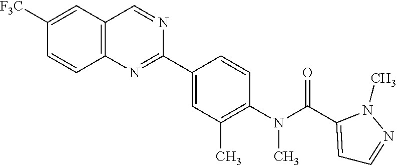 [SMILES: Cc1cc(-c2ncc3cc(C(F)(F)F)ccc3n2)ccc1N(C)C(=O)c1ccnn1C]

The title compound was prepared analogously to Example 002, where 1-methyl-N-[2-methyl-4-[6-(trifluoromethyl)quinazolin-2-yl]phenyl]-1H-pyrazole-5-carboxamide was substituted in place of 1-methyl-N-(2-methyl-4-(6-(trifluoromethyl)quinolin-2-yl)phenyl)-1H-pyrazole-5-carboxamide. Yield=57%. LCMS (ES, m/z): [M+H]⁺ 426; ¹H-NMR (400 MHz, DMSO-d₆, ppm): δ9.89 (s, 1H), δ8.72 (s, 1H), δ8.48-8.40 (m, 2H), δ8.30-8.23 (q, 2H), δ7.51-7.48 (d, 1H), δ7.14 (s, 1H), δ5.53 (s, 1H), δ4.01 (s, 3H), δ3.33 (s, 3H), δ2.27 (s, 3H)

### Example 141: 1-(2-methyl-4-(6-(trifluoromethyl)quinazolin-2-yl)phenyl)imidazolidin-2-one

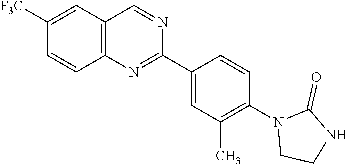 [SMILES: Cc1cc(-c2ncc3cc(C(F)(F)F)ccc3n2)ccc1N1CCNC1=O]

1-(2-chloroethyl)-3-(2-methyl-4-(6-(trifluoromethyl)quinazolin-2-yl)phenyl)urea (110 mg, 0.27 mmol, 1.00 equiv) was dissolved in DMF (1 mL) and sodium hydride (13 mg, 0.54 mmol, 1.20 equiv) added. The resulting solution was stirred overnight at room temperature. The reaction was then quenched by the addition of a saturated solution of NH₄Cl, extracted with 2×300 mL of ethyl acetate and the organic layers combined and concentrated under reduced pressure. The residue was purified by preparative TLC with dichloromethane/methanol (20:1) as eluent. The crude product was purified by recrystallization from methanol to afford the final product as a yellow solid in 26% yield. LCMS: (ES, m/z): [M+H]⁺ 373; ¹H-NMR (DMSO-d₆, 300 MHz ppm): δ2.37 (s, 3H), 52.45-2.50 (t, 2H), δ3.81-3.86 (t, 2H), δ6.82 (s, 1H), δ7.43-7.46 (d, 1H), δ8.23-8.29 (m, 2H), δ8.41-8.44 (q, 1H), δ8.48-8.49 (d, 1H), δ8.70 (s, 1H), δ9.87 (s, 1H)

### Step 1: 1-(2-chloroethyl)-3-(2-methyl-4-(6-(trifluoromethyl)quinazolin-2-yl)phenyl)urea

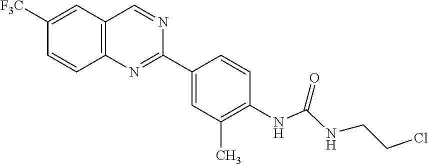 [SMILES: Cc1cc(-c2ncc3cc(C(F)(F)F)ccc3n2)ccc1NC(=O)NCCCl]

A mixture of 2-methyl-4-[6-(trifluoromethyl)quinazolin-2-yl]aniline (100 mg, 0.33 mmol, 1.00 equiv), triethylamine (70 mg, 0.69 mmol, 2.00 equiv), 1-chloro-2-isocyanatoethane (70 mg, 0.66 mmol, 2.00 equiv) in dichloromethane (1 mL) was stirred at room temperature overnight. The resulting solution was diluted with 10 mL of H₂O and extracted with 200 mL of ethyl acetate. The combined organic layers were concentrated under vacuum and the resulting crude product purified by recrystallization from methanol affording the desired product as a yellow solid in 96% yield.

### Example 142: 2-methoxy-N-(2-methyl-4-(6-(trifluoromethyl)quinazolin-2-yl)phenyl)isonicotinamide

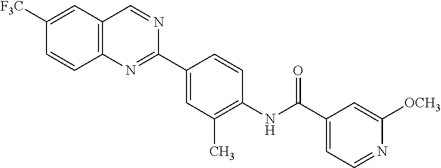 [SMILES: COc1cc(C(=O)Nc2ccc(-c3ncc4cc(C(F)(F)F)ccc4n3)cc2C)ccn1]

The title compound was prepared analogously to Example 001, where 2-methoxyisonicotinic acid was substituted in place of 1-methyl-1H-pyrazole-5-carboxylic acid. LCMS (ES, m/z): [M+H]⁺ 439; ¹H-NMR (400 MHz, DMSO-d₆, ppm): 10.22 (s, 1H), 9.89 (s, 1H), 8.72 (s, 1H), 8.54-8.53 (d, 1H), 8.48-8.46 (q, 1H), 8.39-8.38 (d, 1H), 8.30-8.25 (m, 2H), 7.68-7.66 (d, 1H), 7.50-7.49 (m, 1H), 7.36 (s, 1H), 3.95 (s, 3H), 2.41 (s, 3H)

### Example 143: 2-Methoxy-N-methyl-N-(2-methyl-4-(6-(trifluoromethyl)quinazolin-2-yl)phenyl)isonicotinamide

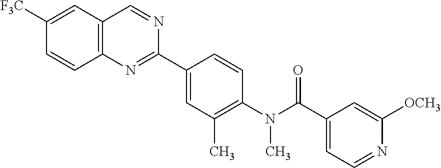 [SMILES: COc1cc(C(=O)N(C)c2ccc(-c3ncc4cc(C(F)(F)F)ccc4n3)cc2C)ccn1]

To a solution of N,2-dimethyl-4-[6-(trifluoromethyl)quinazolin-2-yl]aniline (60 mg, 0.19 mmol, 1 equiv) and Et₃N (57.4 mg, 0.57 mmol, 3 equiv) in dichloromethane kept at 0° C., 2-methoxypyridine-4-carbonyl chloride (48.7 mg, 0.28 mmol, 1.5 equiv) was added. The resulting mixture was stirred at room temperature for 12 hours. The reaction was quenched with saturated NH₄Cl and the aqueous layer extracted with ethyl acetate (2×10 mL). The combined organic layers were combined and concentrated under reduced pressure. The residue was purified by preparative TLC (dichloromethane/MeOH=20:1) to afford the desired final product as a white solid in 45% yield. LCMS (ES, m/z): [M+H]⁺ 453; ¹H-NMR (400 MHz, CDCl₃, ppm): δ9.60 (s, 1H), 8.63 (s, 1H), 8.42-8.40 (d, 1H), 8.26 (s, 1H), 8.23-8.21 (d, 1H), 8.12-8.10 (d, 1H), 7.98-7.97 (d, 1H), 7.31-7.28 (d, 1H), 6.76-6.74 (d, 1H), 6.70 (s, 1H), 4.03 (s, 3H), 3.47 (s, 3H), 2.44 (s, 3H)

### Example 144: 3-methoxy-N-(2-methyl-4-(6-(trifluoromethyl)quinazolin-2-yl)phenyl)isonicotinamide

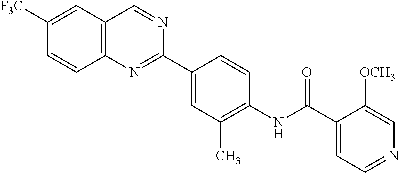 [SMILES: COc1cnccc1C(=O)Nc1ccc(-c2ncc3cc(C(F)(F)F)ccc3n2)cc1C]

A solution of 2-methyl-4-[6-(trifluoromethyl)quinazolin-2-yl]aniline (100 mg, 0.33 mmol, 1 equiv), 3-methoxypyridine-4-carboxylic acid (75.7 mg, 0.49 mmol, 1.5 equiv), DIEA (85.2 mg, 0.66 mmol, 2 equiv), HATU (188.1 mg, 0.49 mmol, 1.5 equiv) in DMF (2 mL) was stirred overnight at 65° C. The reaction was quenched by the addition of 10 mL of water and extracted with ethyl acetate (2×20 mL). The combined organic layers were evaporated under reduced pressure. The crude product was purified under the following conditions: Column=XBridge Prep C18 OBD Column; mobile phase=water (0.1% NH₄HCO₃) and acetonitrile (10% up to 30% in 15 min); Detector=UV 254 nm. The desired final product was isolated as a white solid in 18% yield. LCMS (ES, m/z): [M+H]⁺ 439; ¹H-NMR (400 MHz, DMSO-d₆, ppm): δ10.06 (s, 1H), δ9.88 (s, 1H), δ8.71-8.69 (d, 2H), δ8.53-8.42 (m, 3H), δ8.27-8.18 (m, 3H), δ7.78-7.77 (d, 1H), δ4.14 (s, 3H), δ2.48 (s, 3H)

### Example 145: 3-Methoxy-N-methyl-N-(2-methyl-4-(6-(trifluoromethyl)quinazolin-2-yl)phenyl)isonicotinamide

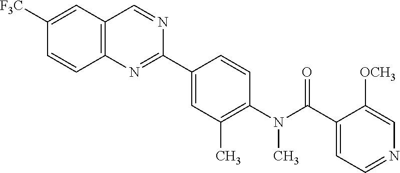 [SMILES: COc1cnccc1C(=O)N(C)c1ccc(-c2ncc3cc(C(F)(F)F)ccc3n2)cc1C]

The title compound was prepared analogously to Example 144, where N,2-dimethyl-4-(6-(trifluoromethyl)quinazolin-2-yl)aniline was substituted in place of 2-methyl-4-[6-(trifluoromethyl)quinazolin-2-yl]aniline. The crude product was purified by reverse phase chromatography with the following conditions: Column=XBridge Prep C18 OBD Column, mobile phase=water (0.01% NH₄HCO₃) and acetonitrile (10% up to 35% in 15 min), detector=UV 254 nm. Yield=19%. LCMS (ES, m/z): [M+H]⁺ 453; ¹H-NMR (400 MHz, CDCl₃, ppm): δ9.60 (s, 1H), δ8.59 (s, 1H), δ8.44-8.29 (m, 2H), δ8.27-8.08 (m, 4H), δ7.28-7.24 (d, 1H), δ7.15-7.14 (d, 1H), δ4.10-3.88 (d, 3H), δ3.46-3.20 (d, 3H), δ2.52-2.46 (d, 3H)

### Example 146: N-methyl-N-(2-methyl-4-(6-(trifluoromethyl)quinazolin-2-yl)phenyl)-1H-1,2,3-triazole-5-carboxamide

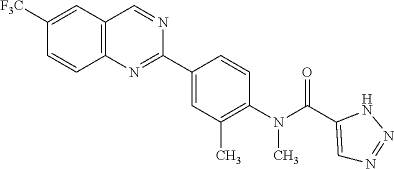 [SMILES: Cc1cc(-c2ncc3cc(C(F)(F)F)ccc3n2)ccc1N(C)C(=O)c1cnn[nH]1]

The title compound was prepared analogously to Example 144, where 1H-1,2,3-triazole-5-carboxylic acid was substituted in place of 3-methoxypyridine-4-carboxylic acid and N,2-dimethyl-4-(6-(trifluoromethyl)quinazolin-2-yl)aniline was substituted in place of 2-methyl-4-[6-(trifluoromethyl)quinazolin-2-yl]aniline. The crude product was purified by preparative HPLC with the following conditions (2#-AnalyseHPLC-SHIMADZU (HPLC-10)): Column, XBridge Prep C18 OBD Column, 5 um, 19*150 mm; mobile phase, water (10 mmol/L, NH₄HCO₃) and CH₃CN (30% Phase B up to 42% in 7 min); Detector, UV. Yield=7%. LCMS (ES, m/z): [M+H]⁺ 413; ¹H-NMR (400 MHz, DMSO-d₆, ppm): δ9.91 (s, 1H), δ8.74 (s, 1H), δ8.51-8.44 (d, 2H), δ8.42 (s, 2H), δ8.32-8.26 (m, 1H), δ7.42-7.40 (d, 1H), δ7.30 (s, 1H), δ3.32 (s, 3H), δ2.33 (s, 3H)

### Example 147: 1-methyl-N-(2-methyl-4-(8-methyl-6-(trifluoromethyl)pyrido[3,2-d]pyrimidin-2-yl)phenyl)-1H-pyrazole-5-carboxamide

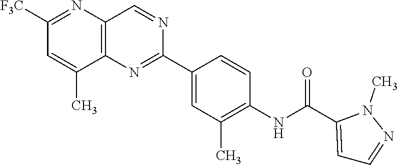 [SMILES: Cc1cc(-c2ncc3nc(C(F)(F)F)cc(C)c3n2)ccc1NC(=O)c1ccnn1C]

The title compound was prepared analogously to Example 009, where 2-chloro-8-methyl-6-(trifluoromethyl)pyrido[3,2-d]pyrimidine was substituted in place of 2-chloro-6-(trifluoromethyl)quinazoline and 1-methyl-N-[2-methyl-4-(tetramethyl-1,3,2-dioxaborolan-2-yl)phenyl]-1H-pyrazole-5-carboxamide was substituted in place of tert-butyl N-methyl-N-[2-methyl-4-(tetramethyl-1,3,2-dioxaborolan-2-yl)phenyl]carbamate. LCMS (ES, m/z): [M+H]⁺ 427; ¹H-NMR (400 MHz, DMSO-d₆, ppm): δ2.41 (s, 3H), δ2.95 (s, 3H), δ4.12 (s, 3H), δ7.12-7.13 (d, 1H), δ7.57-7.58 (d, 1H), δ7.65-7.67 (d, 1H), δ8.39 (s, 1H), δ8.50-8.52 (q, 1H), δ8.56-8.57 (d, 1H), δ9.89 (s, 1H), δ10.02 (s, 1H)

### Step 1: 3-amino-4-methyl-6-(trifluoromethyl)picolinonitrile

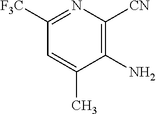

Zn(CN)₂ (4.6 g, 39.17 mmol, 1.998 equiv) and Pd(PPh₃)₄(2.3 g, 1.99 mmol, 0.102 equiv) were added over a solution of 2-bromo-4-methyl-6-(trifluoromethyl)pyridin-3-amine (5 g, 19.61 mmol, 1 equiv) in DMF (50 mL). The resulting solution was stirred overnight at 80° C., diluted with 150 mL of water, extracted with 2×50 ml of ethyl acetate and concentrated under reduced pressure. The residue was purified by silica gel chromatgraphy with ethyl acetate/petroleum ether (1:2) to afford the desired final product as a white solid in 94% yield.

### Step 2: 8-methyl-6-(trifluoromethyl)pyrido[3,2-d]pyrimidine-2,4(1H,3H)-dione

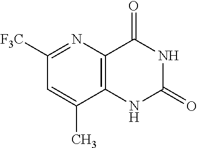 [SMILES: Cc1cc(C(F)(F)F)nc2c(=O)[nH]c(=O)[nH]c12]

3-amino-4-methyl-6-(trifluoromethyl)pyridine-2-carbonitrile (1.4 g, 6.96 mmol, 1 equiv) was dissolved in DMF (30 mL) and DBU (3.2 g, 21.02 mmol, 3.020 equiv) was added. CO₂ was introduced into the flask and the resulting solution stirred overnight at 100° C. After quenching with 90 mL of water, the pH value of the solution was adjusted to 5 with 5% HCl. The solids were collected by filtration to afford the desired final product as a white solid in 88% yield.

### Step 3: 2,4-dichloro-8-methyl-6-(trifluoromethyl)pyrido[3,2-d]pyrimidine

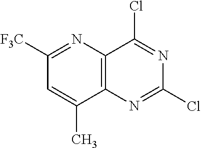 [SMILES: Cc1cc(C(F)(F)F)nc2c(Cl)nc(Cl)nc12]

The title compound was prepared analogously to Example 135, step 4, where 8-methyl-6-(trifluoromethyl)pyrido[3,2-d]pyrimidine-2,4(1H,3H)-dione was substituted in place of 6-(trifluoromethyl)-1H,2H,3H,4H-pyrido[3,2-d]pyrimidine-2,4-dione. Yield=30%.

### Step 4: 2-chloro-8-methyl-6-(trifluoromethyl)pyrido[3,2-d]pyrimidine

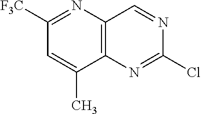 [SMILES: Cc1cc(C(F)(F)F)nc2cnc(Cl)nc12]

A mixture of 2,4-dichloro-8-methyl-6-(trifluoromethyl)pyrido[3,2-d]pyrimidine (300 mg, 1.06 mmol, 1 equiv), PPh₃ (418.5 mg, 1.60 mmol, 1.5 equiv), Bu₃SnH (325.0 mg, 1.12 mmol, 1.050 equiv) and Pd(PPh₃)₄(122.9 mg, 0.11 mmol, 0.100 equiv) in 5 mL of DMF was stirred for 5 hours at room temperature. The volatiles were removed under reduced pressure to afford a residue that was purified by silica gel chromatography with ethyl acetate/petroleum ether (50/1) affording the desired final product as a white solid in 65% yield.

### Example 148: N,1-Dimethyl-N-(2-methyl-4-(8-methyl-6-(trifluoromethyl)pyrido[3,2-d]pyrimidin-2-yl)phenyl)-1H-pyrazole-5-carboxamide

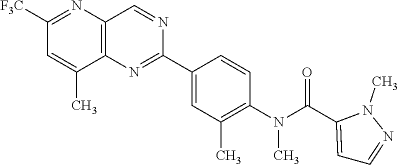 [SMILES: Cc1cc(-c2ncc3nc(C(F)(F)F)cc(C)c3n2)ccc1N(C)C(=O)c1ccnn1C]

The title compound was prepared analogously to Example 009, where 2-chloro-8-methyl-6-(trifluoromethyl)pyrido[3,2-d]pyrimidine was substituted in place of 2-chloro-6-(trifluoromethyl)quinazoline and N,1-dimethyl-N-(2-methyl-4-(4,4,5,5-tetramethyl-1,3,2-dioxaborolan-2-yl)phenyl)-1H-pyrazole-5-carboxamide was substituted in place of tert-butyl N-methyl-N-[2-methyl-4-(tetramethyl-1,3,2-dioxaborolan-2-yl)phenyl]carbamate. Yield=50%. LCMS (ES, m/z): [M+H]⁺ 441; ¹H-NMR (400 MHz, DMSO-d₆, ppm): δ2.29 (s, 3H), δ2.95 (s, 3H), δ3.35 (s, 3H), δ3.99 (s, 3H), δ5.54-5.55 (d, 1H), δ7.14-7.15 (d, 1H), δ7.52-7.54 (d, 1H), δ8.39-8.40 (d, 1H), δ8.45-8.47 (m, 1H), δ8.53 (s, 1H), δ9.89 (s, 1H)

### Example 149: 2-methyl-N-(2-methyl-4-(8-methyl-6-(trifluoromethyl)pyrido[3,2-d]pyrimidin-2-yl)phenyl)isonicotinamide

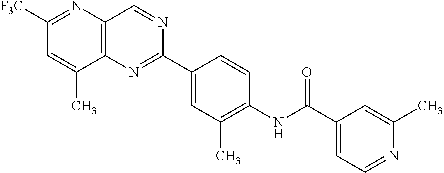 [SMILES: Cc1cc(C(=O)Nc2ccc(-c3ncc4nc(C(F)(F)F)cc(C)c4n3)cc2C)ccn1]

The title compound was prepared analogously to Example 122, where 2-methyl-4-(8-methyl-6-(trifluoromethyl)pyrido[3,2-d]pyrimidin-2-yl)aniline was substituted in place of N,2-dimethyl-4-[6-(trifluoromethyl)quinazolin-2-yl]aniline and 2-methylisonicotinic acid was substituted in place of 3-methylpyridine-4-carboxylic acid. Yield=40%. LCMS (ES, m/z): [M+H]⁺ 438; ¹H-NMR (400 MHz, DMSO-d₆, ppm): δ2.42 (s, 3H), δ2.60 (s, 3H), δ2.95 (s, 3H), δ7.68-7.73 (m, 2H), δ7.80 (s, 1H), δ8.39 (s, 1H), δ8.50-8.52 (d, 1H), δ8.57 (s, 1H), δ8.67-8.68 (d, 1H), δ9.89 (s, 1H), δ10.26 (s, 1H)

### Step 1: 8-methyl-2-(3-methyl-4-nitrophenyl)-6-(trifluoromethyl)pyrido[3,2-d]pyrimidine

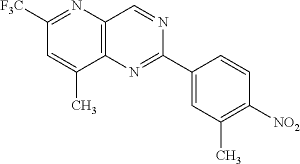 [SMILES: Cc1cc(-c2ncc3nc(C(F)(F)F)cc(C)c3n2)ccc1[N+](=O)[O-]]

The title compound was prepared analogously to Example 009, where 2-chloro-8-methyl-6-(trifluoromethyl)pyrido[3,2-d]pyrimidine was substituted in place of 2-chloro-6-(trifluoromethyl)quinazoline and 4,4,5,5-tetramethyl-2-(3-methyl-4-nitrophenyl)-1,3,2-dioxaborolane was substituted in place of tert-butyl N-methyl-N-[2-methyl-4-(tetramethyl-1,3,2-dioxaborolan-2-yl)phenyl]carbamate. Yield=99%

### Step 2: 2-methyl-4-(8-methyl-6-(trifluoromethyl)pyrido[3,2-d]pyrimidin-2-yl)aniline

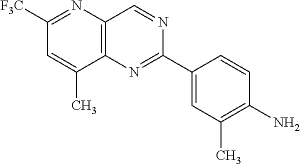 [SMILES: Cc1cc(-c2ncc3nc(C(F)(F)F)cc(C)c3n2)ccc1N]

The title compound was prepared analogously to Example 135, step 7, where 8-methyl-2-(3-methyl-4-nitrophenyl)-6-(trifluoromethyl)pyrido[3,2-d]pyrimidine was substituted in place 2-(3-methyl-4-nitrophenyl)-6-(trifluoromethyl)pyrido[3,2-d]pyrimidine. Yield=59%.

### Example 150: N,2-dimethyl-N-(2-methyl-4-(8-methyl-6-(trifluoromethyl)pyrido[3,2-d]pyrimidin-2-yl)phenyl)isonicotinamide

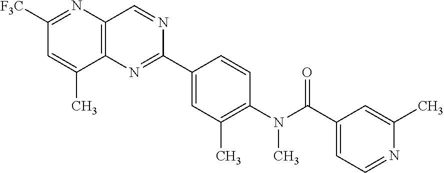 [SMILES: Cc1cc(C(=O)N(C)c2ccc(-c3ncc4nc(C(F)(F)F)cc(C)c4n3)cc2C)ccn1]

LiHMDS (0.24 mL, 1.5 equiv, 1 M) was added over a solution of 2-methyl-N-[2-methyl-4-[8-methyl-6-(trifluoromethyl)pyrido[3,2-d]pyrimidin-2-yl]phenyl]pyridine-4-carboxamide (70 mg, 0.16 mmol, 1 equiv) in THF (5 mL). After 0.5 hours, methyl iodide (22.7 mg, 0.16 mmol, 1 equiv) was added. The resulting solution was stirred overnight at room temperature. The reaction was quenched by addition of 10 mL of saturated NH₄Cl, extracted with ethyl acetate (2×5 mL) and the combined organic layers concentrated under reduced pressure. The residue was purified by preparative TLC with dichloromethane/methanol (25:1) as eluent to afford the desired final product as a white solid in 72% yield. LCMS (ES, m/z): [M+H]⁺ 452; ¹H-NMR (400 MHz, DMSO-d₆, ppm): δ2.35-2.38 (d, 6H), δ2.90 (s, 3H), δ3.33 (s, 3H), δ6.96-6.97 (d, 1H), δ7.21 (s, 1H), δ7.47-7.49 (d, 1H), δ8.25-8.27 (d, 1H), δ8.30-8.37 (m, 2H), δ8.45 (s, 1H), δ9.84 (s, 1H)

### Example 151: N4-(2-methyl-4-(6-(trifluoromethyl)quinazolin-2-yl)phenyl)pyridine-2,4-dicarboxamide

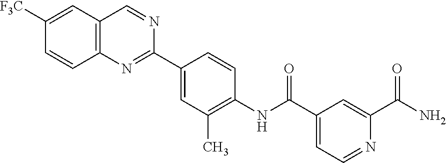 [SMILES: Cc1cc(-c2ncc3cc(C(F)(F)F)ccc3n2)ccc1NC(=O)c1ccnc(C(N)=O)c1]

A solution of 2-cyano-N-[2-methyl-4-[6-(trifluoromethyl)quinazolin-2-yl]phenyl]pyridine-4-carboxamide (30 mg, 0.07 mmol, 1 equiv) in concentrated H₂SO₄ (0.2 mL) was stirred for 30 min at room temperature. The reaction was quenched by the addition of 5 mL of ice/water, extracted with ethyl acetate (3×5 mL) and the combined organic layers concentrated under reduced pressure. Addition of MeOH afforded the desired final product as a solid, that was isolated after filtration in 57% yield. LCMS (ES, m/z): [M+H]⁺ 452; ¹H-NMR (300 MHz, DMSO-d₆, ppm): δ2.42 (s, 3H), δ7.65-7.68 (d, 1H), δ7.82 (s, 1H), δ8.09-8.11 (d, 1H), δ8.28 (s, 3H), δ8.47-8.50 (d, 1H), δ8.54-8.59 (d, 2H), δ8.72 (s, 1H), δ8.86-8.88 (d, 1H), δ9.90 (s, 1H), δ10.52 (s, 1H)

### Step 1: 2-Cyano-N-(2-methyl-4-(6-(trifluoromethyl)quinazolin-2-yl)phenyl)isonicotinamide

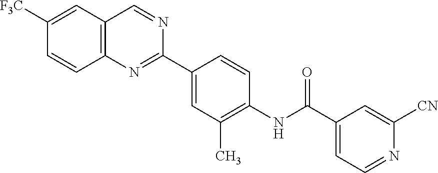 [SMILES: Cc1cc(-c2ncc3cc(C(F)(F)F)ccc3n2)ccc1NC(=O)c1ccnc(C#N)c1]

2-cyanopyridine-4-carbonyl chloride (54.9 mg, 0.33 mmol, 1 equiv) was added over a solution of 2-methyl-4-[6-(trifluoromethyl)quinazolin-2-yl]aniline (100 mg, 0.33 mmol, 1 equiv) in 2 mL of dichloromethane, followed by Et₃N (66.7 mg, 0.66 mmol, 2 equiv). The resulting solution was stirred for 3 hr at room temperature. The reaction was quenched by the addition of 5 mL of water, extracted with dichloromethane (2×5 mL) and the combined organic layers concentrated. The residue was purified by preparative TLC with dichloromethane/methanol (20/1) to afford the desired final product as a white solid in 24% yield.

### Example 152: N4-methyl-N4-(2-methyl-4-(6-(trifluoromethyl)quinazolin-2-yl)phenyl)pyridine-2,4-dicarboxamide

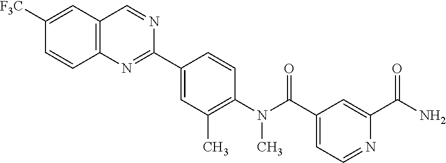 [SMILES: Cc1cc(-c2ncc3cc(C(F)(F)F)ccc3n2)ccc1N(C)C(=O)c1ccnc(C(N)=O)c1]

The title compound was prepared analogously to Example 151, where 2-cyano-N-methyl-N-(2-methyl-4-(6-(trifluoromethyl)quinazolin-2-yl)phenyl)isonicotinamide was substituted in place of 2-cyano-N-[2-methyl-4-[6-(trifluoromethyl)quinazolin-2-yl]phenyl]pyridine-4-carboxamide. Yield=89%. LCMS (ES, m/z): [M+H]⁺ 466; ¹H-NMR (300 MHz, DMSO-d₆, ppm): δ2.36 (s, 3H), δ3.36 (s, 3H), δ7.40-7.43 (m, 1H), δ7.48-7.50 (d, 1H), δ7.62 (s, 1H), δ7.90 (s, 1H), δ8.02 (s, 1H), δ8.20-8.31 (m, 3H), δ8.40-8.41 (d, 1H), δ8.47-8.49 (d, 1H), δ8.70 (s, 1H), δ9.85 (s, 1H).

### Step 1: 2-Cyano-N-methyl-N-(2-methyl-4-(6-(trifluoromethyl)quinazolin-2-yl)phenyl)isonicotinamide

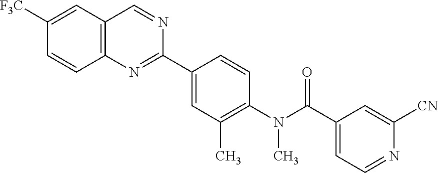 [SMILES: Cc1cc(-c2ncc3cc(C(F)(F)F)ccc3n2)ccc1N(C)C(=O)c1ccnc(C#N)c1]

The title compound was prepared analogously to Example 151, step 1, where N,2-dimethyl-4-[6-(trifluoromethyl)quinazolin-2-yl]aniline was substituted in place of 2-methyl-4-[6-(trifluoromethyl)quinazolin-2-yl]aniline. Yield=46%.

### Example 153: N-(4-(4-amino-8-methyl-6-(trifluoromethyl)pyrido[3,2-d]pyrimidin-2-yl)-2-methylphenyl)-1-methyl-1H-pyrazole-5-carboxamide

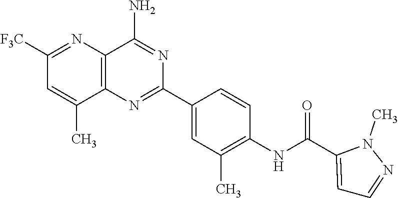 [SMILES: Cc1cc(-c2nc(N)c3nc(C(F)(F)F)cc(C)c3n2)ccc1NC(=O)c1ccnn1C]

A mixture of 2-chloro-8-methyl-6-(trifluoromethyl)pyrido[3,2-d]pyrimidin-4-amine (40 mg, 0.15 mmol, 1 equiv), 1-methyl-N-[2-methyl-4-(tetramethyl-1,3,2-dioxaborolan-2-yl)phenyl]-1H-pyrazole-5-carboxamide (52.0 mg, 0.15 mmol, 1 equiv), K₂CO₃ (42.1 mg, 0.30 mmol, 2 equiv) and Pd(PPh₃)₄(17.6 mg, 0.02 mmol, 0.1 equiv) in dioxane (1 mL) and H₂O (0.2 mL) was stirred at 100° C. overnight. The resulting solution was diluted with 10 mL of water, extracted with 2×10 ml of ethyl acetate, the combined organic layers concentrated and the resulting residue purified by preparative TLC with dichloromethane/methanol (20/1) as eluent to afford the desired final product as a white solid in 58% yield. LCMS (ES, m/z): [M+H]⁺ 442; ¹H-NMR (400 MHz, DMSO-d₆, ppm): δ2.36 (s, 3H), δ2.80 (s, 3H), δ4.11 (s, 3H), δ7.11-7.12 (d, 1H), δ7.53-7.56 (m, 2H), δ7.99 (s, 1H), δ8.14-8.15 (d, 1H), δ8.27 (s, 1H), δ8.36-8.39 (m, 1H), δ8.43 (s, 1H), δ9.97 (s, 1H)

### Step 1: 2-chloro-8-methyl-6-(trifluoromethyl)pyrido[3,2-d]pyrimidin-4-amine

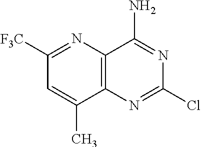 [SMILES: Cc1cc(C(F)(F)F)nc2c(N)nc(Cl)nc12]

A solution of 2,4-dichloro-8-methyl-6-(trifluoromethyl)pyrido[3,2-d]pyrimidine (200 mg, 1 equiv) in NH₃/MeCN (2 mL) was stirred for 30 min at room temperature. The resulting mixture was concentrated and the residue purified by preparative TLC with ethyl acetate/petroleum ether (1:2) as eluent to afford the desired final product as a white solid in 59% yield.

### Example 154: N-(4-(4-amino-8-methyl-6-(trifluoromethyl)pyrido[3,2-d]pyrimidin-2-yl)-2-methylphenyl)-N,1-dimethyl-1H-pyrazole-5-carboxamide

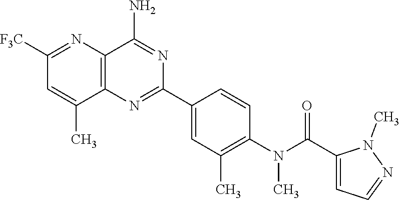 [SMILES: Cc1cc(-c2nc(N)c3nc(C(F)(F)F)cc(C)c3n2)ccc1N(C)C(=O)c1ccnn1C]

The title compound was prepared analogously to Example 153, where 1-methyl-N-[2-methyl-4-(tetramethyl-1,3,2-dioxaborolan-2-yl)phenyl]-1H-pyrazole-5-carboxamide was substituted in place of 1-methyl-N-[2-methyl-4-(tetramethyl-1,3,2-dioxaborolan-2-yl)phenyl]-1H-pyrazole-5-carboxamide. Yield=58%. LCMS (ES, m/z): [M+H]⁺ 456; ¹H-NMR (400 MHz, CD₃OD, ppm): δ2.29 (s, 3H), δ2.84 (s, 3H), δ3.44 (s, 3H), δ4.10 (s, 3H), δ5.61-5.62 (d, 1H), δ7.15-7.16 (d, 2H), δ7.34-7.37 (d, 1H), δ7.99 (s, 1H), δ8.42-8.47 (m, 2H)

### Example 155: 1-(Cyanomethyl)-N-(2-methyl-4-(6-(trifluoromethyl)quinazolin-2-yl)phenyl)-1H-pyrazole-5-carboxamide

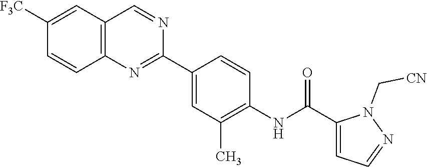 [SMILES: Cc1cc(-c2ncc3cc(C(F)(F)F)ccc3n2)ccc1NC(=O)c1ccnn1CC#N]

The title compound was prepared analogously to Example 116, where 1-(cyanomethyl)-1H-pyrazole-5-carbonyl chloride was substituted in place of isonicotinoyl chloride hydrochloride and 2-methyl-4-(6-(trifluoromethyl)quinazolin-2-yl)aniline was substituted in place of N,2-dimethyl-4-[6-(trifluoromethyl)quinazolin-2-yl]aniline. Yield=30%. LCMS (ES, m/z): [M+H]⁺ 451; ¹H-NMR (400 MHz, DMSO-d₆, ppm): δ2.31 (s, 3H), δ3.36 (s, 3H), δ5.48-5.49 (d, 1H), δ5.63-5.75 (m, 2H), δ7.35-7.36 (d, 1H), δ7.53-7.55 (d, 1H), δ8.25-8.32 (m, 2H), δ8.44-8.46 (d, 1H), δ8.54 (s, 1H), δ8.74 (s, 1H), δ9.91 (s, 1H).

### Step 1: Methyl 1-(cyanomethyl)-1H-pyrazole-5-carboxylate

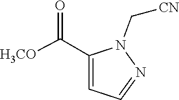 [SMILES: COC(=O)c1ccnn1CC#N]

A mixture of methyl 1H-pyrazole-5-carboxylate (10 g, 79.29 mmol, 1.00 equiv), 2-bromoacetonitrile (9.43 g, 78.62 mmol, 1.10 equiv) and potassium carbonate (10.84 g, 78.43 mmol, 1.10 equiv) in acetonitrile (100 mL) was stirred for 6 h at 80° C. The solids were filtered out and the solution concentrated under vacuum. The residue was purified by silica gel chromatography with ethyl acetate/petroleum ether (1:20) to afford the desired final product as a white solid in 42% yield.

### Step 2: 1-(Cyanomethyl)-1H-pyrazole-5-carboxylic acid

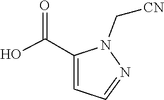 [SMILES: N#CCn1nccc1C(=O)O]

LiGH (704 mg, 29.39 mmol, 1.00 equiv) in water (7 mL) was added over a solution of ethyl 1-(cyanomethyl)-1H-pyrazole-5-carboxylate (3 g, 16.74 mmol, 1.00 equiv) in THE (7 mL). The mixture was stirred at room temperature overnight. Addition of 30 mL of H₂O was followed by neutralization with 1 M HCl. The aqueous solution was extracted three times with ethyl acetate and the combined organic layers concentrated. The crude product was purified by Flash-Prep-HPLC with the following conditions (IntelFlash-1): Column=C18 silica gel; mobile phase=acetonitrile:water=0:100 increasing to acetonitrile:water=30:70 within 30 min; Detector=UV 220 nm. The final product was isolated as a white solid in 22% yield.

### Step 3: 1-(Cyanomethyl)-1H-pyrazole-5-carbonyl chloride

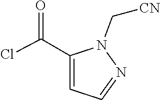 [SMILES: N#CCn1nccc1C(=O)Cl]

1-(cyanomethyl)-1H-pyrazole-5-carboxylic acid (100 mg, 0.66 mmol, 1 equiv) was dissolved in CH₂Cl₂ (2 mL) followed by addition of DMF (4.8 mg, 0.07 mmol, 0.1 equiv) and oxalyl chloride (92.4 mg, 0.73 mmol, 1.1 equiv). The resulting solution was stirred for 2 hours at room temperature. Evaporation of volatiles afforded the desired acid chloride as a solid, which was used in the next step without further purification.

### Example 156: 1-(2-amino-2-oxoethyl)-N-methyl-N-(2-methyl-4-(6-(trifluoromethyl)quinazolin-2-yl)phenyl)-1H-pyrazole-5-carboxamide

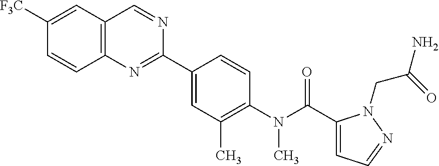 [SMILES: Cc1cc(-c2ncc3cc(C(F)(F)F)ccc3n2)ccc1N(C)C(=O)c1ccnn1CC(N)=O]

A mixture of 1-(cyanomethyl)-N-methyl-N-[2-methyl-4-[6-(trifluoromethyl)quinazolin-2-yl]phenyl]-1H-pyrazole-5-carboxamide (100 mg, 0.22 mmol, 1 equiv) and conc. H₂SO₄ (0.5 mL) was stirred at room temperature for 1 hour. The resulting solution was diluted with 20 mL of ice/water, extracted with (2×10 mL) of ethyl acetate and the combined organic layers concentrated. The residue was purified by preparative TLC with dichloromethane/methanol (20:1) as eluent to afford the desired final product as a white solid in 58% yield. LCMS (ES, m/z): [M+H]⁺ 469; ¹H-NMR: (300 MHz, DMSO-d₆, ppm): δ2.39 (s, 3H), δ3.28 (s, 3H), δ5.13-5.14 (d, 2H), δ5.41-5.42 (d, 1H), δ7.17-7.21 (m, 2H), δ7.55-7.58 (d, 2H), δ8.27-8.29 (m, 2H), δ8.37-8.39 (d, 1H), δ8.55 (s, 1H), δ8.74 (s, 1H), δ9.91 (s, 1H)

### Example 157: N-methyl-N-(2-methyl-4-(6-(trifluoromethyl)quinazolin-2-yl)phenyl)-1-(2-morpholinoethyl)-1H-pyrazole-5-carboxamide

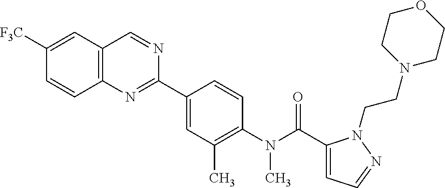 [SMILES: Cc1cc(-c2ncc3cc(C(F)(F)F)ccc3n2)ccc1N(C)C(=O)c1ccnn1CCN1CCOCC1]

To a solution of N,2-dimethyl-4-[6-(trifluoromethyl)quinazolin-2-yl]aniline (150 mg, 0.47 mmol, 1 equiv) in CH₂Cl₂ (3 mL) was added 1-(2-morpholinoethyl)-1H-pyrazole-5-carbonyl chloride (230.4 mg, 0.95 mmol, 2 equiv) and triethylamine (143.5 mg, 1.42 mmol, 3 equiv). The resulting mixture was stirred for 3 h at room temperature and quenched by the addition of MeOH (0.5 mL). The resulting mixture was concentrated under reduced pressure and the residue purified by preparative TLC (CH₂Cl₂/MeOH=30:1) to afford the desired final product as a yellow solid in 24% yield. LCMS: (ES, m/z): [M+H]⁺ 525; ¹H-NMR (400 MHz, DMSO-d₆, ppm): δ2.35 (s, 3H), 52.45-2.50 (m, 2H), 52.50-2.52 (m, 2H), 52.70-2.76 (m, 2H), δ3.32 (s, 3H), 53.55-3.65 (m, 4H), 54.48-4.50 (m, 1H), 4.64-4.66 (m, 1H), 5.46-5.47 (d, 1H), 7.17 (s, 1H), 7.51-7.53 (d, 1H), 8.24-8.31 (m, 2H), 8.37-8.39 (d, 1H), 8.52 (s, 1H), 8.73 (s, 1H), 9.90 (s, 1H)

### Step 1: Methyl 1-(2-morpholinoethyl)-1H-pyrazole-5-carboxylate

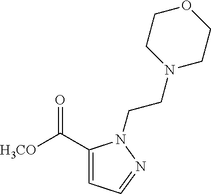 [SMILES: COC(=O)c1ccnn1CCN1CCOCC1]

To a solution of methyl 1H-pyrazole-5-carboxylate (2 g, 15.86 mmol, 1 equiv) and 2-(morpholin-4-yl)ethan-1-ol (2.5 g, 19.03 mmol, 1.2 equiv) in THE (20 mL), was added DIAD (4.8 g, 23.79 mmol, 1.5 equiv) and PPh₃ (8.3 g, 31.72 mmol, 2 equiv). The reaction was stirred at room temperature overnight. The resulting mixture was concentrated under reduced pressure and the residue purified by silica gel column chromatography with petroleum ether/EtOAc (3:1) to afford a crude product. This crude product was purified by flash chromatography with the following conditions (CH₃CN: NH₄HCO₃ (aq)=0%-20%, 20 min) affording the pure final product as a white solid in 40% yield.

### Step 2: 1-(2-Morpholinoethyl)-1H-pyrazole-5-carboxylic acid

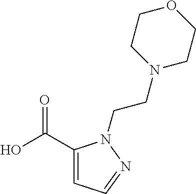 [SMILES: O=C(O)c1ccnn1CCN1CCOCC1]

To a stirred solution of methyl 1-(2-morpholinoethyl)-1H-pyrazole-5-carboxylate (880 mg, 3.68 mmol, 1 equiv) in THF (20 mL) was added LiOH.H₂O (186 mg, 4.42 mmol, 1.2 equiv) dissolved in 10 mL of H₂O. The resulting mixture was stirred for 2 h at room temperature and acidified to pH 6 with 1M HCl. The mixture was concentrated under reduced pressure to afford the desired final product as a white solid in 99% yield.

### Step 3: 1-(2-Morpholinoethyl)-1H-pyrazole-5-carbonyl chloride

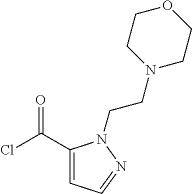 [SMILES: O=C(Cl)c1ccnn1CCN1CCOCC1]

To a stirred solution of 1-(2-morpholinoethyl)-1H-pyrazole-5-carboxylic acid and DMF (16.2 mg, 0.22 mmol, 0.1 equiv) in CH₂Cl₂ (10 mL), oxalyl chloride (422.6 mg, 3.33 mmol, 1.5 equiv) was added. The resulting mixture was stirred at room temperature for 5 hours and concentrated under reduced pressure to afford the acid chloride as a white solid. This material was immediately used in the next step without further purification.

### Example 158: 1-((1H-tetrazol-5-yl)methyl)-N-methyl-N-(2-methyl-4-(6-(trifluoromethyl)-quinazolin-2-yl)phenyl)-1H-pyrazole-5-carboxamide

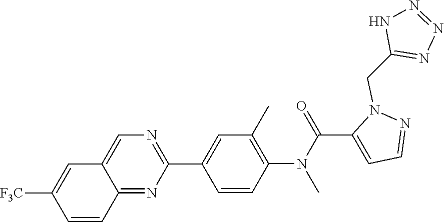 [SMILES: Cc1cc(-c2ncc3cc(C(F)(F)F)ccc3n2)ccc1N(C)C(=O)c1ccnn1Cc1nnn[nH]1]

Into an 8-mL vial, was placed 1-(cyanomethyl)-N-methyl-N-(2-methyl-4-(6-(trifluoro-methyl)-quinazolin-2-yl)phenyl)-1H-pyrazole-5-carboxamide (100 mg, 0.22 mmol, 1 equiv.), DMF (2 mL), NaN₃ (43.3 mg, 0.67 mmol, 3 equiv.), NH₄Cl (47.5 mg, 0.89 mmol, 4 equiv.) and the resulting solution was stirred for 4 h at 100° C. The crude product was purified by Flash-Prep-HPLC (Column, C18; mobile phase, MeCN/H₂O=0:100 increasing to 70:30 within 25 mins; Detector, UV 254 nm) to give the title compound as a white solid in 25% yield. LC-MS: (ES, m/z): [M+H]⁺ 494; ¹H-NMR: (400 MHz, DMSO, ppm): δ 2.30 (s, 3H), 3.34 (s, 3H), 5.50-5.51 (d, 1H), 5.86-5.99 (m, 2H), 7.20-7.21 (d, 1H), 7.42-7.44 (d, 1H), 8.24-8.32 (m, 3H), 8.50 (s, 1H), 8.73 (s, 1H), 9.89 (s, 1H)

### Example 159: N,1-dimethyl-N-(2-methyl-4-(8-methyl-6-(trifluoromethoxy)pyrido[3,2-d]-pyrimidin-2-yl)phenyl)-1H-pyrazole-5-carboxamide

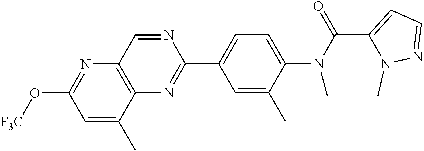 [SMILES: Cc1cc(-c2ncc3nc(OC(F)(F)F)cc(C)c3n2)ccc1N(C)C(=O)c1ccnn1C]

To a stirred mixture of 2-chloro-8-methyl-6-(trifluoromethoxy)pyrido[3,2-d]pyrimidine (100 mg, 0.38 mmol, 1 equiv.) and N-[2-fluoro-4-(tetramethyl-1,3,2-dioxaborolan-2-yl)phenyl]-N,1-dimethyl-1H-pyrazole-5-carboxamide (136.3 mg, 0.38 mmol, 1 equiv.) in t-BuOH (2 mL) was added K₂CO₃ (104.9 mg, 0.76 mmol, 2 equiv.) and Xantphos G3 Pd (32 mg, 0.038 mmol, 0.1 equiv.) at room temperature under nitrogen atmosphere. The resulting mixture was stirred overnight at 70° C. under nitrogen atmosphere and then concentrated under vacuum. The residue was purified by Prep-TLC (CH₂Cl₂/MeOH=20:1) to afford the title compound as a white solid in 42.60% yield. LC-MS: (ES, m/z): [M+H]⁺ 457; ¹H-NMR: (400 MHz, DMSO, ppm): δ 2.27 (s, 1H), 2.84 (s, 3H), 3.31 (s, 3H), 4.11 (s, 3H), 5.54 (s, 1H), 7.14 (s, 1H), 7.48-7.50 (d, 1H), 7.81 (s, 1H), 8.39-8.41 (d, 1H), 8.47 (s, 1H), 9.61 (s, 1H)

### Example 160: 2-methyl-N-(2-methyl-4-(8-methyl-6-(trifluoromethoxy)pyrido[3,2-d]-pyrimidin-2-yl)phenyl)isonicotinamide

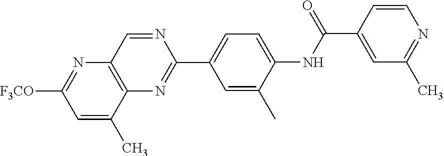 [SMILES: Cc1cc(C(=O)Nc2ccc(-c3ncc4nc(OC(F)(F)F)cc(C)c4n3)cc2C)ccn1]

To a stirred solution of 2-chloro-8-methyl-6-(trifluoromethoxy)pyrido[3,2-d]pyrimidine (100 mg, 0.38 mmol, 1 equiv.) and 2-methyl-N-[2-methyl-4-(tetramethyl-1,3,2-dioxaborolan-2-yl)phenyl]pyridine-4-carboxamide (133.6 mg, 0.38 mmol, 1 equiv.) in t-BuOH (2 mL) were added XPhos Pd G3 (32.1 mg, 0.04 mmol, 0.1 equiv.) and K₂CO₃ (104.9 mg, 0.76 mmol, 2 equiv.) at room temperature under nitrogen atmosphere. The resulting mixture was stirred overnight at 70° C. under nitrogen atmosphere. The resulting mixture was concentrated under vacuum and the residue was purified by Prep-TLC (CH₂Cl₂/MeOH=20:1) to the title compound as a white solid in 11.10% yield. LC-MS: (ES, m/z): [M+H]⁺ 454; ¹H-NMR: (400 MHz, DMSO, ppm): δ2.40 (s, 3H), 2.60 (s, 3H), δ2.86-2.87 (d, 3H), 7.64-7.66 (d, 1H), 7.70-7.71 (d, 1H), 7.79-7.81 (d, 2H), 8.44-8.45 (d, 1H), 8.51 (s, 1H), 8.66-8.67 (d, 1H), 9.62 (s, 1H), 10.23 (s, 1H)

### Step 1: 4-methyl-5-nitro-2-(trifluoromethoxy)pyridine

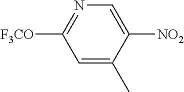

To a stirred solution of 4-methyl-5-nitropyridin-2-ol (48.7 g, 316 mmol, 2 equiv.) in CH₃NO₂ (500 mL) was added 1-trifluoromethyl-1, 2-benziodoxol-3-(1H)-one (50 g, 158 mmol, 1 equiv.) in portions at room temperature under nitrogen atmosphere. The resulting mixture was stirred for 12 h at 100° C. under nitrogen atmosphere. The mixture was allowed to cool down to room temperature, filtered, and the filter cake was washed with DCM. The filtrate was concentrated under reduced pressure and the residue was purified by silica gel column chromatography by eluting with PE/EA (10:1) to afford the title compound as a white solid in 38.4% yield.

### Step 2: 4-methyl-6-(trifluoromethoxy)pyridin-3-amine

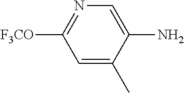

To a stirred suspension of 4-methyl-5-nitro-2-(trifluoromethoxy)pyridine (13.5 g, 60.79 mmol, 1 equiv.) and NH₄Cl (19.6 g, 364.76 mmol, 6 equiv.) in H₂O (135 mL) was added Fe (10.02 g, 182.38 mmol, 3 equiv.) at room temperature. The resulting mixture was stirred for 2 h at 100° C. under nitrogen atmosphere and then allowed to cool down to room temperature. The resulting mixture was filtered and the filter cake was washed with EA. The filtrate was extracted with EA and the organic layer was concentrated under reduced pressure. The residue was purified by silica gel column chromatography by eluting with PE/EA (5:1) to afford the title compound as a white solid in 78.83% yield.

### Step 3: 2-bromo-4-methyl-6-(trifluoromethoxy)pyridin-3-amine

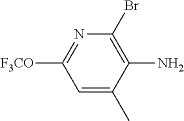

To a stirred solution of 4-methyl-6-(trifluoromethoxy)pyridin-3-amine (9.2 g, 47.88 mmol, 1 equiv.) in DCM (100 mL) was added NBS (8.36 g, 47.88 mmol, 1 equiv.) in portions at 0° C. under nitrogen atmosphere. The resulting mixture was stirred for 1 h at 0° C. under nitrogen atmosphere and then quenched with sat. NH₄Cl (aq.) at 0° C. The resulting mixture was extracted with ethyl acetate, dried over anhydrous Na₂SO₄. After filtration, the filtrate was concentrated under reduced pressure to afford the title compound as a yellow solid in 92.45% yield.

### Step 4: 3-amino-4-methyl-6-(trifluoromethoxy)pyridine-2-carbonitrile

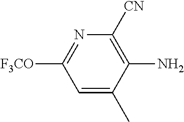

To a stirred solution of 2-bromo-4-methyl-6-(trifluoromethoxy)pyridin-3-amine (12 g, 44.25 mmol, 1 equiv.) and Zn(CN)₂ (10.24 g, 88.49 mmol, 1.97 equiv.) in DMF (120 mL) were added Pd(PPh₃)₄(5.12 g, 4.44 mmol, 0.10 equiv.). The final reaction mixture was irradiated with microwave radiation for 2 h at 200° C. and then quenched with water. The resulting mixture was extracted with EA and the combined organic layer was concentrated under reduced pressure. The residue was purified by silica gel column chromatography by eluting with PE/EA (5:1) to afford the title compound as a white solid in 36.72% yield.

### Step 5: 8-methyl-6-(trifluoromethoxy)pyrido[3,2-d]pyrimidine-2,4(1H,3H)-dione

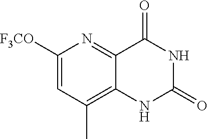 [SMILES: Cc1cc(OC(F)(F)F)nc2c(=O)[nH]c(=O)[nH]c12]

To a stirred solution of 3-amino-4-methyl-6-(trifluoromethoxy)pyridine-2-carbonitrile (3 g, 13.8 mmol, 1 equiv.) in DMF (30 mL) was added DBU (6.29 g, 41.4 mmol, 3 equiv.). The resulting mixture was stirred overnight at 100° C. under CO₂ atmosphere. The reaction was quenched with water and the resulting mixture was extracted with EA. The combined organic layer was concentrated under reduced pressure and the residue was purified by silica gel column chromatography with PE/EA (5:1) as the eluent to afford the title compound as a white solid in 86.11% yield.

### Step 6: 2,4-dichloro-8-methyl-6-(trifluoromethoxy)pyrido[3,2-d]pyrimidine

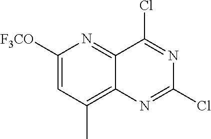 [SMILES: Cc1cc(OC(F)(F)F)nc2c(Cl)nc(Cl)nc12]

To a stirred solution of 8-methyl-6-(trifluoromethoxy)pyrido[3,2-d]pyrimidine-2,4(1H,3H)-dione (3.1 g, 11.88 mmol, 1 equiv.) in POCl₃ (30 mL) was added PCl₅ (12.35 g, 59.39 mmol, 5 equiv.) in portions at room temperature under nitrogen atmosphere. The reaction mixture was then heated to reflux for 120 min under nitrogen atmosphere. The reaction was quenched with water/ice at 0° C. and the resulting mixture was extracted with EA and then concentrated under reduced pressure. The residue was purified by silica gel column chromatography, eluted with petroleum ether/ethyl acetate (5:1) to afford the title compound as a white solid in 34.30% yield.

### Step 7: 2-chloro-8-methyl-6-(trifluoromethoxy)pyrido[3,2-d]pyrimidine

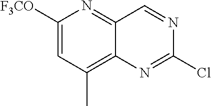 [SMILES: Cc1cc(OC(F)(F)F)nc2cnc(Cl)nc12]

To a stirred solution of 2,4-dichloro-8-methyl-6-(trifluoromethoxy)pyrido[3,2-d]-pyrimidine (1.37 g, 4.60 mmol, 1 equiv.) and Pd(PPh₃)₄(0.5 g, 0.46 mmol, 0.1 equiv.) in THF (20 mL) was added PPh₃ (1.8 g, 6.89 mmol, 1.5 equiv.) under nitrogen atmosphere. To the above mixture was added SnBu₃H (2.8 g, 4.83 mmol, 1.05 equiv.) dropwise over 15 min at 0° C. The resulting mixture was stirred for addition 2 h at 0° C. and then quenched with water/ice (20 mL) at 0° C. The aqueous layer was extracted with EA and the combined organic layer was concentrated under vacuum. The residue was purified by silica gel column chromatography by eluting with PE/EA (5:1) to afford the title compound as a white solid in 55.30% yield.

### Step 8: N-(4-bromo-2-methylphenyl)-2-methylpyridine-4-carboxamide

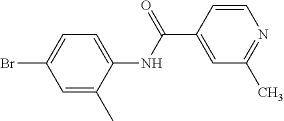 [SMILES: Cc1cc(C(=O)Nc2ccc(Br)cc2C)ccn1]

To a stirred mixture of 2-methylpyridine-4-carboxylic acid (1.47 g, 10.72 mmol, 1 equiv.) and 4-bromo-2-methylaniline (2.0 g, 10.72 mmol, 1 equiv.) in DMF (50 mL) were added HATU (6.1 g, 16.08 mmol, 1.50 equiv.) and DIEA (2.8 g, 21.66 mmol, 2.02 equiv.). The resulting mixture was stirred overnight at room temperature and then quenched with water and extracted with EA. The combined organic layer was concentrated under reduced pressure and the residue was purified by silica gel column chromatography, eluted with CH₂Cl₂/MeOH (100:1) to afford the title compound as a white solid in 91.71% yield.

### Step 9: 2-methyl-N-[2-methyl-4-(tetramethyl-1,3,2-dioxaborolan-2-yl)phenyl]pyridine-4-carboxamide

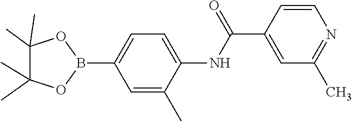

To a stirred solution of N-(4-bromo-2-methylphenyl)-2-methylpyridine-4-carboxamide (1.5 g, 4.92 mmol, 1 equiv.) and Pd(dppf)Cl₂ (0.4 g, 0.54 mmol, 0.11 equiv.) in dioxane (20 mL) were added KOAc (964.8 mg, 9.83 mmol, 2 equiv.) and B₂Pin₂ (0.528 g, 5.41 mmol, 1.1 equiv.) under N₂ atmosphere. The resulting mixture was stirred for 4 h at 80° C. under N₂ atmosphere. The resulting mixture was concentrated under reduced pressure and the residue was purified by silica gel column chromatography, eluted with CH₂Cl₂/MeOH (100:1) to afford the title compound as a white solid in 11.55% yield.

### Example 161: N,2-dimethyl-N-(2-methyl-4-(8-methyl-6-(trifluoromethoxy)pyrido[3,2-d]pyrimidin-2-yl)phenyl)isonicotinamide

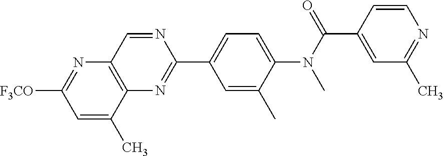 [SMILES: Cc1cc(C(=O)N(C)c2ccc(-c3ncc4nc(OC(F)(F)F)cc(C)c4n3)cc2C)ccn1]

The title compound was prepared analogously to Example 160 by substituting 2-methyl-N-(2-methyl-4-(4,4,5-trimethyl-1,3,2-dioxaborolan-2-yl)phenyl)isonicotinamide with N,2-dimethyl-N-[2-methyl-4-(tetramethyl-1,3,2-dioxaborolan-2-yl)phenyl]pyridine-4-carboxamide as a white solid. LC-MS: (ES, m/z): [M+H]⁺ 468; ¹H-NMR: (300 MHz, DMSO, ppm): δ 2.35 (s, 6H), 2.81 (s, 3H), 3.37 (s, 3H), 6.97-7.01 (d, 1H), 7.21 (s, 1H), 7.44-7.47 (d, 1H), 7.77 (s, 1H), 8.25-8.27 (m, 2H), 8.39 (s, 1H), 9.57 (s, 1H)

### Step 1: N-(4-bromo-2-methylphenyl)-N,2-dimethylpyridine-4-carboxamide

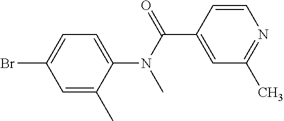 [SMILES: Cc1cc(C(=O)N(C)c2ccc(Br)cc2C)ccn1]

To a stirred solution of N-(4-bromo-2-methylphenyl)-2-methylpyridine-4-carboxamide (1 g, 3.28 mmol, 1 equiv.) in DMF (10 mL) was added NaH (0.0787 g, 3.28 mmol, 1 equiv.) and the resulting mixture was stirred for 30 min. Mel (0.5 g, 3.52 mmol, 1.08 equiv.) was added dropwise at 0° C. and the resulting mixture was stirred for additional 1 h at 0° C. The reaction mixture was quenched with NH₄Cl (aq.) and then extracted with EA and dried over anhydrous Na₂SO₄. After filtration, the filtrate was concentrated under reduced pressure to give the title compound as a white solid in (920 mg, 87.96%).

### Step 2: N,2-dimethyl-N-[2-methyl-4-(tetramethyl-1,3,2-dioxaborolan-2-yl)phenyl]pyridine-4-carboxamide

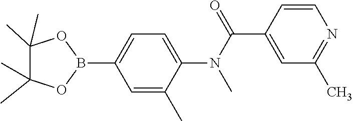

To a stirred solution of N-(4-bromo-2-methylphenyl)-N,2-dimethylpyridine-4-carboxamide (920 mg, 2.88 mmol, 1 equiv.) and B₂Pin₂ (309.4 mg, 3.17 mmol, 1.10 equiv.) in 1,4-dioxane (10 mL) were added KOAc (565.7 mg, 5.76 mmol, 2 equiv.) and Pd(dppf)Cl₂ (210.9 mg, 0.29 mmol, 0.1 equiv.) in portions at room temperature under nitrogen atmosphere. The resulting mixture was stirred for overnight at 80° C. under nitrogen atmosphere and then concentrated under reduced pressure. The residue was purified by silica gel column chromatography by eluting with PE/EA (10:1) to afford the title compound as a white solid in 76.73% yield.

### Example 162: N-(2-fluoro-4-(8-methyl-6-(trifluoromethyl)pyrido[3,2-d]pyrimidin-2-yl)-phenyl)-N,1-dimethyl-1H-pyrazole-5-carboxamide

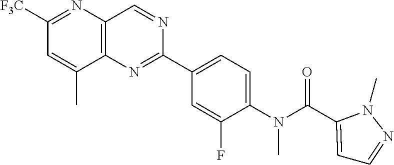 [SMILES: Cc1cc(C(F)(F)F)nc2cnc(-c3ccc(N(C)C(=O)c4ccnn4C)c(F)c3)nc12]

Into a 8-mL sealed tube purged and maintained with an inert atmosphere of nitrogen, was placed 2-chloro-8-methyl-6-(trifluoromethyl)pyrido[3,2-d]pyrimidine (100 mg, 0.40 mmol, 1 equiv.), N-(2-fluoro-4-(4,4,5,5-tetramethyl-1,3,2-dioxaborolan-2-yl)phenyl)-N,1-dimethyl-1H-pyrazole-5-carboxamide (174.1 mg, 0.48 mmol, 1.2 equiv.), toluene (2 mL), EtOH (1 mL), K₂CO₃ (167.5 mg, 1.21 mmol, 3 equiv.), and Pd(PPh₃)₄(93.3 mg, 0.08 mmol, 0.2 equiv.). The resulting solution was stirred overnight at 80° C. and then quenched with NH₄Cl (aq.). The resulting solution was extracted with ethyl acetate and the organic layers were combined, and concentrated. The residue was purified by Flash-Prep-HPLC ((IntelFlash-1): Column, C18; mobile phase, MeCN:H₂O (30:70) increasing to MeCN: H₂O (70:30) within 30 min; Detector, 254 nm) to give the title compound as a white solid in 41.18% yield. LC-MS: (ES, m/z): [M+H]⁺ 445; ¹H-NMR: (300 MHz, DMSO, ppm): δ2.92 (s, 3H), 3.42 (s, 3H), 3.98 (s, 3H), 5.84 (s, 1H), 7.22-7.23 (d, 1H), 7.77-7.82 (t, 1H), 8.31-8.35 (m, 1H), 8.41 (d, 1H), 8.46-8.49 (m, 1H), 9.90 (s, 1H)

### Step 1: N-(2-fluoro-4-(4,4,5,5-tetramethyl-1,3,2-dioxaborolan-2-yl)phenyl)-N,1-dimethyl-1H-pyrazole-5-carboxamide

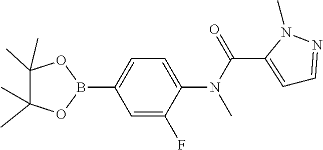 [SMILES: CN(C(=O)c1ccnn1C)c1ccc(B2OC(C)(C)C(C)(C)O2)cc1F]

Into a 50-mL 3-necked round-bottom flask purged and maintained with an inert atmosphere of nitrogen, was placed N-[2-fluoro-4-(tetramethyl-1,3,2-dioxaborolan-2-yl)phenyl]-1-methyl-1H-pyrazole-5-carboxamide (2 g, 5.79 mmol, 1 equiv.) in THF (20 mL). LiHMDS (11.59 mL, 1M, 2 equiv.) was added at 0° C., followed by addition of Mel (904.7 mg, 6.37 mmol, 1.100 equiv.) after 30 min. The resulting solution was stirred for 2 h at room temperature and then quenched with NH₄Cl (aq.). The resulting solution was extracted with ethyl acetate and the combined organic layer was concentrated under reduced pressure. The residue was applied onto a silica gel column and eluted with ethyl acetate/petroleum ether (1/10) to give the title compound as a pale-yellow solid in 20.47% yield.

### Example 163: N-[2-fluoro-4-[8-methyl-6-(trifluoromethyl)pyrido[3,2-d]pyrimidin-2-yl]phenyl]-1-methyl-1H-pyrazole-5-carboxamide

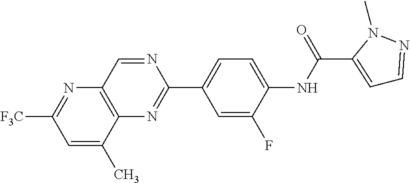 [SMILES: Cc1cc(C(F)(F)F)nc2cnc(-c3ccc(NC(=O)c4ccnn4C)c(F)c3)nc12]

To a stirred solution of 2-chloro-8-methyl-6-(trifluoromethyl)pyrido[3,2-d]pyrimidine (100 mg, 0.40 mmol, 1 equiv.) and N-[2-fluoro-4-(tetramethyl-1,3,2-dioxaborolan-2-yl)phenyl]-1-methyl-1H-pyrazole-5-carboxamide (139.4 mg, 0.40 mmol, 1 equiv.) in toluene (0.8 mL) and EtOH (0.4 mL) was added Pd(PPh₃)₄(46.7 mg, 0.04 mmol, 0.1 equiv.) and K₂CO₃ (111.6 mg, 0.81 mmol, 2 equiv.) at room temperature under nitrogen atmosphere. The resulting mixture was stirred overnight at 100° C. under nitrogen atmosphere. The mixture cooled to room temperature and then concentrated under reduced pressure. The residue was purified by Prep-TLC (CH₂Cl₂/MeOH=20:1) to afford the title compound as a yellow solid in 28% yield. LC-MS: (ES, m/z): [M+H]⁺ 431; ¹H-NMR: (400 MHz, DMSO, ppm): δ 2.94 (s, 3H), 4.11 (s, 3H), 7.16 (d, 1H), 7.57 (d, 1H), 7.91-7.95 (t, 1H), 8.39-8.45 (m, 2H), 8.49-8.51 (m, 1H), 9.89 (s, 1H), 10.33 (s, 1H)

### Example 164: N-(2-fluoro-4-(8-methyl-6-(trifluoromethyl)pyrido[3,2-d]pyrimidin-2-yl)phenyl)-2-methylisonicotinamide

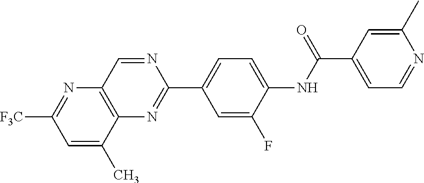 [SMILES: Cc1cc(C(=O)Nc2ccc(-c3ncc4nc(C(F)(F)F)cc(C)c4n3)cc2F)ccn1]

The title compound was prepared analogously to Example 163 by substituting N-[2-fluoro-4-(tetramethyl-1,3,2-dioxaborolan-2-yl)phenyl]-1-methyl-1H-pyrazole-5-carboxamide with N-(2-fluoro-4-(4,4,5,5-tetramethyl-1,3,2-dioxaborolan-2-yl)phenyl)-2-methyl-isonicotinamide to give crude product which was purified by Flash-Prep-HPLC ((IntelFlash-1): Column, C18 silica gel; mobile phase, MeCN:H₂O (40:60) increasing to MeCN:H₂O (60:40) within 50 min; Detector, 254 nm) to give a white solid in 18.10%) yield. LC-MS: (ES, m/z): [M+H]⁺ 442; ¹H-NMR: (300 MHz, CDCl₃, ppm): δ 2.71 (s, 3H), 2.97 (s, 3H), 7.56-7.58 (d, 1H), 7.61-7.65 (d, 1H), 7.96 (s, 1H), 8.26-8.27 (d, 1H), 8.47-8.52 (m, 1H), 8.56-8.59 (d, 1H), 8.67-8.72 (m, 2H), 9.74 (s, 1H)

### Step 1: N-(2-fluoro-4-(4,4,5,5-tetramethyl-1,3,2-dioxaborolan-2-yl)phenyl)-2-methylisonicotinamide

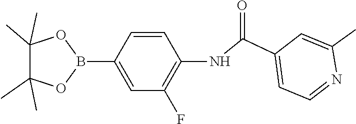

The title compound was prepared analogously to Example 160, Step 8 by substituting 4-bromo-2-methylaniline with 2-fluoro-4-(4,4,5,5-tetramethyl-1,3,2-dioxaborolan-2-yl)aniline to provide a crude product which was purified by silica gel column with dichloromethane/methanol (80/1) as eluent to give the title compound as a yellow solid in 50.60% yield.

### Example 165: N-(2-fluoro-4-(8-methyl-6-(trifluoromethyl)pyrido[3,2-d]pyrimidin-2-yl)phenyl)-2-methylisonicotinamide

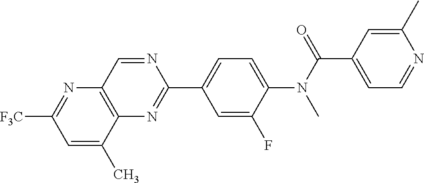 [SMILES: Cc1cc(C(=O)N(C)c2ccc(-c3ncc4nc(C(F)(F)F)cc(C)c4n3)cc2F)ccn1]

Into a 8-mL sealed tube purged and maintained with an inert atmosphere of nitrogen, was placed 2-chloro-8-methyl-6-(trifluoromethyl)pyrido[3,2-d]pyrimidine (132 mg, 0.53 mmol, 1 equiv.), N,2-dimethyl-N-[2-fluoro-4-(tetramethyl-1,3,2-dioxaborolan-2-yl)phenyl]pyridine-4-carboxamide (217.1 mg, 0.59 mmol, 1.1 equiv.), toluene (2.64 mL), EtOH (1.32 mL), K₂CO₃ (221.0 mg, 1.60 mmol, 3 equiv.), Pd(PPh₃)₄(92.4 mg, 0.08 mmol, 0.15 equiv.). The resulting solution was stirred overnight at 85° C. The reaction was then quenched with NH₄Cl (aq.) and extracted with ethyl acetate. The residue was applied onto Prep-TLC with dichloromethane/methanol (50/1). The crude product was purified by Flash-Prep-HPLC with the following conditions (IntelFlash-1): Column, C18 silica gel; mobile phase, MeCN:H₂O=40:60 increasing to MeCN:H₂O=70:30 within 60 min; Detector, 254 nm to give the title compound in 32.46% yield as a yellow solid. LC-MS: (ES, m/z): [M+H]^{+ 1}H-NMR: (400 MHz, DMSO, ppm): δ 2.39 (s, 3H), 2.91 (s, 3H), 3.40 (s, 3H), 7.05 (s, 1H), 7.25 (s, 1H), 7.71-7.76 (t, 1H), 8.28-8.34 (m, 2H), 8.39-8.42 (m, 2H), 9.88 (s, 1H)

### Step 1: N-(4-bromo-2-fluorophenyl)-2-methylisonicotinamide

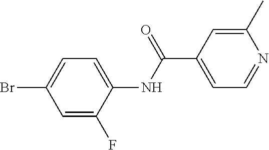 [SMILES: Cc1cc(C(=O)Nc2ccc(Br)cc2F)ccn1]

The title compound was prepared analogously to Example 160, Step 8 by substituting 4-bromo-2-fluoroaniline with 4-bromo-2-fluoroaniline to give crude product which was purified by re-crystallization from dichloromethane to give the title compound as a yellow solid in 59.90% yield.

### Step 2: N-(4-bromo-2-fluorophenyl)-N,2-dimethylisonicotinamide

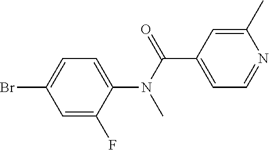 [SMILES: Cc1cc(C(=O)N(C)c2ccc(Br)cc2F)ccn1]

Into a 50-mL 3-necked round-bottom flask purged and maintained with an inert atmosphere of nitrogen, was placed N-(4-bromo-2-fluorophenyl)-2-methylisonicotinamide (1.08 g, 3.49 mmol, 1 equiv.) and DMF (11 mL). NaH (167.7 mg, 6.99 mmol, 2 equiv.) was added at 0° C., followed by addition of Mel (520.7 mg, 3.67 mmol, 1.05 equiv.) after 30 min. The resulting solution was stirred for 6 h at room temperature. The reaction was then quenched with NH₄Cl (aq.). The resulting solution was extracted with ethyl acetate and the combined organic layer was concentrated under reduced pressure. The residue was applied onto a silica gel column and eluted with ethyl acetate/petroleum ether (1/10) to give the title compound as a yellow solid in 40.74% yield.

### Step 3: N,2-dimethyl-N-[2-fluoro-4-(tetramethyl-1,3,2-dioxaborolan-2-yl)phenyl]pyridine-4-carboxamide

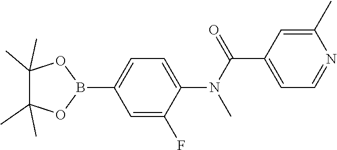

Proceeding as described in Example 161, Step 2 but substituting N-(4-bromo-2-methylphenyl)-N,2-dimethylpyridine-4-carboxamide with N-(4-bromo-2-fluorophenyl)-N,2-dimethylpyridine-4-carboxamide provided the title compound as a yellow solid in 41.27% yield.

### Example 166: N4-(2-methyl-4-(8-methyl-6-(trifluoromethyl)pyrido[3,2-d]pyrimidin-2-yl)phenyl)pyridine-2,4-dicarboxamide

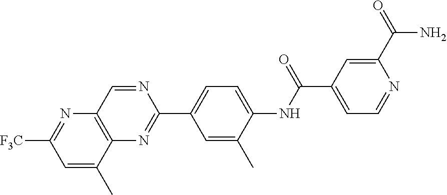 [SMILES: Cc1cc(-c2ncc3nc(C(F)(F)F)cc(C)c3n2)ccc1NC(=O)c1ccnc(C(N)=O)c1]

To a stirred solution of 2-cyano-N-[2-methyl-4-[8-methyl-6-(trifluoromethyl)pyrido[3,2-d]pyrimidin-2-yl]phenyl]pyridine-4-carboxamide (100 mg, 0.22 mmol, 1 equiv.) in MeOH (10 mL) were added NaOH (17.8 mg, 0.45 mmol, 2.00 equiv.) and H₂O₂ (0.5 mL). The resulting mixture was stirred for 5 h at room temperature and then diluted with water. The resulting mixture was extracted with ethyl acetate and the combined organic layer was concentrated under reduced pressure. The residue was purified by Prep-TLC (CH₂Cl₂/MeOH=10:1) to afford the title compound as a yellow solid in 23.27% yield. LC-MS: (ES, m/z): [M−H]⁻ 465; ¹H-NMR: (400 MHz, DMSO, ppm): δ 2.43 (s, 3H), 2.95 (s, 3H), 7.68-7.70 (d, 1H), 7.81 (s, 1H), 8.10-8.12 (d, 1H), 8.26 (s, 1H), 8.38-8.39 (d, 1H), 8.50-8.53 (m, 1H), 8.57-8.60 (d, 1H), 8.86-8.88 (d, 1H), 9.89 (s, 1H), 10.53 (s, 1H).

### Step 1: 2-cyanopyridine-4-carbonyl chloride

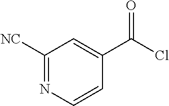 [SMILES: N#Cc1cc(C(=O)Cl)ccn1]

To a stirred solution of 2-cyanopyridine-4-carboxylic acid (250 mg, 1.69 mmol, 1 equiv.) in DCM (5 mL) were added DMF (12.3 mg, 0.17 mmol, 0.1 equiv.) and oxalyl chloride (431.9 mg, 3.38 mmol, 2.00 equiv.) at 0° C. The resulting mixture was stirred for 3 h at 0° C. and then concentrated under reduced pressure. The crude product was used in the next step directly without further purification.

### Step 2: 2-cyano-N-[2-methyl-4-[8-methyl-6-(trifluoromethyl)pyrido[3,2-d]pyrimidin-2-yl]-phenyl]pyridine-4-carboxamide

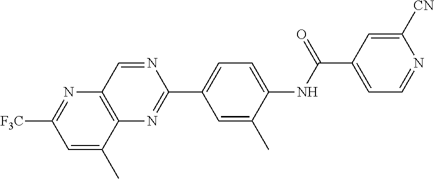 [SMILES: Cc1cc(-c2ncc3nc(C(F)(F)F)cc(C)c3n2)ccc1NC(=O)c1ccnc(C#N)c1]

To a stirred solution of 2-cyanopyridine-4-carbonyl chloride (300 mg, 1.80 mmol, 1 equiv.) and 2-methyl-4-[8-methyl-6-(trifluoromethyl)pyrido[3,2-d]pyrimidin-2-yl]aniline (516.0 mg, 1.62 mmol, 0.90 equiv.) in DCM (5 mL) was added TEA (240.6 mg, 2.38 mmol, 1.32 equiv.). The resulting mixture was stirred for 1 h at 0° C. and then quenched with NH₄Cl (aq.). The resulting mixture was extracted with CH₂Cl₂ and the combined organic layer was concentrated under reduced pressure. The residue was purified by Prep-TLC (CH₂Cl₂/MeOH=30:1) to afford the title compound as a yellow solid in 38.38% yield.

### Example 167: N2-methyl-N4-(2-methyl-4-(8-methyl-6-(trifluoromethyl)pyrido[3,2-d]pyrimidin-2-yl)phenyl)pyridine-2,4-dicarboxamide

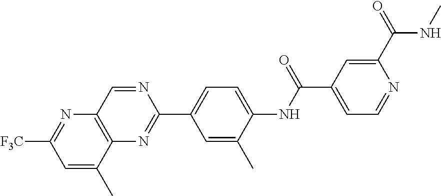 [SMILES: CNC(=O)c1cc(C(=O)Nc2ccc(-c3ncc4nc(C(F)(F)F)cc(C)c4n3)cc2C)ccn1]

To a stirred mixture of 2-chloro-8-methyl-6-(trifluoromethyl)pyrido[3,2-d]pyrimidine (100 mg, 0.40 mmol, 1 equiv.) and N2-methyl-N4-(2-methyl-4-(4,4,5,5-tetramethyl-1,3,2-dioxaborolan-2-yl)phenyl)pyridine-2,4-dicarboxamide (176.0 mg, 0.45 mmol, 1.10 equiv.) in dioxane (2 mL) were added H₂O (0.5 mL), Na₂CO₃ (128.7 mg, 1.21 mmol, 3 equiv.), and Pd(PPh₃)₄(93.6 mg, 0.08 mmol, 0.2 equiv.). The resulting mixture was stirred overnight at 80° C. under N₂ and then quenched with water. The resulting mixture was extracted with EA and the combined organic layer was concentrated under reduced pressure. The residue was purified by Prep-TLC (CH₂Cl₂/MeOH=30:1) to afford the title compound as a yellow solid in 13.98% yield. LC-MS: (ES, m/z): [M+H]⁺ 481; ¹H-NMR: (300 MHz, DMSO, ppm): δ 2.43 (s, 3H), 2.86-2.88 (d, 3H), 2.95 (s, 3H), 7.68-7.70 (d, 1H), 8.10-8.12 (m, 1H), 8.39 (s, 1H), 8.50-8.53 (d, 1H), 8.58 (s, 2H), 8.86-8.88 (d, 1H), 8.91-8.94 (m, 1H), 9.88 (s, 1H), 10.55 (s, 1H).

### Step 1: 4-[(4-bromo-2-methylphenyl)carbamoyl]pyridine-2-carboxylic acid

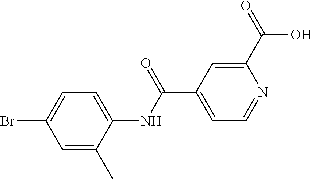 [SMILES: Cc1cc(Br)ccc1NC(=O)c1ccnc(C(=O)O)c1]

To a stirred solution of N-(4-bromo-2-methylphenyl)-2-cyanopyridine-4-carboxamide (1 g, 3.16 mmol, 1 equiv.) in EtOH (20 mL) was added NaOH (20 mL, 2 M). The resulting mixture was stirred for 2 h at 60° C. and then concentrated under reduced pressure. The aqueous layer was extracted with EA and the residue was acidified to pH 6 with HCl (1M). The resulting mixture was filtered, the filter cake was washed with water and the resulting solid was dried under infrared light to give the title compound as a yellow solid in 57.92% yield.

### Step 2: 4-N-(4-bromo-2-methylphenyl)-2-N-methylpyridine-2,4-dicarboxamide

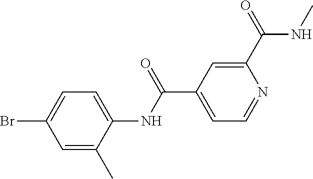 [SMILES: CNC(=O)c1cc(C(=O)Nc2ccc(Br)cc2C)ccn1]

To a stirred mixture of 4-[(4-bromo-2-methylphenyl)carbamoyl]pyridine-2-carboxylic acid (614 mg, 1.83 mmol, 1 equiv.) in DMF (10 mL) were added methanamine hydrochloride (185.5 mg, 2.75 mmol, 1.50 equiv.), DIEA (473.5 mg, 3.66 mmol, 2 equiv.) and HATU (1044.9 mg, 2.75 mmol, 1.5 equiv.). The resulting mixture was stirred overnight at room temperature and then washed with water and extracted with EA. The combined organic layer was concentrated under reduced pressure and the residue was purified by Prep-TLC (CH₂Cl₂/MeOH=20:1) to afford the title compound as a white solid in 69.45% yield.

### Step 3: N2-methyl-N4-(2-methyl-4-(4,4,5,5-tetramethyl-1,3,2-dioxaborolan-2-yl)phenyl)pyridine-2,4-dicarboxamide

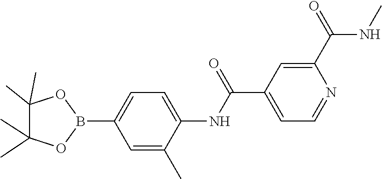

Proceeding analogously as described in Example 161, Step 2 but substituting N-(4-bromo-2-methylphenyl)-N,2-dimethylpyridine-4-carboxamide with 4-N-(4-bromo-2-methylphenyl)-2-N-methylpyridine-2,4-dicarboxamide afforded the title compound as a yellow solid after purification by Prep-TLC (PE/EA=2:1) in 69.60% yield.

### Example 168: N4-methyl-N4-(2-methyl-4-(8-methyl-6-(trifluoromethyl)pyrido[3,2-d]pyrimidin-2-yl)phenyl)pyridine-2,4-dicarboxamide

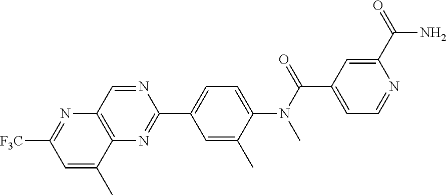 [SMILES: Cc1cc(-c2ncc3nc(C(F)(F)F)cc(C)c3n2)ccc1N(C)C(=O)c1ccnc(C(N)=O)c1]

Proceeding analogously as described in Example 167 but substituting N2-methyl-N4-(2-methyl-4-(4,4,5,5-tetramethyl-1,3,2-dioxaborolan-2-yl)phenyl)pyridine-2,4-dicarboxamide with N4-methyl-N4-(2-methyl-4-(4,4,5,5-tetramethyl-1,3,2-dioxaborolan-2-yl)phenyl)pyridine-2,4-dicarboxamide provided the title compound as a yellow solid 13.3% yield. LC-MS: (ES, m/z): [M+H]⁺ 481; ¹H-NMR: (300 MHz, DMSO, ppm): δ 2.38 (s, 3H), 2.89 (s, 3H), 3.37 (s, 3H), 7.42-7.45 (m, 1H), 7.51-7.53 (d, 1H), 7.63-7.64 (d, 1H), 7.90 (s, 1H), 8.02-8.03 (d, 1H), 8.33-8.37 (m, 2H), 8.45-8.50 (m, 2H), 9.83 (s, 1H)

### Step 1: N-(4-bromo-2-methylphenyl)-2-cyano-N-methylpyridine-4-carboxamide

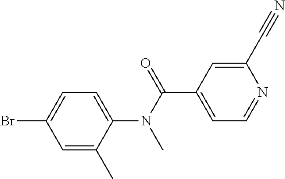 [SMILES: Cc1cc(Br)ccc1N(C)C(=O)c1ccnc(C#N)c1]

To a stirred mixture of N-(4-bromo-2-methylphenyl)-2-cyanopyridine-4-carboxamide (2 g, 6.33 mmol, 1 equiv.) in DMF (20 mL) was added NaH (305 mg, 12.71 mmol, 2.01 equiv.) in portions at 0° C. The resulting mixture was stirred for 3 h and Mel (1 g, 7.05 mmol, 1.11 equiv.) was added. The resulting mixture was stirred for additional 2 h at room temperature and then quenched with NH₄Cl (aq.). The resulting mixture was extracted with EA and the combined organic layer was concentrated under reduced pressure. The residue was purified by silica gel column chromatography, eluted with PE/EA (5:1) to afford the title compound as a yellow solid in 71.81% yield.

### Step 2: 4-N-(4-bromo-2-methylphenyl)-4-N-methylpyridine-2,4-dicarboxamide

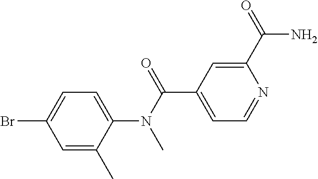 [SMILES: Cc1cc(Br)ccc1N(C)C(=O)c1ccnc(C(N)=O)c1]

To a stirred mixture of N-(4-bromo-2-methylphenyl)-2-cyano-N-methylpyridine-4-carboxamide (1.5 g, 4.54 mmol, 1 equiv.) in MeOH (110 mL) were added NaOH (0.4 g, 10.00 mmol, 2.20 equiv.), H₂O (11 mL) and H₂O₂(5.5 mL). The resulting mixture was stirred overnight at room temperature and then neutralized to pH 7 with HCl (aq.). MeOH was evaporated under reduced pressure and the resulting mixture was extracted with EA. The combined organic layer was concentrated under reduced pressure and the residue was purified on a silica gel column with CH₂Cl₂/MeOH=30:1 as eluent to provide the title compound as a white solid in 85.98% yield.

### Step 3: N4-methyl-N4-(2-methyl-4-(4,4,5,5-tetramethyl-1,3,2-dioxaborolan-2-yl)phenyl)pyridine-2,4-dicarboxamide

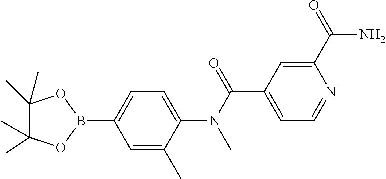 [SMILES: Cc1cc(B2OC(C)(C)C(C)(C)O2)ccc1N(C)C(=O)c1ccnc(C(N)=O)c1]

Proceeding analogously as described in Example 160, Step 9 but substituting N-(4-bromo-2-methylphenyl)-2-methylpyridine-4-carboxamide with 4-N-(4-bromo-2-methylphenyl)-4-N-methylpyridine-2,4-dicarboxamide, followed by purification of crude product on a silica gel column by eluting with (PE/EA=2:1) provided the title compound as a yellow solid in 64.77% yield.

### Example 169: N2,N4-dimethyl-N4-(2-methyl-4-(8-methyl-6-(trifluoromethyl)pyrido-[3,2-d]pyrimidin-2-yl)phenyl)pyridine-2,4-dicarboxamide

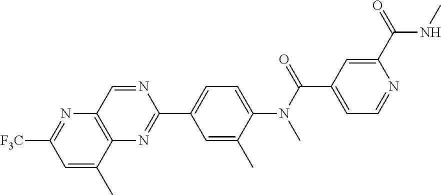 [SMILES: CNC(=O)c1cc(C(=O)N(C)c2ccc(-c3ncc4nc(C(F)(F)F)cc(C)c4n3)cc2C)ccn1]

Proceeding analogously as described in Example 167 but substituting N2-methyl-N4-(2-methyl-4-(4,4,5,5-tetramethyl-1,3,2-dioxaborolan-2-yl)phenyl)pyridine-2,4-dicarboxamide with 2-N,4-N-dimethyl-4-N-[2-methyl-4-(tetramethyl-1,3,2-dioxaborolan-2-yl)phenyl]pyridine-2,4-dicarboxamide provided the title compound as a white solid in 23.28% yield. LC-MS: (ES, m/z): [M+H]⁺ 495; ¹H-NMR: (300 MHz, DMSO, ppm): δ 2.38 (s, 3H), 2.71-2.73 (d, 3H), 2.89 (s, 3H), 3.65 (s, 3H), 7.43-7.45 (q, 1H), 7.50-7.53 (d, 1H), 7.89 (s, 1H), 8.32-8.37 (m, 2H), 8.45-8.51 (m, 2H), 8.68-8.72 (m, 1H), 9.84 (s, 1H)

### Step 1: 4-[(4-bromo-2-methylphenyl)(methyl)carbamoyl]pyridine-2-carboxylic acid

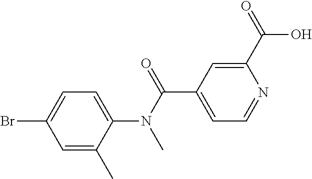 [SMILES: Cc1cc(Br)ccc1N(C)C(=O)c1ccnc(C(=O)O)c1]

Proceeding analogously as described in Example 167, Step 1 but substituting N-(4-bromo-2-methylphenyl)-2-cyanopyridine-4-carboxamide with N-(4-bromo-2-methylphenyl)-2-cyano-N-methylpyridine-4-carboxamide provided the title compound as a yellow solid in 63.04% yield.

### Step 2: N4-(4-bromo-2-methylphenyl)-N2,N4-dimethylpyridine-2,4-dicarboxamide

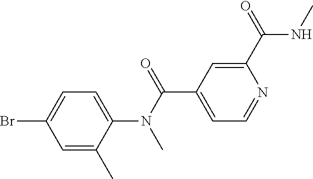 [SMILES: CNC(=O)c1cc(C(=O)N(C)c2ccc(Br)cc2C)ccn1]

Proceeding analogously as described in Example 167, Step 2 but substituting 4-[(4-bromo-2-methylphenyl)carbamoyl]pyridine-2-carboxylic acid with 4-[(4-bromo-2-methylphenyl)(methyl)carbamoyl]pyridine-2-carboxylic acid provided crude product Purification by silica gel column chromatography, with CH₂Cl₂/MeOH (50:1) as eluent provided the title compound as a yellow oil in 96.40% yield.

### Step 3: 2-N,4-N-dimethyl-4-N-[2-methyl-4-(tetramethyl-1,3,2-dioxaborolan-2-yl)phenyl]pyridine-2,4-dicarboxamide

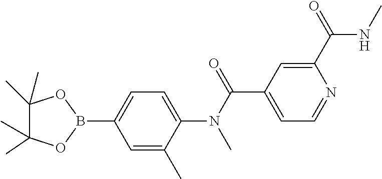

Proceeding analogously as described in Example 168, Step 3 but substituting 4-N-(4-bromo-2-methylphenyl)-2-N-methylpyridine-2,4-dicarboxamide with 4-N-(4-bromo-2-methylphenyl)-2-N,4-N-dimethylpyridine-2,4-dicarboxamide provided the title compound as a yellow solid in 64.61% yield.

### Example 170: 1-(2-amino-2-oxoethyl)-N-(2-methyl-4-(8-methyl-6-(trifluoromethyl)-pyrido[3,2-d]pyrimidin-2-yl)phenyl)-1H-pyrazole-5-carboxamide

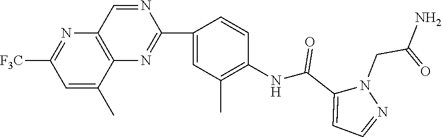 [SMILES: Cc1cc(-c2ncc3nc(C(F)(F)F)cc(C)c3n2)ccc1NC(=O)c1ccnn1CC(N)=O]

To a stirred mixture of 2-(5-((2-methyl-4-(8-methyl-6-(trifluoromethyl)pyrido[3,2-d]pyrimidin-2-yl)phenyl)carbamoyl)-1H-pyrazol-1-yl)acetic acid (75 mg, 0.16 mmol, 1 equiv.) and NH₄Cl (17.1 mg, 0.32 mmol, 2 equiv.) in DMF (2 mL) were added HATU (90.9 mg, 0.24 mmol, 1.5 equiv.) and DIEA (82.4 mg, 0.64 mmol, 4 equiv.). The resulting mixture was stirred overnight at room temperature and then washed with water. The resulting mixture was extracted with CH₂Cl₂ and the combined organic layers were concentrated under reduced pressure. The residue was purified by Prep-TLC (CH₂Cl₂/MeOH=20:1) to afford the title compound as a white solid in 58.12% yield. LC-MS: (ES, m/z): [M+H]⁺ 470; ¹H-NMR: (300 MHz, DMSO, ppm): δ 2.29-2.32 (d, 3H), 2.79 (s, 3H), 4.46-4.51 (d, 1H), 4.75-4.81 (m, 1H), 5.37-5.58 (m, 1H), 6.88-6.89 (d, 1H), 7.00-7.06 (m, 1H), 7.49-7.56 (m, 1H), 7.67 (s, 1H), 8.04 (s, 1H), δ8.17 (s, 1H), 8.32 (s, 1H), 8.38-8.44 (m, 2H).

### Step 1: N-(2-methyl-4-(8-methyl-6-(trifluoromethyl)pyrido[3,2-d]pyrimidin-2-yl)phenyl)-1-(2-oxoethyl)-1H-pyrazole-5-carboxamide

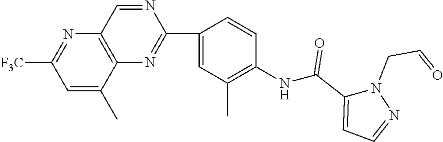 [SMILES: Cc1cc(-c2ncc3nc(C(F)(F)F)cc(C)c3n2)ccc1NC(=O)c1ccnn1CC=O]

To a stirred mixture of 1-(2-hydroxyethyl)-N-[2-methyl-4-[8-methyl-6-(trifluoromethyl)-pyrido[3,2-d]pyrimidin-2-yl]phenyl]-1H-pyrazole-5-carboxamide (120 mg, 0.26 mmol, 1 equiv.) in DCM (2.0 mL) was added Dess-Martin reagent (223.0 mg, 0.53 mmol, 2 equiv.). The resulting mixture was stirred for 6 h at room temperature and then filtered, and the filter cake was washed with water. The resulting mixture was extracted with CH₂Cl₂ and the combined organic layers were concentrated under reduced pressure. The residue was purified by Prep-TLC (PE/EtOAc=1:1) to afford the title compound as a white solid in 83.70% yield.

### Step 2: 2-(5-((2-methyl-4-(8-methyl-6-(trifluoromethyl)pyrido[3,2-d]pyrimidin-2-yl)phenyl)carbamoyl)-1H-pyrazol-1-yl)acetic acid

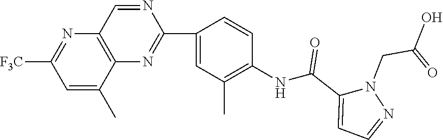 [SMILES: Cc1cc(-c2ncc3nc(C(F)(F)F)cc(C)c3n2)ccc1NC(=O)c1ccnn1CC(=O)O]

To a stirred mixture of N-(2-methyl-4-(8-methyl-6-(trifluoromethyl)pyrido[3,2-d]pyrimidin-2-yl)phenyl)-1-(2-oxoethyl)-1H-pyrazole-5-carboxamide (100 mg, 0.22 mmol, 1 equiv.) in DMF (1 mL) was added Oxone (111.0 mg, 0.66 mmol, 3 equiv.). The resulting mixture was stirred overnight at room temperature and then washed with water. The resulting mixture was extracted with EtOAc and the combined organic layers were concentrated under reduced pressure. The residue was purified by Prep-TLC (CH₂Cl₂/MeOH=15:1) to afford the title compound as a white solid in 82.11% yield.

### Example 171: 1-(2-amino-2-oxoethyl)-N-methyl-N-(2-methyl-4-(8-methyl-6-(trifluoromethyl)pyrido[3,2-d]pyrimidin-2-yl)phenyl)-1H-pyrazole-5-carboxamide

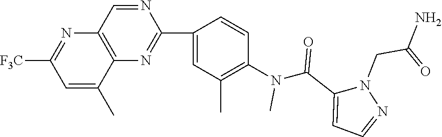 [SMILES: Cc1cc(-c2ncc3nc(C(F)(F)F)cc(C)c3n2)ccc1N(C)C(=O)c1ccnn1CC(N)=O]

Proceeding analogously as described in Example 167, Step 2 but substituting 4-[(4-bromo-2-methylphenyl)carbamoyl]pyridine-2-carboxylic acid with 2-(5-(methyl(2-methyl-4-(8-methyl-6-(trifluoromethyl)pyrido[3,2-d]pyrimidin-2-yl)phenyl)carbamoyl)-1H-pyrazol-1-yl)acetic acid and methanamine with ammonium chloride provided the title compound as a white solid in 43.55% yield. LC-MS: (ES, m/z): [M+H]⁺ 484; ¹H-NMR: (400 MHz, DMSO, ppm): δ 2.40 (s, 3H), 2.93 (s, 3H), 3.28 (s, 3H), 5.12-5.14 (d, 2H), 5.42-5.43 (d, 1H), 7.18 (d, 2H), 7.58-7.60 (d, 2H), 8.40 (d, 2H), 8.58 (s, 1H), 9.89 (s, 1H).

### Step 1: 1-(2-(benzyloxy)ethyl)-N-methyl-N-(2-methyl-4-(8-methyl-6-(trifluoromethyl)-pyrido[3,2-d]pyrimidin-2-yl)phenyl)-1H-pyrazole-5-carboxamide

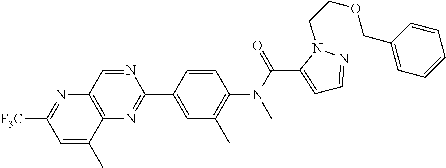 [SMILES: Cc1cc(-c2ncc3nc(C(F)(F)F)cc(C)c3n2)ccc1N(C)C(=O)c1ccnn1CCOCc1ccccc1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-[2-(benzyloxy)ethyl]-N-methyl-N-[2-methyl-4-(tetramethyl-1,3,2-dioxaborolan-2-yl)phenyl]-1H-pyrazole-5-carboxamide provided crude product which was purified by silica gel column chromatography, eluting with CH₂Cl₂/MeOH (20:1) to afford the title compound as a white solid in 57.42% yield.

### Step 2: 1-(2-hydroxyethyl)-N-methyl-N-(2-methyl-4-(8-methyl-6-(trifluoromethyl)pyrido-[3,2-d]pyrimidin-2-yl)phenyl)-1H-pyrazole-5-carboxamide

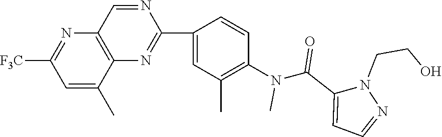 [SMILES: Cc1cc(-c2ncc3nc(C(F)(F)F)cc(C)c3n2)ccc1N(C)C(=O)c1ccnn1CCO]

To a stirred solution of 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide (520 mg, 0.93 mmol, 1 equiv.) in DCM (30 mL) was added BBr₃ (697.2 mg, 2.78 mmol, 3 equiv.) dropwise at 0° C. under nitrogen atmosphere. The resulting mixture was stirred for 1 h at 0° C. under nitrogen atmosphere and then quenched with sat. NH₄Cl (aq.) at 0° C. The resulting mixture was extracted with EA and the residue was purified by Prep-TLC (CH₂Cl₂/MeOH=20:1) to afford the title compound as a white solid in 61.87% yield.

### Step 3: N-methyl-N-(2-methyl-4-(8-methyl-6-(trifluoromethyl)pyrido[3,2-d]pyrimidin-2-yl)phenyl)-1-(2-oxoethyl)-1H-pyrazole-5-carboxamide

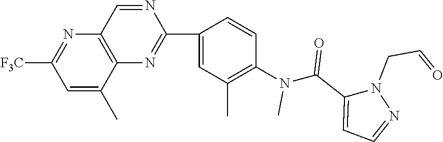 [SMILES: Cc1cc(-c2ncc3nc(C(F)(F)F)cc(C)c3n2)ccc1N(C)C(=O)c1ccnn1CC=O]

To a stirred solution of 1-(2-hydroxyethyl)-N-methyl-N-(2-methyl-4-(8-methyl-6-(trifluoromethyl)pyrido-[3,2-d]pyrimidin-2-yl)phenyl)-1H-pyrazole-5-carboxamide (270 mg, 0.57 mmol, 1 equiv.) in DCM (5 mL) was added Dess-Martin (730.3 mg, 1.72 mmol, 3 equiv.) in portions at room temperature. The resulting mixture was stirred for 3 h at room temperature. The resulting mixture was filtered, the filter cake was washed with DCM and the filtrate was concentrated under reduced pressure. The residue was purified by Prep-TLC (CH₂Cl₂/MeOH=20:1) to afford the title compound as a white solid in 37.20% yield.

### Step 4: 2-(5-(methyl(2-methyl-4-(8-methyl-6-(trifluoromethyl)pyrido[3,2-d]pyrimidin-2-yl)phenyl)carbamoyl)-1H-pyrazol-1-yl)acetic acid

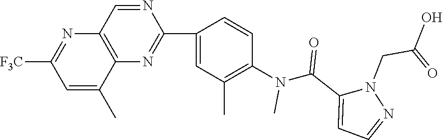 [SMILES: Cc1cc(-c2ncc3nc(C(F)(F)F)cc(C)c3n2)ccc1N(C)C(=O)c1ccnn1CC(=O)O]

To a stirred solution of N-methyl-N-(2-methyl-4-(8-methyl-6-(trifluoromethyl)pyrido-[3,2-d]pyrimidin-2-yl)phenyl)-1-(2-oxoethyl)-1H-pyrazole-5-carboxamide (100 mg, 0.21 mmol, 1 equiv.), DMF (1 mL) was added Oxone (71.8 mg, 0.43 mmol, 2 equiv.) at room temperature. The resulting mixture was stirred for 2 h at room temperature under air atmosphere and then extracted with EA. The residue was purified by Prep-TLC (CH₂Cl₂/MeOH=20:1) to afford the title compound as a white solid in 50.28% yield.

### Example 172: 1-(2-hydroxyethyl)-N-[2-methyl-4-[8-methyl-6-(trifluoromethyl)pyrido-[3,2-d]pyrimidin-2-yl]phenyl]-1H-pyrazole-5-carboxamide

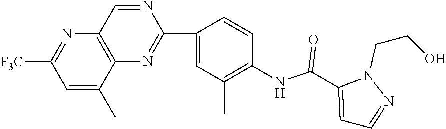 [SMILES: Cc1cc(-c2ncc3nc(C(F)(F)F)cc(C)c3n2)ccc1NC(=O)c1ccnn1CCO]

Into a 50-mL round-bottom flask, was placed 1-[2-(benzyloxy)ethyl]-N-[2-methyl-4-[8-methyl-6-(trifluoromethyl)pyrido[3,2-d]pyrimidin-2-yl]phenyl]-1H-pyrazole-5-carboxamide (100 mg, 0.18 mmol, 1 equiv.) and DCM (1 mL) and BBr₃ (275.0 mg, 1.10 mmol, 6 equiv.) was added at 0° C. The resulting solution was stirred for 1 h at room temperature and then quenched with NH₄Cl (aq.). The resulting solution was extracted with ethyl acetate and the organic layers were combined and concentrated. The residue was applied onto Prep-TLC and eluted with dichloromethane/methanol (40/1) to give the title compound as an off-white solid in 47.90% yield. LC-MS: (ES, m/z): [M+H]⁺ 457; ¹H-NMR: (400 MHz, DMSO, ppm): δ 2.41 (s, 3H), 2.95 (s, 3H), 3.72-3.76 (m, 2H), 4.58-4.61 (t, 2H), 4.91-4.93 (t, 1H), 7.06-7.07 (d, 1H), 7.59 (s, 1H), 7.65-7.67 (d, 1H), 8.38 (s, 1H), 8.49-8.52 (d, 1H), 8.56 (s, 1H), 9.87 (s, 1H), 10.04 (s, 1H)

### Step 1: ethyl 1-[2-(benzyloxy)ethyl]-1H-pyrazole-5-carboxylate

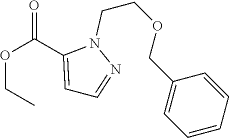 [SMILES: CCOC(=O)c1ccnn1CCOCc1ccccc1]

Into a 100-mL 3-necked round-bottom flask, was placed ethyl 1H-pyrazole-5-carboxylate (5.0 g, 35.68 mmol, 1 equiv.), THE (50 mL), 2-(benzyloxy)ethan-1-ol (5.97 g, 39.25 mmol, 1.1 equiv.), DIAD (10.82 g, 53.52 mmol, 1.5 equiv.), PPh₃ (18.7 g, 71.36 mmol, 2 equiv.). The resulting solution was stirred for 3 h at room temperature and then concentrated under vacuum. The residue was applied onto a silica gel column and eluted with ethyl acetate/petroleum ether (1/50) to give the title compound as a yellow liquid in 64.37% yield.

### Step 2: 1-[2-(benzyloxy)ethyl]-1H-pyrazole-5-carboxylic acid

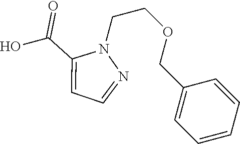 [SMILES: O=C(O)c1ccnn1CCOCc1ccccc1]

Into a 250-mL 3-necked round-bottom flask, was placed ethyl 1-[2-(benzyloxy)ethyl]-1H-pyrazole-5-carboxylate (6 g, 21.87 mmol, 1 equiv.), THE (60 mL), LiOH.H₂O (1.84 g, 43.75 mmol, 2 equiv.), and H₂O (12 mL). The resulting solution was stirred overnight at room temperature and the pH was adjusted to 5-6 with acetic acid. The solids were collected by filtration to give 5.5 g of the title compound as a white solid.

### Step 3: 1-[2-(benzyloxy)ethyl]-N-(4-bromo-2-methylphenyl)-1H-pyrazole-5-carboxamide

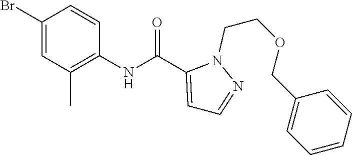 [SMILES: Cc1cc(Br)ccc1NC(=O)c1ccnn1CCOCc1ccccc1]

Proceeding analogously as described in Example 160, Step 8 but substituting 2-methylpyridine-4-carboxylic acid with 1-[2-(benzyloxy)ethyl]-1H-pyrazole-5-carboxylic acid provided crude product. Crystallization from petroleum and ether provided the title compound as an off-white solid in 77.27% yield.

### Step 4: 1-(2-(benzyloxy)ethyl)-N-(2-methyl-4-(4,4,5,5-tetramethyl-1,3,2-dioxaborolan-2-yl)phenyl)-1H-pyrazole-5-carboxamide

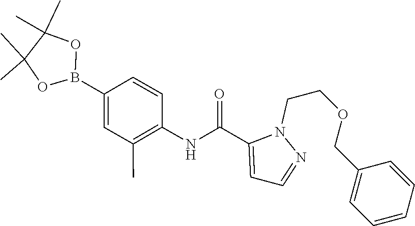 [SMILES: Cc1cc(B2OC(C)(C)C(C)(C)O2)ccc1NC(=O)c1ccnn1CCOCc1ccccc1]

Proceeding analogously as described in Example 161, Step 2 but substituting N-(4-bromo-2-methylphenyl)-N,2-dimethylpyridine-4-carboxamide with 1-[2-(benzyloxy)ethyl]-N-(4-bromo-2-methylphenyl)-1H-pyrazole-5-carboxamide provided the title compound as a yellow oil in 74.83% yield after purification with silica gel column chromatography with ethyl acetate/petroleum ether (1/5).

### Step 5: 1-[2-(benzyloxy)ethyl]-N-[2-methyl-4-[8-methyl-6-(trifluoromethyl)pyrido[3,2-d]pyrimidin-2-yl]phenyl]-1H-pyrazole-5-carboxamide

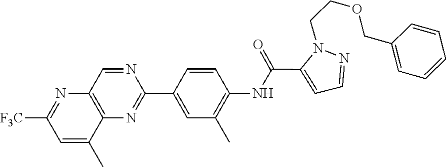 [SMILES: Cc1cc(-c2ncc3nc(C(F)(F)F)cc(C)c3n2)ccc1NC(=O)c1ccnn1CCOCc1ccccc1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-[2-(benzyloxy)ethyl]-N-[2-methyl-4-(tetramethyl-1,3,2-dioxaborolan-2-yl)phenyl]-1H-pyrazole-5-carboxamide provided crude product. Purification by silica gel column with ethyl acetate/petroleum ether (1/10) as the eluent provided the title compound as a yellow oil in 42.28% yield.

### Example 173: 1-(2-hydroxyethyl)-N-methyl-N-(2-methyl-4-(8-methyl-6-(trifluoromethyl)-pyrido[3,2-d]pyrimidin-2-yl)phenyl)-1H-pyrazole-5-carboxamide

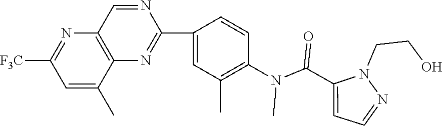 [SMILES: Cc1cc(-c2ncc3nc(C(F)(F)F)cc(C)c3n2)ccc1N(C)C(=O)c1ccnn1CCO]

Proceeding analogously as described in Example 172 but substituting 1-[2-(benzyloxy)-ethyl]-N-[2-methyl-4-[8-methyl-6-(trifluoromethyl)pyrido[3,2-d]pyrimidin-2-yl]phenyl]-1H-pyrazole-5-carboxamide with 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide gave crude product. The crude product was purified by Flash chromatography (Column, C18 silica gel; mobile phase, CH₃CN/H₂O=0/100 up to CH₃CN/H₂O=60/100 in 30 min; Detector, 254 nm) to give the title compound as an off-white solid in 23.32% yield. LC-MS: (ES, m/z): [M+H]⁺ 471; ¹H-NMR: (400 MHz, DMSO, ppm): δ 2.36 (s, 3H), 2.92 (s, 3H), 3.33 (s, 3H), 3.75-3.79 (m, 2H), 4.40-4.43 (m, 1H), 4.52-4.53 (m, 1H), 4.95-4.98 (t, 1H), 5.53 (s, 1H), 7.18 (s, 1H), 7.48-7.50 (d, 1H), 8.38-8.41 (d, 2H), 8.54 (s, 1H), 9.87 (s, 1H)

### Step 1: 1-(2-(benzyloxy)ethyl)-N-(4-bromo-2-methylphenyl)-N-methyl-1H-pyrazole-5-carboxamide

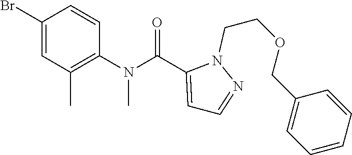 [SMILES: Cc1cc(Br)ccc1N(C)C(=O)c1ccnn1CCOCc1ccccc1]

Proceeding analogously as described in Example 161, Step 1 but substituting N-(4-bromo-2-methylphenyl)-2-methylpyridine-4-carboxamide with 1-[2-(benzyloxy)ethyl]-N-(4-bromo-2-methylphenyl)-1H-pyrazole-5-carboxamide provided crude product which was purified by silica gel column chromatography using ethyl acetate/petroleum ether (1/10) as eluent to give the title compound as an off-white solid in 73.70% yield.

### Step 2: 1-(2-(benzyloxy)ethyl)-N-methyl-N-(2-methyl-4-(4,4,5,5-tetramethyl-1,3,2-dioxaborolan-2-yl)phenyl)-1H-pyrazole-5-carboxamide

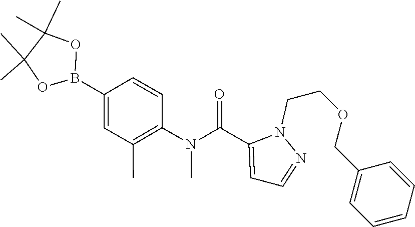 [SMILES: Cc1cc(B2OC(C)(C)C(C)(C)O2)ccc1N(C)C(=O)c1ccnn1CCOCc1ccccc1]

Proceeding analogously as described in Example 161, Step 2 but substituting N-(4-bromo-2-methylphenyl)-N,2-dimethylpyridine-4-carboxamide with 1-(2-(benzyloxy)ethyl)-N-(4-bromo-2-methylphenyl)-N-methyl-1H-pyrazole-5-carboxamide provided crude product. Purification by silica gel column chromatography with ethyl acetate/petroleum ether (1/5) as the eluent provided the title compound as a yellow oil in 67.57% yield.

### Step 3: 1-(2-(benzyloxy)ethyl)-N-methyl-N-(2-methyl-4-(8-methyl-6-(trifluoromethyl)-pyrido[3,2-d]pyrimidin-2-yl)phenyl)-1H-pyrazole-5-carboxamide

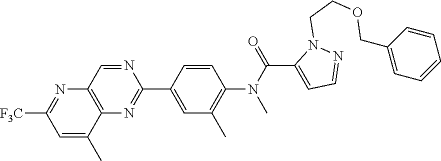 [SMILES: Cc1cc(-c2ncc3nc(C(F)(F)F)cc(C)c3n2)ccc1N(C)C(=O)c1ccnn1CCOCc1ccccc1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-(2-(benzyloxy)ethyl)-N-methyl-N-(2-methyl-4-(4,4,5,5-tetramethyl-1,3,2-dioxaborolan-2-yl)phenyl)-1H-pyrazole-5-carboxamide provided crude product. Purification with Prep-TLC using dichloromethane/methanol (40/1) as eluent gave the title compound as an off-white solid in 66.26% yield.

### Example 174: N-(3-fluoro-2-methyl-4-(8-methyl-6-(trifluoromethyl)pyrido[3,2-d]pyrimidin-2-yl)phenyl)-1-methyl-1H-pyrazole-5-carboxamide

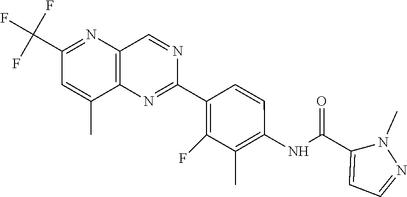 [SMILES: Cc1c(NC(=O)c2ccnn2C)ccc(-c2ncc3nc(C(F)(F)F)cc(C)c3n2)c1F]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-(3-fluoro-2-methyl-4-(4,4,5,5-tetramethyl-1,3,2-dioxaborolan-2-yl)phenyl)-1-methyl-1H-pyrazole-5-carboxamide provided crude product. Purification by Prep-TLC with dichloromethane/methanol (80:1) gave the title compound as a yellow solid in 50.15% yield. LC-MS: (ES, m/z): [M+H]⁺ 445; ¹H-NMR: (300 MHz, DMSO, ppm): δ 2.26 (s, 3H), 2.90 (s, 3H), 4.11 (s, 3H), 7.13 (s, 1H), 7.48-7.48 (d, 1H), 7.58 (s, 1H), 8.15-8.19 (m, 1H), 8.41 (s, 1H), 9.92 (s, 1H), 10.21 (s, 1H)

### Step 1: 4-bromo-3-fluoro-2-methylaniline

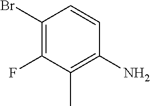

Into a 100-mL 3-necked round-bottom flask purged and maintained with an inert atmosphere of nitrogen, was placed 3-fluoro-2-methylaniline (5 g, 39.95 mmol, 1 equiv.) and DMF (50 mL) and NBS (7.1 g, 39.89 mmol, 0.998 equiv.) was added in three times at 0° C. Then the resulting solution was stirred overnight at room temperature and then extracted with ethyl acetate. The organic layers were combined and dried over anhydrous sodium sulfate. The solids were filtered and the filtrate was concentrated under vacuum to give the title compound as a purple solid in 96.91% yield.

### Step 2: N-(4-bromo-3-fluoro-2-methylphenyl)-1-methyl-1H-pyrazole-5-carboxamide

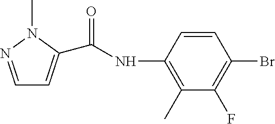 [SMILES: Cc1c(NC(=O)c2ccnn2C)ccc(Br)c1F]

Proceeding analogously as described in Example 160, Step 8 but substituting 4-bromo-2-methylaniline with 4-bromo-3-fluoro-2-methylaniline and 2-methylpyridine-4-carboxylic acid with 1-methyl-1H-pyrazole-5-carboxylic acid provided crude product. The residue was applied onto a silica gel column and eluted with CH₂Cl₂. Evaporation of volatiles afforded a solid that was re-crystallized from EA/PE (1:5) resulting in 1.6 g (52.29%) of the desired product as a white solid.

### Step 3: N-(3-fluoro-2-methyl-4-(4,4,5,5-tetramethyl-1,3,2-dioxaborolan-2-yl)phenyl)-1-methyl-1H-pyrazole-5-carboxamide

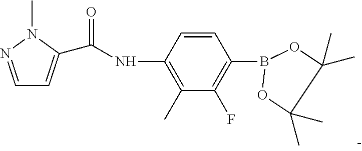 [SMILES: Cc1c(NC(=O)c2ccnn2C)ccc(B2OC(C)(C)C(C)(C)O2)c1F]

Proceeding analogously as described in Example 161, Step 2 but substituting N-(4-bromo-2-methylphenyl)-N,2-dimethylpyridine-4-carboxamide with N-(4-bromo-3-fluoro-2-methylphenyl)-1-methyl-1H-pyrazole-5-carboxamide provided crude product. The residue was applied onto a silica gel column and eluted with CH₂Cl₂. Evaporation of volatiles afforded a solid that was re-crystallized from EA/PE (1:5). This resulted in 0.9 g (111.73%) of the desired product as a white solid.

### Example 175: N-(3-fluoro-2-methyl-4-(8-methyl-6-(trifluoromethyl)pyrido[3,2-d]pyrimidin-2-yl)phenyl)-N,1-dimethyl-1H-pyrazole-5-carboxamide

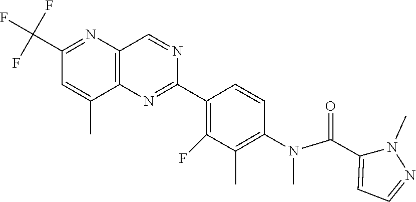 [SMILES: Cc1c(N(C)C(=O)c2ccnn2C)ccc(-c2ncc3nc(C(F)(F)F)cc(C)c3n2)c1F]

Proceeding analogously as described in Example 163 but substituting N-[2-fluoro-4-(tetramethyl-1,3,2-dioxaborolan-2-yl)phenyl]-1-methyl-1H-pyrazole-5-carboxamide with N-(3-fluoro-2-methyl-4-(4,4,5,5-tetramethyl-1,3,2-dioxaborolan-2-yl)phenyl)-N,1-dimethyl-1H-pyrazole-5-carboxamide provided crude product. Purification with Prep-TLC with dichloromethane/methanol (50:1) as eluent gave the title compound as a yellow solid in 35.59% yield. LC-MS: (ES, m/z): [M+H]⁺ 459; ¹H-NMR: (300 MHz, DMSO, ppm): δ 2.18 (s, 3H), 2.88 (s, 3H), 3.35 (s, 3H), 4.01 (s, 3H), 5.65 (s, 1H), 7.21 (s, 1H), 7.37-7.40 (d, 1H), 8.08-8.13 (t, 1H), 8.41 (s, 1H), 9.90 (s, 1H)

### Step 1: N-(4-bromo-3-fluoro-2-methylphenyl)-N,1-dimethyl-1H-pyrazole-5-carboxamide

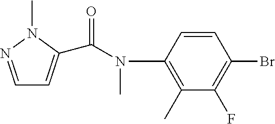 [SMILES: Cc1c(N(C)C(=O)c2ccnn2C)ccc(Br)c1F]

Into a 25-mL 3-necked round-bottom flask purged and maintained with an inert atmosphere of nitrogen was placed N-(4-bromo-3-fluoro-2-methylphenyl)-1-methyl-1H-pyrazole-5-carboxamide (0.87 g, 2.79 mmol, 1 equiv.), DMF (9 mL), and NaH (0.1 g, 3.34 mmol, 1.2 equiv.) was added at 0° C. The resulting mixture was stirred for 0.5 h and Mel (0.4 g, 2.93 mmol, 1.05 equiv.) was added. The resulting solution was stirred overnight at room temperature and then quenched with NH₄Cl (aq.). The resulting solution was extracted with ethyl acetate and the organic layers were combined and concentrated under vacuum to give the title compound as a white solid in 88.00% yield.

### Step 2: N-(3-fluoro-2-methyl-4-(4,4,5,5-tetramethyl-1,3,2-dioxaborolan-2-yl)phenyl)-N,1-dimethyl-1H-pyrazole-5-carboxamide

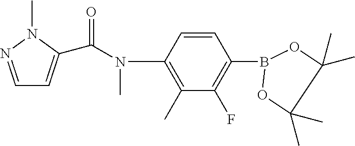 [SMILES: Cc1c(N(C)C(=O)c2ccnn2C)ccc(B2OC(C)(C)C(C)(C)O2)c1F]

Proceeding analogously as described in Example 161, Step 2 but substituting N-(4-bromo-2-methylphenyl)-N,2-dimethylpyridine-4-carboxamide with N-(4-bromo-3-fluoro-2-methylphenyl)-N,1-dimethyl-1H-pyrazole-5-carboxamide provided crude product. Purification on a silica gel column with ethyl acetate/petroleum ether (1:20) as eluent gave the title compound as a white solid in 81.93% yield.

### Example 176: N-(2-fluoro-3-methyl-4-(8-methyl-6-(trifluoromethyl)pyrido[3,2-d]pyrimidin-2-yl)phenyl)-1-methyl-1H-pyrazole-5-carboxamide

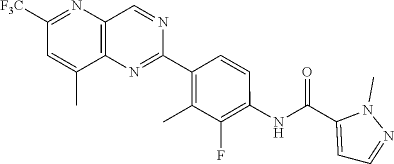 [SMILES: Cc1c(-c2ncc3nc(C(F)(F)F)cc(C)c3n2)ccc(NC(=O)c2ccnn2C)c1F]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-(2-fluoro-3-methyl-4-(4,4,5,5-tetramethyl-1,3,2-dioxaborolan-2-yl)phenyl)-1-methyl-1H-pyrazole-5-carboxamide provided the crude product. Purification on Prep-TLC with ethyl acetate/hexane (1/1) as eluent provided the title compound as a yellow solid in 11.81 yield.

LC-MS: (ES, m/z): [M+H]⁺ 445; ¹H-NMR: (300 MHz, DMSO, ppm): δ 2.64-2.65 (d, 3H), 2.90 (s, 3H), 4.11 (s, 3H), 7.15-7.16 (d, 1H), 7.57-7.56 (d, 1H), 7.69-7.75 (t, 1H), 8.03-8.05 (d, 1H), 8.42 (s, 1H), 9.93 (s, 1H), 10.25 (s, 1H)

### Step 1: 4-bromo-2-fluoro-3-methylaniline

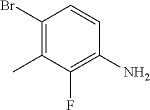

Proceeding as described in Example 174, Step 1 but substituting 3-fluoro-2-methylaniline with 2-fluoro-3-methylaniline provided the title compound as a red solid in 97.89% yield.

### Step 2: N-(4-bromo-2-fluoro-3-methylphenyl)-1-methyl-1H-pyrazole-5-carboxamide

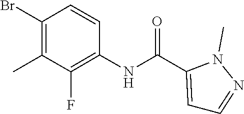 [SMILES: Cc1c(Br)ccc(NC(=O)c2ccnn2C)c1F]

Proceeding analogously as described in Example 160, Step 8 but substituting 4-bromo-2-methylaniline with 4-bromo-2-fluoro-3-methylaniline (2 g, 9.80 mmol, 1 equiv.) and 2-methylpyridine-4-carboxylic acid with 1-methyl-1H-pyrazole-5-carboxylic acid provided the crude product. Purification on a silica gel column with dichloromethane/methanol (100/1) as eluent provided the title compound as a red solid in 76.81% yield.

### Step 3: N-(2-fluoro-3-methyl-4-(4,4,5,5-tetramethyl-1,3,2-dioxaborolan-2-yl)phenyl)-1-methyl-1H-pyrazole-5-carboxamide

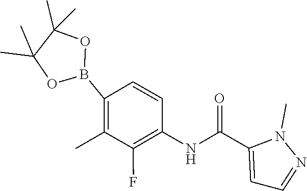 [SMILES: Cc1c(B2OC(C)(C)C(C)(C)O2)ccc(NC(=O)c2ccnn2C)c1F]

Proceeding analogously as described in Example 161, Step 2 but substituting N-(4-bromo-2-methylphenyl)-N,2-dimethylpyridine-4-carboxamide with N-(4-bromo-2-fluoro-3-methylphenyl)-1-methyl-1H-pyrazole-5-carboxamide provided the title compound as a white solid in 65.85% yield.

### Example 177: N-(2-fluoro-3-methyl-4-(8-methyl-6-(trifluoromethyl)pyrido[3,2-d]pyrimidin-2-yl)phenyl)-N,1-dimethyl-1H-pyrazole-5-carboxamide

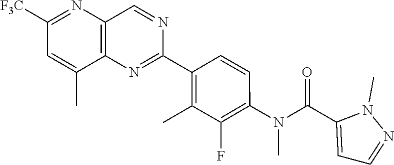 [SMILES: Cc1c(-c2ncc3nc(C(F)(F)F)cc(C)c3n2)ccc(N(C)C(=O)c2ccnn2C)c1F]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-(2-fluoro-3-methyl-4-(4,4,5,5-tetramethyl-1,3,2-dioxaborolan-2-yl)phenyl)-N,1-dimethyl-1H-pyrazole-5-carboxamide gave crude product. Purification by Prep-TLC with ethyl acetate/hexane (1/5) and re-crystallization from hexane gave the title compound as a yellow solid. LC-MS: (ES, m/z): [M+H]⁺ 459; ¹H-NMR: (300 MHz, DMSO, ppm): δ 2.51 (s, 3H), 2.87 (s, 3H), 3.39 (s, 3H), 3.98 (s, 3H), 5.86 (s, 1H), 7.26 (s, 1H), 7.53-7.59 (t, 1H), 7.95-7.98 (d, 1H), 8.42 (s, 1H), 9.91 (s, 1H)

### Step 1: N-(4-bromo-2-fluoro-3-methylphenyl)-N,1-dimethyl-1H-pyrazole-5-carboxamide

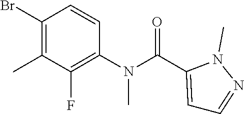 [SMILES: Cc1c(Br)ccc(N(C)C(=O)c2ccnn2C)c1F]

Proceeding analogously as described in Example 175, Step 1 but substituting N-(4-bromo3-fluoro-2-methylphenyl)-2-methylpyridine-4-carboxamide with N-(4-bromo-2-fluoro-3-methylphenyl)-1-methyl-1H-pyrazole-5-carboxamide provided the title compound as a yellow solid in 78.15% yield.

### Step 2: N-(2-fluoro-3-methyl-4-(4,4,5,5-tetramethyl-1,3,2-dioxaborolan-2-yl)phenyl)-N,1-dimethyl-1H-pyrazole-5-carboxamide

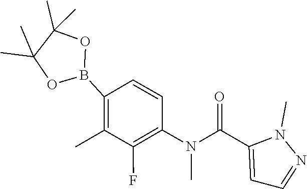 [SMILES: Cc1c(B2OC(C)(C)C(C)(C)O2)ccc(N(C)C(=O)c2ccnn2C)c1F]

Proceeding analogously as described in Example 161, Step 2 but substituting N-(4-bromo-2-methylphenyl)-N,2-dimethylpyridine-4-carboxamide with N-(4-bromo-2-fluoro-3-methylphenyl)-N,1-dimethyl-1H-pyrazole-5-carboxamide provided crude product which was purified by column chromatography by eluting with ethyl acetate/petroleum ether (1/10). The crude product was purified by re-crystallization from PE to give the title compound as a white solid in 54.69% yield.

### Example 178: N-(2-fluoro-5-methyl-4-(8-methyl-6-(trifluoromethyl)pyrido[3,2-d]pyrimidin-2-yl)phenyl)-1-methyl-1H-pyrazole-5-carboxamide

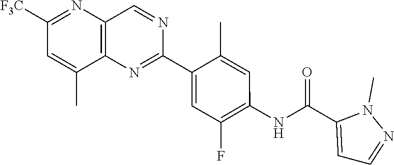 [SMILES: Cc1cc(NC(=O)c2ccnn2C)c(F)cc1-c1ncc2nc(C(F)(F)F)cc(C)c2n1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-[2-fluoro-5-methyl-4-(tetramethyl-1,3,2-dioxaborolan-2-yl)phenyl]-1-methyl-1H-pyrazole-5-carboxamide provided the crude product. Purification by Prep-TLC (CH₂Cl₂/MeOH=20:1) afforded the title compound as a white solid 38.45% yield. LC-MS: (ES, m/z): [M+H]⁺ 445; ¹H-NMR: (400 MHz, DMSO, ppm): δ 2.74 (s, 3H), 2.89 (s, 3H), 4.11 (s, 3H), 7.15-7.16 (d, 1H), 7.56-7.57 (d, 1H), 7.71-7.73 (d, 1H), 8.10-8.13 (d, 1H), 9.90 (s, 1H), 10.28 (s, 1H).

### Step 1: 4-bromo-2-fluoro-5-methylaniline

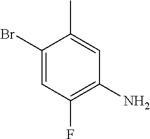

Proceeding analogously as described in Example 174, Step 1 but substituting 3-fluoro-2-methylaniline provided the title compound as a white solid in 90.77% yield.

### Step 2: N-(4-bromo-2-fluoro-5-methylphenyl)-1-methyl-1H-pyrazole-5-carboxamide

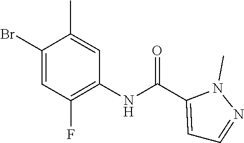 [SMILES: Cc1cc(NC(=O)c2ccnn2C)c(F)cc1Br]

Proceeding analogously as described in Example 160, Step 8 but substituting 4-bromo-2-methylaniline with 4-bromo-2-fluoro-5-methylaniline (2 g, 9.80 mmol, 1 equiv.) and 2-methylpyridine-4-carboxylic acid with 1-methyl-1H-pyrazole-5-carboxylic acid provided crude product. Purification by column chromatography by eluting with PE/EA (provided the title compound as a white solid in 94.78% yield.

### Step 3: N-[2-fluoro-5-methyl-4-(tetramethyl-1,3,2-dioxaborolan-2-yl)phenyl]-1-methyl-1H-pyrazole-5-carboxamide

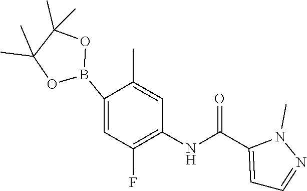 [SMILES: Cc1cc(NC(=O)c2ccnn2C)c(F)cc1B1OC(C)(C)C(C)(C)O1]

Proceeding analogously as described in Example 161, Step 2 but substituting N-(4-bromo-2-methylphenyl)-N,2-dimethylpyridine-4-carboxamide with N-(4-bromo-2-fluoro-5-methylphenyl)-1-methyl-1H-pyrazole-5-carboxamide provided crude product. Purification by column chromatography by eluting with petroleum ether/ethyl acetate (5:1) provided the title compound as a white solid in 84.29% yield.

### Example 179: N-(2-fluoro-5-methyl-4-(8-methyl-6-(trifluoromethyl)pyrido[3,2-d]pyrimidin-2-yl)phenyl)-N,1-dimethyl-1H-pyrazole-5-carboxamide

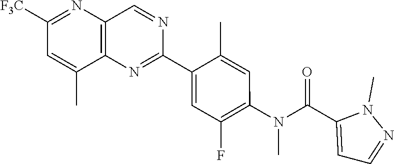 [SMILES: Cc1cc(N(C)C(=O)c2ccnn2C)c(F)cc1-c1ncc2nc(C(F)(F)F)cc(C)c2n1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-[2-fluoro-5-methyl-4-(tetramethyl-1,3,2-dioxaborolan-2-yl)phenyl]-N,1-dimethyl-1H-pyrazole-5-carboxamide provided crude product. Purification by Prep-TLC (CH₂Cl₂/MeOH=20:1) provided the title compound as a white solid in 58.71% yield. LC-MS: (ES, m/z): [M+H]⁺ 459; ¹H-NMR: (300 MHz, DMSO, ppm): δ2.69 (s, 3H), 2.89 (s, 3H), 3.41 (s, 3H), 3.97 (s, 3H), 5.90 (s, 1H), 7.21 (s, 1H), 7.64-7.67 (d, 1H), 7.94-7.98 (d, 1H), 8.43 (s, 1H), 9.90 (s, 1H).

### Step 1: N-(4-bromo-2-fluoro-5-methylphenyl)-N,1-dimethyl-1H-pyrazole-5-carboxamide

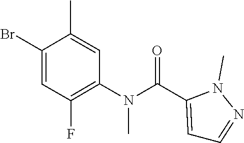 [SMILES: Cc1cc(N(C)C(=O)c2ccnn2C)c(F)cc1Br]

Proceeding analogously as described in Example 161, Step 1 but substituting N-(4-bromo-2-methylphenyl)-2-methylpyridine-4-carboxamide with N-(4-bromo-2-fluoro-5-methylphenyl)-1-methyl-1H-pyrazole-5-carboxamide provided the title compound as a white solid in 93.79% yield.

### Step 2: N-[2-fluoro-5-methyl-4-(tetramethyl-1,3,2-dioxaborolan-2-yl)phenyl]-N,1-dimethyl-1H-pyrazole-5-carboxamide

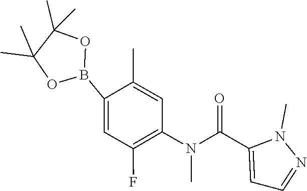 [SMILES: Cc1cc(N(C)C(=O)c2ccnn2C)c(F)cc1B1OC(C)(C)C(C)(C)O1]

Proceeding analogously as described in Example 161, Step 2 but substituting N-(4-bromo-2-methylphenyl)-N,2-dimethylpyridine-4-carboxamide with N-(4-bromo-2-fluoro-5-methylphenyl)-N,1-dimethyl-1H-pyrazole-5-carboxamide provided crude product. Purification by column chromatography by eluting with PE/EA (10:1) gave the title compound as a white solid in 74.01% yield.

### Example 180: N-(5-fluoro-2-methyl-4-(8-methyl-6-(trifluoromethyl)pyrido[3,2-d]pyrimidin-2-yl)phenyl)-1-methyl-1H-pyrazole-5-carboxamide

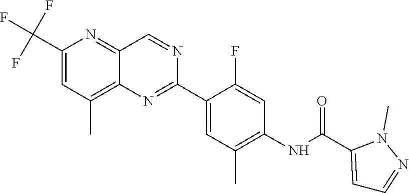 [SMILES: Cc1cc(-c2ncc3nc(C(F)(F)F)cc(C)c3n2)c(F)cc1NC(=O)c1ccnn1C]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-(5-fluoro-2-methyl-4-(4,4,5,5-tetramethyl-1,3,2-dioxaborolan-2-yl)phenyl)-1-methyl-1H-pyrazole-5-carboxamide gave crude product. Purification by Prep-TLC (CH₂Cl₂/MeOH=50:1) gave the title compound as a yellow solid in 31.15% yield. LC-MS: (ES, m/z): [M+H]⁺ 445; ¹H-NMR: (300 MHz, DMSO, ppm): δ2.38 (s, 3H), 2.90 (s, 3H), 4.12 (s, 3H), 7.13 (s, 1H), 7.57-7.61 (m, 2H), 8.25-8.27 (d, 1H), 8.41 (s, 1H), 9.91 (s, 1H), 10.00 (s, 1H).

### Step 1: N-(4-bromo-5-fluoro-2-methylphenyl)-1-methyl-1H-pyrazole-5-carboxamide

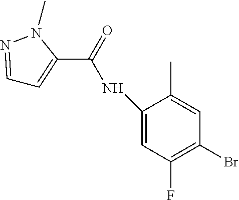 [SMILES: Cc1cc(Br)c(F)cc1NC(=O)c1ccnn1C]

Proceeding analogously as described in Example 160, Step 8 but substituting 4-bromo-2-methylaniline with 4-bromo-5-fluoro-2-methylaniline (2 g, 9.80 mmol, 1 equiv.) and 2-methylpyridine-4-carboxylic acid with 1-methyl-1H-pyrazole-5-carboxylic acid provided crude product. Purification column chromatography with CH₂C₂, followed by re-crystallization from EA/PE gave the title compound as a white solid in 42.49% yield).

### Step 2: N-(5-fluoro-2-methyl-4-(4,4,5,5-tetramethyl-1,3,2-dioxaborolan-2-yl)phenyl)-1-methyl-1H-pyrazole-5-carboxamide

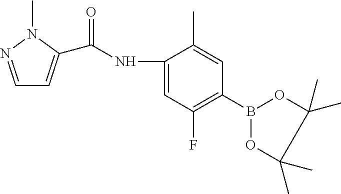 [SMILES: Cc1cc(B2OC(C)(C)C(C)(C)O2)c(F)cc1NC(=O)c1ccnn1C]

Proceeding analogously as described in Example 161, Step 2 but substituting N-(4-bromo-2-methylphenyl)-N,2-dimethylpyridine-4-carboxamide with N-(4-bromo-5-fluoro-2-methylphenyl)-1-methyl-1H-pyrazole-5-carboxamide provided crude product. Purification by column chromatograph with ethyl acetate/petroleum ether (1:10) as eluent gave the title compound as a white solid in 26.74% yield.

### Example 181: N-(5-fluoro-2-methyl-4-(8-methyl-6-(trifluoromethyl)pyrido[3,2-d]pyrimidin-2-yl)phenyl)-N,1-dimethyl-1H-pyrazole-5-carboxamide

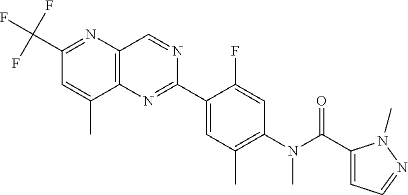 [SMILES: Cc1cc(-c2ncc3nc(C(F)(F)F)cc(C)c3n2)c(F)cc1N(C)C(=O)c1ccnn1C]

Proceeding analogously as in Example 163 but substituting N-[2-fluoro-4-(tetramethyl-1,3,2-dioxaborolan-2-yl)phenyl]-1-methyl-1H-pyrazole-5-carboxamide with N-(5-fluoro-2-methyl-4-(4,4,5,5-tetramethyl-1,3,2-dioxaborolan-2-yl)phenyl)-N,1-dimethyl-1H-pyrazole-5-carboxamide provided crude product. Purification by Prep-TLC with dichloromethane/methanol (50:1) gave the title compound in 52.01% yield. LC-MS: (ES, m/z): [M+H]⁺ 459; ¹H-NMR: (300 MHz, DMSO, ppm): δ2.22 (s, 3H), 2.88 (s, 3H), 3.33 (s, 3H), 4.01 (s, 3H), 5.68 (s, 1H), 7.23 (s, 1H), 7.53-7.56 (d, 1H), 8.20 (d, 1H), 8.41 (s, 1H), 9.90 (s, 1H).

### Step 1: N-(4-bromo-5-fluoro-2-methylphenyl)-N,1-dimethyl-1H-pyrazole-5-carboxamide

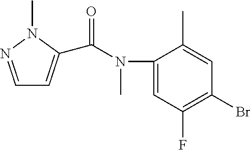 [SMILES: Cc1cc(Br)c(F)cc1N(C)C(=O)c1ccnn1C]

Proceeding analogously as described in Example 175, Step 1 but substituting N-(4-bromo-3-fluoro-2-methylphenyl)-1-methyl-1H-pyrazole-5-carboxamide with N-(4-bromo-5-fluoro-2-methylphenyl)-1-methyl-1H-pyrazole-5-carboxamide provided the title compound as a white solid in 99.38%.

### Step 2: N-(5-fluoro-2-methyl-4-(4,4,5,5-tetramethyl-1,3,2-dioxaborolan-2-yl)phenyl)-N,1-dimethyl-1H-pyrazole-5-carboxamide

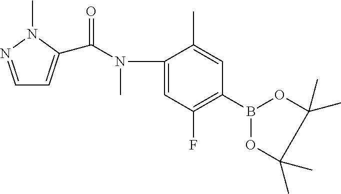 [SMILES: Cc1cc(B2OC(C)(C)C(C)(C)O2)c(F)cc1N(C)C(=O)c1ccnn1C]

Proceeding analogously as described in Example 161, Step 2 but substituting N-(4-bromo-2-methylphenyl)-N,2-dimethylpyridine-4-carboxamide with N-(4-bromo-5-fluoro-2-methylphenyl)-N,1-dimethyl-1H-pyrazole-5-provided crude product. Purification by column chromatography with ethyl acetate/petroleum ether as eluent, followed by re-crystallization from EA/PE (1:5) provided the title compound as a white solid in 38.52% yield.

### Example 182: N-methyl-N-(2-methyl-4-(8-methyl-6-(trifluoromethyl)pyrido[3,2-d]pyrimidin-2-yl)phenyl)-1-(2-(pyrrolidin-1-yl)ethyl)-1H-pyrazole-5-carboxamide

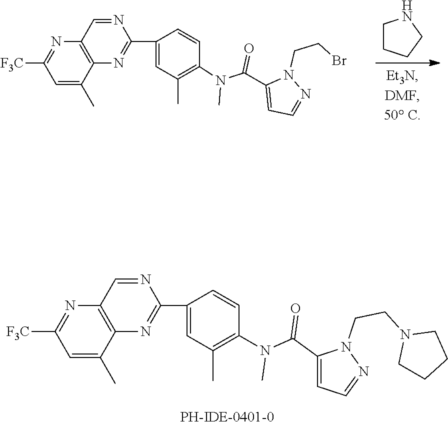 [SMILES: Cc1cc(-c2ncc3nc(C(F)(F)F)cc(C)c3n2)ccc1N(C)C(=O)c1ccnn1CCBr ; C1CCNC1 ; Cc1cc(-c2ncc3nc(C(F)(F)F)cc(C)c3n2)ccc1N(C)C(=O)c1ccnn1CCN1CCCC1]

Into an 8-mL vial, was placed 1-(2-bromoethyl)-N-methyl-N-[2-methyl-4-[8-methyl-6-(trifluoromethyl)pyrido[3,2-d]pyrimidin-2-yl]phenyl]-1H-pyrazole-5-carboxamide (78 mg, 0.15 mmol, 1 equiv.), DMF (1 mL), Et₃N (29.6 mg, 0.29 mmol, 2 equiv.), and pyrrolidine (41.6 mg, 0.58 mmol, 4 equiv.). The resulting solution was stirred for 6 h at 50° C. and then quenched with water. The resulting solution was extracted with ethyl acetate and the organic layers combined. The residue was purified by prep-TLC with dichloromethane/methanol (40/1). The resulting material was further purified by prep-HPLC with the following conditions: Column, XBridge Prep OBD C18 Column; mobile phase, water (0.05% NH₃H₂O) and ACN (60% Phase B up to 72% in 8 min) to afford the title compound as a white solid in 10.97% yield. LC-MS: (ES, m/z): [M+H]⁺ 524; ¹H-NMR: (300 MHz, DMSO, ppm): δ9.88 (s, 1H), 8.56 (d, 1H), 8.41-8.38 (m, 2H), 7.52-7.50 (d, 2H), 7.17-7.18 (d, 1H), 5.49-5.48 (d, 2H), 4.69-4.60 (m, 1H), 4.51-4.43 (m, 1H), 3.32 (s, 6H), 2.93 (s, 4H), 2.74-2.73 (d, 2H), 2.37 (s, 3H), 1.73 (s, 3H)

### Example 183: 3-methoxy-N-methyl-N-(2-methyl-4-(8-methyl-6-(trifluoromethyl)-pyrido[3,2-d]pyrimidin-2-yl)phenyl)isonicotinamide

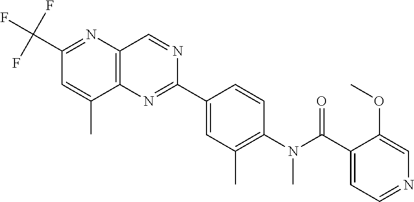 [SMILES: COc1cnccc1C(=O)N(C)c1ccc(-c2ncc3nc(C(F)(F)F)cc(C)c3n2)cc1C]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 3-methoxy-N-methyl-N-(2-methyl-4-(4,4,5,5-tetramethyl-1,3,2-dioxaborolan-2-yl)phenyl)isonicotinamide provided crude product. Purification by Prep-TLC (CH₂Cl₂/MeOH=20:1) afforded the title compound as a white solid in 34.91% yield. LC-MS: (ES, m/z): [M+H]⁺ 468; ¹H-NMR: (300 MHz, DMSO, ppm): δ2.41 (s, 3H), 2.90-2.96 (d, 3H), 3.10 (s, 3H), 3.81 (s, 3H), 7.29-7.39 (m, 2H), 8.07-8.09 (d, 1H), 8.21 (s, 1H) 8.25 (d, 1H), 8.37 (s, 1H), 8.42 (s, 1H), 9.83 (s, 1H).

### Step 1: N-(4-bromo-2-methylphenyl)-3-methoxy-N-methylpyridine-4-carboxamide

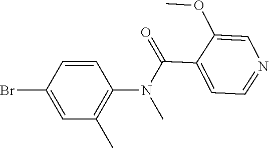 [SMILES: COc1cnccc1C(=O)N(C)c1ccc(Br)cc1C]

Proceeding analogously as described in Example 161, Step 1 but substituting N-(4-bromo-2-methylphenyl)-2-methylpyridine-4-carboxamide with N-(4-bromo-2-methylphenyl)-3-methoxypyridine-4-carboxamide (1000 mg, 3.11 mmol, 1 equiv.) in DMF (10.0 mL) was added NaH (124.5 mg, 3.11 mmol, 1.000 equiv., 60%) in portions at 0° C. with stirring for 30 min under nitrogen atmosphere. Then Mel (441.9 mg, 3.11 mmol, 1 equiv.) was added dropwise at 0° C. The resulting mixture was stirred for 1 h at room temperature under nitrogen atmosphere. The reaction was quenched by the addition of 10 mL of NH₄Cl (aq.) at 0° C. The resulting mixture was extracted with EA. The combined organic layers were washed with water. After filtration, the filtrate was concentrated under reduced pressure to afford (1.01 g, 96.77%) as a white solid.

### Step 2: 3-methoxy-N-methyl-N-[2-methyl-4-(tetramethyl-1,3,2-dioxaborolan-2-yl)phenyl]pyridine-4-carboxamide

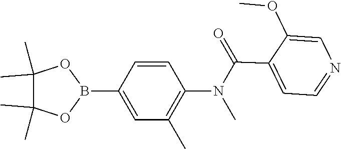 [SMILES: COc1cnccc1C(=O)N(C)c1ccc(B2OC(C)(C)C(C)(C)O2)cc1C]

Proceeding analogously as described in Example 161, Step 2 but substituting N-(4-bromo-2-methylphenyl)-N,2-dimethylpyridine-4-carboxamide with N-(4-bromo-2-methylphenyl)-3-methoxy-N-methylpyridine-4-carboxamide provided crude product. Purification by silica gel column chromatography by eluting with PE/EA (10:1) afforded the title compound as a white solid in 76.07% yield.

### Example 184: 3-methoxy-N-(2-methyl-4-(8-methyl-6-(trifluoromethyl)pyrido[3,2-d]pyrimidin-2-yl)phenyl)isonicotinamide

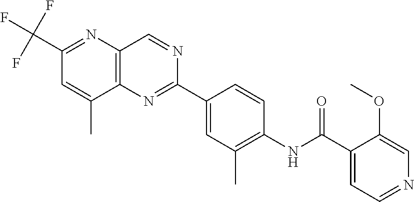 [SMILES: COc1cnccc1C(=O)Nc1ccc(-c2ncc3nc(C(F)(F)F)cc(C)c3n2)cc1C]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 3-methoxy-N-[2-methyl-4-(tetramethyl-1,3,2-dioxaborolan-2-yl)phenyl]pyridine-4-carboxamide provided crude product. Purification by Prep-TLC (CH₂Cl₂/MeOH=20:1) gave the title compound in 27.09%. LC-MS: (ES, m/z): [M+H]⁺ 454; ¹H-NMR: (300 MHz, DMSO, ppm): δ 2.95 (s, 3H), 4.14 (s, 3H), 7.78-7.89 (s, 1H), 8.21-8.23 (s, 1H), 8.38 (s, 1H), 8.42-8.43 (d, 1H), 8.51-8.56 (m, 1H), 8.69 (s, 1H), 9.86 (s, 1H), 10.08 (s, 1H)

### Step 1: N-(4-bromo-2-methylphenyl)-3-methoxypyridine-4-carboxamide

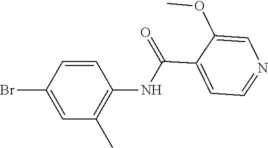 [SMILES: COc1cnccc1C(=O)Nc1ccc(Br)cc1C]

Proceeding analogously as described in Example 160, Step 8 but substituting 3-methylpyridine-4-carboxylic acid with 3-methoxypyridine-4-carboxylic acid provided the title compound as a solid in 90.59% yield.

### Step 2: 3-methoxy-N-[2-methyl-4-(tetramethyl-1,3,2-dioxaborolan-2-yl)phenyl]pyridine-4-carboxamide

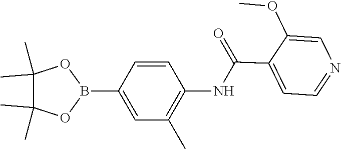 [SMILES: COc1cnccc1C(=O)Nc1ccc(B2OC(C)(C)C(C)(C)O2)cc1C]

Proceeding analogously as described in Example 161, Step 2 but substituting N-(4-bromo-2-methylphenyl)-N,2-dimethylpyridine-4-carboxamide with N-(4-bromo-2-methylphenyl)-3-methoxypyridine-4-carboxamide provided crude product. Purification by silica gel column chromatography by eluting with PE/EtOAc (10:1) provided the title compound as a white solid in 81.99% yield.

### Example 185: N-(2-fluoro-4-(8-methyl-6-(trifluoromethyl)pyrido[3,2-d]pyrimidin-2-yl)-phenyl)morpholine-4-carboxamide

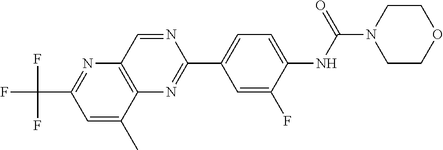 [SMILES: Cc1cc(C(F)(F)F)nc2cnc(-c3ccc(NC(=O)N4CCOCC4)c(F)c3)nc12]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-[2-fluoro-4-(tetramethyl-1,3,2-dioxaborolan-2-yl)phenyl]morpholine-4-carboxamide provided crude product. Purification by Prep-TLC with ethyl acetate/petroleum ether (1:2) provided the title compound as a yellow solid in 36.45% yield.

### Step 1: N-(4-bromo-2-fluorophenyl)morpholine-4-carboxamide

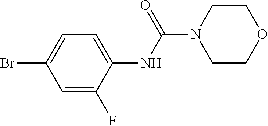 [SMILES: O=C(Nc1ccc(Br)cc1F)N1CCOCC1]

Into a 100-mL 3-necked round-bottom flask purged and maintained with an inert atmosphere of nitrogen, was placed 4-bromo-2-fluoroaniline (3 g, 15.79 mmol, 1 equiv.), DCM (30 mL), TRIPHOS (1.5 g, 5.21 mmol, 0.33 equiv.), Et₃N (4.8 g, 47.37 mmol, 3 equiv.), and morpholine (1.4 g, 15.79 mmol, 1 equiv.). The resulting solution was stirred overnight at room temperature. The crude product was purified by re-crystallization from EA/PE (1:5) to give the title compound as a white solid in 53.70% yield.

### Step 2: N-[2-fluoro-4-(tetramethyl-1,3,2-dioxaborolan-2-yl)phenyl]morpholine-4-carboxamide

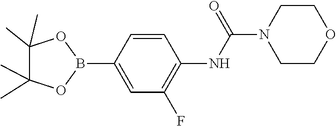 [SMILES: CC1(C)OB(c2ccc(NC(=O)N3CCOCC3)c(F)c2)OC1(C)C]

Proceeding analogously as described in Example 161, Step 2 but substituting N-(4-bromo-2-methylphenyl)-N,2-dimethylpyridine-4-carboxamide with N-(4-bromo-2-fluorophenyl)morpholine-4-carboxamide gave crude product. Purification by column chromatograph with ethyl acetate/petroleum ether as eluent, followed by re-crystallization from EA/PE (1:5) provided the title compound as a white solid in 35.37% yield. LC-MS: (ES, m/z): [M+H]⁺ 436; ¹H-NMR: (300 MHz, DMSO, ppm): δ 2.92 (s, 3H), 3.47-3.49 (d, 4H), 3.62-3.63 (d, 4H), 7.81-7.85 (t, 1H), 8.32-8.41 (m, 3H), 8.64 (s, 1H), 9.85 (s, 1H).

### Example 186: N-(2-fluoro-4-(8-methyl-6-(trifluoromethyl)pyrido[3,2-d]pyrimidin-2-yl)phenyl)tetrahydro-2H-pyran-4-carboxamide

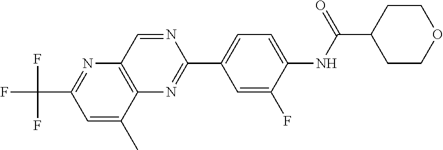 [SMILES: Cc1cc(C(F)(F)F)nc2cnc(-c3ccc(NC(=O)C4CCOCC4)c(F)c3)nc12]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with N-[2-fluoro-4-(tetramethyl-1,3,2-dioxaborolan-2-yl)phenyl]oxane-4-carboxamide gave crude product. Purification by Prep-HPLC (2#SHIMADZU (HPLC-01)): Column, Xselect CSH OBD Column 30*150 mm 5 um, n; mobile phase, water (0.1% FA) and ACN (59% Phase B up to 69% in 7 min); Detector, UV) provided the title compound as a white solid in 6.50% yield. LC-MS: (ES, m/z): [M+H]⁺ 435; ¹H-NMR: (300 MHz, DMSO, ppm): δ 1.69-1.73 (m, 4H), 2.87 (s, 1H), 2.92 (s, 3H), 3.33-3.40 (m, 2H), 3.91-3.94 (t, 2H), 8.27-8.31 (t, 1H), 8.35-8.36 (d, 2H), 8.38-8.44 (m, 1H), 9.86 (s, 1H), 10.00 (s, 1H).

### Step 1: N-(4-bromo-2-fluorophenyl)oxane-4-carboxamide

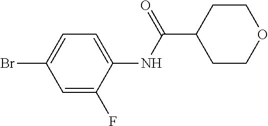 [SMILES: O=C(Nc1ccc(Br)cc1F)C1CCOCC1]

Proceeding analogously as described in Example 160, Step 8 but substituting 4-bromo-2-methylaniline with 4-bromo-2-fluoroaniline and 3-methylpyridine-4-carboxylic acid with oxane-4-carboxylic acid gave crude product. Purification by column chromatograph with ethyl acetate/petroleum ether (1:20) gave the title compound as a white solid in 48.21% yield.

### Step 2: N-(2-fluoro-4-(4,4,5,5-tetramethyl-1,3,2-dioxaborolan-2-yl)phenyl)tetrahydro-2H-pyran-4-carboxamide

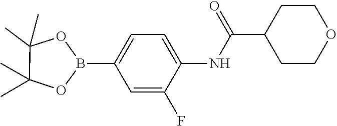 [SMILES: CC1(C)OB(c2ccc(NC(=O)C3CCOCC3)c(F)c2)OC1(C)C]

Proceeding analogously as described in Example 161, Step 2 but substituting N-(4-bromo-2-methylphenyl)-N,2-dimethylpyridine-4-carboxamide with N-(4-bromo-2-fluorophenyl)oxane-4-carboxamide provided crude product. Purification by column chromatography with ethyl acetate/petroleum ether (1:5), followed by re-crystallization from EA/PE (1:5) gave the title compound as a white solid in 48.15% yield.

### Example 187: N,1-dimethyl-N-(3-methyl-4-(6-(trifluoromethyl)pyrido[3,2-d]pyrimidin-2-yl)phenyl)-1H-pyrazole-5-carboxamide

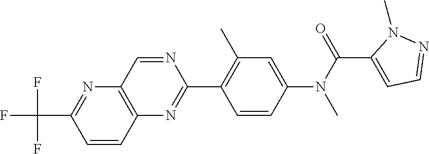 [SMILES: Cc1cc(N(C)C(=O)c2ccnn2C)ccc1-c1ncc2nc(C(F)(F)F)ccc2n1]

Proceeding analogously as described in Example 161, Step 1 but substituting N-(4-bromo-2-methylphenyl)-2-methylpyridine-4-carboxamide with 1-methyl-N-[3-methyl-4-[6-(trifluoromethyl)pyrido[3,2-d]pyrimidin-2-yl]phenyl]-1H-pyrazole-5-carboxamide provided crude product. Purification by prep-TLC with ethyl acetate/petroleum ether (1:2) as eluent gave the title compound as a white solid in 29.36% yield. LC-MS: (ES, m/z): [M+H]⁺ 427; ¹H-NMR: (300 MHz, CDCl₃, ppm): δ 2.01 (s, 1H), 2.66 (s, 3H), 3.51 (s, 3H), 4.15 (s, 3H), 5.70 (d, 1H), 7.10-7.12 (t, 2H), 7.17-7.18 (d, 1H), 8.07-8.10 (m, 1H), 8.15-8.18 (d, 1H), 8.57-8.60 (d, 1H), 9.84 (d, 1H).

### Step 1: 2-(2-methyl-4-nitrophenyl)-6-(trifluoromethyl)pyrido[3,2-d]pyrimidine

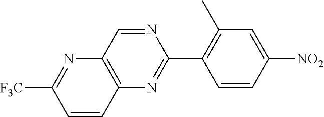 [SMILES: Cc1cc([N+](=O)[O-])ccc1-c1ncc2nc(C(F)(F)F)ccc2n1]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 4,4,5,5-tetramethyl-2-(2-methyl-4-nitrophenyl)-1,3,2-dioxaborolane gave crude product. Purification by Prep-TLC (PE/EA=5:1) gave the title compound as a yellow solid in 20.96% yield.

### Step 2: 3-methyl-4-(6-(trifluoromethyl)pyrido[3,2-d]pyrimidin-2-yl)aniline

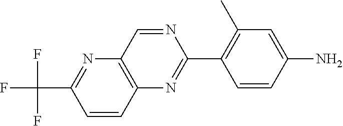 [SMILES: Cc1cc(N)ccc1-c1ncc2nc(C(F)(F)F)ccc2n1]

Into an 8-mL sealed tube, was placed 2-(2-methyl-4-nitrophenyl)-6-(trifluoro-methyl)pyrido[3,2-d]pyrimidine (180 mg, 0.54 mmol, 1 equiv.), Fe (90.2 mg, 1.62 mmol, 2.999 equiv.), NH₄Cl (144.0 mg, 2.69 mmol, 5 equiv.), and H₂O (1 mL, 55.51 mmol, 103.077 equiv.). The resulting solution was stirred for 2 h at 80° C. and then extracted with ethyl acetate. The residue was applied onto a prep-TLC and eluted with ethyl acetate/petroleum ether (1:3) to give the tile compound as a yellow solid in 67.13% yield.

### Step 3: 1-methyl-N-[3-methyl-4-[6-(trifluoromethyl)pyrido[3,2-d]pyrimidin-2-yl]phenyl]-1H-pyrazole-5-carboxamide

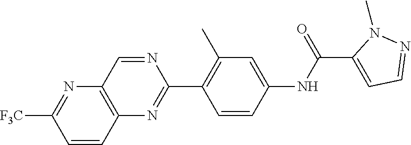 [SMILES: Cc1cc(NC(=O)c2ccnn2C)ccc1-c1ncc2nc(C(F)(F)F)ccc2n1]

Proceeding analogously as described in Example 160, Step 8 but substituting 4-bromo-2-methylaniline with 3-methyl-4-(6-(trifluoromethyl)pyrido[3,2-d]pyrimidin-2-yl)aniline (70 mg, 0.23 mmol, 1 equiv.) and 3-methylpyridine-4-carboxylic acid with 1-methyl-1H-pyrazole-5-carboxylic provided crude product. Purification by prep-TLC with ethyl acetate/petroleum ether (1:2) provided the title compound as a white solid in 59.03% yield.

### Example 188: N-(2,5-dimethyl-4-(6-(trifluoromethyl)pyrido[3,2-d]pyrimidin-2-yl)phenyl)-N,1-dimethyl-1H-pyrazole-5-carboxamide

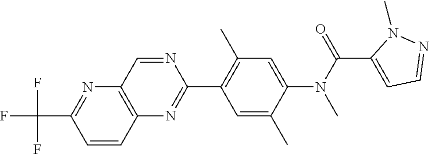 [SMILES: Cc1cc(N(C)C(=O)c2ccnn2C)c(C)cc1-c1ncc2nc(C(F)(F)F)ccc2n1]

Proceeding analogously as described in Example 161, Step 1 but substituting N-(4-bromo-2-methylphenyl)-2-methylpyridine-4-carboxamide with N-[2,5-dimethyl-4-[6-(trifluoromethyl)pyrido[3,2-d]pyrimidin-2-yl]phenyl]-1-methyl-1H-pyrazole-5-carboxamide provide crude product. Purification by Prep. TLC with ethyl acetate/petroleum ether (1/2) gave the title compound as a yellow solid in 40.95% yield. LC-MS: (ES, m/z): [M+H]⁺ 441; ¹H-NMR: (400 MHz, DMSO, ppm): δ2.17 (s, 3H), δ2.57 (s, 3H), δ3.33 (s, 3H), δ4.01 (s, 3H), δ5.57-5.58 (d, 1H), δ7.19-7.20 (d, 1H), δ7.37 (s, 1H), δ7.93 (s, 1H), δ8.48-8.50 (d, 1H), δ8.80-8.82 (d, 1H), δ9.95 (s, 1H).

### Example 189: N-(2,5-dimethyl-4-(6-(trifluoromethyl)pyrido[3,2-d]pyrimidin-2-yl)phenyl)-1-methyl-1H-pyrazole-5-carboxamide

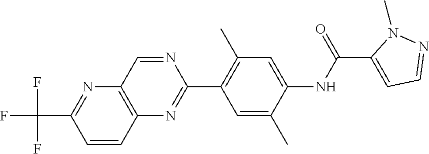 [SMILES: Cc1cc(-c2ncc3nc(C(F)(F)F)ccc3n2)c(C)cc1NC(=O)c1ccnn1C]

Proceeding analogously as described in Example 160, Step 8 but substituting 4-bromo-2-methylaniline with 2,5-dimethyl-4-[6-(trifluoromethyl)pyrido[3,2-d]pyrimidin-2-yl]aniline (70 mg, 0.23 mmol, 1 equiv.) and 3-methylpyridine-4-carboxylic acid with 1-methyl-1H-pyrazole-5-carboxylic acid gave crude product. Purification by Prep. TLC with ethyl acetate/petroleum ether (1/2) gave the title compound as a yellow solid in 93.31% yield. LC-MS: (ES, m/z): [M+H]⁺ 427; ¹H-NMR: (400 MHz, DMSO, ppm): δ 2.32 (s, 3H), 2.64 (s, 3H), 4.11 (s, 3H), 7.10-7.11 (d, 1H), 7.43 (s, 1H), 7.55-7.56 (d, 1H), 8.02 (s, 1H), 8.47-8.49 (d, 1H), 8.80-8.82 (d, 1H), 9.96-9.98 (d, 2H).

### Example 190: N-(2,3-dimethyl-4-(6-(trifluoromethyl)pyrido[3,2-d]pyrimidin-2-yl)phenyl)-1-methyl-1H-pyrazole-5-carboxamide

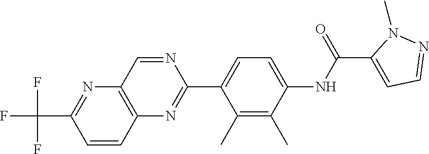 [SMILES: Cc1c(NC(=O)c2ccnn2C)ccc(-c2ncc3nc(C(F)(F)F)ccc3n2)c1C]

To a stirred solution of 2,3-dimethyl-4-[6-(trifluoromethyl)pyrido[3,2-d]pyrimidin-2-yl]aniline (120 mg, 0.38 mmol, 1 equiv.) and 1-methyl-1H-pyrazole-5-carboxylic acid (52.3 mg, 0.41 mmol, 1.1 equiv.) in DMF (1.2 mL) were added DIEA (97.5 mg, 0.75 mmol, 2.00 equiv.) and HATU (215.0 mg, 0.57 mmol, 1.5 equiv.) at room temperature. The resulting mixture was stirred for overnight at 40° C. under nitrogen atmosphere and then quenched with water at room temperature. The resulting mixture was extracted with EA and the combined organic layer were washed with NaCl(aq.). After concentrating the organic layer, the residue was purified by Prep. TLC with ethyl acetate/hexane. The resulting material was further purified by HPLC under following conditions: Column, C18 silica gel; mobile phase, MeCN: NH₄HCO₃(aq.)=0% increasing to MeCN:NH₄HCO₃(aq.)=50% within 20 min; Detector, 254 nm to give the title compound as a yellow solid in 34.84% yield. LC-MS: (ES, m/z): [M+H]⁺ 427; ¹H-NMR: (300 MHz, CDCL₃, ppm): δ 2.35 (s, 3H), 2.54 (s, 3H), 4.26 (s, 3H), 6.70-6.71 (d, 1H), 7.54-7.55 (d, 1H), 7.67 (s, 1H), 7.80-7.82 (d, 1H), 7.87-7.92 (m, 1H), 8.15-8.18 (d, 1H), 8.59-8.62 (d, 1H), 9.86 (s, 1H).

### Example 191: N-(2,3-dimethyl-4-(6-(trifluoromethyl)pyrido[3,2-d]pyrimidin-2-yl)phenyl)-N,1-dimethyl-1H-pyrazole-5-carboxamide

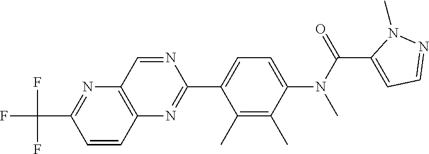 [SMILES: Cc1c(-c2ncc3nc(C(F)(F)F)ccc3n2)ccc(N(C)C(=O)c2ccnn2C)c1C]

Proceeding analogously as described in Example 161, Step 1 but substituting N-(4-bromo-2-methylphenyl)-2-methylpyridine-4-carboxamide with N-(2,3-dimethyl-4-(6-(trifluoromethyl)pyrido[3,2-d]pyrimidin-2-yl)phenyl)-1-methyl-1H-pyrazole-5-carboxamide provided crude product. Purification by Prep.TLC (hexane/EA=5:1) gave the title compound as a white solid in 56.92% yield. LC-MS: (ES, m/z): [M+H]⁺ 441; ¹H-NMR: (300 MHz, DMSO, ppm): δ2.20 (s, 3H), 2.37 (s, 3H), 3.31 (s, 3H), 4.00 (s, 3H), 5.56-5.57 (d, 1H), 7.19-7.20 (d, 1H), 7.28-7.31 (d, 1H), 7.62-7.64 (d, 1H), 8.49-8.52 (d, 1H), 8.79-8.82 (d, 1H), 9.96 (d, 1H).

### Example 192: 1-(2-hydroxypropyl)-N-methyl-N-(2-methyl-4-(8-methyl-6-(trifluoromethyl)pyrido[3,2-d]pyrimidin-2-yl)phenyl)-1H-pyrazole-5-carboxamide

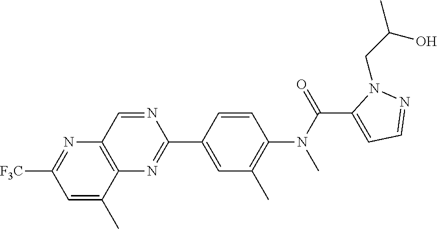 [SMILES: Cc1cc(-c2ncc3nc(C(F)(F)F)cc(C)c3n2)ccc1N(C)C(=O)c1ccnn1CC(C)O]

Proceeding analogously as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-(2-hydroxypropyl)-N-methyl-N-(2-methyl-4-(4,4,5,5-tetramethyl-1,3,2-dioxaborolan-2-yl)phenyl)-1H-pyrazole-5-carboxamide provided crude product. Purification by Prep.TLC (CH₂Cl₂/MeOH=20:1) gave the title compound as a white solid in 24.24% yield. LC-MS: (ES, m/z): [M+H]⁺ 485; ¹H-NMR: (400 MHz, DMSO, ppm): δ1.02-1.12 (d, 3H), 2.35 (s, 1H), 2.42 (s, 2H), 2.92 (s, 3H), 3.31 (s, 2H), 3.36 (s, 1H), 4.01-4.16 (m, 2H), 4.17-4.21 (m, 1H), 4.37-4.53 (m, 1H), 4.96-5.01 (m, 1H), 5.52-5.54 (m, 1H), 7.17 (s, 1H), 7.43-7.59 (m, 1H), 8.35-8.43 (m, 2H), 8.54-8.57 (d, 2H), 9.88 (s, 1H)

### Step 1: N-methyl-N-(2-methyl-4-(4,4,5,5-tetramethyl-1,3,2-dioxaborolan-2-yl)phenyl)-1-(2-oxopropyl)-1H-pyrazole-5-carboxamide

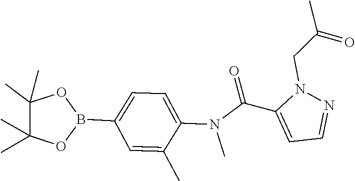 [SMILES: CC(=O)Cn1nccc1C(=O)N(C)c1ccc(B2OC(C)(C)C(C)(C)O2)cc1C]

Into an 8 mL sealed tube were added N-(4-bromo-2-methylphenyl)-N-methyl-1-(2-oxopropyl)-1H-pyrazole-5-carboxamide (200 mg, 0.57 mmol, 1 equiv.), XPhos Pd G₃ (48.3 mg, 0.06 mmol, 0.1 equiv.), KOAc (112.1 mg, 1.14 mmol, 2 equiv.), B₂Pin₂(159.6 mg, 0.63 mmol, 1.1 equiv.) and 1,4-dioxane (2 mL) under nitrogen atmosphere. The resulting mixture was stirred overnight at 80° C. under nitrogen atmosphere and then concentrated under reduced pressure. The residue was purified by silica gel column (PE/EA=2:1) to afford the title compound as a yellow solid in 88.15% yield.

### Step 2: 1-(2-hydroxypropyl)-N-methyl-N-(2-methyl-4-(4,4,5,5-tetramethyl-1,3,2-dioxaborolan-2-yl)phenyl)-1H-pyrazole-5-carboxamide

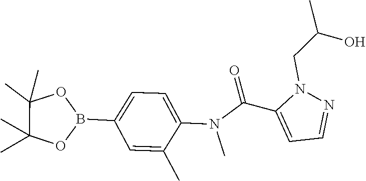 [SMILES: Cc1cc(B2OC(C)(C)C(C)(C)O2)ccc1N(C)C(=O)c1ccnn1CC(C)O]

To a stirred mixture of N-methyl-N-(2-methyl-4-(4,4,5,5-tetramethyl-1,3,2-dioxaborolan-2-yl)phenyl)-1-(2-oxopropyl)-1H-pyrazole-5-carboxamide (200 mg, 0.50 mmol, 1 equiv.) in MeOH (2 mL) was added NaBH₄ (19.0 mg, 0.50 mmol, 1.00 equiv.) and the resulting mixture was stirred for 2 h at room temperature. The reaction mixture was quenched with NH₄Cl (aq.) and extracted with EA. The combined organic layers were concentrated under reduced pressure and the residue was purified by silica gel column (PE/EA=2:1) to afford the title compound as a white solid in 59.70% yield.

### Example 193: 1-(2-hydroxypropyl)-N-(2-methyl-4-(6-(trifluoromethyl)pyrido[3,2-d]pyrimidin-2-yl)phenyl)-1H-pyrazole-5-carboxamide

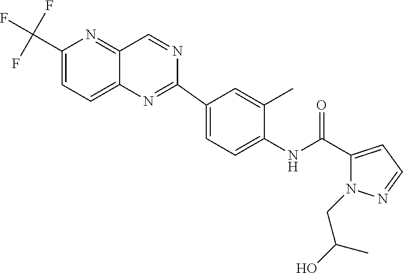 [SMILES: Cc1cc(-c2ncc3nc(C(F)(F)F)ccc3n2)ccc1NC(=O)c1ccnn1CC(C)O]

Into a 8-mL sealed tube purged and maintained with an inert atmosphere of nitrogen, was placed 1-(2-hydroxypropyl)-N-[2-methyl-4-(4,4,5,5-tetramethyl-1,3,2-dioxaborolan-2-yl)phenyl]-1H-pyrazole-5-carboxamide (100 mg, 0.26 mmol, 1 equiv.), 2-chloro-6-(trifluoromethyl)pyrido[3,2-d]pyrimidine (60.6 mg, 0.26 mmol, 1 equiv.), K₂CO₃ (107.6 mg, 0.78 mmol, 3 equiv.), t-BuOH (0.9 mL), H₂O (0.1 mL), and AmphosPdCl₂ (55.0 mg, 0.08 mmol, 0.3 equiv.). The resulting solution was stirred for 12 min at 80° C. and then quenched with water. The resulting solution was extracted with ethyl acetate and after removal of the organics, the residue was applied onto Prep-TLC with ethyl acetate/petroleum ether (1:3) to give the title compound as a yellow solid in 19.92% yield. LC-MS: (ES, m/z): [M+H]⁺ 457; ¹H-NMR: (300 MHz, DMSO, ppm): δ1.01-1.03 (d, 3H), 1.23 (s, 1H), 2.80-2.88 (d, 3H), 2.40 (s, 3H), 3.98-4.04 (m, 1H), 4.39-4.40 (m, 1H), 4.42-4.44 (m, 1H), 4.90-4.94 (d, 1H), 7.04-7.05 (d, 1H), 7.57-7.59 (d, 1H), 7.66-7.68 (d, 1H), 8.45-8.49 (d, 1H), 8.54 (s, 1H), 8.78-8.81 (d, 1H), 9.81 (s, 1H), 10.08 (s, 1H).

### Step 1: methyl 1-(2-oxopropyl)-1H-pyrazole-5-carboxylate

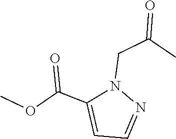

Into a 250-mL 3-necked round-bottom flask purged and maintained with an inert atmosphere of nitrogen, was placed methyl 1H-pyrazole-5-carboxylate (10 g, 79.29 mmol, 1 equiv.), 1-chloropropan-2-one (8.1 g, 87.22 mmol, 1.1 equiv.), K₂CO₃ (32.9 g, 237.88 mmol, 3 equiv.), and ACN (100 mL). The resulting solution was stirred for 12 h at 80° C. and then concentrated. After adding water, the resulting solution was extracted with ethyl acetate. The residue was applied onto a silica gel column and eluted with ethyl acetate/petroleum ether (1:20) to give the title compound as a yellow solid in 22.84% yield.

### Step 2: 1-(2-oxopropyl)-1H-pyrazole-5-carboxylic acid

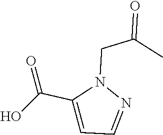

Into a 100-mL 3-necked round-bottom flask purged and maintained with an inert atmosphere of nitrogen, was placed methyl 1-(2-oxopropyl)-1H-pyrazole-5-carboxylate (3.3 g, 18.11 mmol, 1 equiv.), THE (33 mL), LiOH.H₂O (0.8 g, 18.11 mmol, 1 equiv.), and H₂O (20 mL). The resulting solution was stirred for 1 h at 25° C. and the pH of the solution was adjusted to 6 with HCl. The resulting mixture was concentrated and the residue was applied onto a silica gel column and eluted with dichloromethane/methanol (20:1) to give the title compound as a yellow solid in 91.93% yield.

### Step 3: N-(4-bromo-2-methylphenyl)-1-(2-oxopropyl)-1H-pyrazole-5-carboxamide

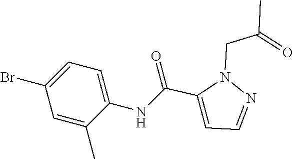 [SMILES: CC(=O)Cn1nccc1C(=O)Nc1ccc(Br)cc1C]

Proceeding analogously as described in Example 160, Step 8 but substituting 2-methylpyridine-4-carboxylic acid with 1-(2-oxopropyl)-1H-pyrazole-5-carboxylic acid provided crude product. The crude product was applied onto a silica gel column and eluted with ethyl acetate/petroleum ether (1:20) to give the title compound as a yellow solid in 17.86% yield.

### Step 4: N-(4-bromo-2-methylphenyl)-1-(2-hydroxypropyl)-1H-pyrazole-5-carboxamide

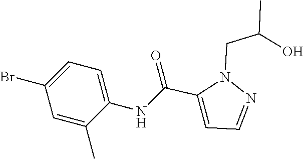 [SMILES: Cc1cc(Br)ccc1NC(=O)c1ccnn1CC(C)O]

Proceeding analogously as described in Example 192, Step 2 but substituting N-methyl-N-(2-methyl-4-(4,4,5,5-tetramethyl-1,3,2-dioxaborolan-2-yl)phenyl)-1-(2-oxopropyl)-1H-pyrazole-5-carboxamide with N-(4-bromo-2-methylphenyl)-1-(2-oxopropyl)-1H-pyrazole-5-carboxamide provided crude product. The crude product was applied onto a silica gel column and eluted with CH₂C2 give the title compound as a white solid in 89.46% yield.

### Step 5: 1-(2-hydroxypropyl)-N-[2-methyl-4-(4,4,5,5-tetramethyl-1,3,2-dioxaborolan-2-yl)phenyl]-1H-pyrazole-5-carboxamide

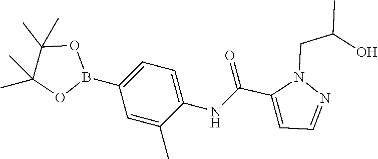 [SMILES: Cc1cc(B2OC(C)(C)C(C)(C)O2)ccc1NC(=O)c1ccnn1CC(C)O]

Proceeding as described in Example 161, Step 2 but substituting N-(4-bromo-2-methylphenyl)-N,2-dimethylpyridine-4-carboxamide with N-(4-bromo-2-methylphenyl)-1-(2-hydroxypropyl)-1H-pyrazole-5-carboxamide (0.9 g, 2.66 mmol, 1 equiv.) provided crude product. The crude product was applied onto a silica gel column and eluted with ethyl acetate/petroleum ether (1:10) to give the title compound as a yellow solid in 78.03% Yield.

### Example 194: 2-[5-[methyl([2-methyl-4-[8-methyl-6-(trifluoromethyl)pyrido[3,2-d]pyrimidin-2-yl]phenyl])carbamoyl]-1H-pyrazol-1-yl]ethyl disodium phosphate

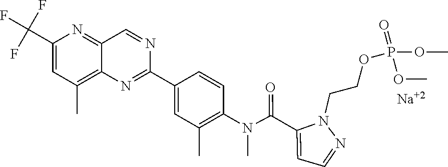

Into a 25 mL round-bottom flask were added (2-[5-[methyl([2-methyl-4-[8-methyl-6-(trifluoromethyl)pyrido[3,2-d]pyrimidin-2-yl]phenyl])carbamoyl]-1H-pyrazol-1-yl]ethoxy)phosphonic acid (11.7 mg, 0.02 mmol, 1 equiv.) and NaOH (0.04 mL, 1M, 2.00 equiv.). After lyophilization, the title compound was obtained in 98.94% yield as a yellow solid. LC-MS: (ES, m/z): 551; ¹H-NMR: (300 MHz, D₂O, ppm): δ 2.24-2.30 (m, 3H), 2.60-2.70 (m, 3H), 3.3.37-3.42 (m, 3H), 3.95-4.02 (m, 2H), 4.40-4.53 (m, 2H), 5.76-5.77 (d, 1H), 6.78 (s, 1H), 7.18 (s, 1H), 7.35-7.38 (m, 1H), 7.86-8.22 (m, 3H), 8.25-9.39 (m, 1H).

### Step 1: (2-[5-[methyl([2-methyl-4-[8-methyl-6-(trifluoromethyl)pyrido[3,2-d]pyrimidin-2-yl]phenyl])carbamoyl]-1H-pyrazol-1-yl]ethoxy)phosphonic acid

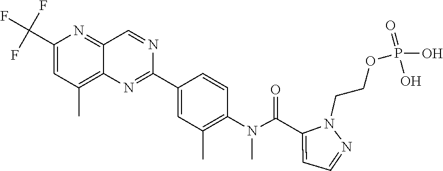 [SMILES: Cc1cc(-c2ncc3nc(C(F)(F)F)cc(C)c3n2)ccc1N(C)C(=O)c1ccnn1CCOP(=O)(O)O]

Into an 8 mL vial were added POCl₃ (163.0 mg, 1.06 mmol, 5 equiv.) and Et₃N (322.6 mg, 3.19 mmol, 15 equiv.) in CHCl₃ (2 mL). The resulting mixture was stirred for 10 min and 1-(2-hydroxyethyl)-N-methyl-N-[2-methyl-4-[8-methyl-6-(trifluoromethyl)pyrido-[3,2-d]pyrimidin-2-yl]phenyl]-1H-pyrazole-5-carboxamide (100 mg, 0.21 mmol, 1 equiv.) was added. The resulting mixture was stirred for 2 h at 0° C. and then with water. The resulting mixture was extracted with EtOAc and the combined organic layers were concentrated under reduced pressure. The crude product was purified by Prep-HPLC (Column: SunFire C18 OBD Prep Column, 100*5 um, 19 mm×250 mm; Mobile Phase A:Water (0.05% TFA), Mobile Phase B: ACN; Flow rate: 25 mL/min; Gradient: 48% B to 58% B in 8 min; 254/220 nm; Rt: 6.68 min) to afford the title compound as a yellow solid in 10.00% yield.

### Example 195: 1-(2-amino-2-oxoethyl)-5-(methyl(2-methyl-4-(8-methyl-6-(trifluoro-methyl)pyrido[3,2-d]pyrimidin-2-yl)phenyl)carbamoyl)-1H-pyrazole-3-carboxylic acid

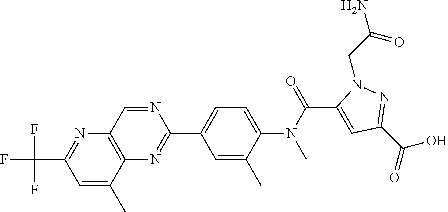 [SMILES: Cc1cc(-c2ncc3nc(C(F)(F)F)cc(C)c3n2)ccc1N(C)C(=O)c1cc(C(=O)O)nn1CC(N)=O]

Proceeding analogously as described in Example 195, Step 4 but substituting 1-(cyanomethyl)-1H-pyrazole-3,5-dicarboxylate with methyl 1-(cyanomethyl)-5-(methyl(2-methyl-4-(8-methyl-6-(trifluoromethyl)pyrido[3,2-d]pyrimidin-2-yl)phenyl)carbamoyl)-1H-pyrazole-3-carboxylate provided crude product. The crude product was purified by Prep-TLC (CH₂C₂/MeOH 30/1), followed by purification by reverse flash chromatography column, C18 silica gel; mobile phase, MeCN in water, 0% to 50% gradient in 30 min; detector, UV 254 nm) to give the title compound as a white solid in 3.76% yield. LC-MS: (ES, m/z): [M+H]⁺ 528; ¹H-NMR: (400 MHz, DMSO, ppm): (400 MHz, DMSO, ppm): δ 2.40 (s, 3H), 2.94 (s, 3H), 3.26 (s, 1H), 5.13-5.19 (m, 2H), 5.64 (s, 1H), 7.22-7.28 (m, 1H), 7.59-7.66 (m, 2H), 8.38 (s, 1H), 8.43-8.45 (d, 1H), 8.57 (s, 1H), 9.89 (s, 1H).

### Step 1: tert-butyl (4-bromo-2-methylphenyl)(methyl)carbamate

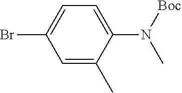 [SMILES: Cc1cc(Br)ccc1N(C)C(=O)OC(C)(C)C]

Proceeding analogously as described in Example 165, Step 2 but substituting N-(4-bromo-2-fluorophenyl)-2-methylisonicotinamide with tert-butyl N-(4-bromo-2-methylphenyl)carbamate (28 g, 97.85 mmol, 1.00 equiv.) provide crude product. The crude product was applied onto a silica gel column and eluted with ethyl acetate/petroleum ether (1/20) to give the title compound as a yellow oil in 92% yield.

### Step 2: 4-bromo-N,2-dimethylaniline

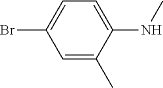

To a stirred solution of tert-butyl (4-bromo-2-methylphenyl)(methyl)carbamate (7.9 g, 26.32 mmol, 1 equiv.) in 1,4-dioxane was added 1,4-dioxane hydrochloride (40 mL) at room temperature. The resulting mixture was stirred overnight at room temperature under nitrogen atmosphere. The residue was basified to pH 8 with NaOH (aq.) and the resulting mixture was extracted with EtOAc. The combined organic layers were concentrated under reduced pressure and the residue was purified by silica gel column chromatography by eluting with PE/EtOAc (40/1) to give the title compound as a white solid in 98.76% yield.

### Step 3: dimethyl 1-(cyanomethyl)-1H-pyrazole-3,5-dicarboxylate

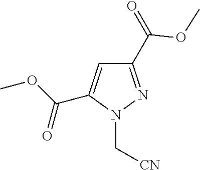

Into a 1-L 3-necked round-bottom flask purged and maintained with an inert atmosphere of nitrogen, was placed 3,5-dimethyl 1H-pyrazole-3,5-dicarboxylate (20 g, 108.61 mmol, 1 equiv.), K₂CO₃ (22.5 g, 162.91 mmol, 1.5 equiv.), CH₃CN (400 mL), and 2-bromoacetonitrile (14.3 g, 119.22 mmol, 1.10 equiv.). The resulting solution was stirred for 1 h at 85° C. The solids were filtered out and the filtrate was concentrated. The residue was applied onto a silica gel column and eluted with ethyl acetate/petroleum ether (1/5) to give the title compound as a yellow solid in 86.63% yield.

### Step 4: 1-(cyanomethyl)-3-(methoxycarbonyl)-1H-pyrazole-5-carboxylic acid

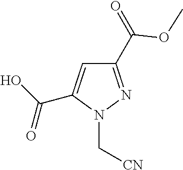

Proceeding analogously as described in Example 232, Step 4 but substituting methyl 1-(2-oxopropyl)-1H-pyrazole-5-carboylate with dimethyl 1-(cyanomethyl)-1H-pyrazole-3,5-dicarboxylate and stirring the reaction mixture overnight provided the title compound as a white solid in 87.78% yield.

### Step 5: methyl 5-(chlorocarbonyl)-1-(cyanomethyl)-1H-pyrazole-3-carboxylate

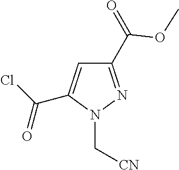

Proceeding analogously as described in Example 166, Step 1 but substituting 2-cyanopyridine-4-carboxylic acid with 1-(cyanomethyl)-3-(methoxycarbonyl)-1H-pyrazole-5-carboxylic acid and stirring the reaction mixture after addition of oxalyl chloride for 3h at room temperature provided the title compound as a yellow solid in 98.46% yield.

### Step 6: methyl 5-((4-bromo-2-methylphenyl)(methyl)carbamoyl)-1-(cyanomethyl)-1H-pyrazole-3-carboxylate

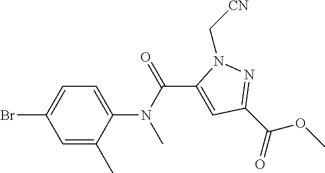 [SMILES: COC(=O)c1cc(C(=O)N(C)c2ccc(Br)cc2C)n(CC#N)n1]

To a stirred solution of 4-bromo-N,2-dimethylaniline (7.5 g, 37.49 mmol, 1 equiv.) and Et₃N (6.76 g, 66.81 mmol, 1.78 equiv.) in DCM (140 mL) were added methyl 5-(chlorocarbonyl)-1-(cyanomethyl)-1H-pyrazole-3-carboxylate (6.67 g, 29.31 mmol, 0.78 equiv.) dissolved in 50 mL of DCM dropwise at 0° C. The resulting mixture was stirred for 2 h at room temperature and then quenched with water. The resulting mixture was extracted with CH₂C₂ and the combined organic layers were concentrated under reduced pressure. The residue was purified by silica gel column chromatography, eluting with PE/EtOAc (5/1) to afford the title compound as a white solid in 32.73% yield.

### Step 7: methyl 1-(cyanomethyl)-5-(methyl(2-methyl-4-(4,4,5,5-tetramethyl-1,3,2-dioxaborolan-2-yl)phenyl)carbamoyl)-1H-pyrazole-3-carboxylate

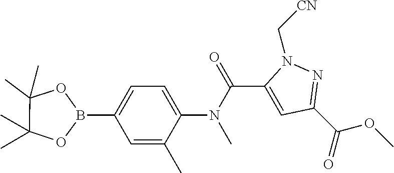

Proceeding as described in Example 161, Step 2 but substituting N-(4-bromo-2-methylphenyl)-N,2-dimethylpyridine-4-carboxamide with methyl 5-((4-bromo-2-methylphenyl)(methyl)carbamoyl)-1-(cyanomethyl)-1H-pyrazole-3-carboxylate provided crude product. The crude product was purified by silica gel column chromatography, eluting with PE/EtOAc (10/1) to afford the title compound in 74.38% yield as a yellow solid.

### Step 8: methyl 1-(cyanomethyl)-5-(methyl(2-methyl-4-(8-methyl-6-(trifluoro-methyl)pyrido[3,2-d]pyrimidin-2-yl)phenyl)carbamoyl)-1H-pyrazole-3-carboxylate

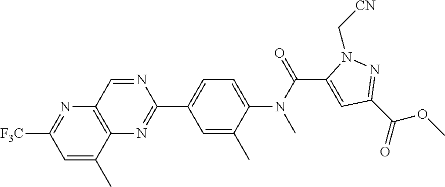 [SMILES: COC(=O)c1cc(C(=O)N(C)c2ccc(-c3ncc4nc(C(F)(F)F)cc(C)c4n3)cc2C)n(CC#N)n1]

Proceeding analogously as described in Example 193 but substituting 1-(2-hydroxypropyl)-N-[2-methyl-4-(4,4,5,5-tetramethyl-1,3,2-dioxaborolan-2-yl)phenyl]-1H-pyrazole-5-carboxamide with methyl 1-(cyanomethyl)-5-(methyl(2-methyl-4-(4,4,5,5-tetramethyl-1,3,2-dioxaborolan-2-yl)phenyl)carbamoyl)-1H-pyrazole-3-carboxylate and 2-chloro-6-(trifluoromethyl)pyrido[3,2-d]pyrimidine with 2-chloro-8-methyl-6-(trifluoromethyl)pyrido[3,2-d]pyrimidine provided crude product. The crude product was purified by Prep-TLC (PE/EtOAc 5/1) to afford the title compound as a yellow solid in 61.49% yield.

### Example 196: 1-(2-amino-2-oxoethyl)-N5-methyl-N5-(2-methyl-4-(8-methyl-6-(trifluoromethyl)pyrido[3,2-d]pyrimidin-2-yl)phenyl)-1H-pyrazole-3,5-dicarboxamide

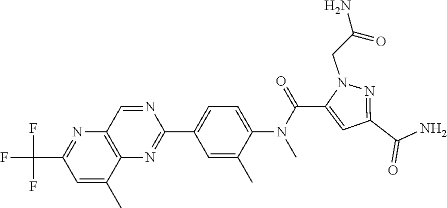 [SMILES: Cc1cc(-c2ncc3nc(C(F)(F)F)cc(C)c3n2)ccc1N(C)C(=O)c1cc(C(N)=O)nn1CC(N)=O]

Proceeding analogously as described in Example 167, Step 2 but substituting 4-[(4-bromo-2-methylphenyl)carbamoyl]pyridine-2-carboxylic acid with 1-(carbamoylmethyl)-5-[methyl([2-methyl-4-[8-methyl-6-(trifluoromethyl)pyrido[3,2-d]pyrimidin-2-yl]phenyl])carbamoyl]-1H-pyrazole-3-carboxylic acid and methamine hydrochloride with NH₄Cl provided crude product. The crude product was purified by Prep-TLC (CH₂Cl₂/MeOH 40/1), followed by reverse flash chromatography (column, C18 silica gel; mobile phase, MeCN in water, 0% to 55% gradient in 30 min; detector, UV 254 nm) to provide the title compound as a yellow solid in 25.58% yield. LC-MS: (ES, m/z): [M+H]⁺ 527; ¹H-NMR: (400 MHz, DMSO, ppm): δ2.42 (s, 3H), δ2.94 (s, 3H), δ3.29 (s, 3H), δ5.13-5.25 (m, 2H), δ5.79 (s, 1H), δ7.21 (s, 1H), δ7.32 (s, 1H), δ7.47 (s, 1H), δ7.60-7.62 (d, 1H), δ7.67 (s, 1H), δ8.38 (s, 1H), δ8.43-8.46 (d, 1H), δ8.62 (s, 1H), δ9.88 (s, 1H).

### Example 197: 1-(cyanomethyl)-5-(methyl(2-methyl-4-(8-methyl-6-(trifluoromethyl)-pyrido[3,2-d]pyrimidin-2-yl)phenyl)carbamoyl)-1H-pyrazole-3-carboxylic acid

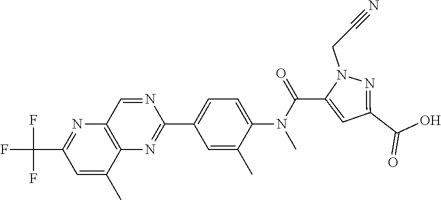 [SMILES: Cc1cc(-c2ncc3nc(C(F)(F)F)cc(C)c3n2)ccc1N(C)C(=O)c1cc(C(=O)O)nn1CC#N]

Proceeding analogously as described in Example 195, Step 4 but substituting N-(4-bromo-2-methylphenyl)-1-(2-oxopropyl)-1H-pyrazole-5-carboxamide with methyl 1-(cyanomethyl)-5-[methyl([2-methyl-4-[8-methyl-6-(trifluoromethyl)pyrido[3,2-d]pyrimidin-2-yl]phenyl])carbamoyl]-1H-pyrazole-3-carboxylate provided crude product. The crude product was purified by Prep-TLC (CH₂Cl₂/MeOH 30/1), followed by reverse flash chromatography column, C18 silica gel; mobile phase, MeCN in water, 0% to 40% gradient in 25 min; detector, UV 254 nm) to give the title compound as a white solid in 5.76% yield. LC-MS: (ES, m/z): [M+H]⁺ 510; ¹H-NMR: (400 MHz, DMSO, ppm): δ 9.75 (s, 1H), 8.72 (s, 1H), 8.63 (d, 1H), 8.20 (s, 1H), 6.01 (s, 1H), 5.68 (q, 2H), 3.47 (s, 3H), 3.00 (s, 3H), 2.42 (s, 3H)

### Example 198: 1-(cyanomethyl)-N5-methyl-N5-(2-methyl-4-(8-methyl-6-(trifluoro-methyl)pyrido[3,2-d]pyrimidin-2-yl)phenyl)-1H-pyrazole-3,5-dicarboxamide

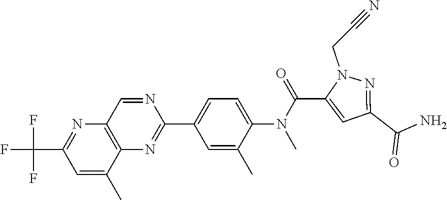 [SMILES: Cc1cc(-c2ncc3nc(C(F)(F)F)cc(C)c3n2)ccc1N(C)C(=O)c1cc(C(N)=O)nn1CC#N]

Proceeding analogously as described in Example 167, Step 2 but substituting 4-[(4-bromo-2-methylphenyl)carbamoyl]pyridine-2-carboxylic acid with 1-(cyanomethyl)-5-[methyl([2-methyl-4-[8-methyl-6-(trifluoromethyl)pyrido[3,2-d]pyrimidin-2-yl]phenyl])carbamoyl]-1H-pyrazole-3-carboxylic acid provided crude product. The crude product was purified by Prep-TLC (CH₂Cl₂/MeOH 40/1), followed by reverse flash chromatography (column, C18 silica gel; mobile phase, MeCN in water, 0% to 65% gradient in 35 min; detector, UV 254 nm) to give the title compound as a white solid in 31.46% yield. LC-MS: (ES, m/z): [M+H]⁺ 509; ¹H-NMR: (400 MHz, DMSO, ppm): δ2.36 (s, 3H), δ2.94 (s, 3H), δ3.37 (s, 3H), δ5.65-5.80 (m, 2H), δ5.85 (s, 1H), δ7.36 (s, 1H), δ7.58-7.60 (m, 2H), δ8.39 (s, 1H), δ8.49-8.52 (m, 1H), δ8.62 (s, 1H), δ9.89 (s, 1H).

### Example 199: 1-(2-hydroxyethyl)-3-(hydroxymethyl)-N-methyl-N-[2-methyl-4-[8-methyl-6-(trifluoromethyl)pyrido[3,2-d]pyrimidin-2-yl]phenyl]-1H-pyrazole-5-carboxamide

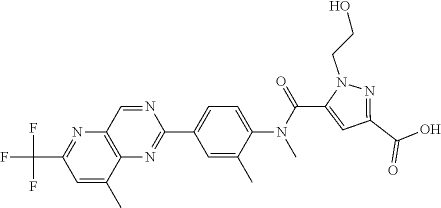 [SMILES: Cc1cc(-c2ncc3nc(C(F)(F)F)cc(C)c3n2)ccc1N(C)C(=O)c1cc(C(=O)O)nn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-[2-(benzyloxy)ethyl]-5-[methyl([2-methyl-4-[8-methyl-6-(trifluoromethyl)pyrido[3,2-d]pyrimidin-2-yl]phenyl])carbamoyl]-1H-pyrazole-3-carboxylic acid provided crude product. The crude product was purified by reverse flash chromatography (C18 silica gel; mobile phase, MeCN in water, 0% to 55% gradient in 35 min; detector, UV 254 nm) to give the title compound as a white solid in 14.50% yield. LC-MS: (ES, m/z): [M+H]⁺ 515; ¹H-NMR: (400 MHz, DMSO, ppm): δ 9.90-9.86 (d, 1H), 8.57 (s, 1H), 8.43-8.38 (m, 2H), 7.53-7.50 (d, 1H), 5.83 (s, 1H), 5.32-5.05 (m, 1H), 4.56-4.39 (m, 2H), 3.79 (s, 2H), 3.32 (s, 3H), 2.92 (s, 3H), 2.38 (s, 3H).

### Step 1: 1-[2-(benzyloxy)ethyl]-5-[methyl([2-methyl-4-[8-methyl-6-(trifluoromethyl)pyrido-[3,2-d]pyrimidin-2-yl]phenyl])carbamoyl]-1H-pyrazole-3-carboxylic acid

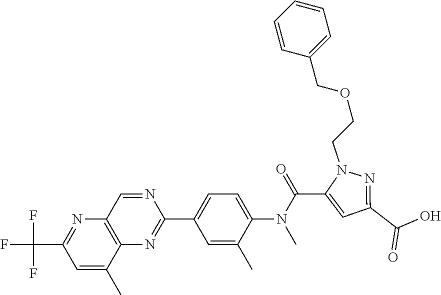 [SMILES: Cc1cc(-c2ncc3nc(C(F)(F)F)cc(C)c3n2)ccc1N(C)C(=O)c1cc(C(=O)O)nn1CCOCc1ccccc1]

Proceeding analogously as described in Example 195, Step 4 but substituting 1-(cyanomethyl)-1H-pyrazole-3,5-dicarboxylate with methyl 1-[2-(benzyloxy)ethyl]-5-[methyl([2-methyl-4-[8-methyl-6-(trifluoromethyl)pyrido[3,2-d]pyrimidin-2-yl]phenyl])carbamoyl]-1H-pyrazole-3-carboxylate provided crude product. The crude product was applied onto a prep-TLC and eluted with EA:PE=1:1 to give the title compound as a white solid in 38.81% yield.

### Example 200: 1-(2-hydroxyethyl)-N5-methyl-N5-(2-methyl-4-(8-methyl-6-(trifluoromethyl)pyrido[3,2-d]pyrimidin-2-yl)phenyl)-1H-pyrazole-3,5-dicarboxamide

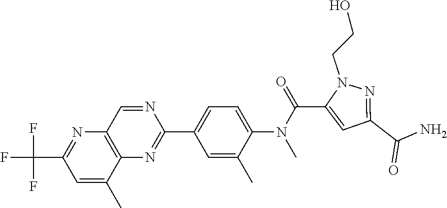 [SMILES: Cc1cc(-c2ncc3nc(C(F)(F)F)cc(C)c3n2)ccc1N(C)C(=O)c1cc(C(N)=O)nn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-[2-(benzyloxy)ethyl]-N5-methyl-N5-[2-methyl-4-[8-methyl-6-(trifluoro-methyl)pyrido[3,2-d]pyrimidin-2-yl]phenyl]-1H-pyrazole-3,5-dicarboxamide provided crude product. The crude product was purified by prep-TLC with EA:PE (1:5) to give the title compound as a white solid in 39.15% yield. LC-MS: (ES, m/z): [M+H]⁺ 514; ¹H-NMR: (400 MHz, DMSO, ppm): δ 9.88 (s, 1H), 8.58 (d, 1H), 8.33-8.44 (m, 2H), 7.54-7.57 (d, 1H), 7.46 (s, 1H), 7.18 (s, 1H), 5.87 (s, 1H), 5.04 (s, 1H), 4.56-4.66 (m, 1H), 4.38-4.50 (m, 1H), 3.81 (s, 2H), 3.38 (s, 3H), 2.91 (s, 3H), 2.38 (s, 3H).

### Step 1: 3,5-dimethyl 1-[2-(benzyloxy)ethyl]-1H-pyrazole-3,5-dicarboxylate

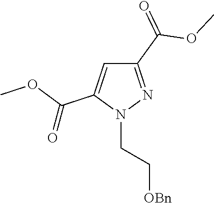

To a stirred solution of 3,5-dimethyl 1H-pyrazole-3,5-dicarboxylate (10 g, 54.30 mmol, 1 equiv.) and [(2-bromoethoxy)methyl]benzene (12.8 g, 59.51 mmol, 1.096 equiv.) in MeCN (200 mL) was added K₂CO₃ (11.3 g, 81.46 mmol, 1.5 equiv.) at room temperature under nitrogen atmosphere. The resulting mixture was stirred for 4 h at 90° C. The mixture was allowed to cool down to room temperature and then quenched with water. The resulting mixture was extracted with EtOAc and the combined organic layers were dried over anhydrous Na₂SO₄. After filtration, the filtrate was concentrated under reduced pressure and the residue was purified by silica gel column chromatography, eluting with PE/EtOAc (10:1) to afford the title compound as a yellow oil in 96.22% yield.

### Step 2: 1-[2-(benzyloxy)ethyl]-3-(methoxycarbonyl)-1H-pyrazole-5-carboxylic acid

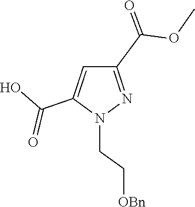 [SMILES: COC(=O)c1cc(C(=O)O)n(CCOCc2ccccc2)n1]

Proceeding analogously as described in Example 167, Step 1 but substituting N-(4-bromo-2-methylphenyl)-2-cyanopyridine-4-carboxamide with 3,5-dimethyl 1-[2-(benzyloxy)ethyl]-1H-pyrazole-3,5-dicarboxylate and stirring the reaction mixture in methanol at room temperature for 3 h provided the title compound as a colorless oil in 68.57% yield.

### Step 3: methyl 1-[2-(benzyloxy)ethyl]-5-[(4-bromo-2-methylphenyl)carbamoyl]-1H-pyrazole-3-carboxylate

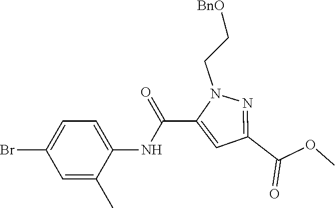 [SMILES: COC(=O)c1cc(C(=O)Nc2ccc(Br)cc2C)n(CCOCc2ccccc2)n1]

Proceeding analogously as described in Example 160, Step 8 but substituting 2-methylpyridine-4-carboxylic acid with 1-[2-(benzyloxy)ethyl]-3-(methoxycarbonyl)-1H-pyrazole-5-carboxylic acid provided crude product. The crude product was purified by a silica gel column by eluting with CH₂Cl₂/MeOH (15:1), followed by re-crystallization from DCM to afford the title compound as a white solid in 53.15% yield.

### Step 4: methyl 1-[2-(benzyloxy)ethyl]-5-[(4-bromo-2-methylphenyl)(methyl)-carbamoyl]-1H-pyrazole-3-carboxylate

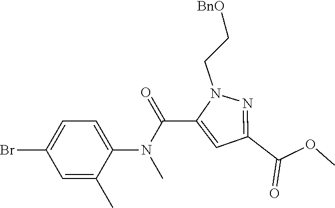 [SMILES: COC(=O)c1cc(C(=O)N(C)c2ccc(Br)cc2C)n(CCOCc2ccccc2)n1]

Proceeding analogously as described in Example 161, Step 1 but substituting N-(4-bromo-2-methylphenyl)-2-methylpyridine-4-carboxamide with methyl 1-[2-(benzyloxy)ethyl]-5-[(4-bromo-2-methylphenyl)carbamoyl]-1H-pyrazole-3-carboxylate provided crude product. The crude product was purified by silica gel column chromatography by eluting with PE/EtOAc (5:1) to afford the title compound in 77.69% yield.

### Step 5: methyl 1-[2-(benzyloxy)ethyl]-5-[methyl[2-methyl-4-(4,4,5,5-tetramethyl-1,3,2-dioxaborolan-2-yl)phenyl]carbamoyl]-1H-pyrazole-3-carboxylate

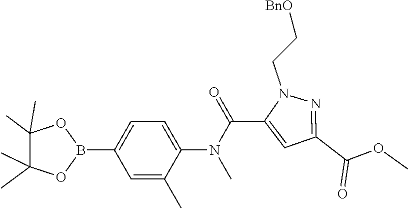

Proceeding as described in Example 161, Step 2 but substituting N-(4-bromo-2-methylphenyl)-N,2-dimethylpyridine-4-carboxamide with methyl 1-[2-(benzyloxy)ethyl]-5-[(4-bromo-2-methylphenyl)(methyl)carbamoyl]-1H-pyrazole-3-carboxylate provided crude product. The crude product was purified by silica gel column chromatography by eluting with PE/EtOAc (5:1) to afford the title compound as a yellow oil in 95.98% yield.

### Step 6: methyl 1-[2-(benzyloxy)ethyl]-5-[methyl([2-methyl-4-[8-methyl-6-(trifluoro-methyl)pyrido[3,2-d]pyrimidin-2-yl]phenyl])carbamoyl]-1H-pyrazole-3-carboxylate

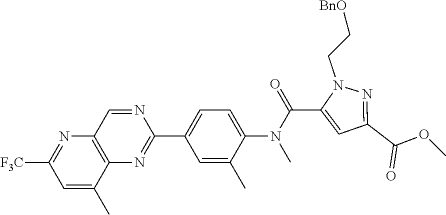 [SMILES: COC(=O)c1cc(C(=O)N(C)c2ccc(-c3ncc4nc(C(F)(F)F)cc(C)c4n3)cc2C)n(CCOCc2ccccc2)n1]

Proceeding analogously as described in Example 193 but substituting 1-(2-hydroxypropyl)-N-[2-methyl-4-(4,4,5,5-tetramethyl-1,3,2-dioxaborolan-2-yl)phenyl]-1H-pyrazole-5-carboxamide with methyl 1-[2-(benzyloxy)ethyl]-5-[methyl[2-methyl-4-(4,4,5,5-tetramethyl-1,3,2-dioxaborolan-2-yl)phenyl]carbamoyl]-1H-pyrazole-3-carboxylate provided crude product. The crude product was applied onto a silica gel column and eluted with ethyl acetate/petroleum ether (1:10) to give the title compound as a white solid in 50.43% yield.

### Step 7: 1-[2-(benzyloxy)ethyl]-N5-methyl-N5-[2-methyl-4-[8-methyl-6-(trifluoro-methyl)pyrido[3,2-d]pyrimidin-2-yl]phenyl]-1H-pyrazole-3,5-dicarboxamide

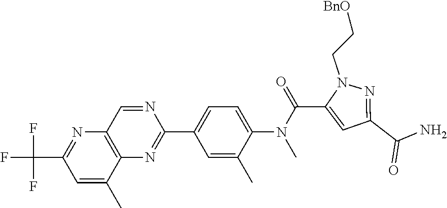 [SMILES: Cc1cc(-c2ncc3nc(C(F)(F)F)cc(C)c3n2)ccc1N(C)C(=O)c1cc(C(N)=O)nn1CCOCc1ccccc1]

Into an 8-mL sealed tube, was placed methyl 1-[2-(benzyloxy)ethyl]-5-[methyl([2-methyl-4-[6-(trifluoromethyl)pyrido[3,2-d]pyrimidin-2-yl]phenyl])carbamoyl]-1H-pyrazole-3-carboxylate (200 mg, 0.33 mmol, 1 equiv.) and NH₃ solution in MeCN (2 mL). The resulting solution was stirred overnight at 50° C. and then concentrated. The residue was applied onto a Prep-TLC and eluted with ethyl acetate/petroleum ether (1:1) to give the title compound as a white solid in 52.08% yield.

### Example 201: 1-(2-hydroxyethyl)-N-methyl-N-(2-methyl-4-(8-methyl-6-(trifluoromethyl)-quinolin-2-yl)phenyl)-1H-pyrazole-5-carboxamide

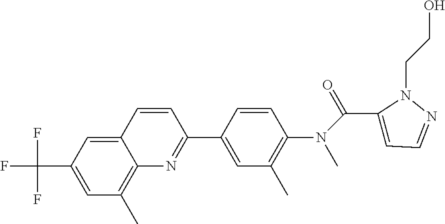 [SMILES: Cc1cc(-c2ccc3cc(C(F)(F)F)cc(C)c3n2)ccc1N(C)C(=O)c1ccnn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-[2-(benzyloxy)ethyl]-N-methyl-N-[2-methyl-4-[8-methyl-6-(trifluoromethyl)quinolin-2-yl]phenyl]-1H-pyrazole-5-carboxamide provided crude product. The crude product was applied onto Prep-TLC and eluted with acetate/petroleum ether (1:2) to give the title compound as a yellow solid in 79.73% yield. LC-MS: (ES, m/z): [M+H]⁺ 469; ¹H-NMR: (300 MHz, DMSO, ppm): (300 MHz, DMSO, ppm): δ 7.66-7.69 (m, 1H), 7.53 (s, 1H), 7.35-7.47 (m, 3H), 7.00 (s, 1H), 6.61-6.63 (d, 1H), 6.40 (s, 1H), 4.85 (s, 1H), 3.91-3.99 (m, 1H), 3.71-3.74 (m, 1H), 3.15 (s, 2H), 2.63 (s, 3H), 2.12 (s, 3H), 1.61 (s, 3H)

### Step 1: methyl 1-(2-(benzyloxy)ethyl)-1H-pyrazole-5-carboxylate

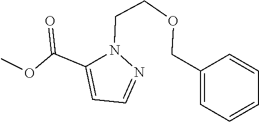 [SMILES: COC(=O)c1ccnn1CCOCc1ccccc1]

To a stirred solution of methyl 1H-pyrazole-5-carboxylate (10 g, 79.29 mol, 1 equiv.) and 2-(benzyloxy)ethan-1-ol (14.5 g, 95.28 mol, 1.2 equiv.) in THE (100 mL) was added DIAD (24.1 g, 119.18 mmol, 1.5 equiv.) and PPH₃ (41.6 g, 158.61 mmol, 2 equiv.) in portions at 0-10° C. under nitrogen atmosphere. The resulting mixture was stirred overnight at room temperature under nitrogen atmosphere and then concentrated under reduced pressure. The residue was purified by silica gel column chromatography by eluting with PE/EtOAc (10/1) to afford the title compound as a yellow oil in 61.05% yield.

### Step 2: 1-(2-(benzyloxy)ethyl)-1H-pyrazole-5-carboxylic acid

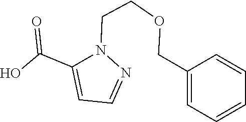 [SMILES: O=C(O)c1ccnn1CCOCc1ccccc1]

Proceeding analogously as described in Example 232, Step 4 but substituting methyl 1-(2-oxopropyl)-1H-pyrazole-5-carboylate with methyl 1-(2-(benzyloxy)ethyl)-1H-pyrazole-5-carboxylate provided the title compound as a white solid in 90.60% yield.

### Step 3: 1-(2-(benzyloxy)ethyl)-N-(4-bromo-2-methylphenyl)-1H-pyrazole-5-carboxamide

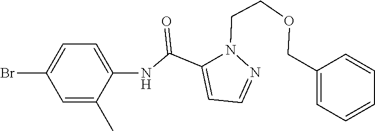 [SMILES: Cc1cc(Br)ccc1NC(=O)c1ccnn1CCOCc1ccccc1]

Proceeding analogously as described in Example 160, Step 8 but substituting 2-methylpyridine-4-carboxylic acid with 1-(2-(benzyloxy)ethyl)-1H-pyrazole-5-carboxylic acid provided title compound as a white solid in 88.06% yield

### Step 4: 1-(2-(benzyloxy)ethyl)-N-(4-bromo-2-methylphenyl)-N-methyl-1H-pyrazole-5-carboxamide

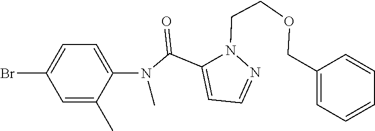 [SMILES: Cc1cc(Br)ccc1N(C)C(=O)c1ccnn1CCOCc1ccccc1]

Proceeding analogously as described in Example 165, Step 2 but substituting N-(4-bromo-2-fluorophenyl)-2-methylisonicotinamide with 1-(2-(benzyloxy)ethyl)-N-(4-bromo-2-methylphenyl)-1H-pyrazole-5-carboxamide provided crude product. The crude product was purified by silica gel column chromatography by eluting with PE/EtOAc (30/1) to afford the title compound as a yellow oil in 94.79% yield.

### Step 5: 1-(2-(benzyloxy)ethyl)-N-methyl-N-(2-methyl-4-(4,4,5,5-tetramethyl-1,3,2-dioxaborolan-2-yl)phenyl)-1H-pyrazole-5-carboxamide

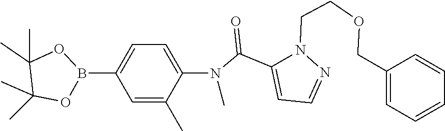 [SMILES: Cc1cc(B2OC(C)(C)C(C)(C)O2)ccc1N(C)C(=O)c1ccnn1CCOCc1ccccc1]

Proceeding as described in Example 161, Step 2 but substituting N-(4-bromo-2-methylphenyl)-N,2-dimethylpyridine-4-carboxamide with 1-(2-(benzyloxy)ethyl)-N-(4-bromo-2-methylphenyl)-N-methyl-1H-pyrazole-5-carboxamide provided crude product. The crude product was purified by silica gel column chromatography by eluting with PE/EtOAc (20/1) to afford the title compound as a yellow oil in 64.36% yield.

### Step 6: 1-(2-(benzyloxy)ethyl)-N-(4-(8-bromo-6-(trifluoromethyl)quinolin-2-yl)-2-methylphenyl)-N-methyl-1H-pyrazole-5-carboxamide

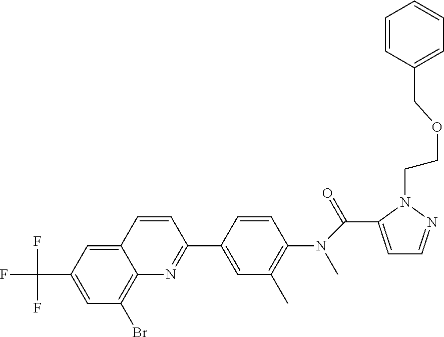 [SMILES: Cc1cc(-c2ccc3cc(C(F)(F)F)cc(Br)c3n2)ccc1N(C)C(=O)c1ccnn1CCOCc1ccccc1]

Into a 25-mL 3-necked round-bottom flask purged and maintained with an inert atmosphere of nitrogen, was placed 8-bromo-2-chloro-6-(trifluoromethyl)quinoline (300 mg, 0.97 mmol, 1 equiv.), 1-[2-(benzyloxy)ethyl]-N-methyl-N-[2-methyl-4-(tetramethyl-1,3,2-dioxaborolan-2-yl)phenyl]-1H-pyrazole-5-carboxamide (459.3 mg, 0.97 mmol, 1 equiv.), Na₂CO₃ (307.2 mg, 2.90 mmol, 3 equiv.), DME (6 mL), H₂O (1.5 mL), Pd(PPh₃)₄(111.6 mg, 0.10 mmol, 0.1 equiv.). The resulting solution was stirred for 12 h at 80° C. and then extracted with ethyl acetate. The combined organic layers were concentrated under reduced pressure and the residue was applied onto Prep-TLC and eluted with ethyl acetate/petroleum ether (1:2) to give the title compound as a yellow oil in 74.70% yield.

### Step 7: 1-(2-(benzyloxy)ethyl)-N-methyl-N-(2-methyl-4-(8-methyl-6-(trifluoromethyl)-quinolin-2-yl)phenyl)-1H-pyrazole-5-carboxamide

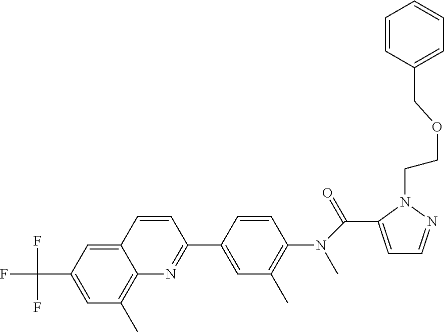 [SMILES: Cc1cc(-c2ccc3cc(C(F)(F)F)cc(C)c3n2)ccc1N(C)C(=O)c1ccnn1CCOCc1ccccc1]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with methylboronic acid and 2-chloro-8-methyl-6-(trifluoromethyl)pyrido[3,2-d]pyrimidine with 1-[2-(benzyloxy)ethyl]-N-[4-[8-bromo-6-(trifluoromethyl)quinolin-2-yl]-2-methylphenyl]-N-methyl-1H-pyrazole-5-carboxamide provided crude product. The product was applied onto Prep-TLC and eluted with ethyl acetate/petroleum ether (1:3) to give the title compound as a yellow solid in 62.01% yield.

### Example 202: N,1-dimethyl-N-(2-methyl-4-(8-methyl-6-(trifluoromethyl)quinolin-2-yl)phenyl)-1H-pyrazole-5-carboxamide

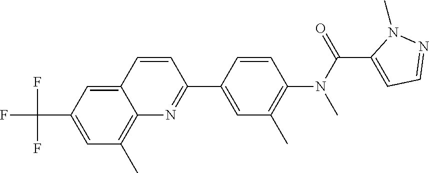 [SMILES: Cc1cc(-c2ccc3cc(C(F)(F)F)cc(C)c3n2)ccc1N(C)C(=O)c1ccnn1C]

Proceeding analogously as described in Example 165, Step 2 but substituting N-(4-bromo-2-fluorophenyl)-2-methylisonicotinamide with 1-methyl-N-[2-methyl-4-[8-methyl-6-(trifluoromethyl)quinolin-2-yl]phenyl]-1H-pyrazole-5-carboxamide provided crude product. The crude product was purified by Prep-TLC (CH₂Cl₂/MeOH 60/1) to afford the title compound as an off-white solid in 69.77% yield. LC-MS: (ES, m/z): [M+H]⁺ 439; ¹H-NMR: (400 MHz, DMSO, ppm): δ 2.28 (s, 3H), 2.89 (s, 3H), 3.33 (s, 3H), 4.01 (s, 3H), 5.57 (s, 1H), 7.15-7.16 (d, 1H), 7.48-7.50 (d, 1H), 7.91 (s, 1H), 8.21-8.24 (m, 1H), 8.28 (s, 1H), 8.32-8.34 (d, 2H), 8.65-8.65 (d, 1H).

### Example 203: 1-methyl-N-(2-methyl-4-(8-methyl-6-(trifluoromethyl)quinolin-2-yl)phenyl)-1H-pyrazole-5-carboxamide

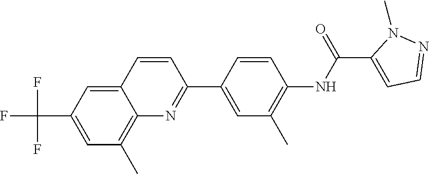 [SMILES: Cc1cc(-c2ccc3cc(C(F)(F)F)cc(C)c3n2)ccc1NC(=O)c1ccnn1C]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with methylboronic acid and 2-chloro-8-methyl-6-(trifluoromethyl)pyrido[3,2-d]pyrimidine with N-(4-(8-bromo-6-(trifluoromethyl)quinolin-2-yl)-2-methylphenyl)-1-methyl-1H-pyrazole-5-carboxamide provided crude product. The crude product was purified by Prep-TLC (CH₂Cl₂/MeOH 50/1) to afford the title compound as an off-white solid in 55.14% yield. LC-MS: (ES, m/z): [M+H]⁺ 425; ¹H-NMR (400 MHz, DMSO, ppm): δ 2.39 (s, 3H), 2.89 (s, 3H), 4.11 (s, 3H), 7.11-7.12 (d, 1H), 7.56-7.59 (m, 2H), 7.90 (s, 1H), 8.23-8.35 (m, 4H), 8.63-8.65 (d, 1H), 10.00 (s, 3H)

### Step 1: N-(4-(8-bromo-6-(trifluoromethyl)quinolin-2-yl)-2-methylphenyl)-1-methyl-1H-pyrazole-5-carboxamide

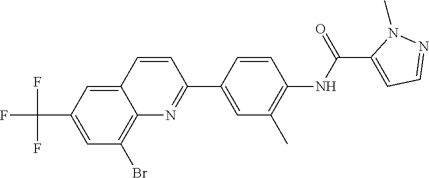 [SMILES: Cc1cc(-c2ccc3cc(C(F)(F)F)cc(Br)c3n2)ccc1NC(=O)c1ccnn1C]

Proceeding analogously as described in Example 167 but substituting N2-methyl-N4-(2-methyl-4-(4,4,5,5-tetramethyl-1,3,2-dioxaborolan-2-yl)phenyl)pyridine-2,4-dicarboxamide with methyl-N-[2-methyl-4-(4,4,5,5-tetramethyl-1,3,2-dioxaborolan-2-yl)phenyl]-1H-pyrazole-5-carboxamide and 2-chloro-8-methyl-6-(trifluoromethyl)pyrido[3,2-d]pyrimidine with 8-bromo-2-chloro-6-(trifluoromethyl)quinoline provided crude product. The crude product was purified by Prep-TLC (PE/EtOAc 2/1) to afford the title compound as a yellow solid in 63.46% yield.

### Example 204: N-[2-methyl-4-[6-(trifluoromethyl)quinolin-2-yl]phenyl]-1H-pyrazole-5-carboxamide

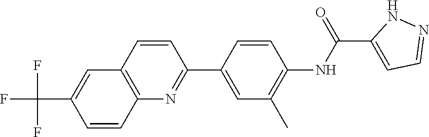 [SMILES: Cc1cc(-c2ccc3cc(C(F)(F)F)ccc3n2)ccc1NC(=O)c1ccn[nH]1]

Proceeding analogously as described in Example 160, Step 8 but substituting 2-methylpyridine-4-carboxylic acid with 1H-pyrazole-5-carboxylic acid and 4-bromo-2-methylaniline with 2-methyl-4-[6-(trifluoromethyl)quinolin-2-yl]aniline provided crude product. (The crude product was applied onto a prep-TLC and eluted with ethyl acetate/petroleum ether (1:5) to give the title compound as a white solid in 18.38% yield. ¹H-NMR: (400 MHz, DMSO, ppm): δ 13.47 (s, 1H), 9.57 (s, 1H), 8.65-8.66 (d, 1H), 8.64 (s, 1H), 8.8.32-8.34 (d, 1H), 8.8.26-8.27 (d, 1H), 8.20-8.24 (m, 2H), 8.18-8.20 (m, 1H), 7.94-8.03 (m, 3H), 6.81 (s, 1H), 2.43 (s, 3H)

### Example 205: N-methyl-N-[2-methyl-4-[6-(trifluoromethyl)quinolin-2-yl]phenyl]-1H-pyrazole-5-carboxamide

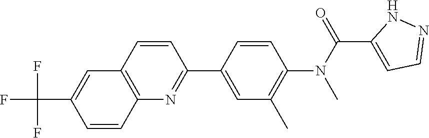 [SMILES: Cc1cc(-c2ccc3cc(C(F)(F)F)ccc3n2)ccc1N(C)C(=O)c1ccn[nH]1]

Into a 25-mL 3-necked round-bottom flask, was placed N,2-dimethyl-4-[6-(trifluoromethyl)quinolin-2-yl]aniline (200 mg, 0.63 mmol, 1 equiv.), 1H-pyrazole-5-carboxylic acid (212.6 mg, 1.90 mmol, 3 equiv.), HATU (721.2 mg, 1.90 mmol, 3 equiv.), and DIEA (245.1 mg, 1.90 mmol, 3 equiv.) in DMF (5 mL). The resulting solution was stirred overnight at 100° C. and then quenched with water. The resulting solution was extracted with ethyl acetate and the organics were evaporated. The residue was applied onto prep-TLC and eluted with ethyl acetate/petroleum ether (1:5) to give the title compound as a white solid in 57.81% yield as a white solid. LC-MS: (ES, m/z):[M+H]⁻ 409; ¹H-NMR: (300 MHz, DMSO, ppm): δ 13.65 (s, 1H), 12.94 (s, 1H), 8.68-8.73 (m, 1H), 8.55-8.66 (m, 1H), 8.24-8.41 (m, 3H), 8.01-8.19 (m, 1H), 7.50-7.57 (m, 2H), 7.31-7.34 (d, 1H), 7.23 (s, 1H), 6.23 (s, 1H), 4.94 (s, 1H), 3.29 (s, 3H), 2.25-2.27 (d, 3H)

### Step 1: N,2-dimethyl-4-[6-(trifluoromethyl)quinolin-2-yl]aniline

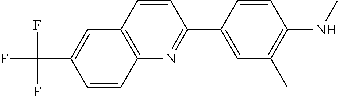 [SMILES: CNc1ccc(-c2ccc3cc(C(F)(F)F)ccc3n2)cc1C]

Proceeding analogously as described in Example 165, Step 2 but substituting N-(4-bromo-2-fluorophenyl)-2-methylisonicotinamide with 2-methyl-4-[6-(trifluoromethyl)quinolin-2-yl]aniline provided crude product. The crude product was applied onto a silica gel column and eluted with ethyl acetate/petroleum ether (1:10) to give the title compound as a white solid in 19.11% yield.

### Example 206: N-(2,5-dimethyl-4-(6-(trifluoromethyl)quinolin-2-yl)phenyl)-1,4-dimethyl-1H-pyrazole-5-carboxamide

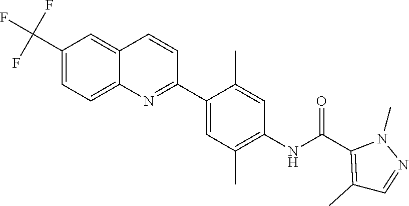 [SMILES: Cc1cc(-c2ccc3cc(C(F)(F)F)ccc3n2)c(C)cc1NC(=O)c1c(C)cnn1C]

Into an 8-mL sealed tube purged and maintained with an inert atmosphere of nitrogen, was placed 2,5-dimethyl-4-[6-(trifluoromethyl)quinolin-2-yl]aniline (100 mg, 0.32 mmol, 1 equiv.), 1,4-dimethyl-1H-pyrazole-5-carboxylic acid (53.2 mg, 0.38 mmol, 1.2 equiv.), DIEA (81.7 mg, 0.63 mmol, 2 equiv.), DMF (2 mL), and HATU (180.3 mg, 0.47 mmol, 1.5 equiv.). The resulting solution was stirred for 12 hat 50° C. and then quenched with water. The resulting solution was extracted with ethyl acetate. The combined organic layers were concentrated under reduced pressure and the residue was applied onto Prep-TLC and eluted with ethyl acetate/petroleum ether (1:5) to give the title compound as a white solid in 57.72% yield. LC-MS: (ES, m/z): [M+H]⁺ 439; ¹H-NMR: (400 MHz, DMSO, ppm): δ 9.77 (s, 1H), 8.65-8.68 (d, 1H), 8.58 (s, 1H), 8.23-8.26 (d, 1H), 8.02-8.05 (dd, 1H), 7.89-7.91 (d, 1H), 7.49-7.52 (d, 2H), 7.36 (s, 1H), 3.96 (s, 3H), 2.45 (s, 3H), 2.32 (s, 3H), 2.26 (s, 3H).

### Step 1: ethyl 1,4-dimethyl-1H-pyrazole-5-carboxylate

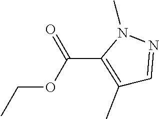

Into a 100-mL 3-necked round-bottom flask purged and maintained with an inert atmosphere of nitrogen, was placed ethyl 4-methyl-1H-pyrazole-5-carboxylate (3 g, 19.46 mmol, 1 equiv.), DIAD (7.9 g, 38.92 mmol, 2 equiv.), THE (30 mL), and MeOH (3.1 g, 97.30 mmol, 5 equiv.), and PPh₃ (10.2 g, 38.92 mmol, 2 equiv.) was added at 0° C. The resulting solution was stirred for 5 h at 25° C. and then concentrated under vacuum. The residue was applied onto a silica gel column and eluted with ethyl acetate/petroleum ether (1:50) to give the title compound as a yellow liquid in 76.38% yield.

### Step 2: 1,4-dimethyl-1H-pyrazole-5-carboxylic acid

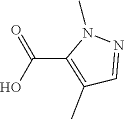

Proceeding analogously as described in Example 167, Step 1 but substituting N-(4-bromo-2-methylphenyl)-2-cyanopyridine-4-carboxamide with ethyl 1,4-dimethyl-1H-pyrazole-5-carboxylate provided the title compound as a white solid 96.01% yield.

### Step 3: 2,5-dimethyl-4-(4,4,5,5-tetramethyl-1,3,2-dioxaborolan-2-yl)aniline

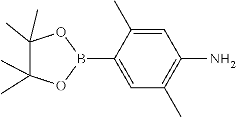 [SMILES: Cc1cc(B2OC(C)(C)C(C)(C)O2)c(C)cc1N]

Proceeding as described in Example 161, Step 2 but substituting N-(4-bromo-2-methylphenyl)-N,2-dimethylpyridine-4-carboxamide with 4-bromo-2,5-dimethylaniline provided the title compound as a white solid in 34.33% yield.

### Step 4: 2,5-dimethyl-4-(6-(trifluoromethyl)quinolin-2-yl)aniline

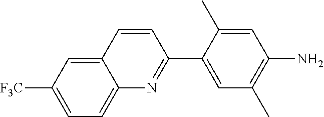 [SMILES: Cc1cc(-c2ccc3cc(C(F)(F)F)ccc3n2)c(C)cc1N]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 2,5-dimethyl-4-(4,4,5,5-tetramethyl-1,3,2-dioxaborolan-2-yl)aniline and 2-chloro-8-methyl-6-(trifluoromethyl)pyrido[3,2-d]pyrimidine with 2-bromo-6-(trifluoromethyl)quinoline provided the title compound as a yellow solid in 23.44% yield.

### Example 207: N-(2-fluoro-5-methyl-4-(6-(trifluoromethyl)quinolin-2-yl)phenyl)-1,4-dimethyl-1H-pyrazole-5-carboxamide

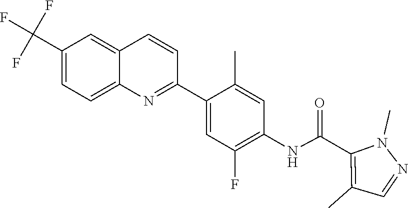 [SMILES: Cc1cc(NC(=O)c2c(C)cnn2C)c(F)cc1-c1ccc2cc(C(F)(F)F)ccc2n1]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 5-methyl-4-(4,4,5,5-tetramethyl-1,3,2-dioxaborolan-2-yl)phenyl]-1,4-dimethyl-1H-pyrazole-5-carboxamide and 2-chloro-8-methyl-6-(trifluoromethyl)pyrido[3,2-d]pyrimidine with 2-bromo-6-(trifluoromethyl)quinoline provided crude product. The crude product was applied onto Prep-TLC and eluted with ethyl acetate/petroleum ether (1:1) to give the title compound as a yellow solid in 42.52% yield. LC-MS: (ES, m/z): [M+H]⁺ 443; ¹H-NMR: (400 MHz, DMSO, ppm): δ 2.23 (s, 3H), 2.42 (s, 3H), 3.95 (s, 3H), 7.37 (s, 1H), 7.49-7.55 (d, 1H), 7.79-7.81 (d, 1H), 7.92-7.95 (d, 1H), 8.04-8.06 (d, 1H), 8.25-8.27 (d, 1H), 8.59 (s, 1H), 8.68-8.70 (d, 1H), 10.07 (s, 1H).

### Step 1: N-(4-bromo-2-fluoro-5-methylphenyl)-1,4-dimethyl-1H-pyrazole-5-carboxamide

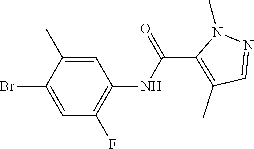 [SMILES: Cc1cc(NC(=O)c2c(C)cnn2C)c(F)cc1Br]

Proceeding analogously as described in Example 206 but substituting 2,5-dimethyl-4-[6-(trifluoromethyl)quinolin-2-yl]aniline with 4-bromo-2-fluoro-5-methylaniline provided crude product. The crude product was applied onto a silica gel column and eluted with ethyl acetate/petroleum ether (1:50) to give the title compound as a yellow solid in 42.97% yield.

### Step 2: 5-methyl-4-(4,4,5,5-tetramethyl-1,3,2-dioxaborolan-2-yl)phenyl]-1,4-dimethyl-1H-pyrazole-5-carboxamide

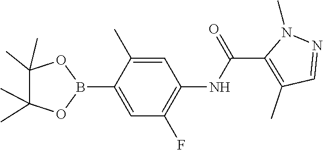 [SMILES: Cc1cc(NC(=O)c2c(C)cnn2C)c(F)cc1B1OC(C)(C)C(C)(C)O1]

Proceeding as described in Example 161, Step 2 but substituting N-(4-bromo-2-methylphenyl)-N,2-dimethylpyridine-4-carboxamide with N-(4-bromo-2-fluoro-5-methylphenyl)-1,4-dimethyl-1H-pyrazole-5-carboxamide provided crude product. The crude product was applied onto Prep-TLC and eluted with ethyl acetate/petroleum ether (1:20) to give the title compound as a yellow solid in 87.39% yield.

### Example 208: N-(2,5-dimethyl-4-(6-(trifluoromethyl)quinolin-2-yl)phenyl)-1,4-dimethyl-1H-pyrazole-3-carboxamide

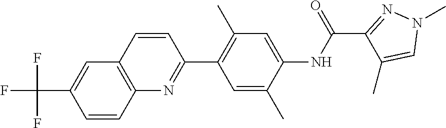 [SMILES: Cc1cc(-c2ccc3cc(C(F)(F)F)ccc3n2)c(C)cc1NC(=O)c1nn(C)cc1C]

Proceeding analogously as described in Example 206 but substituting 1,4-dimethyl-1H-pyrazole-5-carboxylic acid with 1-methyl-1H-pyrazole-3-carboxylic acid provided crude product. The crude product was applied onto a Prep-TLC and eluted with ethyl acetate/petroleum ether (1:1) gave the title compound as a yellow solid in 8.05% yield. LC-MS: (ES, m/z): [M+H]⁺ 439; ¹H-NMR: (400 MHz, DMSO, ppm): δ 2.07 (s, 3H), 2.26 (s, 3H), 2.31 (s, 3H), 3.90 (s, 3H), 7.46 (s, 1H), 7.66 (s, 1H), 7.77 (s, 1H), 7.87-7.90 (d, 1H), 8.00-8.04 (d, 1H), 8.21-8.24 (d, 1H), 8.56 (s, 1H), 8.63-8.66 (d, 1H), 9.31 (s, 1H).

### Step 1: ethyl 1-methyl-1H-pyrazole-3-carboxylate

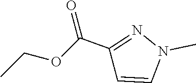

Proceeding as described in Example 206, Step 1 but substituting ethyl 1H-pyrazole-5-carboxylate with ethyl 4-methyl-1H-pyrazole-5-carboxylate provided crude product. The crude product was applied onto a silica gel column and eluted with ethyl acetate/petroleum ether (1:10) to give the title compound as a yellow solid in 4.58% yield.

### Step 2: 1,4-dimethyl-1H-pyrazole-3-carboxylic acid

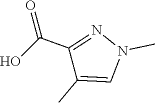

Proceeding analogously as described in Example 167, Step 1 but substituting N-(4-bromo-2-methylphenyl)-2-cyanopyridine-4-carboxamide with ethyl 1-methyl-1H-pyrazole-3-carboxylate provided the title compound as a white solid in 80.01% yield.

### Example 209: 1-(2-hydroxyethyl)-N-methyl-N-(2-methyl-4-(6-(trifluoromethyl)quinolin-2-yl)phenyl)-1H-pyrazole-5-carboxamide

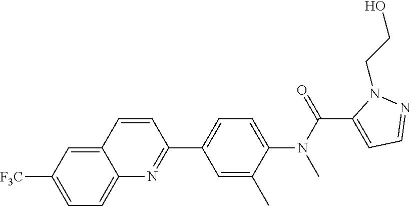 [SMILES: Cc1cc(-c2ccc3cc(C(F)(F)F)ccc3n2)ccc1N(C)C(=O)c1ccnn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-(2-(benzyloxy)ethyl)-N-methyl-N-(2-methyl-4-(6-(trifluoromethyl)quinolin-2-yl)phenyl)-1H-pyrazole-5-carboxamide provided crude product. The residue was purified by Prep-TLC (hexane/EtOAc=1:1) to afford the title compound as a white solid in 43.80% yield. LC-MS: (ES, m/z): [M+H]⁺ 455; ¹H-NMR: (300 MHz, CDCl₃, ppm): δ2.32 (s, 3H), δ3.42 (s, 3H), 54.05-4.08 (t, 2H), 54.61-4.71 (m, 2H), δ5.56 (s, 1H), δ7.18 (s, 1H), δ7.29-7.32 (d, 1H), δ7.96-8.05 (m, 3H), δ8.14-8.18 (d, 2H), δ8.34-8.39 (t, 2H).

### Step 1: 1-(2-(benzyloxy)ethyl)-N-methyl-N-(2-methyl-4-(6-(trifluoromethyl)quinolin-2-yl)phenyl)-1H-pyrazole-5-carboxamide

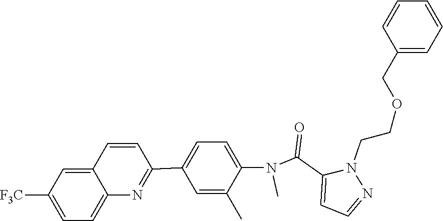 [SMILES: Cc1cc(-c2ccc3cc(C(F)(F)F)ccc3n2)ccc1N(C)C(=O)c1ccnn1CCOCc1ccccc1]

Proceeding analogously as described in Example 201, Step 6 but substituting 8-bromo-2-chloro-6-(trifluoromethyl)quinoline with 2-bromo-6-(trifluoromethyl)quinoline provided crude product which was purified by Prep-TLC (PE/EtOAc=5/1) to afford the title compound as a light yellow oil in 73.33% yield.

### Example 210: 1-(2-hydroxyethyl)-N-methyl-N-(2-methyl-4-(6-(trifluoromethyl)-1,5-naphthyridin-2-yl)phenyl)-1H-pyrazole-5-carboxamide

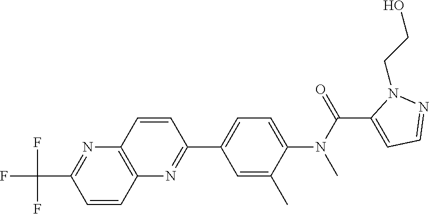 [SMILES: Cc1cc(-c2ccc3nc(C(F)(F)F)ccc3n2)ccc1N(C)C(=O)c1ccnn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-(2-(benzyloxy)ethyl)-N-methyl-N-(2-methyl-4-(6-(trifluoromethyl)-1,5-naphthyridin-2-yl)phenyl)-1H-pyrazole-5-carboxamide provided crude product. Purification by prep. TLC (PE/EA=3/1) gave the title compound as a white solid in 63.70% yield. LC-MS: (ES, m/z): [M+H]⁺ 456; ¹H-NMR: (300 MHz, CDCl₃, ppm): δ2.32 (s, 3H), δ3.47 (s, 3H), δ3.95-4.07 (m, 2H), δ4.60-4.74 (m, 2H), δ5.55 (s, 1H), δ7.18 (s, 1H), δ7.31-7.33 (d, 1H), δ8.00-8.03 (m, 2H), δ8.07 (s, 1H), δ8.20-8.23 (d, 1H), δ8.58-8.67 (m, 2H).

### Step 1: 1-(2-(benzyloxy)ethyl)-N-methyl-N-(2-methyl-4-(6-(trifluoromethyl)-1,5-naphthyridin-2-yl)phenyl)-1H-pyrazole-5-carboxamide

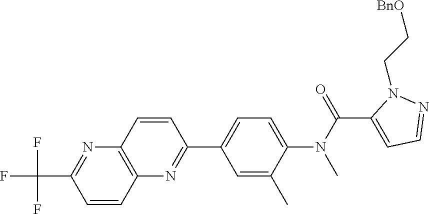 [SMILES: Cc1cc(-c2ccc3nc(C(F)(F)F)ccc3n2)ccc1N(C)C(=O)c1ccnn1CCOCc1ccccc1]

Proceeding analogously as described in Example 201, Step 6 but substituting 8-bromo-2-chloro-6-(trifluoromethyl)quinoline with 2-chloro-6-(trifluoromethyl)-1,5-naphthyridine provided crude product which was purified by Prep.TLC (PE/EA=3/1) to the title compound as a white solid in 85.27% yield.

### Example 211: 1-(2-hydroxyethyl)-N-methyl-N-(2-methyl-4-(6-(trifluoromethyl)quinazolin-2-yl)phenyl)-1H-pyrazole-5-carboxamide

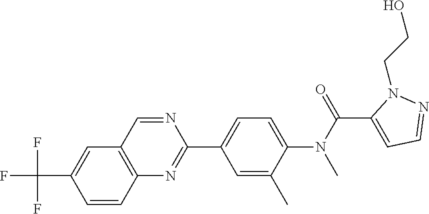 [SMILES: Cc1cc(-c2ncc3cc(C(F)(F)F)ccc3n2)ccc1N(C)C(=O)c1ccnn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-[2-(benzyloxy)ethyl]-N-methyl-N-[4-[6-(trifluoromethyl)quinazolin-2-yl]phenyl]-1H-pyrazole-5-carboxamide provided crude product. Purification by prep-TLC with ethyl acetate/petroleum ether (1:2) provided the title compound as a white solid in 43.01% yield. LC-MS: (ES, m/z):[M+H]⁺ 456. ¹H-NMR: (300 MHz, CDCl3, ppm): δ 9.57 (s, 1H), 8.54-8.49 (m, 2H), 8.28-8.20 (d, 1H), 8.12-8.11 (d, 1H), 8.09-8.08 (d, 1H), 7.32-7.29 (m, 1H), 7.16-7.15 (d, 1H), 5.55-5.40 (d, 1H), 4.79-4.60 (m, 2H), 4.08-4.05 (t, 2H), 3.43 (s, 3H), 2.31 (s, 3H).

### Step 1: 1-[2-(benzyloxy)ethyl]-N-methyl-N-[2-methyl-4-[6-(trifluoromethyl)quinazolin-2-yl]phenyl]-1H-pyrazole-5-carboxamide

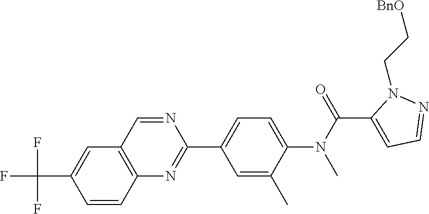 [SMILES: Cc1cc(-c2ncc3cc(C(F)(F)F)ccc3n2)ccc1N(C)C(=O)c1ccnn1CCOCc1ccccc1]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-[2-(benzyloxy)ethyl]-N-methyl-N-[2-methyl-4-(4,4,5,5-tetramethyl-1,3,2-dioxaborolan-2-yl)phenyl]-1H-pyrazole-5-carboxamide and 2-chloro-8-methyl-6-(trifluoromethyl)pyrido[3,2-d]pyrimidine with 2-chloro-6-trifluoromethylquinazoline provided crude product. The crude product was applied onto a prep-TLC and eluted with ethyl acetate/petroleum ether (1:2) to give the title compound as a white solid in 60.82% yield.

### Example 212: 1-methyl-N-(methyl-d3)-N-(2-methyl-4-(6-(trifluoromethyl)quinolin-2-yl)phenyl)-1H-pyrazole-5-carboxamide

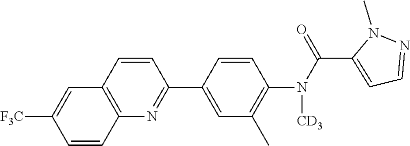 [SMILES: [2H]C([2H])([2H])N(C(=O)c1ccnn1C)c1ccc(-c2ccc3cc(C(F)(F)F)ccc3n2)cc1C]

Proceeding analogously as described in Example 165, Step 2 but substituting N-(4-bromo-2-fluorophenyl)-2-methylisonicotinamide with 1-methyl-N-[2-methyl-4-(6-(trifluoro-methyl)-quinolin-2-yl)phenyl)-1H-pyrazole-5-carboxamide and Mel with CD₃Cl, the title compound was obtained as an off-white solid in 77% yield. LC-MS: (ES, m/z): [M+H]⁺ 428. ¹H-NMR: (300 MHz, DMSO, ppm): δ2.27 (s, 3H), 4.02 (s, 3H), 5.55 (s, 1H), 7.16 (s, 1H), 7.48 (d, 1H), 8.03 (d, 1H), 8.16 (d, 1H), 8.25 (s, 1H), 8.26 (d, 1H), 8.33 (d, 1H), 8.55 (s, 1H), 8.69 (d, 1H).

### Example 213: 2-(2-hydroxypropan-2-yl)-N-methyl-N-(2-methyl-4-(6-(trifluoromethyl)-quinazolin-2-yl)phenyl)isonicotinamide

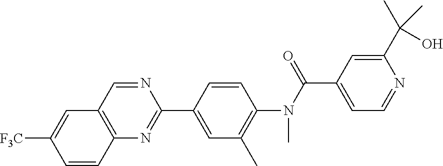 [SMILES: Cc1cc(-c2ncc3cc(C(F)(F)F)ccc3n2)ccc1N(C)C(=O)c1ccnc(C(C)(C)O)c1]

Into a round-bottom flask, was N,2-dimethyl-4-(6-(trifluoromethyl)quinazolin-2-yl)aniline (0.12 g, 0.38 mmol, 1 equiv.), 2-(2-hydroxypropan-2-yl)isonicotinic acid (0.09 g, 0.495 mmol, 1.3 equiv.), T3P, 50% solution in EtOAc (0.30 g, 0.95 mmol, 2.5 equiv.), DIPEA (0.074 g, 0.57 mmol, 1.5 equiv.) in THE (5 mL). The reaction mixture was stirred at 70° C. for 72 h and then diluted with EtOAc and washed with sodium bicarbonate. The organic layer was separated, dried and concentrated. The residue was chromatographed using ethyl acetate: hexanes mixture (10-100%) to give the title compound as a yellow solid in 4.4% yield. LC-MS: (ES, m/z): [M+H]⁺ 482. ¹H-NMR: (300 MHz, DMSO, ppm): δ1.23 (s, 3H), 1.26 (s, 3H), 2.33 (s, 3H), 3.36 (s, 3H), 5.15 (s, 1H), 7.04 (dd, 1H), 7.45 (d, 1H), 7.55 (m, 1H),

### Step 1: 2-(2-hydroxypropan-2-yl)isonicotinic acid

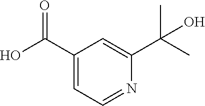

Into a round-bottom flask, was placed methyl 2-(2-hydroxypropan-2-yl)isonicotinate (0.435 g, 2.23 mmol, 1 equiv.) and KOH (0.14 g, 2.45 mmol, 1.1 equiv.) in methanol (5 mL) and the reaction mixture was stirred overnight at room temperature. After removal of the solvent under vacuo, the residue was diluted with water and extracted with ethyl ether. The aqueous phase was acidified to pH 1 with conc. HCl. The organics were extracted with ethyl acetate. The combined organic layer was washed with brine, dried over MgSO₄, filtered, and concentrated to give the title compound as a white solid in 89% yield.

### Example 214: N,1,4-trimethyl-N-(2-methyl-4-(6-(trifluoromethyl)quinazolin-2-yl)phenyl)-1H-pyrazole-3-carboxamide

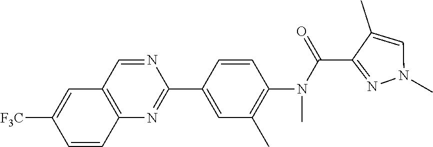 [SMILES: Cc1cc(-c2ncc3cc(C(F)(F)F)ccc3n2)ccc1N(C)C(=O)c1nn(C)cc1C]

Proceeding analogously as described in Example 165, Step 2 but substituting N-(4-bromo-2-fluorophenyl)-2-methylisonicotinamide with 1,4-dimethyl-N-(2-methyl-4-(6-(trifluoromethyl)quinazolin-2-yl)phenyl)-1H-pyrazole-3-carboxamide, the title compound was obtained as a white solid in 33% yield. LC-MS: (ES, m/z): [M+H]⁺ 440; ¹H-NMR: (300 MHz, CDCl3, ppm): δ 1.67 (s, 3H), 2.41 (s, 3H), 3.49 (s, 3H), 3.97 (s, 3H), 7.01 (s, 1H), 7.06 (d, 1H), 8.10 (d, 1H), 8.20 (d, 1H), 8.28 (s, 1H), 8.37 (d, 1H), 8.51 (s, 1H), 9.56 (s, 1H).

### Example 215: 1,4-dimethyl-N-(2-methyl-4-(6-(trifluoromethyl)quinazolin-2-yl)phenyl)-1H-pyrazole-5-carboxamide

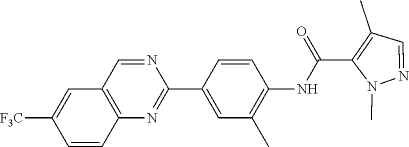 [SMILES: Cc1cc(-c2ncc3cc(C(F)(F)F)ccc3n2)ccc1NC(=O)c1c(C)cnn1C]

Proceeding as described in Example 162 but substituting 2-chloro-8-methyl-6-(trifluoromethyl)pyrido[3,2-d]pyrimidine with 2-chloro-6-(trifluoromethyl)quinazoline and N-(2-fluoro-4-(4,4,5,5-tetramethyl-1,3,2-dioxaborolan-2-yl)phenyl)-N,1-dimethyl-1H-pyrazole-5-carboxamide with 1,4-dimethyl-N-(2-methyl-4-(4,4,5,5-tetramethyl-1,3,2-dioxaborolan-2-yl)phenyl)-1H-pyrazole-5-carboxamide provided crude product. Purification by chromatography using 20-80% EtOAc-Hexanes as eluent gave the title compound as an off-white in 18% yield. LC-MS: (ES, m/z): [M+H]⁺ 426; ¹H-NMR: (300 MHz, DMSO, ppm): δ2.27 (s, 3H), 2.43 (s, 3H), 3.92 (s, 3H), 7.68 (s, 1H), 8.09 (d, 1H), 8.22-8.30 (m, 2H), 8.45 (dd, 1H), 8.50 (s, 1H), 8.70 (s, 1H), 9.39 (s, 1H), 9.87 (s, 1H).

### Example 216: 1,4-dimethyl-N-(2-methyl-4-(6-(trifluoromethyl)quinazolin-2-yl)phenyl)-1H-pyrazole-3-carboxamide

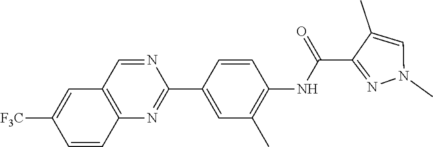 [SMILES: Cc1cc(-c2ncc3cc(C(F)(F)F)ccc3n2)ccc1NC(=O)c1nn(C)cc1C]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1,4-dimethyl-N-(2-methyl-4-(4,4,5,5-tetramethyl-1,3,2-dioxaborolan-2-yl)phenyl)-1H-pyrazole-3-carboxamide and 2-chloro-8-methyl-6-(trifluoromethyl)pyrido[3,2-d]pyrimidine with 2-chloro-6-(trifluoromethyl)quinazoline provided crude product. Purification by chromatography using 20-80% EtOAc-Hexanes as eluent gave the title compound as an off-white in 17% yield. LC-MS: (ES, m/z): [M+H]⁺ 426. ¹H-NMR: (300 MHz, DMSO, ppm): δ2.27 (s, 3H), 2.44 (s, 3H), 3.97 (s, 3H), 7.38 (s, 1H), 7.83 (d, 1H), 8.24-8.31 (m, 2H), 8.48 (dd, 1H), 8.53 (s, 1H), 8.72 (s, 1H), 9.84 (s, 1H), 9.89 (s, 1H).

### Step 1: 1,4-dimethyl-N-(2-methyl-4-(4,4,5,5-tetramethyl-1,3,2-dioxaborolan-2-yl)phenyl)-1H-pyrazole-3-carboxamide

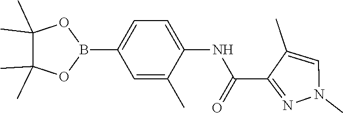

Proceeding analogously as described in Example 213, but substituting N,2-dimethyl-4-(6-(trifluoromethyl)quinazolin-2-yl)aniline with 2-methyl-4-(4,4,5,5-tetramethyl-1,3,2-dioxaboronlan-2-yl)aniline and 2-(2-hydroxypropan-2-yl)isonicotinic acid with 1,4-dimethyl-1H-pyrazole-3-carboxylic acid, the title compound was obtained as an off-white solid in 54% yield.

### Example 217: N,1,4-trimethyl-N-(2-methyl-4-(6-(trifluoromethyl)quinazolin-2-yl)phenyl)-1H-pyrazole-5-carboxamide

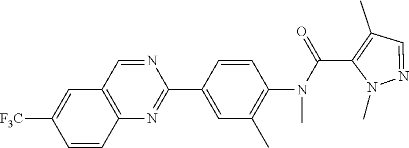 [SMILES: Cc1cc(-c2ncc3cc(C(F)(F)F)ccc3n2)ccc1N(C)C(=O)c1c(C)cnn1C]

Proceeding analogously as described in Example 165, Step 2 but substituting N-(4-bromo-2-fluorophenyl)-2-methylisonicotinamide with 1,4-dimethyl-N-[2-methyl-4-(6-(trifluoromethyl)-quinazolin-2-yl)phenyl)-1H-pyrazole-5-carboxamide provided the title compound as a white solid in 42% yield. LC-MS: (ES, m/z): [M+H]⁺ 440; ¹H-NMR: (300 MHz, CDCl3, ppm): δ 1.27 (s, 3H), 2.17 (s, 3H), 2.38 (s, 3H), 3.42 (s, 3H), 3.53 (s, 3H), 8.10 (dd, 1H), 8.24 (d, 1H), 8.28 (s, 1H), 8.40 (d, 1H), 8.46 (s, 1H).

### Example 218: N,1-dimethyl-N-(4-(6-(trifluoromethyl)quinazolin-2-yl)phenyl)-1H-pyrazole-5-carboxamide

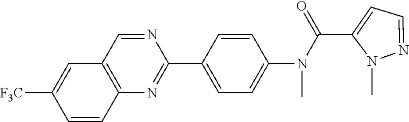 [SMILES: CN(C(=O)c1ccnn1C)c1ccc(-c2ncc3cc(C(F)(F)F)ccc3n2)cc1]

Proceeding analogously as described in Example 165, Step 2 but substituting N-(4-bromo-2-fluorophenyl)-2-methylisonicotinamide with 1-methyl-N-(4-(6-(trifluoromethyl)-quinazolin-2-yl)phenyl)-1H-pyrazole-5-carboxamide, the title compound was obtained as a white solid in 26% yield. LC-MS: (ES, m/z): [M+H]⁺ 412; ¹H-NMR: (300 MHz, CDCl3, ppm): δ 3.56 (s, 3H), 4.19 (s, 3H), 5.68 (d, 1H), 7.19 (s, 1H), 7.30 (d, 2H), 8.12 (dd, 1H), 8.25 (dd, 1H), 8.30 (s, 1H), 8.67 (d, 2H), 9.59 (s, 1H).

### Example 219: 1,3-dimethyl-N-(2-methyl-4-(6-(trifluoromethyl)quinazolin-2-yl)phenyl)-1H-pyrazole-4-carboxamide

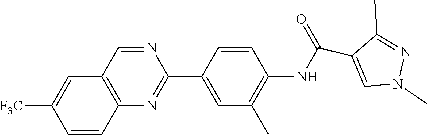 [SMILES: Cc1cc(-c2ncc3cc(C(F)(F)F)ccc3n2)ccc1NC(=O)c1cn(C)nc1C]

Proceeding analogously as described in Example 167 but substituting 2-chloro-8-methyl-6-(trifluoromethyl)pyrido[3,2-d]pyrimidine with 2-chloro-6-(trifluoromethyl)quinazoline and N2-methyl-N4-(2-methyl-4-(4,4,5,5-tetramethyl-1,3,2-dioxaborolan-2-yl)phenyl)pyridine-2,4-dicarboxamide with 1,3-dimethyl-N-(2-methyl-4-(4,4,5,5,-tetramethyl-1,3,2-dioxaborolan-2-yl)phenyl)-1H-pyrazole-4-carboxamide provided the title compound as an off-white solid 21% yield. LC-MS: (ES, m/z): [M+H]⁺ 426. ¹H-NMR: (300 MHz, DMSO, ppm): δ 2.38 (s, 3H), 2.40 (s, 3H), 3.84 (s, 3H), 7.73 (d, 1H), 8.22-8.30 (m, 2H), 8.33 (s, 1H), 8.43 (dd, 1H), 8.50 (s, 1H), 8.71 (s, 1H), 9.29 (s, 1H), 9.87 (s, 1H).

### Example 220: 1-methyl-N-(4-(6-(trifluoromethyl)quinazolin-2-yl)phenyl)-1H-pyrazole-5-carboxamide

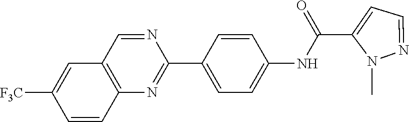 [SMILES: Cn1nccc1C(=O)Nc1ccc(-c2ncc3cc(C(F)(F)F)ccc3n2)cc1]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1,4-dimethyl-N-(4-(4,4,5,5-tetramethyl-1,3,2-dioxaborolan-2-yl)phenyl)-1H-pyrazole-5-carboxamide and 2-chloro-8-methyl-6-(trifluoromethyl)pyrido[3,2-d]pyrimidine with 2-chloro-6-(trifluoromethyl)quinazoline provided crude product. Purification by chromatography using 5-80% EtOAc-Hexanes as eluent gave the title compound as an off-white in 16% yield. LC-MS: (ES, m/z): [M+H]⁺ 398; ¹H-NMR: (300 MHz, DMSO, ppm): δ4.13 (s, 3H), 7.15 (d, 1H), 7.57 (d, 1H), 8.00 (d, 2H), 8.20-8.30 (m, 2H), 8.62 (d, 2H), 8.71 (s, 1H), 9.87 (s, 1H), 10.48 (s, 1H).

### Example 221: 1-methyl-5-(methyl-(2-methyl-4-(6-(trifluoromethyl)quinazolin-2-yl)phenyl)-carbamoyl)-1H-pyrazole-3-carboxylic acid

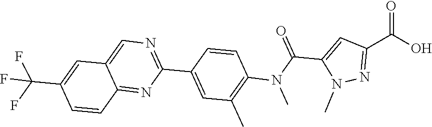 [SMILES: Cc1cc(-c2ncc3cc(C(F)(F)F)ccc3n2)ccc1N(C)C(=O)c1cc(C(=O)O)nn1C]

Proceeding analogously as described in Example 213, step 1 but substituting 2-(2-hydroxypropan-2-yl)isonicotinic acid with methyl 1-methyl-5-(methyl-(2-methyl-4-(6-(trifluoro-methyl)quinazolin-2-yl)phenyl)carbamoyl)-1H-pyrazole-3-carboxylate, the title compound was obtained as a yellow solid in 54% yield. LC-MS: (ES, m/z): [M+H]⁺ 470.

### Step 1: 3-(methoxycarbonyl)-1-methyl-1H-pyrazole-5-carboxylic acid

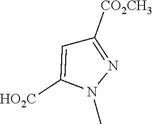

Proceeding analogously as described in Example 213, step 1 but substituting methyl 2-(2-hydroxypropan-2-yl)isonicotinate with dimethyl 1-methyl-1H-pyrazole-3,5-dicarboxylate, the title compound was obtained as a white solid in 31% yield.

### Step 2: methyl 1-methyl-5-(methyl-(2-methyl-4-(6-(trifluoromethyl)quinazolin-2-yl)phenyl)-carbamoyl)-1H-pyrazole-3-carboxylate

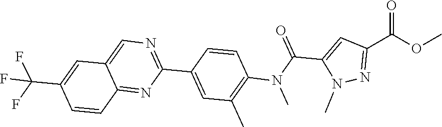 [SMILES: COC(=O)c1cc(C(=O)N(C)c2ccc(-c3ncc4cc(C(F)(F)F)ccc4n3)cc2C)n(C)n1]

Proceeding analogously as described in Example 213, but substituting 2-(2-hydroxy-propan-2-yl)isonicotinic acid with 3-(methoxycarbonyl)-1-methyl-1H-pyrazole-5-carboxylic acid, the title compound was obtained as a yellow solid in 54% yield.

### Example 222: N5,1-dimethyl-N5-(2-methyl-4-(6-(trifluoromethyl)quinazolin-2-yl)phenyl)-1H-pyrazole-3,5-dicarboxamide

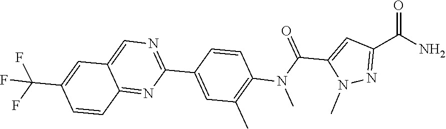 [SMILES: Cc1cc(-c2ncc3cc(C(F)(F)F)ccc3n2)ccc1N(C)C(=O)c1cc(C(N)=O)nn1C]

To a solution of 1-methyl-5-(methyl-(2-methyl-4-(6-(trifluoromethyl)quinazolin-2-yl)phenyl)-carbamoyl)-1H-pyrazole-3-carboxylic acid in DCM and 1 drop of DMF at 0° C., oxalyl chloride was added dropwise. The reaction mixture was brought to room temperature and stirred for 90 min. The reaction mixture was concentrated in vacuo, and the residue was dissolved in THF and the solution was cooled to 0° C. Conc. NH₄OH was added and the reaction mixture allowed to warm to room temperature. After 18 h, the reaction mixture was concentrated in vacuo and the residue was chromatographed on a silica gel column eluting with 20%-100% EtOAc-Hexanes to obtain crude product. The crude product was re-chromatography eluting with 1%-10% MeOH-DCM to obtain the title compound as an off-white solid which was triturated with diethyl ether, filtered, and then dried to give the pure product in 18% yield. LC-MS: (ES, m/z): [M+H]⁺ 469; ¹H-NMR: (300 MHz, DMSO, ppm): δ2.25 (s, 3H), 3.27 (s, 3H), 4.00 (s, 3H), 5.83 (s, 1H), 7.10 (s, 1H), 7.36 (s, 1H), 7.46 (d, 1H), 8.15-8.25 (m, 2H), 8.36 (dd, 1H), 8.44 (d, 1H), 8.66 (s, 1H), 9.82 (s, 1H).

### Example 223: 1-(2-hydroxyethyl)-N-methyl-N-[2-methyl-4-[8-methyl-6-(trifluoromethyl)-1,5-naphthyridin-2-yl]phenyl]-1H-pyrazole-5-carboxamide

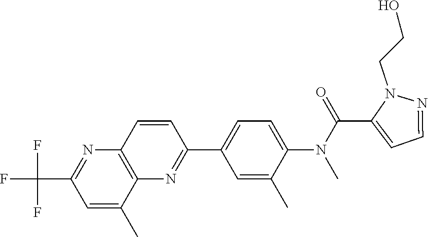 [SMILES: Cc1cc(-c2ccc3nc(C(F)(F)F)cc(C)c3n2)ccc1N(C)C(=O)c1ccnn1CCO]

Proceeding analogously as described in Example 171, Step 2 but substituting 1-(2-(benzyloxy)ethyl)-N-methyl-N-(2-methyl-4-(8-methyl-6-(trifluoromethyl)pyrido[3,2-d]pyrimidin-2-yl)phenyl)-1H-pyrazole-5-carboxamide with 1-[2-(benzyloxy)ethyl]-N-methyl-N-[2-methyl-4-[8-methyl-6-(trifluoromethyl)-1,5-naphthyridin-2-yl]phenyl]-1H-pyrazole-5-carboxamide provided crude product. The crude product was applied onto a prep-TLC and eluted with ethyl acetate/petroleum ether (1:2) to give the title compound in 57.83% yield. LC-MS: (ES, m/z): [M+H]⁺ 470; ¹H-NMR: (300 MHz, CDCl₃, ppm): δ 8.56-8.53 (d, 1H), 8.23-8.12 (m, 3H), 7.84-7.83 (d, 1H), 7.32-7.29 (d, 1H), 7.21-7.20 (d, 1H), 5.57-5.56 (d, 1H), 4.78-4.63 (m, 2H), 4.09-4.08 (t, 2H), 3.43 (s, 3H), 3.00 (s, 3H), 2.32 (s, 3H).

### Step 1: 1-[2-(benzyloxy)ethyl]-N-methyl-N-[2-methyl-4-[8-methyl-6-(trifluoromethyl)-1,5-naphthyridin-2-yl]phenyl]-1H-pyrazole-5-carboxamide

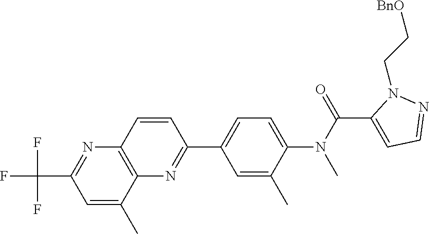 [SMILES: Cc1cc(-c2ccc3nc(C(F)(F)F)cc(C)c3n2)ccc1N(C)C(=O)c1ccnn1CCOCc1ccccc1]

Proceeding analogously as described in Example 201, Step 6 but substituting 8-bromo-2-chloro-6-(trifluoromethyl)quinoline with 6-chloro-4-methyl-2-(trifluoromethyl)-1,5-naphthyridine provided the title compound in 86.67% yield.

### Example 224: 1-(2-hydroxy-2-methylpropyl)-N-methyl-N-(2-methyl-4-(8-methyl-6-(trifluoromethyl)pyrido[3,2-d]pyrimidin-2-yl)phenyl)-1H-pyrazole-5-carboxamide

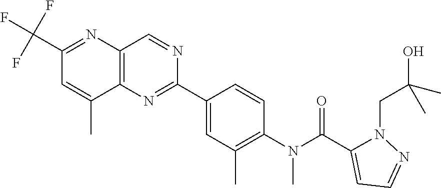 [SMILES: Cc1cc(-c2ncc3nc(C(F)(F)F)cc(C)c3n2)ccc1N(C)C(=O)c1ccnn1CC(C)(C)O]

Into an 8-mL sealed tube was placed 1-[2-(benzyloxy)-2-methylpropyl]-N-methyl-N-[2-methyl-4-[8-methyl-6-(trifluoromethyl)pyrido[3,2-d]pyrimidin-2-yl]phenyl]-1H-pyrazole-5-carboxamide (120 mg, 0.20 mmol, 1 equiv.), DCM (1.5 mL), and BCl₃ (26.0 mg, 0.22 mmol, 1.10 equiv.). The resulting solution was stirred for 1 min at 0° C. and then quenched with NaHCO₃. The resulting solution was extracted with ethyl acetate and after removal of the organics, the residue was applied onto Prep-TLC and eluted with ethyl acetate/petroleum ether (1:2) to give the title compound as a white solid in 59.14% yield. LC-MS: (ES, m/z): [M+H]⁺ 499; ¹H-NMR: (400 MHz, DMSO, ppm): δ 0.99 (s, 2H), 1.20 (s, 3H), 2.38 (m, 3H), 2.92-2.95 (d, 3H), 3.29-3.32 (m, 2H), 3.36 (s, 1H), 4.16-4.20 (d, 1H), 4.63-4.67 (d, 1H), 4.83 (s, 1H), 5.54 (s, 1H), 7.17 (s, 1H), 7.56-7.57 (d, 1H), 8.31-8.33 (d, 1H), 8.39 (s, 1H), 8.58 (s, 1H), 9.88 (s, 1H).

### Step 1: methyl 2-(benzyloxy)-2-methylpropanoate

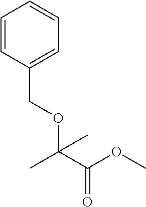

Into a 250-mL 3-necked round-bottom flask purged and maintained with an inert atmosphere of nitrogen, was placed methyl 2-hydroxy-2-methylpropanoate (10 g, 84.65 mmol, 1 equiv.), and DMF (100 mL), and NaH (2.0 g, 84.65 mmol, 1 equiv.) was added at 0° C. The resulting solution was stirred for 1 h at 0° C. and then benzyl bromide (15.9 g, 93.12 mmol, 1.1 equiv.) was added. The resulting solution was stirred for 12 h at 25° C. and then quenched with NH₄Cl. The resulting solution was extracted with ethyl acetate and the organic layer was concentrated. The residue was applied onto a silica gel column with ethyl acetate/petroleum ether (1:10) to give the title compound as a yellow liquid in 17.02% yield.

### Step 2: 2-(benzyloxy)-2-methylpropan-1-ol

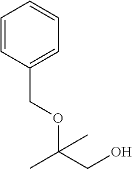

Into a 100-mL 3-necked round-bottom flask purged and maintained with an inert atmosphere of nitrogen, was placed methyl 2-(benzyloxy)-2-methylpropanoate (3 g, 14.41 mmol, 1 equiv.) and THE (30 mL) and LiAlH₄ (0.6 g, 15.81 mmol, 1.10 equiv.) was added at 0° C. The resulting solution was stirred for 1 h at 0° C. and then quenched with NaHCO₃. The resulting solution was extracted with ethyl acetate and the organic layer was concentrated. The residue was applied onto a silica gel column and eluted with ethyl acetate/petroleum ether (1:20) to give the title compound as a yellow oil in 38.51% yield.

### Step 3: methyl 1-[2-(benzyloxy)-2-methylpropyl]-1H-pyrazole-5-carboxylate

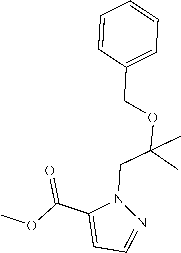 [SMILES: COC(=O)c1ccnn1CC(C)(C)OCc1ccccc1]

Into a 25-mL 3-necked round-bottom flask purged and maintained with an inert atmosphere of nitrogen, was placed 2-(benzyloxy)-2-methylpropan-1-ol (1 g, 5.55 mmol, 1 equiv.), methyl 1H-pyrazole-5-carboxylate (0.7 g, 5.55 mmol, 1 equiv.), and DIAD (2.2 g, 11.10 mmol, 2 equiv.) in THE (10 mL). PPh₃ (2.9 g, 11.10 mmol, 2 equiv.) was added at 0° C. and the resulting solution was stirred for 6 h at 25° C. The resulting mixture was concentrated and the residue was applied onto a silica gel column and eluted with ethyl acetate/petroleum ether (1:4) to give the title compound as a yellow solid in 21.88% yield.

### Step 4: 1-[2-(benzyloxy)-2-methylpropyl]-1H-pyrazole-5-carboxylic acid

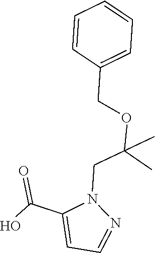 [SMILES: CC(C)(Cn1nccc1C(=O)O)OCc1ccccc1]

Into a 25-mL 3-necked round-bottom flask was placed methyl 1-[2-(benzyloxy)-2-methylpropyl]-1H-pyrazole-5-carboxylate (380 mg, 1.32 mmol, 1 equiv.), THE (8 mL), LiOH.H₂O (60.8 mg, 1.45 mmol, 1.1 equiv.), and H₂O (4 mL). The resulting solution was stirred for 3 h at 25° C. and then concentrated to give crude product.

### Step 5: 1-[2-(benzyloxy)-2-methylpropyl]-N-(4-bromo-2-methylphenyl)-1H-pyrazole-5-carboxamide

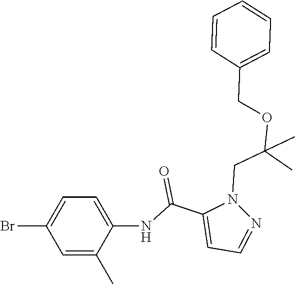 [SMILES: Cc1cc(Br)ccc1NC(=O)c1ccnn1CC(C)(C)OCc1ccccc1]

Proceeding analogously as described in Example 160, Step 8 but substituting 2-methylpyridine-4-carboxylic acid with 1-[2-(benzyloxy)-2-methylpropyl]-1H-pyrazole-5-carboxylic acid provided crude product. Purification by Prep-TLC with ethyl acetate/petroleum ether (1:3) gave the title compound as a yellow solid in 56.38% yield.

### Step 6: 1-[2-(benzyloxy)-2-methylpropyl]-N-(4-bromo-2-methylphenyl)-N-methyl-1H-pyrazole-5-carboxamide

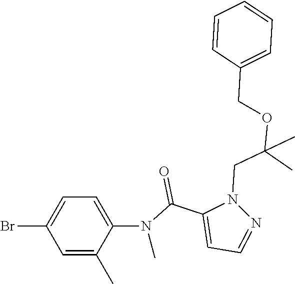 [SMILES: Cc1cc(Br)ccc1N(C)C(=O)c1ccnn1CC(C)(C)OCc1ccccc1]

Proceeding analogously as described in Example 165, Step 2 but substituting N-(4-bromo-2-fluorophenyl)-2-methylisonicotinamide with 1-[2-(benzyloxy)-2-methylpropyl]-N-(4-bromo-2-methylphenyl)-1H-pyrazole-5-carboxamide provided crude product. Purification by Prep-TLC with ethyl acetate/petroleum ether (1:3) as eluent provided the title compound as a yellow solid in 85.85% yield.

### Step 7: 1-[2-(benzyloxy)-2-methylpropyl]-N-methyl-N-[2-methyl-4-(4,4,5,5-tetramethyl-1,3,2-dioxaborolan-2-yl)phenyl]-1H-pyrazole-5-carboxamide

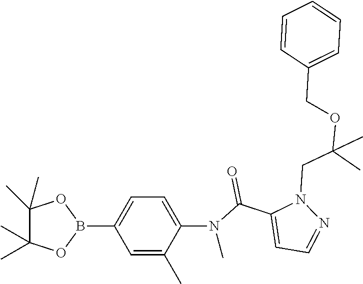 [SMILES: Cc1cc(B2OC(C)(C)C(C)(C)O2)ccc1N(C)C(=O)c1ccnn1CC(C)(C)OCc1ccccc1]

Proceeding as described in Example 161, Step 2 but substituting N-(4-bromo-2-methylphenyl)-N,2-dimethylpyridine-4-carboxamide with 1-[2-(benzyloxy)-2-methylpropyl]-N-(4-bromo-2-methylphenyl)-N-methyl-1H-pyrazole-5-carboxamide provided crude product. Purification by column chromatography with ethyl acetate/petroleum ether (1:10) provided the title compound as a yellow solid in 93.67% yield.

### Step 8: 1-[2-(benzyloxy)-2-methylpropyl]-N-methyl-N-[2-methyl-4-[8-methyl-6-(trifluoromethyl)pyrido[3,2-d]pyrimidin-2-yl]phenyl]-1H-pyrazole-5-carboxamide

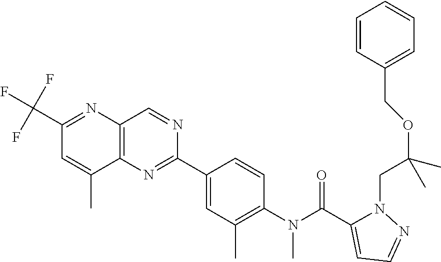 [SMILES: Cc1cc(-c2ncc3nc(C(F)(F)F)cc(C)c3n2)ccc1N(C)C(=O)c1ccnn1CC(C)(C)OCc1ccccc1]

Proceeding as described in Example 162 but substituting N-(2-fluoro-4-(4,4,5,5-tetramethyl-1,3,2-dioxaborolan-2-yl)phenyl)-N,1-dimethyl-1H-pyrazole-5-carboxamide with 1-[2-(benzyloxy)-2-methylpropyl]-N-methyl-N-[2-methyl-4-(4,4,5,5-tetramethyl-1,3,2-dioxaborolan-2-yl)phenyl]-1H-pyrazole-5-carboxamide provided crude product. Purification by Prep-TLC with dichloromethane/methanol (30:1) as eluent provided the title compound as a yellow solid in 42.76% yield.

### Example 225: 2-(5-(methyl(2-methyl-4-(6-(trifluoromethyl)quinazolin-2-yl)phenyl)-carbamoyl)-1H-pyrazol-1-yl)acetic acid

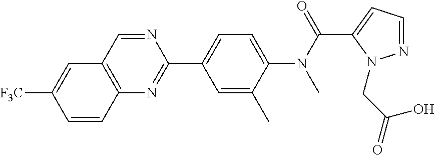 [SMILES: Cc1cc(-c2ncc3cc(C(F)(F)F)ccc3n2)ccc1N(C)C(=O)c1ccnn1CC(=O)O]

Into an 8-mL vial, was placed 1-(cyanomethyl)-N-methyl-N-(2-methyl-4-(6-(trifluoromethyl)-quinazolin-2-yl)phenyl)-1H-pyrazole-5-carboxamide (100 mg, 0.22 mmol, 1 equiv.), HCl (2 mL, 35%) and the resulting solution was stirred for 2 h at 100° C. The solids were collected by filtration and dried in an oven under reduced pressure to give the title compound as a white sold in 73.40% yield. LC-MS: (ES, m/z): [M+H]⁺ 470; ¹H-NMR: (300 MHz, DMSO, ppm): δ2.34-2.44 (m, 3H), 3.25 (s, 3H), 4.63-4.76 (t, 1H), 4.95-5.00 (d, 1H), 5.38 (s, 1H), 7.04 (s, 1H), 7.40-7.78 (m, 1H), 8.23-8.36 (m, 3H), 8.47-8.57 (m, 1H), 8.71 (s, 1H), 9.70 (s, 1H).

### BIOLOGICAL EXAMPLE

HEPG2 and HEPA1C1C7 cells were maintained in MEM and αMEM without nucleosides supplemented with 10% heat inactivated FBS respectively. Stably integrated DRE-luciferase cell lines were generated by transducing the both cell lines with Cignal XRE luciferase reporter (Qiagen) lentiviral particles according to the manufacturer protocol. For both cell lines stably integrated reporter cell lines were selected for the presence of 2 pg/mL puromycin. Following selection of stably integrated cell line pools, clonal cell lines were isolated by limiting dilution in 96-well plates. Transcriptional assays were performed by seeding 100 μL of cells at a density of 250,000 or 100,000 cells/mL, for HEPG2 and HEPA1C1C7 DRE-Luc cells respectively, into 96-well cell culture plates in OptiMEM supplemented with 0.5% heat inactivated FBS and allowed to attach overnight. For modulator assays, the compounds were added in a semi-log dose response using a D300e Digital Dispenser (Tecan) followed normalization with vehicle (DMSO). Immediately following compound addition 10 μL of 2,3,7,8-Tetrachlorodibenzo-p-dioxin (TCDD) was added to the cells to a final concentration of 3 nM or 0.3 nM for the HEPG2 and HEPA1C1C7 DRE-Luc cells respectively. Following 24 hour incubation the medium was removed and the cells were lysed in 25 μL of Reporter Lysis Buffer (Promega). Firefly luciferase activity was measured immediately following the addition of 50 μL Luciferase Assay Reagent (Promega). The percent maximal activity for each point was determined using the following equasion: (RLUₛₐₘₚₗₑ-RLU_{vehicle-TCDD})/(RLU_{vehicle+TCDD} RLU_{vehicle-TCDD})*100. The relative IC₅₀, defined as the compound concentration required to reduce the TCDD induced response between the top and bottom plateau of each individual dose response curve by half, for each compound was determined using Prism 7 (GraphPad Software).

| | IC₅₀ hAhR |
|---|---|
| Example | (antagonist mode) |
| 001 | +++ |
| 002 | ++++ |
| 003 | ++ |
| 004 | ++++ |
| 005 | + |
| 006 | + |
| 007 | ++++ |
| 008 | ++++ |
| 009 | ++ |
| 010 | +++ |
| 011 | +++ |
| 012 | ++++ |
| 013 | + |
| 014 | +++ |
| 015 | + |
| 016 | ++ |
| 017 | + |
| 018 | ++++ |
| 019 | +++ |
| 020 | ++++ |
| 021 | ++ |
| 022 | + |
| 023 | ++ |
| 024 | +++ |
| 025 | + |
| 026 | ++ |
| 027 | ++ |
| 028 | ++ |
| 029 | + |
| 030 | + |
| 031 | ++ |
| 032 | ++ |
| 033 | +++ |
| 034 | ++ |
| 035 | + |
| 036 | ++ |
| 037 | ++ |
| 038 | ++ |
| 039 | ++ |
| 040 | +++ |
| 041 | + |
| 042 | ++ |
| 043 | + |
| 044 | + |
| 045 | + |
| 046 | ++ |
| 047 | + |
| 048 | + |
| 049 | +++ |
| 050 | ++ |
| 051 | ++++ |
| 052 | ++++ |
| 053 | ++ |
| 054 | +++ |
| 055 | + |
| 056 | ++ |
| 057 | +++ |
| 058 | +++ |
| 059 | +++ |
| 060 | +++ |
| 061 | ++ |
| 062 | + |
| 063 | + |
| 064 | +++ |
| 065 | +++ |
| 066 | ++ |
| 067 | ++ |
| 068 | + |
| 069 | + |
| 070 | ++ |
| 071 | +++ |
| 072 | +++ |
| 073 | ++++ |
| 074 | + |
| 075 | + |
| 076 | ++++ |
| 077 | +++ |
| 078 | +++ |
| 079 | ++ |
| 080 | + |
| 081 | + |
| 082 | ++ |
| 083 | ++ |
| 084 | + |
| 085 | + |
| 086 | + |
| 087 | + |
| 088 | + |
| 089 | + |
| 090 | + |
| 091 | +++ |
| 098 | + |
| 099 | ++++ |
| 100 | +++ |
| 101 | +++ |
| 102 | ++++ |
| 103 | ++++ |
| 104 | ++++ |
| 105 | ++++ |
| 106 | ++++ |
| 107 | ++++ |
| 108 | ++++ |
| 109 | ++++ |
| 110 | ++++ |
| 111 | ++++ |
| 112 | ++++ |
| 113 | +++ |
| 114 | ++++ |
| 115 | ++++ |
| 116 | ++++ |
| 117 | ++ |
| 118 | +++ |
| 119 | ++++ |
| 120 | ++++ |
| 121 | ++++ |
| 122 | ++++ |
| 123 | ++ |
| 124 | ++++ |
| 125 | ++++ |
| 126 | ++++ |
| 127 | ++++ |
| 128 | ++++ |
| 129 | +++ |
| 130 | ++++ |
| 131 | +++ |
| 132 | ++++ |
| 133 | + |
| 134 | +++ |
| 135 | ++++ |
| 136 | ++++ |
| 137 | ++++ |
| 138 | ++++ |
| 139 | ++ |
| 140 | ++++ |
| 141 | ++++ |
| 142 | ++++ |
| 143 | ++++ |
| 144 | ++++ |
| 145 | ++++ |
| 146 | + |
| 147 | ++++ |
| 148 | ++++ |
| 149 | ++++ |
| 150 | ++++ |
| 151 | ++++ |
| 152 | ++++ |
| 153 | ++++ |
| 154 | ++++ |
| 155 | +++ |
| 155 | ++++ |
| 156 | ++++ |
| 157 | ++++ |
| 158 | + |
| 159 | na |
| 160 | ++++ |
| 161 | +++ |
| 162 | ++++ |
| 163 | ++++ |
| 164 | ++++ |
| 165 | − |
| 166 | ++++ |
| 167 | ++++ |
| 168 | ++++ |
| 169 | ++++ |
| 170 | ++++ |
| 171 | +++ |
| 172 | ++++ |
| 173 | ++++ |
| 174 | ++++ |
| 175 | ++++ |
| 176 | Na |
| 177 | ++++ |
| 178 | Na |
| 179 | +++ |
| 180 | Na |
| 181 | ++++ |
| 182 | ++++ |
| 183 | ++++ |
| 184 | ++++ |
| 185 | ++++ |
| 186 | + |
| 187 | ++++ |
| 188 | ++++ |
| 189 | + |
| 190 | + |
| 191 | + |
| 192 | ++++ |
| 193 | ++ |
| 194 | + |
| 195 | + |
| 196 | + |
| 197 | + |
| 198 | ++++ |
| 199 | na |
| 200 | ++++ |
| 201 | ++++ |
| 202 | ++++ |
| 203 | ++++ |
| 204 | + |
| 205 | ++ |
| 206 | Na |
| 207 | +++ |
| 208 | + |
| 209 | ++++ |
| 210 | ++++ |
| 211 | ++++ |
| 212 | ++++ |
| 213 | na |
| 214 | +++ |
| 215 | ++++ |
| 216 | ++++ |
| 217 | ++++ |
| 218 | ++++ |
| 219 | ++++ |
| 220 | + |
| 221 | na |
| 222 | ++++ |
| 223 | ++++ |
| 224 | +++ |
| 225 | na |

(+) IC₅₀ = 10 uM-1 uM
(++) IC₅₀ = 1 uM-500 nM
(+++) IC₅₀ = 500 nM-200 nM
(++++) IC₅₀ < 200 nM

### Example 2

The ability of compounds of formula I to facilitate hematopoietic stem cell expansion was determined by testing two representative compounds of formula I (compounds in Table 1 having hAhR IC₅₀++++) as follows.

Isolated human CD34⁺ hematopoietic stem cells (HSC) were obtained from AllCells (Alameda, Calif., USA). 5,000 HSC per well were cultivated in 100 uL HSC expansion media StemSpan SFEM (StemCell Technologies; Vancouver, BC, Canada) containing 1× antibiotics (Life Technologies, Carlsbad, Calif., USA), 100 ng/mL thrombopoietin, 100 ng/mL IL-6, 100 ng/mL Flt3 ligand and 100 ng/mL stem cell factor (all R&D Systems, Minneapolis, Minn., USA) in a flat bottom 96 well tissue culture plate. 100 uL of 3-fold serial diluted compounds (final concentration range 0.169-10,000 nM) were added at 2× of the final concentration to HSC culture and HSC culture plates were incubated in a humidity-controlled incubator at 37° C., 5% CO₂. After 12 days, the absolute viable cell counts from each well were determined. HSC were then stained in staining buffer composed of Hank's balanced salt solution with 2% fetal bovine serum and 2 mM EDTA (all Life Technologies) at 4° C. for 1 hour with anti-CD90 APC, anti-CD34 PerCP, anti-CD38 FITC, CD45RA PE-Cy7 and anti-CD133 PE antibodies (all BD Biosciences, San Jose, Calif., USA). HSC were washed with staining buffer and HSC were analyzed by multicolor phenotype flow cytometry. The percentages of CD34⁺ cells were determined as percentage of total population. Absolute counts of each population were calculated from the total number of viable cells multiplied by the percentage of each population. The EC₅₀ value for the frequency of CD34+ cells, defined as the compound concentration with half maximal response on the dose response curve, was calculated using Prism 7 (GraphPad Software, La Jolla, Calif., USA).

### Results:

Human CD34⁺ hematopoietic stem cell cultures were incubated in HSC expansion media containing cytokines and AhR inhibitors. After 12-day culture, AhR inhibitors increased the CD34⁺ cell counts in a concentration-dependent manner, i.e. 5.3-fold to 5.9-fold expansion at compound concentrations of 3,333 nM or 10,000 nM. In addition, the frequency of CD34⁺ HSC increased in a concentration-dependent manner. i.e. 3.9-fold to 5.0-fold at compound concentrations of 1,111 nM and 3,333 nM, respectively.

What is claimed is:

1. A method for producing an expanded population of stem cells and/or lineage committed progenitor cells in vitro or ex vivo comprising culturing a population of the stem cells and/or lineage committed progenitor cells in a medium comprising a compound of formula I, II, III or IV: 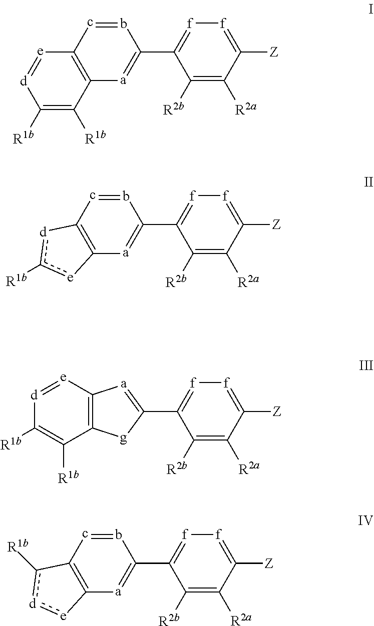
or a pharmaceutically acceptable salt, solvate or hydrate thereof, wherein
one of the dashed bonds is a single bond and the other of the dashed bonds is a double bond;
each of ring vertices a, b and c is independently selected from the group consisting of C(R^{1a}) and N;
each of ring vertices d and e is independently selected from the group consisting of C(R^{1b}), N, NH, N(C₁₋₄ alkyl) and N(C₁₋₄ haloalkyl) provided at least of d and e is other than C(R^{1b});
each ring vertex f is selected from the group consisting of C(R^{2c}), C(R^{2d}) and N;
ring vertex g is selected from the group consisting of O, S and N(R^{1a});
Z is selected from the group consisting of: 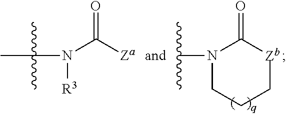
Z^{a} is selected from the group consisting of:
(i) a 5- or 6-membered heteroaryl group having at least one nitrogen atom as a ring member, which is substituted with from 1 to 4 R⁴;
(ii) a 5-, 6- or 7-membered heterocycloalkyl group, which is optionally substituted with hydroxyl, deuterium, C₁₋₄ alkyl, C₁₋₄ haloalkyl, C₁₋₄ deuteroalkyl, and C₁₋₄ alkoxy; and
(iii) a C₁₋₈ alkyl group, C₁₋₈ haloalkyl group, or a C₁₋₈ alkoxy group;
Z^{b} is selected from the group consisting of O, NR^{z} and C(R^{z})₂, wherein each R^{z} is independently selected from the group consisting of H, C₁₋₄ alkyl, C₁₋₄ haloalkyl and C₁₋₄ alkoxy;
the subscript q is 0, 1 or 2;
each R^{1a} and R^{1b} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, C₃₋₆ cycloalkyl, 5- or 6-membered heterocycloalkyl, phenyl and 5- or 6-membered heteroaryl, and wherein the aliphatic and cyclic portions of R^{a}, R^{b} and R^{c} are optionally further substituted with from one to three halogen, hydroxy, methyl, amino, methylamino, dimethylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; and
each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X¹—CN, —X¹—CO₂R^{d}, —X¹—CONR^{d}R^{e}, —X¹—C(O)R^{d}, —X¹—OC(O)NR^{d}R^{e}, —X¹—NR^{e}C(O)R^{d}, —X¹—NR^{b}C(O)₂R, —X¹—NR^{d}C(O)NR^{d}R^{e}, —X¹—NR^{d}R^{e}, —X¹—OR^{d}, —X¹—Y and —X¹—S(O)₂NR^{d}R^{e}; wherein each X¹ is independently C₁₋₆alkylene and Y is selected from the group consisting of pyrazole, imidazole, 1,2,3-triazole, 1,2,4-triazole, tetrazole, piperidine, pyrrolidine, tetrahydrofuran, tetrahydropyran and morpholine; and
each R^{d} and R^{e} is independently selected from hydrogen, C₁₋₈ alkyl, and C₁₋₈ haloalkyl, or when attached to the same nitrogen atom are optionally combined with the nitrogen atom to form a five or six-membered ring having from 0 to 2 additional heteroatoms as ring members selected from N, O or S;
each R^{f} is independently selected from the group consisting of C₁₋₈ alkyl, C₁₋₈ haloalkyl, C₁₋₈ deuteroalkyl, C₃₋₆ cycloalkyl, heterocycloalkyl, phenyl and 5- or 6-membered heteroaryl;
and wherein the aliphatic and cyclic portions of R^{d}, R^{e} and R^{f} are optionally further substituted with from one to three halogen, hydroxy, methyl, amino, methylamino, dimethylamino and carboxylic acid groups; and
wherein the compound of formula I, II, III, or IV antagonizes the activity of aryl hydrocarbon receptor;
and
the stem cells and/or lineage committed progenitor cells are cultured under conditions allowing expansion of the stem cells and/or progenitor cells.

2. The method of claim 1, further comprising differentiating the expanded stem cells to lineage committed progenitor cells thereof under conditions that cause differentiation of the expanded stem cells to lineage committed progenitor cells thereof.

3. The method of claims 1 or 2, wherein the method is carried out ex 80 vivo.

4. The method of any one of claims 1 to 3, wherein the stem cells and lineage committed progenitor cells are human cells.

5. The method of any one of claims 1 to 4, wherein the stem cells and/or lineage committed progenitor cells are hematopoietic stem cells and/or lineage 85 committed hematopoietic progenitor cells.

6. The method of any one of claims 1 to 4, wherein the stem cells and/or lineage committed progenitor cells are genetically modified hematopoietic stem cells and/or lineage committed genetically modified hematopoietic progenitor cells thereof.

7. The method of claim 6, wherein the genetically modified hematopoietic stem cells and/or lineage committed genetically modified hematopoietic progenitor cells thereof comprise an exogenous nucleic acid.

8. The method of any one of claims 5 to 7, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits TGFβ signaling.

9. The method of any one of claims 5 to 8, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone demethylase inhibitor.

10. The method of any one of claims 5 to 9, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone deacetylase inhibitor.

11. The method of any one of claims 5 to 10, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits p38 signaling.

12. The method of any one of claims 5 to 11, further comprising culturing the cells in the presence of a Notch agonist.

13. The method of claim 12, wherein the Notch Agonist is Delta-^{ext-IgG}.

14. The method of any one of claims 5 to 13, wherein the hematopoietic stem cells and genetically modified hematopoietic stem cells are enriched in Endothelial Protein C Receptor (EPCR+) and/or CD34+, CD38+, CD90+, CD45RA+, CD133 and/or CD49f+.

15. The method of claim 14, wherein the hematopoietic stem cells and genetically modified hematopoietic stem cells consist essentially of CD34+ cells.

16. The method of any one of claims 5 to 15, further comprising culturing the population of:
(i) hematopoietic and/or lineage committed hematopoietic progenitor cells; or
(ii) genetically modified hematopoietic stem cells and/or lineage committed genetically modified hematopoietic progenitor cells;
in the presence of a sufficient amount of one or more of IL6, Flt-3-L, TPO, and SCF.

17. The method of any one of claims 1 to 16, wherein the amount of compound of formula I, II, III, or IV in the cell culture is from about 100 pm to about 10 μm.

18. The method of any one of claims 1 to 17, wherein the stem cells and/or lineage committed progenitor cells are cultured in the presence of a compound of formula I, II, III, or IV from about 2 to about 35 days.

19. The method of any one of claims 1 to 17, wherein the starting cell population is cultured in the presence of a compound of formula I, II, III, or IV during a time sufficient for about 2- to 50,000-fold expansion of hematopoietic cells and/or lineage committed hematopoietic progenitor cells or genetically modified hematopoietic stem cells and/or lineage committed genetically modified hematopoietic progenitor cells thereof, preferably the hematopoietic cells are CD34+ cells, as compared to a population of hematopoietic stem cells and/or lineage committed hematopoietic progenitor cells or genetically modified hematopoietic stem cells and/or lineage committed genetically modified hematopoietic progenitor cells thereof cultured under the same conditions in the absence of a compound of formula I, II, III, or IV.

20. The method of any one of claims 1 to 19, wherein the hematopoietic stem cells and/or lineage committed hematopoietic progenitor cells or genetically modified hematopoietic stem cells and/or lineage committed genetically modified hematopoietic progenitor cells are contacted with said one or more agents simultaneously.

21. The method of any one of claims 1 to 19, wherein the hematopoietic stem cells and/or lineage committed hematopoietic progenitor cells or genetically modified hematopoietic stem cells and/or lineage committed genetically modified hematopoietic progenitor cells are contacted with said one or more agents at different times.

22. The method of any one of claims 1 to 21, wherein the compound is a compound of formula I having a structure of formula Ia, Ib, Ic, Id, Ie, If, or Ig:

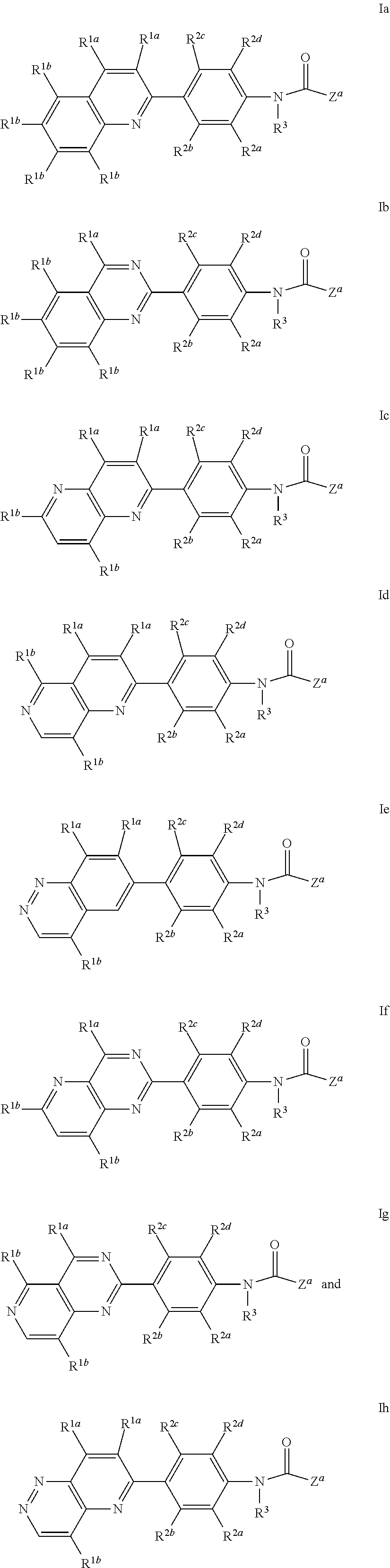

23. The method of claim 22, wherein Z^{a} is pyrazole or pyridine, each of which is optionally substituted with from 1 to 3 R⁴.

24. The method of claim 23, wherein the compound is a compound of formula Ia, Ib, If or Ig having a structure of formula Ia1, Ib1, If1, or Ig1: 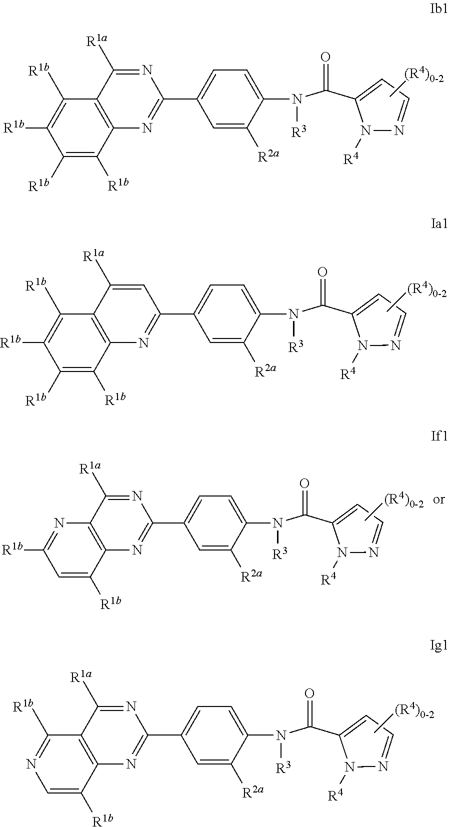
wherein:
each R^{1a} and R^{1b} is independently selected from the group consisting of H, halogen, —R^{c}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b} and —OR^{a};
R^{2a} is selected from the group consisting of H, F and CH₃;
each R⁴ is independently selected from the group consisting of hydrogen, halogen, —CN, —R^{f}, —CO₂R^{d}, —CONR^{d}R^{e}, —NR^{e}C(O)₂R^{f}, —NR^{d}C(O)NR^{d}R^{e}, —NR^{d}R^{e}, —OR^{d}, —X¹—CN, —X¹—CO₂R^{d}, —X¹—CONR^{d}R^{e}, —X¹—NR^{d}R^{e}, —X¹—OR^{d} and —X¹—Y.

25. The method of any one of claims 1 to 21, wherein the compound is selected from Table 1.

26. The method of any one of claims 1 to 21, wherein the compound is selected from Table 1 and has +++ or ++++ activity.

27. An ex vivo or in vitro composition comprising a cell population of expanded hematopoietic stem cells and/or lineage committed hematopoietic progenitor cells and a compound of formula I, II, III, IV, Ia, Ib, Ic, Id, Ie, If Ig, Ih, Ia1, Ib1, If1, Ig1 or a compound disclosed in Table 1.

28. A composition comprising a cell population of expanded hematopoietic stem cells and/or lineage committed hematopoietic progenitor cells thereof obtained or obtainable by culturing ex vivo a starting population of cells comprising hematopoietic stem cells and/or lineage committed hematopoietic progenitor cells according to the method of any one of claims 5 and 8 to 26.

29. The composition of claim 27 or 28 substantially free of a compound of formula I, II, III, IV, Ia, Ib, Ic, Id, Ie, If Ig, Ih, Ia1, Ib1, If1, Ig1 or a compound disclosed in Table 1 and/or any other component of the cell culture medium.

30. The composition any one of claims 27 to 29, further comprising a pharmaceutically acceptable medium.

31. The composition of any one of claims 27 or 29, suspended in a pharmaceutically acceptable medium suitable for transplantation into a patient in need thereof.

32. A method of treating a disease treatable by hematopoietic stem cell and/or lineage committed hematopoietic progenitor cell therapy comprising administering to a patient in need thereof a composition of any one of claims 27 to 31.

33. The method of claim 32, wherein the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

34. The method of claim 32, wherein the disease is selected from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease AR I NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E).
